(19) United States
(12) Patent Application Publication
(10) Pub. No.: US 2024/0277823 A1
(43) Pub. Date: Aug. 22, 2024

(54) LEPTOSPIRAL VIRULENCE MODULATING PROTEINS AND USES THEREOF

(71) Applicant: Yale University, New Haven, CT (US)

(72) Inventor: Joseph Vinetz, New Haven, CT (US)

(21) Appl. No.: 18/681,192

(22) PCT Filed: Aug. 5, 2022
(86) PCT No.: PCT/US22/74601

§ 371 Date: Feb. 5, 2024

Related U.S. Application Data
(60) Provisional application No. 63/230,244, filed on Aug. 6, 2021.

(51) Int. Cl. A61K 39/02; A61K 39/00; C07K 14/195; C07K 16/12

(52) U.S. Cl. CPC A61K 39/0225; C07K 14/195; C07K 16/1207; A61K 2039/55555; A61K 2039/55572; A61K 2039/575; C07K 2317/565; C07K 2319/01

(57) ABSTRACT

Provided are Leptospiral virulence modifying (VM) proteins, or fragments comprising the DNase domain thereof, and their use as vaccines and therapeutic agents. Also provided are pan-vaccines comprising multiple VM proteins.

### CROSS REFERENCE TO RELATED APPLICATIONS

This application claims priority to U.S. Provisional Application No. 63/230,244, filed Aug. 6, 2021 which is hereby incorporated by reference herein in its entirety.

### STATEMENT REGARDING FEDERALLY SPONSORED RESEARCH OR DEVELOPMENT

This invention was made with government support under AI115658, AI108276 and AI064466 awarded by National Institutes of Health. The government has certain rights in the invention.

### BACKGROUND OF THE INVENTION

The PF07598 gene family was identified as belonging solely to pathogenic Leptospira (Fouts et al, 2016, PLOS Negl Trop Dis. 10(2): e0004403; Lehmann et al, 2013, PLOS Negl Trop Dis, 7(10): e2468.) Members of this gene family previously known to be upregulated by osmolarity but the gene function is currently not known (Matsunaga et al, 2007; Infect Immun, 75(6): 2864-2874). In vivo upregulation of PF07598 gene family members has been reported in a hamster model (Lehmann et al, 2013; PLOS Negl Trop Dis, 7(10): e2468), and a human antibody response to one member of this gene family has been reported in vivo (Lessa-Aquino, 2017, PLOS Negl Trop Dis, 11(1):e0005349). Further, random transposon mutagenesis of Leptospira interrogans serovar Manilae has been reported (Marcsisin et al, 2013, J Med Microbiol, 62(Pt 10): 1601-1608), yet the function remains unknown.

Human leptospirosis is common in developing countries, and there is an increased incidence in industrialized countries. Only limited progress has been made towards implementing effective public health responses, and no vaccine is registered for humans.

There remains a need in the art for novel compositions that have vaccine potential against pathogenic Leptospira. The current invention satisfies this unmet need.

### SUMMARY OF THE INVENTION

In one embodiment, the invention relates to a composition comprising at least one Leptospiral virulence modifying (VM) protein or a fragment thereof comprising a DNase domain. In one embodiment, the at least one VM protein is LA_3388, LA_0835, LA_0591, LA_0589 (v), LA_1402, LA_1400, LA_3271, LA_0934, LA_0769, LA_2628, LA_0620, LA_3490, LIC_10778, LIC_12791, LIC_12985, LIC_12986, LIC_12339, LIC_12340, LIC_10870, LIC_12715, LIC_12844, LIC_11358, LIC_10639, LIC_12963, LIC_10695, LMANV2_260038, LMANV2_240079, LMANV2_70075, LMANV2_70078, LMANV2_210058, LMANV2_210056, LMANV2_80114, LMANV2_320010, LMANV2_240142, LMANV2_150103, LMANV2_170032, LMANV2_70050, or LMANV2_170091.

In one embodiment, the invention relates to a fusion protein comprising a Leptospiral VM protein or Leptospiral VM protein DNase domain and a targeting domain specific for binding to a target molecule. In one embodiment, the at least one VM protein is LA_3388, LA_0835, LA_0591, LA_0589 (v), LA_1402, LA_1400, LA_3271, LA_0934, LA_0769, LA_2628, LA_0620, LA_3490, LIC_10778, LIC_12791, LIC_12985, LIC_12986, LIC_12339, LIC_12340, LIC_10870, LIC_12715, LIC_12844, LIC_11358, LIC_10639, LIC_12963, LIC_10695, LMANV2_260038, LMANV2_240079, LMANV2_70075, LMANV2_70078, LMANV2_210058, LMANV2_210056, LMANV2_80114, LMANV2_320010, LMANV2_240142, LMANV2_150103, LMANV2_170032, LMANV2_70050, or LMANV2_170091. In one embodiment, the target molecule is a bacterial antigen, viral antigen, parasitic antigen, cancer antigen, tumor-associated antigen, are a tumor-specific antigen.

In one embodiment, the composition comprises a combination of two or more Leptospiral VM proteins. In one embodiment, the composition comprises a combination of two or more of LA_3388, LA_0835, LA_0591, LA_0589 (v), LA_1402, LA_1400, LA_3271, LA_0934, LA_0769, LA_2628, LA_0620, LA_3490, LIC_10778, LIC_12791, LIC_12985, LIC_12986, LIC_12339, LIC_12340, LIC_10870, LIC_12715, LIC_12844, LIC_11358, LIC_10639, LIC_12963, LIC_10695, LMANV2_260038, LMANV2_240079, LMANV2_70075, LMANV2_70078, LMANV2_210058, LMANV2_210056, LMANV2_80114, LMANV2_320010, LMANV2_240142, LMANV2_150103, LMANV2_170032, LMANV2_70050, and LMANV2_170091. In one embodiment, the composition comprises a combination of LIC_12340 and LIC_12985. In one embodiment, the composition comprises a combination of LIC_12340, LIC_12985, LA_3490, LA_0620, and LA_1402.

In one embodiment, the composition comprises at least one Leptospiral VM protein comprising an amino acid sequence of SEQ ID NO:2, SEQ ID NO:4, SEQ ID NO:6, SEQ ID NO:8, SEQ ID NO: 10 or SEQ ID NO: 12. In one embodiment, the composition comprises a combination of VM proteins comprising sequences as set forth in SEQ ID NO:10 and SEQ ID NO:12. In one embodiment, the composition comprises a combination of VM proteins comprising sequences as set forth in SEQ ID NO:2, SEQ ID NO:4, SEQ ID NO:8, SEQ ID NO: 10 and SEQ ID NO: 12.

In one embodiment, the composition comprises at least one lipid nanoparticle (LNP) comprising at least one VM protein or fragment of a VM protein comprising the DNase domain. In one embodiment, the composition comprises a combination of at least two LNP comprising at least two VM proteins or fragments of a VM protein comprising the DNase domain.

In one embodiment, the invention relates to a composition comprising at least one nucleic acid molecule encoding at least one Leptospiral virulence modifying (VM) protein or fragment thereof comprising a DNase domain of a VM protein. In one embodiment, the VM protein is LA_3388, LA_0835, LA_0591, LA_0589 (v), LA_1402, LA_1400, LA_3271, LA_0934, LA_0769, LA_2628, LA_0620, LA_3490, LIC_10778, LIC_12791, LIC_12985, LIC_12986, LIC_12339, LIC_12340, LIC_10870, LIC_12715, LIC_12844, LIC_11358, LIC_10639, LIC_12963, LIC_10695, LMANV2_260038, LMANV2_240079, LMANV2_70075, LMANV2_70078, LMANV2_210058, LMANV2_210056, LMANV2_80114, LMANV2_320010, LMANV2_240142, LMANV2_150103, LMANV2_170032, LMANV2_70050, or LMANV2_170091.

In one embodiment, the nucleic acid molecule encodes a fusion protein comprising a Leptospiral VM protein or a fragment comprising a DNase domain of a VM protein fused to a targeting domain specific for binding to a target molecule. In one embodiment, the target molecule is a bacterial antigen, viral antigen, parasitic antigen, cancer antigen, tumor-associated antigen, or a tumor-specific antigen.

In one embodiment, the nucleic acid molecule encodes at least one amino acid sequence of SEQ ID NO:2, SEQ ID NO:4, SEQ ID NO:6, SEQ ID NO:8, SEQ ID NO:10 or SEQ ID NO:12. In one embodiment, the nucleic acid molecule comprises at least one nucleotide sequence of SEQ ID NO:1, SEQ ID NO:3, SEQ ID NO:5, SEQ ID NO:7, SEQ ID NO:9, SEQ ID NO: 11, SEQ ID NO: 15, SEQ ID NO: 16, SEQ ID NO:17 or SEQ ID NO:18.

In one embodiment, the composition comprises one or more nucleic acid molecule encoding a combination of LIC_12340 and LIC_12985. In one embodiment, the composition comprises one or more nucleic acid molecule encoding a combination of SEQ ID NO: 10 and SEQ ID NO:12. In one embodiment, the composition comprises one or more nucleic acid molecule comprising a combination of SEQ ID NO:9 and SEQ ID NO:11.

In one embodiment, the composition comprises one or more nucleic acid molecule encoding a combination of LIC_12340, LIC_12985, LA_3490, LA_0620, and LA_1402. In one embodiment, the composition comprises one or more nucleic acid molecule encoding a combination of SEQ ID NO:2, SEQ ID NO:4, SEQ ID NO:8, SEQ ID NO:10 and SEQ ID NO:12. In one embodiment, the composition comprises one or more nucleic acid molecule comprising a combination of SEQ ID NO:1, SEQ ID NO:3, SEQ ID NO:7, SEQ ID NO:9 and SEQ ID NO:11.

In one embodiment, the composition comprises at least one lipid nanoparticle (LNP) comprising at least one nucleic acid molecule encoding at least one VM protein or fragment thereof comprising the DNase domain. In one embodiment, the nucleic acid molecule comprises an mRNA molecule encoding the at least one VM protein or fragment thereof comprising the DNase domain.

In one embodiment, the composition comprises a vaccine.

In one embodiment, the composition comprises an adjuvant. In one embodiment, the adjuvant is Glucopyranosyl Lipid A (GLA), formulated in a stable oil-in-water nano-emulsion (SE).

In one embodiment, the invention relates to a method of inducing an immune response in a subject, the method comprising administering a composition comprising at least one Leptospiral virulence modifying (VM) protein or a fragment thereof comprising a DNase domain or a composition comprising at least one nucleic acid molecule encoding at least one Leptospiral virulence modifying (VM) protein or fragment thereof comprising a DNase domain of a VM protein to the subject. In one embodiment, the subject is currently infected with Leptospira sp and the composition induces an immune response against Leptospira sp.

In one embodiment, the invention relates to a method of treating or preventing a disease or disorder in a subject, comprising administering a composition comprising at least one Leptospiral virulence modifying (VM) protein or a fragment thereof comprising a DNase domain or a composition comprising at least one nucleic acid molecule encoding at least one Leptospiral virulence modifying (VM) protein or fragment thereof comprising a DNase domain of a VM protein to the subject. In one embodiment, the disease or disorder is cancer, a bacterial infection, a viral infection, or a parasitic infection.

In one embodiment, the invention relates to a method of treating or preventing a disease or disorder in a subject, comprising administering a composition comprising at least one Leptospiral virulence modifying (VM) antibody or a composition comprising at least one nucleic acid molecule encoding at least one Leptospiral virulence modifying (VM) antibody to the subject. In one embodiment, the disease or disorder is Leptospirosis.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts the pan-vaccine challenge study design.

FIG. 2 depicts the experimental schedule for the pan-vaccine challenge study.

FIG. 3 depicts exemplary experimental data demonstrating that immunized C3H/HeJ mice are protected from death/weight loss by pan-vaccine after lethal challenge (low passage L. interrogans serovar Canicola). Mix of 5 antigens: full length mCherry fusions of LA3490, LA0620, and LA1402, and full length LIC12340, and LIC12985. Mix of 2 antigens: full length LIC12340, and LIC12985. Comparative genomic analysis indicates a high level of conservation of these proteins among all pathogenic Leptospira, including (not exclusively) L. interrogans, making it likely that these protein homologs will have similar function and susceptibility to preventive and treatment interventions including vaccine, drug and biologics developed again these and homologous proteins.

FIG. 4 depicts a statistical analysis of the death/weight loss in immunized mice by a pan-leptospirosis vaccine after lethal challenge.

FIG. 5 depicts exemplary experimental data demonstrating VM protein vaccines reduced bacterial load in kidney compared to PBS negative control. Data were statistically analyzed either by a Kruskal-Wallis test. accompanied by Dunn's multiple comparison post-test, with all conditions compared to PBS-treated mice to compare multiple groups (**, p<0.01; ***, p<0.001). There was difference in the bacterial burden in kidneys among the different vaccine groups (Kruskal-Wallis test, P=0.0003 ***).

FIG. 6 depicts a statistical analysis of the bacterial load in kidney following immunization with the VM protein vaccines.

FIG. 7 depicts exemplary experimental data demonstrating VM protein vaccines reduced bacterial load in lung compared to PBS negative control.

FIG. 8 depicts a statistical analysis of the bacterial load in lung following immunization with the VM protein vaccines.

FIG. 9 depicts exemplary experimental data demonstrating detection of cross-reactive VM protein antibodies at pre-challenge. t-test, Non-parametric, unpaired, two tailed, Mann Whitney test, p<0.05, p<0.0001.

FIG. 10A and FIG. 10B depict an evaluation and computational validation of AlphaFold derived three-dimensional structure of VM proteins. FIG. 10A depicts a ramachandran plot analysis evaluate the artificial intelligent based derived three dimensional structures of VM proteins using Zlab (zlab.umassmed.edu/bu/rama/) online server. This provides an overview of allowed and disallowed regions of torsion angle values which serve as an important indicator of the quality of protein structure and stability of three-dimensional confirmation. The percentage of the residue 98.211 (LA3490), 96.59 (LA0620). 98.59 (LA1400), 95.11 (LA1402) and 96.32 (LA0591) was in the favored region (core beta), 1.78 (LA3490), 2.50 (LA0620), 1.20 (LA1400), 4.70 (LA1402) and 2.57 (LA0591) was in the allowed (core-alpha) and 0.00 (LA3490), 0.896 (LA0620), 0.20 (LA1400), 0.18 (LA1402) and 1.13 (LA0591) was in the outlier (core left-handed alpha). FIG. 10B depicts the Z-score mean, Z-score stddev and Z-score RMS calculated using online PROVE analysis server.

FIG. 11A and FIG. 11B depict experimental results demonstrating the structural and sequence representation of QxW motif in VM proteins in L. interrogans serovar Lai. FIG. 11A depicts an AlphaFold generated high-resolution 3D structure of LA3490 VM protein shows amino acids encode for surface aromatic patches (red color: Tyrosine, phenylalanine, and tryptophan). Blue color represents QxW motif at N-terminal RBL1 domain. FIG. 11B depicts an RBL1 domain showing three conserved QxW motif (blue color: ₄₀QKP₄₂, ₁₃₄QRW₁₃₆ and ₇₈QCW₈₀), in which ₁₃₄QRW₁₃₆ motif is conserved in ricin B chain as well. Aromatic motif ₁₅₈YGY₁₆₀ is highly conserved in VM. proteins and in ricin B chain.

FIG. 12A and FIG. 12B depict experimental results demonstrating the structural and functional similarity of RBL2 of VM proteins with CARDs toxin (D3 domain). FIG. 12A depicts the RBL2 domain of LA3490 VM protein (green color: 196 aa-335 aa) structurally superimposed with C-terminal of CARDs toxin (PDB: 4TLV_A Chain, pink color) having RMSD 1.218 Å. FIG. 12B depicts the C-terminal of CARDS toxin (D3 domain) encodes for 8 tryptophan and LA3490 VM protein encode 9 tryptophan. Six of this tryptophan are structurally superimposed in both C-terminal of CARDs toxin and RBL2 domain.

FIG. 13A through FIG. 13E depict experimental results demonstrating a representation and similarly of disulfide bond in LA3490 VM protein and in ricin toxin. FIG. 13A depicts an AlphaFold algorithm generated 3D ribbon structure of LA3490 showing five disulfide bonds by pairing 10 cysteine residues. FIG. 13B depicts a ricin toxin (PDB: 2AAI) showing five disulfide bonds by pairing 10 cysteine residues. FIG. 13C depicts a Ricin B-chain superimpose with CBR (RBL1) of LA3490 along with disulfide bind however, ricin A-chain do not superimpose with C-terminal domain of LA3490. FIG. 13D depicts a superimposition of disulfide bond the similar pattern between LA3490 (magenta color: Cys62 aa-Cys79 aa, Cys105 aa-Cys127 aa, Cys244 aa-Cys262 aa, Cys353 aa-Cys608 aa and Cys630 aa-Cys635 aa) and ricin toxin (purple color: Cys4 aa-Cys259 aa, Cys151 aa-Cys164 aa, Cys20 aa-Cys39 aa, Cys62 aa-Cys79 aa, Cys63 aa-Cys80 aa, Cys105 aa-Cys127 aa, Cys244 aa-Cys262 aa). FIG. 13E depicts the presence of single disulfide bond in LA0591 showing at position Cys303-Cys308.

FIG. 14A through FIG. 14D depict Assessment of hot spot and ligand binding residues in CTD (Carboxy Terminal Domain) of LA3490 VM protein. FIG. 14A depicts data demonstrating that an FTMap machine learning based algorithm shows hot spot residues based on high binding energy affinity and their number of interactions with clusters. FIG. 14A depicts data demonstrating that hot spot residues (Arg615, His533, Cys403, Gln486, Thr549 and Gln523) shows binding with ligands in three-dimensional view. FIG. 14C depicts data demonstrating that a surface view of CTD of LA3490 shows binding of ligands with hot spot residues in deep pockets. FIG. 14D depicts data demonstrating that structural superimposition of CTD of LA3490 and Bovine DNase (3DNI) shows overlapping of His533 (LA3490) with catalytic residue His134 of Bovine DNase.

FIG. 15A through FIG. 15C depict a comparative demonstration of FTMap based hot-spot residues in full length and C-terminal domain of VM proteins. FIG. 15A depicts data demonstrating that AlphaFold generated full length VM proteins (LA3490, LA0620, LA1402 and LA1400) structure shows amino acids with high binding energy and having number of interactions with clusters. FIG. 15B depicts a histogram showing carboxy terminal domain (CTD) of VM proteins (LA0620, LA1400, LA1402 and LA0591) with high binding energy amino acid showing high number of interactions with clusters. FIG. 15C depicts a three-dimensional view of CTD of VM proteins showing ligand binding sites.

FIG. 16A through FIG. 16D depict a PrankWeb and Deepsite based assessment of ligand binding sites of LA3490 VM protein. FIG. 16A depicts an AlphaFold algorithm based Full-length LA3490 PDB file was submitted to ParnkWeb. The machine learning based tool identified 14 deep ligand binding pockets and highest score (18.39) pocket 1 shows in blue color with solvent accessible surface (SAS) and the position for evolutionary conserved pockets shows in bottom panel. FIG. 16B depicts LA0591 shown five pockets with highest score 15.64, pocket 1 shows in blue color with solvent accessible surface (SAS) and the position for evolutionary conserved pockets shows in bottom panel. Deepsite machine learning based algorithm showing the His533 residue in deep pocket as an interactive amino acid represent on surface view of LA3490 (FIG. 16C) and LA0591 (FIG. 16D).

FIG. 17 depicts a comparative assessment of hot spot residues and ligand binding sites in LA3490 and Bovine DNase.

FIG. 18A through FIG. 18F demonstrate the effect of divalent cations on DNase activity of VM proteins. HeLa DNA (150 ng) were incubated with 30 nM of purified soluble recombinant VM proteins (t3490, LA3490, LA0620, LA1402, LA1400 and LA0591) in TM buffer (10 mM Tris pH-7.4) containing 3 mM MgCl₂ (FIG. 18A), absence of (divalent cation) MgCl₂ (FIG. 18B) presence of 2 mM ZnCl₂ (FIG. 18C), presence of 3 mM CaCl₂) (FIG. 18D), presence of CaCl₂)+3 mM MgCl₂ (FIG. 18E) for 30 minutes and samples were subjected to 1% agarose gel electrophoresis. The DNase activity by VM proteins is indicated by smearing and disappearance of DNA; t3490, t0620 had no such effect. (FIG. 18F) Docking study shown phosphate and magnesium ion interact with Gln412 (binding energy −0.95 kCal/mol) and Arg615 (binding energy −2.58 kcal/mol) using MGLTools 1.5.7.

FIG. 19A through FIG. 19C demonstrate a DeepMind AlphaFold algorithm derived structure, strategy for cloning, purification, and antigenicity of recombinant His-tagged VM proteins. FIG. 19A depicts an artificial intelligence-based high-resolution structural modeling of (LA3490, LA0620, LA1402, LA1400 and LA0591) using AlphaFold algorithm. FIG. 19B depicts a schematic diagram depicting the organization of the recombinant mCherry (mC) fusion VM proteins used in the current study; t3490, amino acid positions 40 aa-147 aa (minus signal sequence); LA3490 (19 aa-639 aa), LA0620 (32 aa-637 aa), LA1402 (28 aa-641 aa), LA1400 (1 aa-573 aa) and LA0591 (23 aa-313 aa). The clones were designed without signal sequences. LA1400 naturally lack signal sequence. Recombinant fusions include a glycine-serine (Gly4S)3 linker (for flexibility), N- and C-terminal His6 tag (purification), and N-terminal thioredoxin. FIG. 19C depicts AKTA purified soluble His-tagged VM proteins (LA3490, t3490, LA0620, LA1402, LA0591 and LA1400) were analyzed by 4±12% SDS-PAGE followed by Coomassie staining. A replicate gel was run for immunoblot analysis. The proteins were transferred to a nitrocellulose membrane and the blot was probed with mouse anti-His monoclonal-ALP conjugate (1:2,000 dilution; Santa Cruz Biotechnology, USA). M represents molecular weight marker.

FIG. 20 depicts a mouse immunization schedule and sample collection. C3H/HeJ mice were immunized with 25 μg of total antigen along with adjuvant (5 μg GLA-squalene-oil-in-water emulsion) on days 0, 21 and 42 respectively by intramuscular route. They were pre-bled prior to each immunization and prior to challenge infection, and blood was obtained on day of necropsy. Control mice were immunized with PBS buffer plus adjuvant. Following immunization on day 52, mice were infected with live L. interrogans serovar Canicola (˜1×10⁵ leptospires, LD50<100) by the intraperitoneal route. Blood and organs were collected after subsequent infection.

FIG. 21A through FIG. 21C depict data demonstrating the body weight change, bacterial load and pro-inflammatory cytokine response of mice challenged with L. interrogans serovar Canicola. FIG. 21A depicts mouse body weight (% change) was recorded from 0 day to 13 days upon infection; concurrent assessment of clinical status (grooming, eating, drinking, energy level) was also observed. G-I and G-II mice were sacrificed at 6th and 5th day (‡ and †). Statistical analysis was performed to determine statistical significance in body weight between the PBS control and vaccinated groups using two-tailed unpaired, Mann-Whitney T-Test. p values: VM mix vs PBS, 549 p—0.0152 *: VM unlabeled vs PBS, p—0.0005*: VM unlabeled vs VM mix, p<0.0001 ****: t3490 vs PBS, p—0.3869, ns. Error bars indicate the standard error. Total genomic DNA was extracted from kidney (FIG. 21B) and liver (FIG. 21C) and analyzed by qPCR performed in duplicates with lipL32 primers and SYBER Green probes to quantify leptospiral tissue load. Statistical analysis was performed using the Kruskal-Wallis test and Dunn's multiple comparisons test. p<0.0001 was considered significant. FIG. 21D depicts pro-inflammatory cytokine response in pooled serum samples from each groups: G-I (PBS control), G-II (t3490), G-III (mix of 5 VM proteins) and G-IV mice (mix of 2 VM proteins) pre-challenge and post-challenge were used to measure the levels of IL-1β, IL-6, IL-5, IL-10, IFN-y, TNF-a, KC/GRO by V-PLEX Proinflammatory Panel 1 Mouse Kit (Meso Scale Discovery, MD, USA), an immunoassay based on electrochemiluminescence. PIB denotes pre-immunized bleed.

FIG. 22A and FIG. 22B depict data demonstrating the IgG responses to recombinant VM protein immunization. FIG. 22A depicts data demonstrating that antibody titers were measured in each study group pre- and post-challenge against individual VM proteins in triplicate using ELISA. Each data line represents the average IgG response of each animal (n-10). Box and Whiskers plots represent the antibody titers against t3490, LA3490, LA0620, LA1402. LA1400 and LA0591, respectively. The four-study groups include G-I: PBS, G-II: t3490, G-III: VM mix and G-IV: VM unlabeled. The box boundaries indicate the median and interquartile ranges, and the whiskers denote the maximum and minimum values. Statistical analysis was performed by t-test and the non-parametric, unpaired, two tailed Mann Whitney test. p<0.0001 values were considered significant. FIG. 22B depicts data demonstrating that an aliquot from immunized recombinant purified VM proteins were run in 4±12% SDS-PAGE then transferred to nitrocellulose membrane for Western blot analysis. The membrane was probed with 1:500 pooled sera collected post-challenge. PIB denotes pre-immunized bleed, served as control. VM proteins were recognized by sera from G-II, G-III and G-IV. Lane 1 shows VM mix protein (LA3490, LA0620, LA1402, LA1400 and LA0591) and Lane 2 shows VM unlabeled proteins (LA1400 and LA0591). Arrows shows expected size of VM proteins. M represents molecular weight markers.

FIG. 23A and FIG. 23C depict data demonstrating the In vitro and in vivo recognition of VM proteins in Leptospira cell free lysate by sera from immunized mouse groups. Pathogenic L. interrogans serovars Canicola, Lai, Copenhagni, and non-pathogenic L. biflexa serovar Patoc were grown in conditional EMJH medium which was induced with 120 mM NaCl for 4 h in log phase, and unconditional EMJH medium and cells were harvested. Cell-free lysates were analyzed by 4-12% SDS-PAGE, then transferred to a nitrocellulose membrane for Western blot analysis. FIG. 23A depicts data demonstrating that the membrane was probed with polyclonal LA3490 antibodies (1:2,000 dilution) and LipL32 monoclonal antibody (1:10,000) which was served as loading control. FIG. 23B depicts data demonstrating that the other set of membrane were probed with pooled sera (1:100 dilution) collected before immunization (pre-bleed) and after challenge Group I (PBS+ adjuvant), Group II (t3490), Group III (VM Mix) and Group IV (VM Unlabeled. FIGS. 23A and 23B demonstrate that Leptospira grown in EMJH medium without addition of NaCl, represented by minus (−), and Leptospira grown in EMJH medium to log phase, at which time 120 nM NaCl was added, represented by plus (+). Arrows indicate the expression of 70.29 kDa native VM proteins.

FIG. 23C depicts data demonstrating that Anti-Leptospira immunoglobulins generated against with serovars Canicola after experimental infection of C3H/HeJ susceptible mice. Whole cell IgG ELISA were performed with pre-bleed and sera from immunized mice post-challenge. Serovar Patoc served as negative control.

FIG. 24 depicts a table of the orthologs and percentage amino acid similarity of PF07598 gene family members in Group I pathogenic Leptospira.

FIG. 25 depicts data demonstrating the monoclonal supernatant (YUSM001B) reactivity with recombinant VM proteins.

FIG. 26 depicts a table showing the results of scouting performed with five clones from YUMSIB against the target antigen LA0591 at 500 nM concentration.

FIG. 27 depicts a summary of the reactivities of the five clones from YUMS1B.

FIG. 28 depicts data demonstrating the monoclonal supernatant (YUSM001A, LA1400) reactivity with recombinant VM proteins.

FIG. 29 depicts a table of confirmatory screening data.

FIG. 30 depicts a table of YUSM001A and YUSM001B mouse IgG quant data.

FIG. 31A through FIG. 31C depict data demonstrating that Leptospira PF07598 gene family members, represented by LA3490 here, are predicted with high confidence to have two tandemly repeated, N-terminal ricin B-like (RBL) lectin domains. FIG. 31A depicts a visualization of an AlphaFold 3D-generated model of full-length LA3490 (Callaway, 2020; Jumper et al., 2020; Senior et al., 2020) showing four globular domains N-terminal to C-terminal (blue to red color) residues visualized in PyMOL 2.4.0 pymol.org/2/. Phyre2 (Protein Fold Prediction Server; sbg.bio.ic.ac.uk/phyre2/html/page.cgi?id=index) had first predicted, with high (>94%) confidence, that LA3490, as well as all other virulence-modifying (VM) proteins encoded by the PF07598 gene family, contains N-terminal b-trefoil folds identified as ricin B domains. FIG. 31B depicts a Ricin B domain (PBD; 2AAI-B, 7 aa to 129 aa). FIG. 31C depicts a superimposition of 2AAI-B and N-terminal region of LA3490 (i.e., amino acid positions 40-150) was performed using PyMOL™ 2.4.0 showing structural conservation of RBL1 and the B chain of ricin (RMSD=1.796 Å).

FIG. 32A through FIG. 32D depict data demonstrating a three-dimensional metric multidimensional scaling (3DMMDS/Galaxy) plots depicting (orthologous) VM protein clusters. Clusters were identified among 940 PF07598 family VM proteins analyzed using bios2mds (Pele et al., 2012) and visualized using principal component analysis in R. In addition to typical PF07598 paralogs, 42 natural deletion mutants lacking both ricin B-like lectin, RBL, subdomains (i.e., containing amino terminal signal sequence and toxin domain only) were included. FIG. 32A depicts a carbohydrate-binding region (CBR), containing two unidentical tandem RBL subdomains. FIG. 32B depicts a carboxy terminal toxin domain (CTD) encompassing discrete trafficking and DNase subdomains. For both, initial renderings were edited (cosmetic changes only) to aid visualization by enhancing the 3D effect. No coordinates were altered. Clusters containing VM protein variants found in Leptospira interrogans are highlighted (large spheres) and named using the reference L. interrogans serovar Copenhageni strain (PMID 15028702), L1-130 (UniProtKB) protein IDs. Orthologous clusters were grouped into three superclusters comprising VM protein paralogs (A, n=2; B, n=7; and C, n=4) based upon percent identity (PID). The color key uses the following convention: for species, L. interrogans (ins), L. kirschneri (kri), L. noguchii (nii), etc.; for serovar, e.g., Canicola (CLA), Lai (LAI), Hardjo (HJO), etc.; and for strains originating from Sri Lanka, e.g., L. interrogans serovar Unknown strain KW1 (KW1), etc. FIG. 32C depicts a schematic showing a theoretical evolutionary history of the VM protein family, involving lateral transfer (LGT), gene duplication (purple arrows, II) and erosion (solid black arrows), and recombination (blue arrows=donor acquired via lateral gene transfer, I; broken arrows indicate intragenomic donor from closely related paralog). Circles represent theoretical evolving VM proteins over time; squares represent final evolved form at the current time. FIG. 32D depicts a domain organization and junctions of chimeric Leptospira VM proteins resulting from CBR and CTD domain fusions of paralogs belonging to closely related CBR clusters, such as those related to Q72NW3 (e.g., WP.017856587.1) and Q72TZ4 (e.g., QHH71994.1) (˜99.1% PID). These natural VM protein variants occur infrequently (˜2%) in L. interrogans and its sister species, L. kirschneri and L. noguchii. Chimeric VM proteins generally share a common junction regardless of the paralogs represented.

FIG. 33A through FIG. 33D depict data demonstrating VM Protein LA3490 is a bona fide R-type lectin. FIG. 33A depicts a schematic depicting the organization of the recombinant mCherry (mC) fusion proteins used in the current study; t3490, amino acid positions 40-147 aa (minus SS, signal sequence); and rLA3490, 19-639 aa, also lacking SS. Recombinant fusions also include a glycine-serine linker and C-terminal His6 tag (purification), and N-terminal thioredoxin. RBL and CTD denominates ricin B-like lectin and carboxy terminal domain, respectively. FIG. 33B depicts asialofetuin-binding assay demonstrating that truncated (t3490) and full-length (rLA3490) VM proteins bind to asialofetuin in a dose-dependent manner similar to commercially available ricin B chain. FIG. 33C depicts a competition assay showing that truncated (t3490), the ricin B domain of another VM protein, LA0620 (t0620), and full-length (rLA3490) compete for the same binding site as recombinant ricin B chain (25 nM and 50 nM). Assays were performed in microtiter plates using an ELISA format. Mouse polyclonal anti-LA3490 and anti-LA0620 antibodies (1:1,000 dilution) were used as primary and anti-mouse IgG as secondary antibody (used alone as a specificity control, labeled as 2 Ab control). FIG. 33D depicts a native LA3490 (70.29 kDa) secreted by L. interrogans serovar Lai into EMJH culture supernatant in the presence of 120 mM NaCl binds to asialofetuin-coupled Sepharose beads (AFS). Proteins were eluted with 0.5 M lactose. Unconjugated Sepharose beads incubated with L. interrogans serovar Lai-conditioned medium, and AFS beads with PBS served as controls. Assays were run in triplicate, and experiments were repeated twice. The mean absorbance (±SEM) was visualized in GraphPad Prism 8 and considered statistically significant at p<0.05.

FIG. 34A through FIG. 34C depict data demonstrating a western immunoblot and limulus amebocyte lysate assay confirming identity and purity of recombinant protein preparations. FIG. 34A and FIG. 34B depict a western blot of recombinant protein preparations confirming the presence of a single band of the expected size. A, t3490, B. rLA3490. Membranes were probed with anti-His6 (lane 2) and polyclonal anti-LA3490 antibodies (lane 3). M-molecular weight marker. FIG. 34C Limulus Amebocyte Lysate assay with E. coli LPS as positive control indicating no appreciable endotoxin contamination. Data were visualized in GraphPad Prism v8.

FIG. 35A through FIG. 35F depict data demonstrating the cytopathic effect of rLA3490. FIG. 35A depicts a dose-dependent HeLa cell death induced by r3490 as assessed by trypan blue dye exclusion. Negative controls, t3490, BSA, and no treatment, had no such effect. Cell monolayers were treated with graded molar ratio doses (0 to 904 nM) of LA3490, t3490, and BSA for 4 h. Data represent mean±SD of two independent experiments done with each condition in triplicate (paired t-test, *p<0.005).

FIG. 35B depicts a time-lapse phase-contrast microscopy images (40 frames, 5 s intervals) showing HeLa cell cytopathic effect following exposure to 45 nM of rLA3490 and controls. Only with rLA3490 was cell blebbing evident from 1 h onward [seen in zoom view (top left and right panel, black arrow)]. Time-lapse imaging was captured using a ×40 objective lens using a Leica DMi8 inverted microscope. Scale bar, 10 mm.

FIG. 35C depicts an actin depolymerization occurs early after rLA3490 treatment. HeLa cell monolayers were incubated with 45 nM of rLA3490, t3490, and BSA up to 1 h. Monolayers were fixed with 4% paraformaldehyde followed by 0.1% Triton X-100 in PBS permeabilization. The monolayer was incubated with phalloidin-Alexafluor-488 nm conjugate, washed, and then mounted with ProLong™ Gold Antifade Mountant with DAPI. Images were captured using a Leica DMi8 confocal microscope [Alexa_488 nm (green), DAPI (blue)] at ×40 magnification. Untreated HeLa cells served as control. Scale bar, 20 mm. FIG. 35D depicts a HeLa cell death induced by rLA3490 as assessed by fluorescent live/dead staining. Negative controls (t3490, BSA, and no treatment) had no such effect. Live/dead staining of HeLa cell monolayers was carried out after 4-h exposure to 45 nM rLA3490 (top left panel) and t3490 (bottom left). A dramatic decrease in adherent cells and concomitant accumulation of dead cells upon treatment with rLA3490, but not t3490 or BSA, was observed. Images were captured at ×10 magnification using a Leica DMi8 inverted microscope. Scale bar, 100 mm. FIG. 35E depicts a quantification of LA3490-induced detachment of Hela cells from the monolayer following 4-h exposure, compared with negative control exposure (t3490, BSA, and no treatment). Cells were visibly dissociating from the monolayer after 1 h of rLA3490 exposure. FIG. 35F depicts a quantification of time-dependent HeLa cell death by lactate dehydrogenase release after treatment with rLA3490 in comparison with negative controls. Groups were compared using the one-way t-test in GraphPad Prism 8 and considered statistically significant at p<0.05; ns, non-significant. ** means statistically significant with p=0.0054.

FIG. 36A through FIG. 36C depict data demonstrating caspase activation after rLA3490 treatment of HeLa cells. Super-resolution confocal fluorescence microscopy showed that adding the rLA3490-mCherry fusion protein to Hela cells results in caspase-3 activation associated with internalization of the recombinant protein, as evidenced by cleavage of the caspase-3 recognition sequence/substrate (DEVD) producing green fluorescence. This colocalization also showed significant morphological changes in the nucleus, unlike the negative control t3490, or untreated HeLa cells (FIG. 36A-FIG. 36B). Briefly, cell monolayer was treated with 45 nM recombinant fusion proteins for 4 h. Cells were washed and stained with PBS containing 10 μM Nuc View® 488 substrate and then mounted with ProLong™ Gold Antifade Mount plus DAPI. Images were captured using a 63× oil immersion objective using appropriate filters (blue, DAPI; green, caspase-3 active cells; and red, mCherry fusions). FIG. 36B depicts a zoomed view indicating the co-localization of rLA3490 in nucleus and caspase-3 activation leading cell apoptosis, unlike t3490. FIG. 36C depicts data demonstrating the impact of caspase-3 inhibitor and active caspase-3 fluorescence was read on spectrophotometer plate reader at 488/520 nm (excitation/emission). Prior treatment of HeLa cells with a caspase-3 inhibitor moderated the effect of rLA3490 on apoptotic cells. The various treatments were evaluated via t-test in GraphPad Prism 8 and considered significant when p<0.05, ns=non-significant.

FIG. 37A through FIG. 37C depict data demonstrating surface binding and nuclear localization of rLA3490 in Hela cells. Fluorescent confocal microscopy demonstrating kinetics of binding of mCherry-rLA3490 and mCherry-t3490 fusion proteins binding to HeLa cells. FIG. 37A depicts a two-dimensional view, at 60 min, t3490 is visible only on the cell surface (red); rLA3490 is internalized by 60 min (red/pink). FIG. 37B depicts a three-dimensional Z-stack and orthogonal images obtained by high-resolution fluorescent confocal microscopy showing internalization of mCherry-rLA3490 fusion from 30 min onward, with nuclear translocation and chromosomal degradation (shown by patchy DAPI staining, lower right) evident within 60 min. t3490 remained on the cell surface at 30 and 60 min. Visualization of treated cells was done after staining with CellMask™ green plasma membrane stain mounting with ProLong™ Gold Antifade Mountant+DAPI nuclear stain. Images were captured using an oil immersion ×100 objective using appropriate filters (blue, DAPI; green, plasma membrane; red, mCherry fusions). FIG. 37C depicts a time-dependent interactions of mCherry-tagged rLA3490 and t3490 proteins with Hela cells (surface binding plus internalization). Fluorescent confocal microscopy (using ImageJ version 1.53 software) was used to quantify recombinant fusion proteins with HeLa cell monolayers. Monolayers were exposed to 45 nM recombinant fusion proteins or controls up to 60 min. Fluorescence intensities of mCherry-t3490 and -LA3490 fusion proteins were measured in 10-min intervals from 0 to 60 min. Data were visualized in GraphPrism 8.

FIG. 38A through FIG. 38F depict data demonstrating DNase activity of leptospiral VM proteins. FIG. 38A depicts DNase activity of rLA3490 observed upon incubation of 150 ng of DNA from Hela cells in TM buffer containing 3 mM Mg²⁺ for indicated dose and time (absence of Mg⁺² in reaction yielded no DNA degradation). Samples were subjected to 1% agarose gel electrophoresis. The DNase activity rLA3490 is indicated by smearing and disappearance of DNA; t3490 had no such effect. FIG. 38B depicts data demonstrating that other recombinant VM proteins (LA0620, LA1400, LA1402, and LA0591) all had similar DNase activity. FIG. 38C depicts DNase activity of rLA3490 on 400 ng of undigested plasmid pET28 shows partial degradation with uncoiling, linearizing, and partial degradation, and unaffected by t3490, shown by the white arrow. FIG. 38D depicts DNase activity of rLA3490 on linearized plasmid shows complete disappearance of linear and relaxed plasmid, with dose- and time-dependent smearing. L, DNA ladder. FIG. 38E depicts the quantification of rLA3490 DNase activity using real-time PCR and a FAM fluorescence probe. Bovine DNase, 0.02 U/ml, was used as positive control. Data represent the mean±SD of three independent experiments. FIG. 38F depicts a superimposition of AlphaFold generated CTD of LA3490 and LA0591, respectively. While LA3490 represents the vast majority of VM proteins with two RBLs, a CTD, and intervening functional sequences as visualized in FIG. 1A, LA0591 lacks RBL1 and RBL2 but contains the rest of the functional sequences. This paralog represented by LA0591 is only fully present in L. interrogans species but not in other pathogenic Group I Leptospira. The CTDs of LA3490 and LA0591 are predicted to be highly conserved at the structural level despite amino acid sequence divergence, as shown by the RMSD values of 0.532 Å.

FIG. 39A and FIG. 39B depicts CTD of LA3490 possess conserved active site residues identical to bovine DNase I. FIG. 39A depicts a phylogenetic tree based on amino acid sequence alignment from CTD of LA3490, bovine DNaseI (uniport ID: P00639), mouse DNaseI (P49183), rat DNaseI P21704, human DNaseI (P24855), E. coli_CdtB (Q46669), and human endonuclease (P27695) was generated using phylogeny.fr. Scale bar, one substitution per amino acid site. Numerals indicate the statistical reliability of the branching order as determined by bootstrap analysis of 100 alternative trees. FIG. 39B depicts a superimposition of CTD of LA3490 (368-639 aa) and bovine DNase (PDB: 3DNI) are predicted structural similarity in the active sites of bovine DNaseI with RMSD of 9.012 Å.

### DETAILED DESCRIPTION

The present invention relates to Leptospiral virulence modifying (VM) proteins, and variants and fragments thereof, and vaccine compositions comprising the same. It is demonstrated herein that Leptospiral VM proteins are immunogenic and can thus be used as a vaccine or immunogenic composition to treat or prevent leptospirosis in a subject in need.

In one embodiment, the invention provides a composition comprising at least one virulence modifying (VM) protein or a fragment or variant thereof. In one embodiment, at least one VM protein is from L. interrogans serovar Lai, L. interrogans serovar Copenhageni, L. interrogans serovar Manilae, or a combination thereof. In one embodiment, at least one VM protein is LA_3388, LA_0835, LA_0591, LA_0589 (v), LA_1402, LA_1400, LA_3271, LA_0934, LA_0769, LA_2628, LA_0620, LA_3490, LIC_10778, LIC_12791, LIC_12985, LIC_12986, LIC_12339, LIC_12340, LIC_10870, LIC_12715, LIC_12844, LIC_11358, LIC_10639, LIC_12963, LIC_10695, LMANV2_260038, LMANV2_240079, LMANV2_70075, LMANV2_70078, LMANV2_210058, LMANV2_210056, LMANV2_80114, LMANV2_320010, LMANV2_240142, LMANV2_150103, LMANV2_170032, LMANV2_70050, and LMANV2_170091, or a fragment or variant thereof.

In one embodiment, the invention relates to a vaccine comprising a combination of at least two, three, four, five, or more than five VM proteins. In one embodiment, the vaccine comprises a combination of at least two, three, four, five, or more than five of LA_3388, LA_0835, LA_0591, LA_0589 (v), LA_1402, LA_1400, LA_3271, LA_0934, LA_0769, LA_2628, LA_0620, LA_3490, LIC_10778, LIC_12791, LIC_12985, LIC_12986, LIC_12339, LIC_12340, LIC_10870, LIC_12715, LIC_12844, LIC_11358, LIC_10639, LIC_12963, LIC_10695, LMANV2_260038, LMANV2_240079, LMANV2_70075, LMANV2_70078, LMANV2_210058, LMANV2_210056, LMANV2_80114, LMANV2_320010, LMANV2_240142, LMANV2_150103, LMANV2_170032, LMANV2_70050, and LMANV2_170091, or fragments or variants thereof. In one embodiment, the vaccine comprises a combination of LIC_12340 and LIC_12985. In one embodiment, the vaccine comprises a combination of LIC_12340, LIC_12985, LA_3490, LA_0620, and LA_1402.

In another embodiment, the composition of the invention comprises a nucleic acid sequence encoding at least one VM protein, a fragment thereof or a mutant thereof. In another embodiment, the composition of the invention comprises a nucleic acid sequence encoding at least one VM protein is LA_3388, LA_0835, LA_0591, LA_0589 (v), LA_1402, LA_1400, LA_3271, LA_0934, LA_0769, LA_2628, LA_0620, LA_3490, LIC_10778, LIC_12791, LIC_12985, LIC_12986, LIC_12339, LIC_12340, LIC_10870, LIC_12715, LIC_12844, LIC_11358, LIC_10639, LIC_12963, LIC_10695, LMANV2_260038, LMANV2_240079, LMANV2_70075, LMANV2_70078, LMANV2_210058, LMANV2_210056, LMANV2_80114, LMANV2_320010, LMANV2_240142, LMANV2_150103, LMANV2_170032, LMANV2_70050, and LMANV2_170091, or a fragment or variant thereof. In one embodiment, the composition of the invention comprises nucleotide sequences encoding a combination of at least two, three, four, five, or more than five of LA_3388, LA_0835, LA_0591, LA_0589 (v), LA_1402, LA_1400, LA_3271, LA_0934, LA_0769, LA_2628, LA_0620, LA_3490, LIC_10778, LIC_12791, LIC_12985, LIC_12986, LIC_12339, LIC_12340, LIC_10870, LIC_12715, LIC_12844, LIC_11358, LIC_10639, LIC_12963, LIC_10695, LMANV2_260038, LMANV2_240079, LMANV2_70075, LMANV2_70078, LMANV2_210058, LMANV2_210056, LMANV2_80114, LMANV2_320010, LMANV2_240142, LMANV2_150103, LMANV2_170032, LMANV2_70050, and LMANV2_170091, or fragments or variants thereof. In one embodiment, the nucleotide sequences encode a combination of LIC_12340 and LIC_12985. In one embodiment, the nucleotide sequences encode a combination of LIC_12340, LIC_12985, LA_3490, LA_0620, and LA_1402.

In one embodiment the invention provides compositions and methods for inducing or enhancing an immune response. For example, in certain embodiments, the invention relates to inducing or enhancing cell-mediated and/or humoral immunity directed against a desired antigen.

In one embodiment, the composition of the invention serves as an antigen to induce immunity directed against a Leptospira sp. bacterium. In certain embodiments, the compositions and methods are used to prevent, treat and diagnose infection by Leptospira. In certain embodiments, the compositions and methods are used to prevent or treat a disease or disorder associated with infection by Leptospira, including, but not limited to, leptospirosis, kidney damage, meningitis, liver failure, respiratory distress, and even death. In one embodiment, the composition of the invention is a vaccine that induces the cell-mediated and/or humoral immunity directed against at least one Leptospira sp protein.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section. It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample.

As used herein, the term “autologous” is meant to refer to any material derived from an individual to which it is later to be re-introduced into the same individual.

The term “adjuvant” as used herein is defined as any molecule to enhance an antigen-specific adaptive immune response.

The term “agent” includes any substance, metabolite, molecule, element, compound, or a combination thereof. It includes, but is not limited to, e.g., protein, oligopeptide, small organic molecule, glycan, polysaccharide, polynucleotide, and the like. It can be a natural product, a synthetic compound, a chemical compound, or a combination of two or more substances. Unless otherwise specified, the terms “agent,” “substance,” and “compound” can be used interchangeably. Further, a “test agent” or “candidate agent” is generally a subject agent for use in an assay of the invention.

The term “binding” refers to a direct association between at least two molecules, due to, for example, covalent, electrostatic, hydrophobic, ionic and/or hydrogen-bond interactions.

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4th Ed., U.S. Department of Health and Human Services, National Institutes of Health (1987). There are three heavy chain and three light chain CDRs (or CDR regions) in the variable portion of an immunoglobulin. Thus, “CDRs” as used herein refers to all three heavy chain CDRs, or all three light chain CDRs (or both all heavy and all light chain CDRs, if appropriate). The structure and protein folding of the antibody may mean that other residues are considered part of the antigen binding region and would be understood to be so by a skilled person. See for example Chothia et al., (1989) Conformations of immunoglobulin hypervariable regions; Nature 342, p 877-883.

A “chimeric antibody” refers to a type of engineered antibody which contains a naturally-occurring variable region (light chain and heavy chains) derived from a donor antibody in association with light and heavy chain constant regions derived from an acceptor antibody.

“Contacting” refers to a process in which two or more molecules or two or more components of the same molecule or different molecules are brought into physical proximity such that they are able undergo an interaction. Molecules or components thereof may be contacted by combining two or more different components containing molecules, for example by mixing two or more solution components, preparing a solution comprising two or more molecules such as target, candidate or competitive binding reference molecules, and/or combining two or more flowing components.

As used herein, by “combination therapy” is meant that a first agent is administered in conjunction with another agent. “In conjunction with” refers to administration of one treatment modality in addition to another treatment modality. As such, “in conjunction with” refers to administration of one treatment modality before, during, or after delivery of the other treatment modality to the individual. Such combinations are considered to be part of a single treatment regimen or regime.

As used herein, the term “concurrent administration” means that the administration of the first therapy and that of a second therapy in a combination therapy overlap temporally with each other.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “donor antibody” refers to an antibody (monoclonal, and/or recombinant) which contributes the amino acid sequences of its variable regions, CDRs, or other functional fragments or analogs thereof to a first immunoglobulin partner, so as to provide the altered immunoglobulin coding region and resulting expressed altered antibody with the antigenic specificity and neutralizing activity characteristic of the donor antibody.

The term “acceptor antibody” refers to an antibody (monoclonal and/or recombinant) heterologous to the donor antibody, which contributes all (or any portion, but in some embodiments all) of the amino acid sequences encoding its heavy and/or light chain framework regions and/or its heavy and/or light chain constant regions to the first immunoglobulin partner. In certain embodiments a human antibody is the acceptor antibody.

An “effective amount” as used herein, means an amount which provides a therapeutic or prophylactic benefit.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

As used herein, the term “heavy chain antibody” or “heavy chain antibodies” comprises immunoglobulin molecules derived from camelid species, either by immunization with a peptide and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of VH (variable heavy chain immunoglobulin) genes from an animal.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared multiplied by 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see, e.g., 1989, Queen et al., Proc. Natl. Acad Sci USA, 86:10029-10032; 1991, Hodgson et al., Bio/Technology, 9:421). A suitable human acceptor antibody may be one selected from a conventional database, e.g., the KABAT database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanized antibodies (see for example EP-A-0239400 and EP-A-054951).

The term “immunoglobulin” or “Ig,” as used herein, is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

As used herein, the term “immune response” includes T-cell mediated and/or B-cell mediated immune responses. Exemplary immune responses include T cell responses, e.g., cytokine production and cellular cytotoxicity, and B cell responses, e.g., antibody production. In addition, the term immune response includes immune responses that are indirectly affected by T cell activation, e.g., antibody production (humoral responses) and activation of cytokine responsive cells, e.g., macrophages. Immune cells involved in the immune response include lymphocytes, such as B cells and T cells (CD4+, CD8+, Th1 and Th2 cells); antigen presenting cells (e.g., professional antigen presenting cells such as dendritic cells, macrophages, B lymphocytes, Langerhans cells, and non-professional antigen presenting cells such as keratinocytes, endothelial cells, astrocytes, fibroblasts, oligodendrocytes); natural killer cells; myeloid cells, such as macrophages, eosinophils, mast cells, basophils, and granulocytes.

As used herein, an “inhibitory-effective amount” is an amount that results in a detectable (e.g., measurable) amount of inhibition of an activity. In some instance, the activity is its ability to bind with another component.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “mutation,” as used herein, refers to a change in nucleic acid or polypeptide sequence relative to a reference sequence (which is preferably a naturally-occurring normal or “wild-type” sequence), and includes translocations, deletions, insertions, and substitutions/point mutations. A “mutant” as used herein, refers to either a nucleic acid or protein comprising a mutation.

“Parenteral” administration of an immunogenic composition includes, e.g., subcutaneous (s.c.), intravenous (i.v.), intramuscular (i.m.), or intradermal (i.d.) injection, or infusion techniques.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “X,” the presence of a molecule containing epitope X (or free, unlabeled A), in a reaction containing labeled “X” and the antibody, will reduce the amount of labeled X bound to the antibody.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “therapeutic” as used herein means a treatment and/or prophylaxis. A therapeutic effect is obtained by suppression, diminution, remission, or eradication of a disease state.

The term “therapeutically effective amount” refers to the amount of the subject compound that will elicit the biological or clinical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

To “treat” a disease as the term is used herein, means to reduce the frequency or severity of at least one sign or symptom of a disease or disorder experienced by a subject.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention relates to Leptospiral virulence modifying (VM) proteins encoded by the PF07598 Leptospira gene family, and variants and fragments thereof.

In some embodiments, the present invention provides a composition comprising a Leptospiral VM protein, variant thereof, or fragment thereof. In some embodiment, the composition comprises a fragment of a Leptospiral VM protein. For example, in one embodiment, the composition comprises a fragment of a Leptospiral VM protein, wherein the fragment comprises a C-terminal domain comprising nuclease activity (referred to herein as the DNase domain) of a Leptospiral VM protein.

In one embodiment, the composition comprises a fusion protein, comprising a first domain comprising a Leptospiral VM protein, variant thereof, or fragment thereof. In one embodiment, the fusion protein comprises a second domain. In one embodiment, the second domain is a targeting domain, wherein the targeting domain directs the fusion protein to a specific cell or tissue of interest. For example, in one embodiment, the targeting domain comprises an antibody, antibody fragment, or peptide that specifically binds to an antigen (e.g., tumor antigen) thereby directing the fusion protein to a cell or tissue expressing the antigen. In one embodiment, the second domain comprises a detectable protein or peptide (e.g., a fluorescent protein) that allows for the visualization of the fusion protein.

In one embodiment, the present invention provides an isolated nucleic acid molecule encoding a Leptospiral VM protein, variant thereof, or fragment thereof. In some embodiments, the isolated nucleic acid molecule comprises DNA, cDNA, RNA, or mRNA encoding a Leptospiral VM protein, variant thereof, or fragment thereof. In one embodiment, the isolated nucleic acid molecule encodes a fusion protein comprising a Leptospiral VM protein, variant thereof, or fragment thereof.

In one embodiment, the composition comprises an immunological composition comprising (a) a Leptospiral VM protein, variant thereof, or fragment thereof; or (b) a nucleic acid molecule encoding a Leptospiral VM protein, variant thereof, or fragment thereof. As demonstrated herein, in certain embodiments, a Leptospiral VM protein, variant thereof, or fragment thereof induces a protective immune response that can treat or prevent Leptospiral infection or leptospirosis in a subject in need thereof. In one embodiment, the immunological composition comprises a vaccine. In one embodiment, the immunological composition comprises a bacterium (e.g., a bacterium from genus Leptospira) modified to express a Leptospiral VM protein, variant thereof, or fragment thereof. In one embodiment, the bacterium is attenuated in that it has reduced pathogenicity, but is capable of inducing a protective immune response. The compositions are not only useful as a prophylactic therapeutic agent for immunoprotection, but are also useful as a therapeutic agent for treatment of an ongoing infection, disease, or disorder.

In one embodiment, the present invention relates to methods of inducing cell death or damage comprising administering to a cell a composition comprising (a) a Leptospiral VM protein, variant thereof, or fragment thereof; or (b) a nucleic acid molecule encoding a Leptospiral VM protein, variant thereof, or fragment thereof. For example, as demonstrated herein, Leptospiral VM proteins are cytopathic proteins. In one embodiment, the method comprises administering the composition to a tumor, thereby inducing tumor cell death or damage.

The present invention also provides methods of preventing, inhibiting, and treating infection caused by bacteria of genus Leptospira in a subject in need thereof. In one embodiment, the methods of the invention induce immunity against genus Leptospira in the subject, by generating an immune response in the subject directed to a Leptospiral VM protein. In certain embodiments, the method induces broad immunity across genus Leptospira. In one embodiment, the methods of the invention induce production of VM protein-specific antibodies in the subject. In one embodiment, the methods of the invention prevent Leptospira related pathology, such as leptospirosis (also known as Weil's disease) in a subject in need thereof. In one embodiment, the methods of the invention comprise administering to the subject a composition comprising a) a Leptospiral VM protein, variant thereof, or fragment thereof; or (b) a nucleic acid molecule encoding a Leptospiral VM protein, variant thereof, or fragment thereof

### Compositions

The present invention provides compositions comprising or encoding a Leptospiral VM protein, variant thereof, or fragment thereof.

In one embodiment, the composition comprises a Leptospiral VM protein from L. interrogans serovar Lai, L. interrogans serovar Copenhageni, L. interrogans serovar Manilae, or a combination thereof. In one embodiment, at least one VM protein is LA_3388, LA_0835, LA_0591, LA_0589 (v), LA_1402, LA_1400, LA_3271, LA_0934, LA_0769, LA_2628, LA_0620, LA_3490, LIC_10778, LIC_12791, LIC_12985, LIC_12986, LIC_12339, LIC_12340, LIC_10870, LIC_12715, LIC_12844, LIC_11358, LIC_10639, LIC_12963, LIC_10695, LMANV2_260038, LMANV2_240079, LMANV2_70075, LMANV2_70078, LMANV2_210058, LMANV2_210056, LMANV2_80114, LMANV2_320010, LMANV2_240142, LMANV2_150103, LMANV2_170032, LMANV2_70050, and LMANV2_170091, or a fragment or variant thereof.

In one embodiment, the invention relates to a vaccine comprising a combination of at least two, three, four, five, or more than five VM proteins. In one embodiment, the vaccine comprises a combination of at least two, three, four, five, or more than five of LA_3388, LA_0835, LA_0591, LA_0589 (v), LA_1402, LA_1400, LA_3271, LA_0934, LA_0769, LA_2628, LA_0620, LA_3490, LIC_10778, LIC_12791, LIC_12985, LIC_12986, LIC_12339, LIC_12340, LIC_10870, LIC_12715, LIC_12844, LIC_11358, LIC_10639, LIC_12963, LIC_10695, LMANV2_260038, LMANV2_240079, LMANV2_70075, LMANV2_70078, LMANV2_210058, LMANV2_210056, LMANV2_80114, LMANV2_320010, LMANV2_240142, LMANV2_150103, LMANV2_170032, LMANV2_70050, and LMANV2_170091, or fragments or variants thereof. In one embodiment, the vaccine comprises a combination of LIC_12340 and LIC_12985. In one embodiment, the vaccine comprises a combination of LIC_12340, LIC_12985, LA_3490, LA_0620, and LA_1402.

In one embodiment, the invention relates to a toxoid vaccine comprising a at least one, two, three, four, five, or more than five VM proteins. In one embodiment, the toxoid vaccine comprises at least one, two, three, four, five, or more than five of LA_3388, LA_0835, LA_0591, LA_0589 (v), LA_1402, LA_1400, LA_3271, LA_0934, LA_0769, LA_2628, LA_0620, LA_3490, LIC_10778, LIC_12791, LIC_12985, LIC_12986, LIC_12339, LIC_12340, LIC_10870, LIC_12715, LIC_12844, LIC_11358, LIC_10639, LIC_12963, LIC_10695, LMANV2_260038, LMANV2_240079, LMANV2_70075, LMANV2_70078, LMANV2_210058, LMANV2_210056, LMANV2_80114, LMANV2_320010, LMANV2_240142, LMANV2_150103, LMANV2_170032, LMANV2_70050, and LMANV2_170091, or fragments or variants thereof. In one embodiment, the toxoid vaccine comprises a combination of LIC_12340 and LIC_12985. In one embodiment, the toxoid vaccine comprises a combination of LIC_12340, LIC_12985, LA_3490, LA_0620, and LA_1402.

In one embodiment, the composition or vaccine of the invention comprises a VM protein comprising the amino acid sequence of SEQ ID NO:2, SEQ ID NO:4, SEQ ID NO:6, SEQ ID NO:8, SEQ ID NO:10 or SEQ ID NO: 12, or a fragment or variant thereof. In one embodiment, the composition or vaccine of the invention comprises a combination of VM proteins comprising the amino acid sequences of SEQ ID NO: 10 and SEQ ID NO:12. In one embodiment, the composition or vaccine of the invention comprises a combination of VM proteins comprising the amino acid sequences of SEQ ID NO:10, SEQ ID NO:12, SEQ ID NO:2, SEQ ID NO:4, and SEQ ID NO:8.

In certain embodiments, the composition comprises a fragment of a Leptospiral VM protein. For example, in one embodiment, the composition comprises a DNase domain of a Leptospiral VM protein.

In various embodiments, the invention provides a protein, or a fragment, a homolog, a mutant, a variant, a derivative or a salt of a protein as elsewhere described herein, wherein the activity of the various domains of Leptospiral VM proteins (e.g., immunogenic activity or cytopathic activity or activity related to Leptospiral VM protein mechanism of action) is retained.

Proteins or peptides of the present invention can be prepared using well known techniques. For example, the proteins can be prepared synthetically, using either recombinant DNA technology or chemical synthesis. Proteins of the present invention may be synthesized individually or as longer proteins composed of two or more proteins. The proteins of the present invention can be isolated, i.e., substantially free of other naturally occurring host cell proteins and fragments thereof.

The proteins of the present invention may contain modifications, such as glycosylation, aglycosylation, side chain oxidation, or phosphorylation; so long as the modifications do not destroy the immunologic activity of the proteins. Other modifications include incorporation of D-amino acids or other amino acid mimetics that can be used, for example, to increase the serum half-life of the proteins.

The proteins of the invention can be modified whereby the amino acid is substituted for a different amino acid in which the properties of the amino acid side-chain are conserved (a process known as conservative amino acid substitution). Examples of properties of amino acid side chains are hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W). Note that the parenthetic letters indicate the one-letter codes of amino acids. As used herein, X stands for any amino acid.

The present invention should also be construed to encompass “mutants,” “derivatives,” and “variants” of the proteins of the invention (or of the DNA encoding the same) which mutants, derivatives and variants are polypeptides which are altered in one or more amino acids (or, when referring to the nucleotide sequence encoding the same, are altered in one or more base pairs) such that the resulting protein (or DNA) is not identical to the sequences recited herein, but has the same biological property as the protein disclosed herein.

The invention should also be construed to include any form of a protein variant having substantial homology to an amino acid sequence disclosed herein. In one embodiment, a protein variant is at least about 50%, 70%, 80%, 85%, 86%, 87%, 88%, 89%, 90%, 91%, 92%, 93%, 94%, 95%, 96%, 97%, 98%, or 99% homologous to an amino acid sequence disclosed herein.

The invention should also be construed to include any form of a fragment having a substantial length of an amino acid sequence disclosed herein. In one embodiment, a fragment is at least about 50%, 70%, 80%, 85%, 86%, 87%, 88%, 89%, 90%, 91%, 92%, 93%, 94%, 95%, 96%, 97%, 98%, or 99% of the length of an amino acid sequence disclosed herein.

The invention should also be construed to include any form of a fragment of a protein variant, having both substantial homology to and a substantial length of an amino acid sequence disclosed herein. In one embodiment, a fragment of a protein variant is at least about 50%, 70%, 80%, 85%, 86%, 87%, 88%, 89%, 90%, 91%, 92%, 93%, 94%, 95%, 96%, 97%, 98%, or 99% homologous to an amino acid sequence disclosed herein, and is at least about 50%, 70%, 80%, 85%, 86%, 87%, 88%, 89%, 90%, 91%, 92%, 93%, 94%, 95%, 96%, 97%, 98%, or 99% of the length of an amino acid sequence disclosed herein.

The protein may alternatively be made by recombinant means or by cleavage from a longer protein. The protein may be confirmed by amino acid analysis or sequencing.

The variants of the proteins according to the present invention may be (i) one in which one or more of the amino acid residues are substituted with a conserved or non-conserved amino acid residue (e.g., a conserved amino acid residue) and such substituted amino acid residue may or may not be one encoded by the genetic code, (ii) one in which there are one or more modified amino acid residues, e.g., residues that are modified by the attachment of substituent groups, (iii) one in which the protein comprises an alternative splice variant of the proteins or domains described herein, (iv) fragments of the proteins or domains described herein and/or (v) one in which the protein is fused with another protein or peptide, such as a leader or secretory sequence or a sequence which is employed for purification (for example, His-tag) or for detection (for example, Sv5 epitope tag). The fragments include proteins or peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two peptides is determined by comparing the amino acid sequence and its conserved amino acid substitutes of one peptide to a sequence of a second peptide. Variants are defined to include peptide sequences different from the original sequence, e.g., different from the original sequence in less than 40% of residues per segment of interest, different from the original sequence in less than 25% of residues per segment of interest, different by less than 10% of residues per segment of interest, or different from the original protein sequence in just a few residues per segment of interest and at the same time sufficiently homologous to the original sequence to preserve the functionality of the original sequence. The present invention includes amino acid sequences that are at least 60%, 65%, 70%, 72%, 74%, 76%, 78%, 80%, 90%, or 95% similar or identical to the original amino acid sequence. The degree of identity between two peptides may be determined using computer algorithms and methods that are widely known for the persons skilled in the art. The identity between two amino acid sequences may be determined by using the BLASTP algorithm (BLAST Manual, Altschul, S., et al., NCBI NLM NIH Bethesda, Md. 20894, Altschul, S., et al., J. Mol. Biol. 215: 403-410 (1990)).

The protein of the invention may or may not be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction. A polypeptide or protein of the invention may be phosphorylated using conventional methods such as the method described in Reedijk et al. (The EMBO Journal 11(4): 1365, 1992).

The protein of the invention may include unnatural amino acids formed by post-translational modification or by introducing unnatural amino acids during translation. A variety of approaches are available for introducing unnatural amino acids during polypeptide translation.

A protein of the invention may be conjugated with other molecules, such as polyethylene glycol (PEG). This may be accomplished by inserting cysteine mutations or unnatural amino acids that can be modified with a chemically reactive PEG derivative. In one embodiment, the protein is conjugated to other proteins, to prepare fusion proteins. This may be accomplished, for example, by the synthesis of N-terminal or C-terminal fusion proteins provided that the resulting fusion protein retains the functionality of the protein described herein.

Cyclic derivatives of the proteins of the invention are also part of the present invention. Cyclization may allow the protein to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic protein which is more flexible than the cyclic proteins containing peptide bond linkages as described above. A more flexible protein may be prepared by introducing cysteines at the right and left position of the polypeptide and forming a disulfide bridge between the two cysteines. The two cysteines are arranged so as not to deform the beta-sheet and turn. The protein is more flexible as a result of the length of the disulfide linkage and the smaller number of hydrogen bonds in the beta-sheet portion. The relative flexibility of a cyclic protein can be determined by molecular dynamics simulations.

The invention also relates to a fusion protein. For example, in one embodiment, the fusion protein comprises a first domain comprising a Leptospiral VM protein, variant thereof, or fragment thereof. In one embodiment, the fusion protein comprises a second domain. In one embodiment, the second domain is a targeting domain, wherein the targeting domain directs the fusion protein to a specific cell or tissue of interest. For example, in one embodiment, the targeting domain comprises an antibody, antibody fragment, or peptide that specifically binds to an antigen (e.g., tumor antigen) thereby directing the fusion protein to a cell or tissue expressing the antigen. In one embodiment, the second domain comprises a detectable protein or peptide (e.g., a fluorescent protein) that allows for the visualization of the fusion protein.

In one embodiment, the fusion protein comprises a targeting domain capable of directing the resulting protein to a desired cellular component or cell type or tissue. The chimeric or fusion proteins may also contain additional amino acid sequences or domains. The chimeric or fusion proteins are recombinant in the sense that the various components are from different sources, and as such are not found together in nature (i.e., are heterologous).

In one embodiment, the targeting domain can be a membrane spanning domain, a membrane binding domain, or a sequence directing the protein to associate, for example, with vesicles or with the cell surface. In one embodiment, the targeting domain can target a protein to a particular cell type or tissue. For example, the targeting domain can be a cell surface ligand or an antibody against a cell surface antigens of a target tissue. A targeting domain may target a protein of the invention to a cellular component.

In one embodiment, the targeting domain may comprises an antibody or antibody fragment thereof. An antibody may exist in a variety of forms where the antigen binding domain is expressed as part of a contiguous polypeptide chain including, for example, a single domain antibody fragment (sdAb), a single chain antibody (scFv) and a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426). In one embodiment, the targeting domain of a composition of the invention comprises an antibody fragment. In one embodiment, the targeting domain comprises an antibody fragment that comprises a scFv.

The VM-domain containing fusion molecule of the invention can be generated to be reactive to any desirable antigen of interest, or fragment thereof, including, but not limited to a tumor antigen, a bacterial antigen, a viral antigen or a self-antigen. In the context of the present invention, “tumor antigen” or “hyperporoliferative disorder antigen” or “antigen associated with a hyperproliferative disorder,” refers to antigens that are common to specific hyperproliferative disorders, such as cancer. In certain aspects, the hyperproliferative disorder antigens of the present invention are derived from cancers including, but not limited to, primary or metastatic melanoma, mesothelioma, thymoma, lymphoma, sarcoma, lung cancer, liver cancer, non-Hodgkin's lymphoma, Hodgkins lymphoma, leukemias, uterine cancer, cervical cancer, bladder cancer, kidney cancer and adenocarcinomas such as breast cancer, prostate cancer, ovarian cancer, pancreatic cancer, and the like.

The antigens discussed herein are merely included by way of example. The list is not intended to be exclusive and further examples will be readily apparent to those of skill in the art.

A protein of the invention may be synthesized by conventional techniques. For example, the proteins may be synthesized by chemical synthesis using solid phase peptide synthesis. These methods employ either solid or solution phase synthesis methods (see for example, J. M. Stewart, and J. D. Young, Solid Phase Peptide Synthesis, 2nd Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, supra, Vol 1, for classical solution synthesis). By way of example, a polypeptide of the invention may be synthesized using 9-fluorenyl methoxycarbonyl (Fmoc) solid phase chemistry with direct incorporation of phosphothreonine as the N-fluorenylmethoxy-carbonyl-O-benzyl-L-phosphothreonine derivative.

N-terminal or C-terminal fusion proteins comprising a peptide or protein of the invention, conjugated with at least one other molecule, may be prepared by fusing, through recombinant techniques, the N-terminal or C-terminal end of the peptide or protein, and the sequence of a selected protein or selectable marker with a desired biological function. The resultant fusion proteins contain the Leptospiral VM protein, variant thereof, or fragment thereof, fused to the selected protein or marker protein as described herein. Examples of proteins which may be used to prepare fusion proteins include immunoglobulins and regions thereof, glutathione-S-transferase (GST), hemagglutinin (HA), and truncated myc.

A protein of the invention may be developed using a biological expression system. The use of these systems allows the production of large libraries of random sequences and the screening of these libraries for sequences that bind to particular proteins. Libraries may be produced by cloning synthetic DNA that encodes random peptide sequences into appropriate expression vectors (see Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

The protein of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

The present invention further encompasses fusion proteins in which the protein of the invention or fragments thereof, are recombinantly fused or chemically conjugated (including both covalent and non-covalent conjugations) to heterologous proteins (i.e., an unrelated protein or portion thereof, e.g., at least 10, at least 20, at least 30, at least 40, at least 50, at least 60, at least 70, at least 80, at least 90, at least 100, at least 125, at least 150, at least 175, at least 200, at least 225, at least 250, at least 275, at least 300, or at least 500 amino acids of the polypeptide) to generate fusion proteins. The fusion does not necessarily need to be direct, but may occur through linker sequences.

In one example, a fusion protein in which a protein of the invention or a fragment thereof can be fused to sequences derived from various types of immunoglobulins. For example, a polypeptide of the invention can be fused to a constant region (e.g., hinge, CH2, and CH3 domains) of human IgG or IgM molecule, for example, as described herein, so as to make the fused protein or fragments thereof more soluble and stable in vivo. In another embodiment, such fusion proteins can be administered to a subject so as to inhibit interactions between a ligand and its receptors in vivo. Such inhibition of the interaction will block or suppress signal transduction which triggers certain cellular responses.

In one aspect, the fusion protein comprises a polypeptide of the invention which is fused to a heterologous signal sequence at its N-terminus. For example, the signal sequence naturally found in the protein of the invention can be replaced by a signal sequence which is derived from a heterologous origin. Various signal sequences are commercially available. For example, the secretory sequences of melittin and human placental alkaline phosphatase (Stratagene; La Jolla, Calif.) are available as eukaryotic heterologous signal sequences. As examples of prokaryotic heterologous signal sequences, the phoA secretory signal (Sambrook, et al., supra; and Current Protocols in Molecular Biology, 1992, Ausubel, et al., eds., John Wiley & Sons) and the protein A secretory signal (Pharmacia Biotech; Piscataway, N.J.) can be listed. Another example is the gp67 secretory sequence of the baculovirus envelope protein (Current Protocols in Molecular Biology, 1992, Ausubel, et al., eds., John Wiley & Sons).

In another embodiment, a protein of the invention can be fused to tag sequences, e.g., a hexa-histidine peptide, such as the tag provided in a pQE vector (QIAGEN, Inc., 9259 Eton Avenue, Chatsworth, Calif., 91311), among others, many of which are commercially available. As described in Gentz, et al., 1989, Proc. Natl. Acad. Sci. USA 86:821-824, for instance, hexa-histidine provides for convenient purification of the fusion protein. Other examples of peptide tags are the hemagglutinin “HA” tag, which corresponds to an epitope derived from the influenza hemagglutinin protein (Wilson, et al., 1984, Cell 37:767) and the “flag” tag (Knappik, et al., 1994, Biotechniques 17(4):754-761). These tags are especially useful for purification of recombinantly produced proteins of the invention.

In one embodiment, the protein of the invention can be fused to a detectable label, such as a fluorescent tag. Non-limiting examples of fluorescent tags include green fluorescent protein (GFP), cyan fluorescent protein (CFP), yellow fluorescent protein (YFP), red fluorescent protein (RFP), orange fluorescent protein (OFP), eGFP, mCherry, hrGFP, hrGFPII, Alexa 488, Alexa 594, and the like. Fluorescent tags may also be photoconvertible, such as for example kindling red fluorescent protein (KFP-red), PS-CFP2, Dendra2, CoralHue Kaede and CoralHue Kikume. However, the invention should not be limited to a particular label. Rather, any detectable label can be used to tag the expressed protein.

In some embodiments, the invention provides compositions comprising a lipid nanoparticle (LNP) or liposome conjugated to or encapsulating at least one VM protein or peptide of the invention. In one embodiment, the composition comprises a combination of two or more LNPs encapsulating a combination of two or more VM proteins. In some instances, the LNPs enhances cellular uptake of the VM proteins.

In some embodiments, the composition comprises a scaffold, such as a tissue engineering scaffold, comprising the growth factor-encoding nucleic acid molecule. For example, in one embodiment, the scaffold comprises LNP encapsulating the growth factor-encoding nucleic acid molecule. In one embodiment, the scaffold comprises a cell or cell population comprising the growth factor-encoding nucleic acid molecule. In some embodiments, the scaffold comprises a hydrogel, electrospun scaffold or the like comprising a biopolymer, synthetic polymer or combination thereof.

The present invention also provides isolated nucleic acid molecules that encode the proteins described herein. Therefore, in one embodiment, the composition of the invention comprises an isolated nucleic acid molecule encoding a Leptospiral VM protein, variant thereof, or fragment thereof.

In one embodiment, the isolated nucleic acid molecule encodes a protein or toxoid having an amino acid sequence of SEQ ID NO: 2, SEQ ID NO: 4, SEQ ID NO: 6, SEQ ID NO:8, SEQ ID NO: 10 or SEQ ID NO: 12, or a fragment or variant thereof. In one embodiment, the isolated nucleic acid molecule encodes a fragment comprising a DNase domain of SEQ ID NO: 2, SEQ ID NO: 4, SEQ ID NO: 6, SEQ ID NO:8, SEQ ID NO:10 or SEQ ID NO: 12. In one embodiment, the isolated nucleic acid molecule encodes at least two, at least three, at least four, at least five or more than five VM proteins. In one embodiment, the isolated nucleic acid molecule encodes LIC_12340 and LIC_12985. In one embodiment, the isolated nucleic acid molecule encodes SEQ ID NO:10 and SEQ ID NO: 12. In one embodiment, the isolated nucleic acid molecule comprises SEQ ID NO:9 and SEQ ID NO: 11. In one embodiment, the isolated nucleic acid molecule encodes LIC_12340, LIC_12985, LA_3490, LA_0620, and LA_1402. In one embodiment, the isolated nucleic acid molecule encodes SEQ ID NO:2, SEQ ID NO:4, SEQ ID NO:8, SEQ ID NO: 10 and SEQ ID NO: 12. In one embodiment, the isolated nucleic acid molecule comprises SEQ ID NO:1, SEQ ID NO:3, SEQ ID NO:7, SEQ ID NO:9 and SEQ ID NO:11.

In one embodiment, the invention relates to a combination of at least two isolated nucleic acid molecules encoding a combination of at least two, at least three, at least four, at least five or more than five VM proteins or toxoids. In one embodiment, the at least two isolated nucleic acid molecules encode LIC_12340 and LIC_12985. In one embodiment, the composition comprises a combination of at least two isolated nucleic acid molecules encoding a combination of SEQ ID NO: 10 and SEQ ID NO: 12. In one embodiment, the composition comprises a combination of at least two isolated nucleic acid molecules comprising SEQ ID NO:9 and SEQ ID NO:11. In one embodiment, the at least two isolated nucleic acid molecules encode LIC_12340, LIC_12985, LA_3490, LA_0620, and LA_1402. In one embodiment, the composition comprises a combination of at least two isolated nucleic acid molecules encoding a combination of SEQ ID NO:2, SEQ ID NO:4, SEQ ID NO:8, SEQ ID NO: 10 and SEQ ID NO: 12. In one embodiment, the composition comprises a combination of at least two isolated nucleic acid molecules comprising SEQ ID NO: 1, SEQ ID NO:3, SEQ ID NO:7, SEQ ID NO:9 and SEQ ID NO:11.

The one or more isolated nucleic acid molecule may comprise any type of nucleic acid, including, but not limited to DNA, cDNA, and RNA. For example, in one embodiment, the composition comprises an isolated DNA molecule, including for example, an isolated cDNA molecule, encoding a protein or functional fragment thereof. In one embodiment, the composition comprises an isolated RNA molecule encoding a protein or a functional fragment thereof.

The nucleic acid sequences include both the DNA sequence that is transcribed into RNA and the RNA sequence that is translated into a protein. According to other embodiments, the nucleic acid sequences of the invention are inferred from the amino acid sequence of the proteins of the invention. As is known in the art several alternative nucleic acid sequences are possible due to redundant codons, while retaining the biological activity of the translated proteins.

Further, the invention encompasses an isolated nucleic acid molecule encoding a protein having substantial homology to the VM proteins disclosed herein. In some embodiments, the present invention encompasses an isolated nucleic acid molecule encoding a protein comprising an amino acid sequence having at least about 75%, 80%, 85%, 86%, 87%, 88%, 89%, 90%, 91%, 92%, 93%, 94%, 95%, 96%, 97%, 98%, or 99% sequence homology with an amino acid sequence an amino acid sequence of the proteins disclosed herein. In some embodiments, the nucleic acid sequence encoding a protein of the invention is “substantially homologous,” that is about 50% homologous, about 70% homologous, about 80% homologous, about 90% homologous, about 91% homologous, about 92% homologous, about 93% homologous, about 94% homologous, about 95% homologous, about 96% homologous, about 97% homologous, about 98% homologous, or about 99% homologous to a nucleic acid sequence described herein.

It is to be understood explicitly that the scope of the present invention encompasses homologs, analogs, variants, fragments, derivatives and salts, including shorter and longer proteins and nucleic acid molecules, as well as protein and nucleic acid molecule analogs with one or more amino acid or nucleic acid substitution, as well as amino acid or nucleic acid derivatives, non-natural amino or nucleic acids and synthetic amino or nucleic acids as are known in the art, with the stipulation that these modifications must preserve the activity of the original molecule. Specifically, any active fragments of the active proteins and nucleic acid molecules, as well as extensions, conjugates and mixtures are included and are disclosed herein according to the principles of the present invention.

The invention should be construed to include any and all isolated nucleic acid sequences which are homologous to the nucleic acid sequences described and referenced herein, provided these homologous nucleic acid sequences encode proteins having the biological activity of the proteins disclosed herein.

The skilled artisan would understand that the nucleic acid sequences of the invention encompass an RNA or a DNA sequence encoding a protein of the invention, and any modified forms thereof, including chemical modifications of the DNA or RNA which render the sequence more stable when it is cell free or when it is associated with a cell. Chemical modifications of nucleotides may also be used to enhance the efficiency with which a nucleic acid sequence is taken up by a cell or the efficiency with which it is expressed in a cell. Any and all combinations of modifications of the nucleic acid sequences are contemplated in the present invention.

Further, any number of procedures may be used for the generation of mutant, derivative or variant forms of a protein of the invention using recombinant DNA methodology well known in the art such as, for example, that described in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York). Procedures for the introduction of amino acid changes in a polypeptide or polypeptide by altering the DNA sequence encoding the polypeptide are well known in the art and are also described in these, and other, treatises.

The nucleic acid molecules of the present invention can be modified to improve stability in serum or in growth medium for cell cultures. Modifications can be added to enhance stability, functionality, and/or specificity and to minimize immunostimulatory properties of the nucleic acid molecule of the invention. For example, in order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect function of the molecule.

In one embodiment of the present invention the nucleic acid molecule may contain at least one modified nucleotide analogue. For example, the ends may be stabilized by incorporating modified nucleotide analogues.

Non-limiting examples of nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In exemplary backbone-modified ribonucleotides the phosphoester group connecting to adjacent ribonucleotides is replaced by a modified group, e.g., of phosphothioate group. In exemplary sugar-modified ribonucleotides, the 2′ OH-group is replaced by a group selected from H, OR, R, halo, SH, SR, NH2, NHR, NR2 or ON, wherein R is C1-C6 alkyl, alkenyl or alkynyl and halo is F, Cl, Br or I.

Other examples of modifications are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. The above modifications may be combined.

In some instances, the nucleic acid molecule comprises at least one of the following chemical modifications: 2′-H, 2′-O-methyl, or 2′-OH modification of one or more nucleotides. In some embodiments, a nucleic acid molecule of the invention can have enhanced resistance to nucleases. For increased nuclease resistance, a nucleic acid molecule, can include, for example, 2′-modified ribose units and/or phosphorothioate linkages. For example, the 2′ hydroxyl group (OH) can be modified or replaced with a number of different “oxy” or “deoxy” substituents. For increased nuclease resistance the nucleic acid molecules of the invention can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleobase modifications such as 2-amino-A, 2-thio (e.g., 2-thio-U), G-clamp modifications, can also increase binding affinity to a target.

In one embodiment, the nucleic acid molecule includes a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In one embodiment, the nucleic acid molecule includes at least one 2′-O-methyl-modified nucleotide, and in some embodiments, all of the nucleotides of the nucleic acid molecule include a 2′-O-methyl modification.

Nucleic acid agents discussed herein include otherwise unmodified RNA and DNA as well as RNA and DNA that have been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, for example as occur naturally in the human body. The art has referred to rare or unusual, but naturally occurring, RNAs as modified RNAs, see, e.g., Limbach et al. (Nucleic Acids Res., 1994, 22:2183-2196). Such rare or unusual RNAs, often termed modified RNAs, are typically the result of a post-transcriptional modification and are within the term unmodified RNA as used herein. Modified RNA, as used herein, refers to a molecule in which one or more of the components of the nucleic acid, namely sugars, bases, and phosphate moieties, are different from that which occur in nature, for example different from that which occurs in the human body. While they are referred to as “modified RNAs” they will of course, because of the modification, include molecules that are not, strictly speaking, RNAs. Nucleoside surrogates are molecules in which the ribophosphate backbone is replaced with a non-ribophosphate construct that allows the bases to be presented in the correct spatial relationship such that hybridization is substantially similar to what is seen with a ribophosphate backbone, e.g., non-charged mimics of the ribophosphate backbone.

Modifications of the nucleic acid of the invention may be present at one or more of, a phosphate group, a sugar group, backbone, N-terminus, C-terminus, or nucleobase.

The present invention also includes a vector in which the isolated nucleic acid of the present invention is inserted. The art is replete with suitable vectors that are useful in the present invention.

In brief summary, the expression of natural or synthetic nucleic acids encoding a protein is typically achieved by operably linking a nucleic acid encoding the protein or portions thereof to a promoter, and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

The vectors of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The isolated nucleic acid of the invention can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the vector may be provided to a cell in the form of a viral vector. Viral vector technology is well known in the art and is described, for example, in Sambrook et al. (2012, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In one embodiment, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method.

In some embodiments, the vector also includes conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Enhancer sequences found on a vector also regulates expression of the gene contained therein. Typically, enhancers are bound with protein factors to enhance the transcription of a gene. Enhancers may be located upstream or downstream of the gene it regulates. Enhancers may also be tissue-specific to enhance transcription in a specific cell or tissue type. In one embodiment, the vector of the present invention comprises one or more enhancers to boost transcription of the gene present within the vector.

In order to assess the expression of a protein, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a peptide or protein into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York).

Biological methods for introducing a peptide or protein of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a peptide or protein into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular polypeptide, e.g., by immunological means (ELISAs and Western blots) or by assays described herein to identify agents falling within the scope of the invention.

In one embodiment, the present invention provides a delivery vehicle comprising a protein, or a nucleic acid molecule encoding protein. Exemplary delivery vehicles include, but are not limited to, microspheres, microparticles, nanoparticles, polymerosomes, liposomes, and micelles. For example, in some embodiments, the delivery vehicle is loaded with protein, or a nucleic acid molecule encoding a protein. In some embodiments, the delivery vehicle provides for controlled release, delayed release, or continual release of its loaded cargo. In some embodiments, the delivery vehicle comprises a targeting moiety that targets the delivery vehicle to a treatment site.

In one embodiment, the present invention provides an implantable scaffold or device comprising the protein or nucleic acid molecule encoding the protein. For example, in some embodiments, the present invention provides a tissue engineering scaffold, including but not limited to, a hydrogel, electrospun scaffold, polymeric matrix, or the like, comprising the protein or nucleic acid molecule encoding the protein in or on the scaffold.

In some embodiments, the invention provides compositions comprising a lipid nanoparticle (LNP) or liposome conjugated to or encapsulating at least one nucleic acid molecule encoding at least one VM protein or peptide of the invention. In one embodiment, the composition comprises a combination of two or more LNPs encapsulating a combination of two or more nucleic acid molecules encoding at least two or more VM proteins or peptides of the invention. In one embodiment, the nucleic acid molecule comprises an mRNA molecule encoding at least one VM protein or peptide of the invention. Therefore, in some embodiments, the invention provides at least one LNP or liposome conjugated to or encapsulating at least one mRNA molecule encoding at least one VM protein. In one embodiment, the invention provides a combination of LNP or liposomes conjugated to or encapsulating mRNA molecules encoding LIC_12340 and LIC_12985. In one embodiment, the invention provides a combination of LNP or liposomes conjugated to or encapsulating mRNA molecules encoding LIC_12340, LIC_12985, LA_3490, LA_0620, and LA_1402.

In certain aspects, the present invention encompasses compositions, including polypeptides, nucleotides, vectors, bacteria, and vaccines, that when administered to a subject, elicit or enhance an immune response. In certain instances, the composition elicits an immune response directed against a bacteria of genus Leptospira including an immune response directed against a Leptospiral VM protein. Further, when the compositions are administered to a subject, they elicit an immune response that serves to protect the inoculated subject against conditions associated with Leptospira infection.

In one embodiment, the present invention provides compositions that are useful as immunomodulatory agents, for example, for stimulating immune responses and in preventing Leptospira related pathology. In various embodiments, the immunomodulatory agents comprise (a) a Leptospiral VM protein, variant thereof, or fragment thereof; or (b) a nucleic acid molecule encoding a Leptospiral VM protein, variant thereof, or fragment thereof. In one embodiment, the immune response is not detrimental to the host and therefore the compositions of the invention are useful as a vaccine. In one embodiment, the immunomodulatory agents are administered in combination with an adjuvant. In one embodiment, the adjuvant is glucopyranosyl lipid A (GLA), formulated in a stable oil-in-water nano-emulsion (SE), referred to as a GLA-SE adjuvant. In another embodiment, the immunomodulatory agents are administered in the absence of an adjuvant.

In some embodiments, the compositions are used as immunostimulatory agents to induce or enhance the production of specific antibodies. In certain aspects, the immunostimulatory agents protect against Leptospiral induced pathology.

In one embodiment, the composition comprises a bacterium comprising (a) a Leptospiral VM protein, variant thereof, or fragment thereof; or (b) a nucleic acid molecule encoding a Leptospiral VM protein, variant thereof, or fragment thereof. For example, in one embodiment, the composition comprises a bacterium of genus Leptospira comprising (a) a Leptospiral VM protein, variant thereof, or fragment thereof; or (b) a nucleic acid molecule encoding a Leptospiral VM protein, variant thereof, or fragment thereof. In one embodiment, the composition comprises a bacterium that is not a bacterium of genus Leptospira, wherein the bacterium comprises (a) a Leptospiral VM protein, variant thereof, or fragment thereof; or (b) a nucleic acid molecule encoding a Leptospiral VM protein, variant thereof, or fragment thereof.

A bacterium comprising a nucleotide sequence encoding a Leptospiral VM protein, variant thereof, or fragment thereof, can be generated using any method known in the art including, but not limited to allelic exchange and site-directed mutagenesis.

Any bacterium or bacterial strain which has at least one nucleotide sequence encoding a Leptospiral VM protein, variant thereof, or fragment thereof can be selected and used in accordance with the invention. In one embodiment, naturally occurring mutants or variants, or spontaneous mutants can be selected. In another embodiment, mutant bacteria can be generated by exposing the bacteria to mutagens, such as ultraviolet irradiation or chemical mutagens, or by multiple passages and/or passage in non-permissive hosts. Screening in a differential growth system can be used to select for those mutants having a mutation in a Leptospiral VM protein.

In another embodiment, mutations can be engineered into a bacterium, for example a Leptospira bacterium using “reverse genetics” approaches. In this way, natural or other mutations which confer an inactivated or attenuated phenotype can be engineered into strains. For example, deletions, insertions or substitutions of the coding region of the gene responsible for the Leptospiral VM protein can be engineered. Deletions, substitutions or insertions in the non-coding region of the gene responsible for the Leptospiral VM protein are also contemplated. To this end, mutations in the signals responsible for the transcription, replication, polyadenylation and/or packaging of the gene responsible for the Leptospiral VM protein can be engineered.

In one embodiment, the bacterium is engineered to be deficient, in which a Leptospiral VM protein is absent. For example, in certain embodiments, a toxin-deficient mutant bacterium or virus, where one or more Leptospiral VM protein is absent, is unable to cause disease but is able to induce an adaptive immune response against genus Leptospira.

Bacterium generated by the approaches described herein can be used in the vaccine and pharmaceutical formulations described herein. Reverse genetics techniques can also be used to engineer additional mutations to other genes important for vaccine production—i.e., the epitopes of useful vaccine strain variants can be engineered into the bacterium. Alternatively, completely foreign epitopes, including antigens derived from other pathogens can be engineered into the inactivated or attenuated strain.

The inactivated or attenuated bacterium of the present invention can itself be used as the active ingredient in vaccine or pharmaceutical formulations. In certain embodiments, the bacterium can be used as the vector or “backbone” of recombinantly produced vaccines. To this end, the “reverse genetics” technique can be used to engineer mutations or introduce foreign epitopes into the bacterium, which would serve as the “parental” strain. In this way, vaccines can be designed for immunization against strain variants, or in the alternative, against completely different infectious agents or disease antigens.

For example, in one embodiment, the immunological composition of the invention comprises a bacterium, engineered to express one or more epitopes or antigens of a given pathogen. For example, the bacterium can be engineered to express neutralizing epitopes of other preselected strains. Alternatively, epitopes of other pathogens can be built into the mutant bacterium.

In one embodiment, the bacterium is capable of inducing a robust immune response in the host—a feature which contributes to the generation of a strong immune response when used as a vaccine, and which has other biological consequences that make the bacterium useful as pharmaceutical agents for the prevention and/or treatment of an infection, disease, or disorder associated with an antigen. For example, in certain embodiments, the bacterium induces an anti-Leptospiral immune response.

For an antigenic composition to be useful as a vaccine, the antigenic composition must induce an immune response to the antigen in a cell, tissue or subject (e.g., a human). In certain aspects the vaccine induces a protective immune response in the subject. As used herein, an “immunological composition” may comprise, by way of examples, an antigen (e.g., a protein), a nucleic acid molecule encoding an antigen (e.g., an antigen expression vector), or a cell expressing or presenting an antigen. In particular embodiments the antigenic composition comprises or encodes all or part of any protein antigen described herein, or an immunologically functional equivalent thereof. In other embodiments, the antigenic composition is in a mixture that comprises an additional immunostimulatory agent or nucleic acids encoding such an agent. Immunostimulatory agents include but are not limited to an additional antigen, an immunomodulator, an antigen presenting cell or an adjuvant. In other embodiments, one or more of the additional agent(s) is covalently bonded to the antigen or an immunostimulatory agent, in any combination. In certain embodiments, the antigenic composition is conjugated to or comprises an HLA anchor motif amino acids.

In the context of the present invention, the term “vaccine” (also referred to as an immunogenic composition) refers to a substance that induces immunity upon inoculation into an animal. In one embodiment, the vaccine induces anti-Leptospiral immunity. In various embodiments, the vaccine of the invention comprises

In one embodiment, the vaccine is administered in combination with an adjuvant. In another embodiment, the vaccine is administered in the absence of an adjuvant.

A vaccine of the present invention may vary in its composition of nucleic acid and/or cellular components. In a non-limiting example, a nucleic encoding an antigen might also be formulated with an adjuvant. Of course, it will be understood that various compositions described herein may further comprise additional components. For example, one or more vaccine components may be comprised in a lipid or liposome. In another non-limiting example, a vaccine may comprise one or more adjuvants. A vaccine of the present invention, and its various components, may be prepared and/or administered by any method disclosed herein or as would be known to one of ordinary skill in the art, in light of the present disclosure.

In one embodiment, the protein vaccine of the invention includes, but is not limited to at least one Leptospiral VM protein, variant thereof, or fragment thereof, optionally mixed with adjuvant substances. In some embodiments, the protein is introduced together with an antigen presenting cell (APC). The most common cells used for the latter type of vaccine are bone marrow and peripheral blood derived dendritic cells, as these cells express costimulatory molecules that help activation of T cells. WO00/06723 discloses a cellular vaccine composition which includes an APC presenting tumor associated antigen polypeptides. Presenting the protein can be effected by loading the APC with a polynucleotide (e.g., DNA, RNA) encoding the protein or loading the APC with the protein itself.

For example, a method for detecting the induction of cytotoxic T lymphocytes is well known. A foreign substance that enters the living body is presented to T cells and B cells by the action of APCs. T cells that respond to the antigen presented by APC in an antigen-specific manner differentiate into cytotoxic T cells (also referred to as cytotoxic T lymphocytes or CTLs) due to stimulation by the antigen. These antigen stimulated cells then proliferate. This process is referred to herein as “activation” of T cells. Therefore, CTL induction by a certain polypeptide or combination of polypeptides of the invention can be evaluated by presenting the polypeptide to a T cell by APC, and detecting the induction of CTL. Furthermore, APCs have the effect of activating CD4+ T cells, CD8+ T cells, macrophages, cosinophils and NK cells.

A method for evaluating the inducing action of CTL using dendritic cells (DCs) as APC is well known in the art. DC is a representative APC having the strongest CTL inducing action among APCs. In this method, the polypeptide or combination of polypeptides are initially contacted with DC and then this DC is contacted with T cells. Detection of T cells having cytotoxic effects against the cells of interest after the contact with DC shows that the polypeptide or combination of polypeptides have an activity of inducing the cytotoxic T cells. Furthermore, the induced immune response can be also examined by measuring IFN-gamma produced and released by CTL in the presence of antigen-presenting cells that carry immobilized polypeptide or combination of polypeptides by visualizing using anti-IFN-gamma antibodies, such as an ELISPOT assay.

Apart from DC, peripheral blood mononuclear cells (PBMCs) may also be used as the APC. The induction of CTL is reported to be enhanced by culturing PBMC in the presence of GM-CSF and IL-4. Similarly, CTL has been shown to be induced by culturing PBMC in the presence of keyhole limpet hemocyanin (KLH) and IL-7.

The polypeptide, or combination of polypeptides, confirmed to possess CTL inducing activity by these methods are polypeptides having DC activation effect and subsequent CTL inducing activity. Therefore, a polypeptide or combination of polypeptides that induce CTL against a Leptospiral VM protein are useful as vaccines against Leptospira associated pathology. Furthermore, CTL that have acquired cytotoxicity due to presentation of the polypeptide or combination of polypeptides by APC can be also used as vaccines against Leptospiral infection.

Generally, when using a polypeptide for cellular immunotherapy, efficiency of the CTL-induction can be increased by combining a plurality of polypeptides having different structures and contacting them with DC. Therefore, when stimulating DC with protein fragments, it is advantageous to use a mixture of multiple types of fragments.

The induction of immunity by a polypeptide or combination of polypeptides can be further confirmed by observing the induction of antibody production against the specific antigen. For example, when antibodies against a polypeptide or combination of polypeptides are induced in a laboratory animal immunized with the polypeptide or combination of polypeptides, and when Leptospiral associated pathology is suppressed by those antibodies, the polypeptide or combination of polypeptides are determined to induce anti-Leptospiral immunity.

### Methods

In various embodiments, the compositions of the invention can be used in biological assays, including methods of detecting a protein (e.g. asialofetuin). Exemplary biological assays include, but are not limited to, an immunochromatography assay, an immunodot assay, a Luminex assay, an ELISA assay, an ELISPOT assay, a protein microarray assay, a Western blot assay, a mass spectrophotometry assay, a radioimmunoassay (RIA), a radioimmunodiffusion assay, a liquid chromatography-tandem mass spectrometry assay, an ouchterlony immunodiffusion assay, reverse phase protein microarray, a rocket immunoelectrophoresis assay, an immunohistostaining assay, an immunoprecipitation assay, a complement fixation assay, FACS, an enzyme-substrate binding assay, an enzymatic assay, an enzymatic assay employing a detectable molecule, such as a chromophore, fluorophore, or radioactive substrate, a substrate binding assay employing such a substrate, a substrate displacement assay employing such a substrate, and a protein chip assay (see also, 2007, Van Emon, Immunoassay and Other Bioanalytical Techniques, CRC Press; 2005, Wild, Immunoassay Handbook, Gulf Professional Publishing; 1996, Diamandis and Christopoulos, Immunoassay, Academic Press; 2005, Joos, Microarrays in Clinical Diagnosis, Humana Press; 2005, Hamdan and Righetti, Proteomics Today, John Wiley and Sons; 2007). In some embodiments, the level of asialofetuin in the biological sample is measured with an assay that uses at least one Leptospiral VM protein, variant thereof, or fragment thereof; or a nucleic acid molecule encoding a Leptospiral VM protein, variant thereof, or fragment thereof of the invention, as described elsewhere herein.

In various embodiments, the present invention provides methods comprising administering a composition described herein, to a subject in need thereof. For example, in one embodiment, the method comprises administering to a subject a composition comprising a) a Leptospiral VM protein, variant thereof, or fragment thereof; or (b) a nucleic acid molecule encoding a Leptospiral VM protein, variant thereof, or fragment thereof. In various embodiments, the compositions of the invention can be used as agents for inducing Leptospiral immunity or as cytotoxic agents for the treatment of a disease or disorder.

Compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials. When “an effective amount”, or “therapeutic amount” is indicated, the precise amount of the compositions of the present invention to be administered can be determined by a physician with consideration of individual differences in age, weight, disease progression, and condition of the patient (subject). The optimal dosage and treatment regime for a particular patient can readily be determined by one skilled in the art of medicine by monitoring the subject for signs of disease and adjusting the treatment accordingly.

The administration of the subject compositions may be carried out in any convenient manner, including by aerosol inhalation, injection, ingestion, transfusion, implantation or transplantation. The compositions described herein may be administered to a subject subcutaneously, intradermally, intratumorally, intranodally, intramedullary, intramuscularly, by intravenous (i.v.) injection, or intraperitoneally.

Forms of administration that may be useful in the methods described herein include, but are not limited to, direct delivery to a desired organ, oral, inhalation, intranasal, intratracheal, intravenous, intramuscular, intratumoral, subcutaneous, intradermal, and other parental routes of administration. Additionally, routes of administration may be combined, if desired. In one embodiment, the route of administration is intradermal injection or intratumoral injection. In one embodiment, one or more composition is administered to a treatment site during a surgical procedure, for example during surgical resection of all or part of a tumor.

### Methods of Use as a Vaccine

Thus, the present invention also encompasses a method of inducing anti-Leptospiral immunity using one or more of the compositions described herein. Anti-Leptospiral immunity can be induced by administering a composition of the invention, and the induction of anti-Leptospiral immunity enables treatment and prevention of pathologies associated with Leptospiral infection. Thus, the invention provides a method for treating, or preventing infection by genus Leptospira.

When a certain composition induces a Leptospiral immune response upon inoculation into an animal, the composition is determined to have an immunity inducing effect. The induction of immunity by a composition can be detected by observing in vivo or in vitro the response of the immune system in the host against the composition.

In another embodiment, the methods of the invention comprise administering to the subject a bacterium or virus comprising a nucleic acid sequence encoding a nucleic acid molecule encoding a Leptospiral VM protein, variant thereof, or fragment thereof. In another embodiment, the methods of the invention comprise administering to the subject a bacterium or virus, wherein a Leptospiral VM protein is absent. For example, in certain embodiments, administering a toxin-deficient mutant bacterium or virus, where a Leptospiral VM protein is absent, is unable to cause disease but is able to induce an adaptive immune response.

The therapeutic compounds or compositions of the invention may be administered prophylactically or therapeutically to subjects suffering from, or at risk of, or susceptible to, developing an infection, disease, or disorder associated with the antigen. Such subjects may be identified using standard clinical methods. In the context of the present invention, prophylactic administration occurs prior to the manifestation of overt clinical symptoms of disease, such that a disease or disorder is prevented or alternatively delayed in its progression. In the context of the field of medicine, the term “prevent” encompasses any activity which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

The polypeptide or combination of polypeptides of the invention having immunological activity, or a polynucleotide or vector encoding such a polypeptide or combination of polypeptides, may optionally be combined with an adjuvant. An adjuvant refers to a compound that enhances the immune response against the polypeptide or combination of polypeptides when administered together (or successively) with the polypeptide having immunological activity. Examples of suitable adjuvants include a synthetic TLR4-agonist adjuvant, GLA-SE, cholera toxin, salmonella toxin, alum and such, but are not limited thereto. Furthermore, a vaccine of this invention may be combined appropriately with a pharmaceutically acceptable carrier. Examples of such carriers are sterilized water, physiological saline, phosphate buffer, culture fluid and such. Furthermore, the vaccine may contain as necessary, stabilizers, suspensions, preservatives, surfactants and such. The vaccine is administered systemically or locally. Vaccine administration may be performed by single administration or boosted by multiple administrations.

In one embodiment, the methods of the present invention comprise administering a composition comprising a) at least one Leptospiral VM protein, variant thereof, or fragment thereof; or (b) a nucleic acid molecule encoding at least one Leptospiral VM protein, variant thereof, or fragment thereof, to a subject. In some embodiments, the fragment of the VM protein comprises the DNase domain of the VM protein. Administration of the composition can comprise, for example, intramuscular, intravenous, peritoneal, subcutaneous, intradermal, as well as topical administration.

The actual dose and schedule can vary depending on whether the compositions are administered in combination with other pharmaceutical compositions, or depending on inter-individual differences in pharmacokinetics, drug disposition, and metabolism. Similarly, amounts can vary in in vitro applications depending on the particular cell line utilized (e.g., based on the number of vector receptors present on the cell surface, or the ability of the particular vector employed for gene transfer to replicate in that cell line). Furthermore, the amount of vector to be added per cell will likely vary with the length and stability of the therapeutic gene inserted in the vector, as well as also the nature of the sequence, and is particularly a parameter which needs to be determined empirically, and can be altered due to factors not inherent to the methods of the present invention (for instance, the cost associated with synthesis). One skilled in the art can easily make any necessary adjustments in accordance with the exigencies of the particular situation.

These methods described herein are by no means all-inclusive, and further methods to suit the specific application will be apparent to the ordinary skilled artisan. Moreover, the effective amount of the compositions can be further approximated through analogy to compounds known to exert the desired effect.

### Antibodies

In some embodiments, the invention provides compositions that bind to the VM protein of the invention antigen, including, but not limited to, LA_3388, LA_0835, LA_0591, LA_0589 (v), LA_1402, LA_1400, LA_3271, LA_0934, LA_0769, LA_2628, LA_0620, LA_3490, LIC_10778, LIC_12791, LIC_12985, LIC_12986, LIC_12339, LIC_12340, LIC_10870, LIC_12715, LIC_12844, LIC_11358, LIC_10639, LIC_12963, LIC_10695, LMANV2_260038, LMANV2_240079, LMANV2_70075, LMANV2_70078, LMANV2_210058, LMANV2_210056, LMANV2_80114, LMANV2_320010, LMANV2_240142, LMANV2_150103, LMANV2_170032, LMANV2_70050, and LMANV2_170091, or a fragment or variant thereof. In some embodiments, the composition that binds to the VM protein of the invention is an antibody.

The instant invention relates to the design and development of anti-VM protein antibodies and use thereof for immunotherapy of Leptospiral infection, or Leptospirosis. Anti-VM protein antibodies can function as an immune-prophylaxis strategy for Leptospiral infection, or Leptospirosis.

The anti-VM antibody can bind a target antigen (i.e., a VM protein) present in the subject. Such binding can neutralize the antigen, block recognition of the antigen by another molecule, for example, a protein or nucleic acid, and elicit or induce an immune response to the antigen.

In one embodiment, the composition comprises at least one nucleic acid molecule encoding a synthetic antibody, or a fragment thereof. In one embodiment, the nucleic acid molecule comprises a nucleotide sequence encoding a variable heavy chain region and a nucleotide sequence encoding a variable light chain region of an anti-VM protein antibody. In one embodiment, the invention provides a composition comprising a first nucleic acid molecule comprising a nucleotide sequence encoding a variable heavy chain region of an anti-VM protein antibody and a second nucleic acid molecule comprising a nucleotide sequence encoding a variable light chain region of an anti-VM protein antibody.

Antibodies, including anti-VM protein antibody fragments, of the present invention include, in certain embodiments, antibody amino acid sequences disclosed herein encoded by any suitable polynucleotide, or any isolated or formulated antibody. Further, antibodies of the present disclosure comprise antibodies having the structural and/or functional features of an anti-VM protein antibody described herein. In one embodiment, the anti-VM protein antibody binds a Leptospiral VM protein and, thereby partially or substantially alters at least one biological activity of the Leptospiral VM protein.

In one embodiment, anti-VM protein antibody of the invention immunospecifically bind at least one epitope specific to the VM protein and do not specifically bind to other polypeptides. The at least one epitope can comprise at least one antibody binding region that comprises at least one portion of the full-length VM protein. The term “epitope” as used herein refers to a protein determinant capable of binding to an antibody. Epitopes usually consist of chemically active surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents.

In some embodiments, the invention includes compositions comprising an antibody that specifically binds to a VM protein (e.g., binding portion of an antibody). In one embodiment, the anti-VM protein antibody is a polyclonal antibody. In another embodiment, the anti-VM protein antibody is a monoclonal antibody. In some embodiments, the anti-VM protein antibody is a chimeric antibody. In further embodiments, the anti-VM protein antibody is a humanized antibody.

The binding portion of an antibody comprises one or more fragments of an antibody that retain the ability to specifically bind to binding partner molecule (e.g., Leptospiral VM protein). It has been shown that the binding function of an antibody can be performed by fragments of a full-length antibody. Examples of binding fragments encompassed within the term “binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “binding portion” of an antibody. These antibody fragments are obtained using conventional techniques known to those with skill in the art, and the fragments are screened for utility in the same manner as are intact antibodies. Binding portions can be produced by recombinant DNA techniques, or by enzymatic or chemical cleavage of intact immunoglobulins.

An antibody that binds to a Leptospiral VM protein of the invention is an antibody that inhibits, blocks, or interferes with at least one Leptospiral VM protein activity in vitro, in situ and/or in vivo.

In one embodiment, the Leptospiral VM protein antibody comprises at least one, two or three HCDR sequences as set forth in SEQ ID NO:21, SEQ ID NO:22 and SEQ ID NO:23. In one embodiment, the Leptospiral VM protein antibody comprises at least one, two or three HCDR sequences as set forth in SEQ ID NO:45, SEQ ID NO:46 and SEQ ID NO:47. In one embodiment, the Leptospiral VM protein antibody comprises at least one, two or three HCDR sequences as set forth in SEQ ID NO:61, SEQ ID NO:62 and SEQ ID NO:63. In one embodiment, the Leptospiral VM protein antibody comprises at least one, two or three HCDR sequences as set forth in SEQ ID NO:77, SEQ ID NO:78 and SEQ ID NO:79.

In one embodiment, the Leptospiral VM protein antibody comprises at least one, two or three LCDR sequences as set forth in SEQ ID NO:24, SEQ ID NO:25 and SEQ ID NO:26. In one embodiment, the Leptospiral VM protein antibody comprises at least one, two or three LCDR sequences as set forth in SEQ ID NO:37, SEQ ID NO:38 and SEQ ID NO:39. In one embodiment, the Leptospiral VM protein antibody comprises at least one, two or three LCDR sequences as set forth in SEQ ID NO:48, SEQ ID NO:49 and SEQ ID NO:50. In one embodiment, the Leptospiral VM protein antibody comprises at least one, two or three LCDR sequences as set forth in SEQ ID NO:64, SEQ ID NO:65 and SEQ ID NO:66. In one embodiment, the Leptospiral VM protein antibody comprises at least one, two or three LCDR sequences as set forth in SEQ ID NO:80, SEQ ID NO:81 and SEQ ID NO:82.

In one embodiment, the Leptospiral VM protein antibody comprises the HCDR sequences as set forth in SEQ ID NO:21, SEQ ID NO:22 and SEQ ID NO:23 and the LCDR sequences as set forth in SEQ ID NO:24, SEQ ID NO:25 and SEQ ID NO:26. In one embodiment, the Leptospiral VM protein antibody comprises the HCDR sequences as set forth in SEQ ID NO:21, SEQ ID NO:22 and SEQ ID NO:23 and the LCDR sequences as set forth in SEQ ID NO:37, SEQ ID NO:38 and SEQ ID NO:39. In one embodiment, the Leptospiral VM protein antibody comprises the HCDR sequences as set forth in SEQ ID NO:45, SEQ ID NO:46 and SEQ ID NO:47 and the LCDR sequences as set forth in SEQ ID NO:48, SEQ ID NO:49 and SEQ ID NO:50. In one embodiment, the Leptospiral VM protein antibody comprises the HCDR sequences as set forth in SEQ ID NO:61, SEQ ID NO:62 and SEQ ID NO:63 and the LCDR sequences as set forth in SEQ ID NO:64, SEQ ID NO:65 and SEQ ID NO:66. In one embodiment, the Leptospiral VM protein antibody comprises the HCDR sequences as set forth in SEQ ID NO:77, SEQ ID NO:78 and SEQ ID NO:79 and the LCDR sequences as set forth in SEQ ID NO:80, SEQ ID NO:81 and SEQ ID NO:82.

In one embodiment, the Leptospiral VM protein antibody comprises the HC sequence as set forth in SEQ ID NO:27 and the LC sequence as set forth in SEQ ID NO:28. In one embodiment, the Leptospiral VM protein antibody comprises the HC sequence as set forth in SEQ ID NO:27 and the LC sequence as set forth in SEQ ID NO:43. In one embodiment, the Leptospiral VM protein antibody comprises the HC sequence as set forth in SEQ ID NO:51 and the LC sequence as set forth in SEQ ID NO:52. In one embodiment, the Leptospiral VM protein antibody comprises the HC sequence as set forth in SEQ ID NO:67 and the LC sequence as set forth in SEQ ID NO:68. In one embodiment, the Leptospiral VM protein antibody comprises the HC sequence as set forth in SEQ ID NO:83 and the LC sequence as set forth in SEQ ID NO:84.

TABLE 1
Anti-VM Protein Antibody Sequences

| | | SEQ | |
|---|---|---|---|
| Anti- | | ID | |
| body | | NO: | Sequence |
| 5E10/ | HCDR1 | 21 | GYTFTTYG |
| 5F8 | | | |
| | HCDR2 | 22 | INTYSGIP |
| | HCDR3 | 23 | SRSTVYALDQ |
| | LCDR1 | 24 | QDISNY |
| | LCDR2 | 25 | STS |
| | LCDR3 | 26 | QQGYTLPPT |
| | HC | 27 | QIQLVQSGPELKKPGETVKISCKASGYTFTTY |
| | | | GMSWVKQAPGKGLKWMGWINTYSGIPTFADDF |
| | | | KGRLVFSLETSASTAYLQINNLKNEDTATYFC |
| | | | SRSTVYALDQWGQGTSVTVSS |
| | LC1 | 28 | DIQMTQTTSSLSASLGDRVTITCRASQDISNY |
| | | | LNWYQEKPDGTIKLLIYSTSRLHSGVPSRFSG |
| | | | SGSGTDYSLTISNLGQEDIATYFCQQGYTLPP |
| | | | TFGGGTKLEIK |
| | HCDR1 | 29 | GGGTATACCTTCACGACCTATGGA |
| | HCDR2 | 30 | ATAAACACCTACTCTGGAATTCCA |
| | HCDR3 | 31 | TCAAGATCGACGGTTTATGCTTTGGACCAA |
| | LCDR1 | 32 | CAGGACATTAGTAATTAT |
| | LCDR2 | 33 | TCCACATCA |
| | LCDR3 | 34 | CAACAGGGTTATACGCTTCCTCCCACG |
| | HC | 35 | CAGATCCAGTTGGTACAGTCTGGACCTGAGCT |
| | | | GAAGAAACCTGGAGAGACAGTCAAGATCTCCT |
| | | | GCAAGGCTTCTGGGTATACCTTCACGACCTAT |
| | | | GGAATGAGCTGGGTGAAACAGGCTCCAGGAAA |
| | | | GGGTTTAAAGTGGATGGGCTGGATAAACACCT |
| | | | ACTCTGGAATTCCAACATTTGCTGATGACTTC |
| | | | AAGGGACGGCTTGTCTTCTCTTTGGAAACCTC |
| | | | TGCCAGCACTGCCTATTTGCAGATCAACAACC |
| | | | TCAAAAATGAGGACACGGCTACATATTTCTGT |
| | | | TCAAGATCGACGGTTTATGCTTTGGACCAATG |
| | | | GGGTCAAGGAACCTCAGTCACCGTCTCCTCA |
| | LC1 | 36 | GATATCCAGATGACACAGACTACATCCTCCCT |
| | | | GTCTGCCTCTCTGGGAGACAGAGTCACCATCA |
| | | | CTTGCAGGGCAAGTCAGGACATTAGTAATTAT |
| | | | TTAAACTGGTATCAGGAGAAACCAGATGGAAC |
| | | | TATTAAACTCCTGATCTACTCCACATCAAGAT |
| | | | TACACTCAGGAGTCCCATCAAGGTTCAGTGGC |
| | | | AGTGGGTCTGGAACAGATTATTCTCTCACCAT |
| | | | TAGCAACCTGGGGCAAGAAGATATTGCCACTT |
| | | | ACTTTTGCCAACAGGGTTATACGCTTCCTCCC |
| | | | ACGTTCGGAGGGGGGACCAAGTTGGAAATAAA |
| | | | A |
| 5E10 | LCDR1 | 37 | QSIVHSNGNTY |
| | LCDR2 | 38 | KVS |
| | LCDR3 | 39 | FQGSHVPYT |
| | LCDR1 | 40 | CAGAGCATTGTACATAGTAATGGAAACACCTA |
| | | | T |
| | LCDR2 | 41 | AAAGTTTCC |
| | LCDR3 | 42 | TTTCAAGGTTCACATGTTCCGTACACG |
| | LC | 43 | DVLMTQTPLSLPVSLGDQASISCRSSQSIVHS |
| | | | NGNTYLEWYLQKPGQSPKLLIYKVSNRFSGVP |
| | | | DRFSGSGSGTDFTLKISRVEAEDLGVYYCFQG |
| | | | SHVPYTFGGGTKLEIK |
| | LC | 44 | GATGTTTTGATGACCCAAACTCCACTCTCCCT |
| | | | GCCTGTCAGTCTTGGAGATCAAGCCTCCATCT |
| | | | CTTGCAGATCTAGTCAGAGCATTGTACATAGT |
| | | | AATGGAAACACCTATTTAGAATGGTACCTGCA |
| | | | GAAACCAGGCCAGTCTCCAAAGCTCCTGATCT |
| | | | ACAAAGTTTCCAACCGATTTTCTGGGGTCCCA |
| | | | GACAGGTTCAGTGGCAGTGGATCAGGGACAGA |
| | | | TTTCACACTCAAGATCAGCAGAGTGGAGGCTG |
| | | | AGGATCTGGGAGTTTATTACTGCTTTCAAGGT |
| | | | TCACATGTTCCGTACACGTTCGGAGGGGGGAC |
| | | | CAAGCTGGAAATAAAA |
| 5G10 | HCDR1 | 45 | GYTFTAYG |
| | HCDR2 | 46 | INTYSGVP |
| | HCDR3 | 47 | ARGGELFDY |
| | LCDR1 | 48 | SSINY |
| | LCDR2 | 49 | DTS |
| | LCDR3 | 50 | QRSSYPYT |
| | HC | 51 | QIQLVQSGPELKKPGETVKISCKASGYTFTAY |
| | | | GMSWVMQAPGKGLKWKGWINTYSGVPTYADDF |
| | | | KGRFAFSLETSASTAYLQINLLKNEDTATYFC |
| | | | ARGGELFDYWGQGTTLTVSS |
| | LC | 52 | QIVLTQSPAIMSASPGEKVTMTCSASSSINYM |
| | | | HWYQQKPGTSPKRWIYDTSKLASGVPARFSGS |
| | | | GSGTSYSLTISSMEAEDAATYYCHQRSSYPYT |
| | | | FGGGTKLEIK |
| | HCDR1 | 53 | GGGTATACCTTCACAGCCTATGGA |
| | HCDR2 | 54 | ATAAACACCTACTCTGGAGTGCCA |
| | HCDR3 | 55 | GCAAGAGGGGGGGAACTCTTTGACTAC |
| | LCDR1 | 56 | TCAAGTATAAATTAC |
| | LCDR2 | 57 | GACACATCC |
| | LCDR3 | 58 | CATCAGCGGAGTAGTTACCCGTACACG |
| | HC | 59 | CAGATCCAGCTGGTACAGTCTGGACCTGAGCT |
| | | | GAAGAAGCCTGGAGAGACAGTCAAGATCTCCT |
| | | | GCAAGGCTTCTGGGTATACCTTCACAGCCTAT |
| | | | GGAATGAGCTGGGTGATGCAGGCTCCAGGAAA |
| | | | GGGTTTAAAGTGGAAGGGCTGGATAAACACCT |
| | | | ACTCTGGAGTGCCAACATATGCTGATGACTTC |
| | | | AAGGGACGGTTTGCCTTCTCCTTGGAAACCTC |
| | | | TGCCAGCACTGCCTATTTGCAGATCAATCTCC |
| | | | TCAAAAATGAGGACACGGCTACATATTTCTGT |
| | | | GCAAGAGGGGGGGAACTCTTTGACTACTGGGG |
| | | | CCAAGGCACCACTCTCACAGTCTCCTCA |
| | LC | 60 | CAAATTGTTCTCACCCAGTCTCCAGCAATCAT |
| | | | GTCTGCATCTCCAGGGGAGAAGGTCACCATGA |
| | | | CCTGCAGTGCCAGCTCAAGTATAAATTACATG |
| | | | CACTGGTACCAGCAGAAGCCAGGCACCTCCCC |
| | | | CAAAAGATGGATTTATGACACATCCAAACTGG |
| | | | CTTCTGGAGTCCCTGCTCGCTTCAGTGGCAGT |
| | | | GGGTCTGGGACCTCTTATTCTCTCACAATCAG |
| | | | CAGCATGGAGGCTGAAGATGCTGCCACTTATT |
| | | | ACTGCCATCAGCGGAGTAGTTACCCGTACACG |
| | | | TTCGGTGGGGGGACCAAGCTGGAAATAAAA |
| 6A5 | HCDR1 | 61 | GYTFTRYW |
| | HCDR2 | 62 | IDPSGRYG |
| | HCDR3 | 63 | VREQGVLYTTDY |
| | LCDR1 | 64 | QSLLNSGNQKNYL |
| | LCDR2 | 65 | WAS |
| | LCDR3 | 66 | QNDYSDPT |
| | HC | 67 | QVQLQQPGAEFVKTGASVKLSCKASGYTFTRY |
| | | | WMQWVKQRPGQGLEWIGEIDPSGRYGNYNQKF |
| | | | KGKATLTVDPSSRTAYMQLSSLTSEDSAVYYC |
| | | | VREQGVLYTTDYWGQGTSVTVSS |
| | LC | 68 | DIVMTQSPSSLTVTAGEKVTMSCKSSQSLLNS |
| | | | GNQKNYLTWFQQKPGQPPKLLIYWASARQSGV |
| | | | PDRFTGSGSGTDFTLTISSVQAEDLAVYYCQN |
| | | | DYSDPTFGAGTKLELK |
| | HCDR1 | 69 | GGCTACACCTTCACCAGGTACTGG |
| | HCDR2 | 70 | ATTGATCCTTCTGGTAGATATGGT |
| | HCDR3 | 71 | GTAAGAGAGCAGGGGGTCCTCTATACTACGGA |
| | | | CTAC |
| | LCDR1 | 72 | CAGAGTCTGTTAAATAGTGGAAATCAAAAGAA |
| | | | CTAC |
| | LCDR2 | 73 | TGGGCATCC |
| | LCDR3 | 74 | CAGAATGATTATAGTGATCCCACG |
| | HC | 75 | CAGGTCCAACTGCAGCAGCCTGGGGCTGAGTT |
| | | | TGTGAAGACTGGGGCTTCAGTGAAGCTGTCCT |
| | | | GCAAGGCTTCTGGCTACACCTTCACCAGGTAC |
| | | | TGGATGCAGTGGGTAAAACAGAGGCCTGGACA |
| | | | GGGCCTTGAGTGGATCGGAGAGATTGATCCTT |
| | | | CTGGTAGATATGGTAACTACAATCAAAAGTTC |
| | | | AAGGGCAAGGCCACATTGACTGTAGACCCATC |
| | | | CTCCAGAACAGCCTACATGCAGCTCAGCAGCC |
| | | | TGACATCTGAGGACTCTGCGGTCTATTACTGT |
| | | | GTAAGAGAGCAGGGGGTCCTCTATACTACGGA |
| | | | CTACTGGGGTCAAGGAACCTCAGTCACCGTCT |
| | | | CCTCA |
| | LC | 76 | GACATTGTGATGACACAGTCTCCATCCTCCCT |
| | | | GACTGTGACAGCAGGAGAGAAGGTCACTATGA |
| | | | GCTGCAAGTCCAGTCAGAGTCTGTTAAATAGT |
| | | | GGAAATCAAAAGAACTACTTGACCTGGTTCCA |
| | | | GCAGAAACCAGGCCAGCCTCCTAAACTGTTGA |
| | | | TCTACTGGGCATCCGCTAGGCAATCTGGGGTC |
| | | | CCTGATCGCTTCACAGGCAGTGGATCTGGAAC |
| | | | AGATTTTACTCTCACCATCAGCAGTGTGCAGG |
| | | | CTGAAGACCTGGCAGTTTATTACTGTCAGAAT |
| | | | GATTATAGTGATCCCACGTTCGGTGCTGGGAC |
| | | | CAAGCTGGAGCTGAAA |
| 3H7 | HCDR1 | 77 | GFIFSSYG |
| | HCDR2 | 78 | ISTGGSYT |
| | HCDR3 | 79 | ARYGPYGMDY |
| | LCDR1 | 80 | QSIVHSNGNTY |
| | LCDR2 | 81 | RVS |
| | LCDR3 | 82 | LQGSHVPWT |
| | HC | 83 | EVQLVESGGDVVKPGGSLKLSCAASGFIFSSY |
| | | | GMSWVRQTPDKRLEWVATISTGGSYTYYPDSV |
| | | | KGRFTISRDNAKDTLYLQMSSLKSEDTAMYYC |
| | | | ARYGPYGMDYWGQGTSVTVSS |
| | LC | 84 | DVLMTQTPVSLPVSLGDQASISCRSSQSIVHS |
| | | | NGNTYLEWYLQKPGQSPKLLIHRVSNRFSGVP |
| | | | DRFSGSGSGTDFTLKISRVEAEDLGVYYCLQG |
| | | | SHVPWTFGGGTKVEIK |
| | HCDR1 | 85 | GGATTCATTTTCAGTAGCTATGGC |
| | HCDR2 | 86 | ATTAGTACTGGTGGTAGTTACACC |
| | HCDR3 | 87 | GCAAGGTATGGGCCCTATGGTATGGACTAC |
| | LCDR1 | 88 | CAGAGCATTGTACATAGTAATGGAAACACCTA |
| | | | T |
| | LCDR2 | 89 | AGAGTTTCC |
| | LCDR3 | 90 | CTTCAAGGTTCACACGTTCCGTGGACG |
| | HC | 91 | GAGGTGCAGCTGGTGGAGTCTGGGGGAGACGT |
| | | | AGTGAAGCCTGGAGGGTCCCTGAAACTCTCCT |
| | | | GTGCAGCCTCTGGATTCATTTTCAGTAGCTAT |
| | | | GGCATGTCTTGGGTTCGGCAGACTCCAGACAA |
| | | | GAGGCTGGAGTGGGTCGCAACCATTAGTACTG |
| | | | GTGGTAGTTACACCTACTATCCAGACAGTGTG |
| | | | AAGGGGCGATTCACCATTTCCAGAGACAATGC |
| | | | CAAGGATACCCTGTACCTGCAAATGAGCAGTC |
| | | | TGAAGTCTGAGGACACAGCCATGTATTACTGT |
| | | | GCAAGGTATGGGCCCTATGGTATGGACTACTG |
| | | | GGGTCAAGGAACCTCAGTCACCGTCTCCTCA |
| | LC | 92 | GATGTTTTGATGACCCAAACTCCAGTCTCCCT |
| | | | GCCTGTCAGTCTTGGAGATCAAGCCTCCATCT |
| | | | CTTGCAGATCTAGTCAGAGCATTGTACATAGT |
| | | | AATGGAAACACCTATTTAGAATGGTACCTGCA |
| | | | GAAACCAGGCCAGTCTCCAAAGCTCCTGATCC |
| | | | ACAGAGTTTCCAATCGATTTTCTGGGGTCCCA |
| | | | GACAGGTTCAGTGGCAGTGGATCAGGGACAGA |
| | | | TTTCACACTCAAGATCAGCAGAGTGGAGGCTG |
| | | | AGGATCTGGGAGTTTATTACTGCCTTCAAGGT |
| | | | TCACACGTTCCGTGGACGTTCGGTGGAGGCAC |
| | | | CAAGGTGGAAATCAAA |

In one embodiment, the invention relates to a nucleotide sequence encoding an Leptospiral VM protein antibody or a fragment thereof. In one embodiment, the nucleotide sequence encoding an Leptospiral VM protein antibody comprises an RNA sequence encoding the Leptospiral VM protein antibody. In one embodiment, the nucleotide sequence encoding an Leptospiral VM protein antibody comprises a DNA sequence encoding the Leptospiral VM protein antibody.

In one embodiment, the nucleic acid molecule encodes a Leptospiral VM protein antibody comprising at least one, two or three HCDR sequences as set forth in SEQ ID NO:21, SEQ ID NO:22 and SEQ ID NO:23. In one embodiment, the nucleic acid molecule encodes a Leptospiral VM protein antibody comprising at least one, two or three HCDR sequences as set forth in SEQ ID NO:45, SEQ ID NO:46 and SEQ ID NO:47. In one embodiment, the nucleic acid molecule encodes a Leptospiral VM protein antibody comprising at least one, two or three HCDR sequences as set forth in SEQ ID NO:61, SEQ ID NO:62 and SEQ ID NO:63. In one embodiment, the nucleic acid molecule encodes a Leptospiral VM protein antibody comprising at least one, two or three HCDR sequences as set forth in SEQ ID NO:77, SEQ ID NO:78 and SEQ ID NO:79.

In one embodiment, the nucleic acid molecule encodes a Leptospiral VM protein antibody comprising at least one, two or three LCDR sequences as set forth in SEQ ID NO:24, SEQ ID NO:25 and SEQ ID NO:26. In one embodiment, the nucleic acid molecule encodes a Leptospiral VM protein antibody comprising at least one, two or three LCDR sequences as set forth in SEQ ID NO:37, SEQ ID NO:38 and SEQ ID NO:39. In one embodiment, the nucleic acid molecule encodes a Leptospiral VM protein antibody comprising at least one, two or three LCDR sequences as set forth in SEQ ID NO:48, SEQ ID NO:49 and SEQ ID NO:50. In one embodiment, the nucleic acid molecule encodes a Leptospiral VM protein antibody comprising at least one, two or three LCDR sequences as set forth in SEQ ID NO:64, SEQ ID NO:65 and SEQ ID NO:66. In one embodiment, the nucleic acid molecule encodes a Leptospiral VM protein antibody comprising at least one, two or three LCDR sequences as set forth in SEQ ID NO:80, SEQ ID NO:81 and SEQ ID NO:82.

In one embodiment, the nucleic acid molecule encodes a Leptospiral VM protein antibody comprising the HCDR sequences as set forth in SEQ ID NO:21, SEQ ID NO:22 and SEQ ID NO:23 and the LCDR sequences as set forth in SEQ ID NO:24, SEQ ID NO:25 and SEQ ID NO:26. In one embodiment, the nucleic acid molecule encodes a Leptospiral VM protein antibody comprising the HCDR sequences as set forth in SEQ ID NO:21, SEQ ID NO:22 and SEQ ID NO:23 and the LCDR sequences as set forth in SEQ ID NO:37, SEQ ID NO:38 and SEQ ID NO:39. In one embodiment, the nucleic acid molecule encodes a Leptospiral VM protein antibody comprising the HCDR sequences as set forth in SEQ ID NO:45, SEQ ID NO:46 and SEQ ID NO:47 and the LCDR sequences as set forth in SEQ ID NO:48, SEQ ID NO:49 and SEQ ID NO:50. In one embodiment, the nucleic acid molecule encodes a Leptospiral VM protein antibody comprising the HCDR sequences as set forth in SEQ ID NO:61, SEQ ID NO:62 and SEQ ID NO:63 and the LCDR sequences as set forth in SEQ ID NO:64, SEQ ID NO:65 and SEQ ID NO:66. In one embodiment, the nucleic acid molecule encodes a Leptospiral VM protein antibody comprising the HCDR sequences as set forth in SEQ ID NO:77, SEQ ID NO:78 and SEQ ID NO:79 and the LCDR sequences as set forth in SEQ ID NO:80, SEQ ID NO:81 and SEQ ID NO:82.

In one embodiment, the nucleic acid molecule encodes a Leptospiral VM protein antibody comprising the HC sequence as set forth in SEQ ID NO:27 and the LC sequence as set forth in SEQ ID NO:28. In one embodiment, the nucleic acid molecule encodes a Leptospiral VM protein antibody comprising the HC sequence as set forth in SEQ ID NO:27 and the LC sequence as set forth in SEQ ID NO:43. In one embodiment, the nucleic acid molecule encodes a Leptospiral VM protein antibody comprising the HC sequence as set forth in SEQ ID NO:51 and the LC sequence as set forth in SEQ ID NO:52. In one embodiment, the nucleic acid molecule encodes a Leptospiral VM protein antibody comprising the HC sequence as set forth in SEQ ID NO:67 and the LC sequence as set forth in SEQ ID NO:68. In one embodiment, the nucleic acid molecule encodes a Leptospiral VM protein antibody comprising the HC sequence as set forth in SEQ ID NO:83 and the LC sequence as set forth in SEQ ID NO:84.

In one embodiment, the nucleic acid molecule encoding a Leptospiral VM protein antibody comprises at least one, two or three HCDR sequences as set forth in SEQ ID NO:29, SEQ ID NO:30 and SEQ ID NO:31. In one embodiment, the nucleic acid molecule encoding a Leptospiral VM protein antibody comprises at least one, two or three HCDR sequences as set forth in SEQ ID NO:53, SEQ ID NO:54 and SEQ ID NO:55. In one embodiment, the nucleic acid molecule encoding a Leptospiral VM protein antibody comprises at least one, two or three HCDR sequences as set forth in SEQ ID NO:69, SEQ ID NO:70 and SEQ ID NO:71. In one embodiment, the nucleic acid molecule encoding a Leptospiral VM protein antibody comprises at least one, two or three HCDR sequences as set forth in SEQ ID NO:85, SEQ ID NO:86 and SEQ ID NO:87.

In one embodiment, the nucleic acid molecule encoding a Leptospiral VM protein antibody comprises at least one, two or three LCDR sequences as set forth in SEQ ID NO:32, SEQ ID NO:33 and SEQ ID NO:34. In one embodiment, the nucleic acid molecule encoding a Leptospiral VM protein antibody comprises at least one, two or three LCDR sequences as set forth in SEQ ID NO:40, SEQ ID NO:41 and SEQ ID NO:42. In one embodiment, the nucleic acid molecule encoding a Leptospiral VM protein antibody comprises at least one, two or three LCDR sequences as set forth in SEQ ID NO:56, SEQ ID NO:57 and SEQ ID NO:58. In one embodiment, the nucleic acid molecule encoding a Leptospiral VM protein antibody comprises at least one, two or three LCDR sequences as set forth in SEQ ID NO:72, SEQ ID NO:73 and SEQ ID NO:74. In one embodiment, the nucleic acid molecule encoding a Leptospiral VM protein antibody comprises at least one, two or three LCDR sequences as set forth in SEQ ID NO:88, SEQ ID NO:89 and SEQ ID NO:90.

In one embodiment, the nucleic acid molecule encoding a Leptospiral VM protein antibody comprises the HCDR sequences as set forth in SEQ ID NO:29, SEQ ID NO:30 and SEQ ID NO:31 and the LCDR sequences as set forth in SEQ ID NO:32, SEQ ID NO:33 and SEQ ID NO:34. In one embodiment, the nucleic acid molecule encoding a Leptospiral VM protein antibody comprises the HCDR sequences as set forth in SEQ ID NO:29, SEQ ID NO:30 and SEQ ID NO:31 and the LCDR sequences as set forth in SEQ ID NO:40, SEQ ID NO:41 and SEQ ID NO:42. In one embodiment, the nucleic acid molecule encoding a Leptospiral VM protein antibody comprises the HCDR sequences as set forth in SEQ ID NO:53, SEQ ID NO:54 and SEQ ID NO:55 and the LCDR sequences as set forth in SEQ ID NO:56, SEQ ID NO:57 and SEQ ID NO:58. In one embodiment, the nucleic acid molecule encoding a Leptospiral VM protein antibody comprises the HCDR sequences as set forth in SEQ ID NO:69, SEQ ID NO:70 and SEQ ID NO:71 and the LCDR sequences as set forth in SEQ ID NO:72, SEQ ID NO:73 and SEQ ID NO:74. In one embodiment, the nucleic acid molecule encoding a Leptospiral VM protein antibody comprises the HCDR sequences as set forth in SEQ ID NO:85, SEQ ID NO:86 and SEQ ID NO:87 and the LCDR sequences as set forth in SEQ ID NO:88, SEQ ID NO:89 and SEQ ID NO:90.

In one embodiment, the nucleic acid molecule encoding a Leptospiral VM protein antibody comprises the HC sequence as set forth in SEQ ID NO:35 and the LC sequence as set forth in SEQ ID NO:36. In one embodiment, the nucleic acid molecule encoding a Leptospiral VM protein antibody comprises the HC sequence as set forth in SEQ ID NO:35 and the LC sequence as set forth in SEQ ID NO:44. In one embodiment, the nucleic acid molecule encoding a Leptospiral VM protein antibody comprises the HC sequence as set forth in SEQ ID NO:59 and the LC sequence as set forth in SEQ ID NO:60. In one embodiment, the nucleic acid molecule encoding a Leptospiral VM protein antibody comprises the HC sequence as set forth in SEQ ID NO:75 and the LC sequence as set forth in SEQ ID NO:76. In one embodiment, the nucleic acid molecule encoding a Leptospiral VM protein antibody comprises the HC sequence as set forth in SEQ ID NO:91 and the LC sequence as set forth in SEQ ID NO:92.

The composition of the invention can treat, prevent and/or protect against a disease, disorder, or condition associated with Leptospiral infection.

The composition of the present invention can have features required of effective compositions such as being safe so that the composition does not cause illness or death; being protective against illness; and providing ease of administration, few side effects, biological stability and low cost per dose.

In some embodiments, the Leptospiral VM protein binding molecules (e.g., antibodies, etc.) of the present invention, exhibit a high capacity to detect and bind a Leptospiral VM protein in a complex mixture of salts, compounds and other polypeptides, e.g., as assessed by any one of several in vitro and in vivo assays known in the art. The skilled artisan will understand that the Leptospiral VM protein binding molecules (e.g., antibodies, etc.) described herein as useful in the methods of diagnosis and treatment and prevention of disease, are also useful in procedures and methods of the invention that include, but are not limited to, an immunochromatography assay, an immunodot assay, a Luminex assay, an ELISA assay, an ELISPOT assay, a protein microarray assay, a Western blot assay, a mass spectrophotometry assay, a radioimmunoassay (RIA), a radioimmunodiffusion assay, a liquid chromatography-tandem mass spectrometry assay, an ouchterlony immunodiffusion assay, reverse phase protein microarray, a rocket immunoelectrophoresis assay, an immunohistostaining assay, an immunoprecipitation assay, a complement fixation assay, FACS, a protein chip assay, separation and purification processes, and affinity chromatography (see also, 2007, Van Emon, Immunoassay and Other Bioanalytical Techniques, CRC Press; 2005, Wild, Immunoassay Handbook, Gulf Professional Publishing; 1996, Diamandis and Christopoulos, Immunoassay, Academic Press; 2005, Joos, Microarrays in Clinical Diagnosis, Humana Press; 2005, Hamdan and Righetti, Proteomics Today, John Wiley and Sons; 2007).

In some embodiments, the Leptospiral VM protein binding molecules (e.g., antibodies, etc.) of the present invention, exhibit a high capacity to reduce or to neutralize Leptospiral VM protein activity as assessed by any one of several in vitro and in vivo assays known in the art.

In certain embodiments, the antibody comprises a heavy chain constant region, such as an IgG1, IgG2, IgG3, IgG4, IgA, IgE, IgM or IgD constant region. Preferably, the heavy chain constant region is an IgG1 heavy chain constant region or an IgG4 heavy chain constant region. Furthermore, the antibody can comprise a light chain constant region, either a kappa light chain constant region or a lambda light chain constant region. Preferably, the antibody comprises a kappa light chain constant region. Alternatively, the antibody portion can be, for example, a Fab fragment or a single chain Fv fragment.

### Dosage and Formulation

The present invention envisions treating a disease, for example, diseases associated with a Leptospiral pathogen, in a subject by the administration of one or more of the therapeutic agents of the present invention (e.g., the VM-domain fusion constructs of the invention; toxoid vaccine, anti-VM antibodies or nucleic acid molecules encoding anti-VM antibodies).

Administration of the composition in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. In one embodiment, the cytokine composition, the antigen receptor composition, and the integration composition of the invention are administered locally to the same site. The amount administered will vary depending on various factors including, but not limited to, the composition chosen, the particular disease, the weight, the physical condition, and the age of the mammal, and whether prevention or treatment is to be achieved. Such factors can be readily determined by the clinician employing animal models or other test systems which are well known to the art.

One or more suitable unit dosage forms having the therapeutic agent(s) of the invention, which, as discussed below, may optionally be formulated for sustained release (for example using microencapsulation, see WO 94/07529, and U.S. Pat. No. 4,962,091 the disclosures of which are incorporated by reference herein), can be administered by a variety of routes including parenteral, including by intravenous and intramuscular routes, as well as by direct injection into the diseased tissue. For example, the therapeutic agent may be directly injected into a tumor. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

Lipid nanoparticles (LNPs), liposomes or lipoplexes are effective drug delivery systems for biologically active compounds such as therapeutic proteins, peptides or nucleic acid-based therapeutics, which are otherwise cell impermeable. Therefore, in some embodiments, the invention relates to compositions comprising one or more lipid nanoparticles (LNPs), liposomes or lipoplexes comprising at least one VM protein of the invention, or a nucleic acid molecule encoding the same.

In certain embodiments, the therapeutic agent is combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

The expression vectors, transduced cells, polynucleotides and polypeptides (active ingredients) of this invention can be formulated and administered to treat a variety of disease states by any means that produces contact of the active ingredient with the agent's site of action in the body of the organism. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium Ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

The active ingredients of the invention may be formulated to be suspended in a pharmaceutically acceptable composition suitable for use in mammals and in particular, in humans. Such formulations include the use of adjuvants such as muramyl dipeptide derivatives (MDP) or analogs that are described in U.S. Pat. Nos. 4,082,735; 4,082,736; 4,101,536; 4,185,089; 4,235,771; and 4,406,890. Other adjuvants, which are useful, include alum (Pierce Chemical Co.), lipid A, trehalose dimycolate and dimethyldioctadecylammonium bromide (DDA), Freund's adjuvant, and IL-12. Other components may include a polyoxypropylene-polyoxyethylene block polymer (Pluronic®), a non-ionic surfactant, and a metabolizable oil such as squalene (U.S. Pat. No. 4,606,918).

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Accordingly, the composition of the present invention may be delivered via various routes and to various sites in a mammal body to achieve a particular effect (see, e.g., Rosenfeld et al., 1991; Rosenfeld et al., 1991a; Jaffe et al., supra; Berkner, supra). One skilled in the art will recognize that although more than one route can be used for administration, a particular route can provide a more immediate and more effective reaction than another route. In one embodiment, the composition described above is administered to the subject by intratumoral injection. Other forms of administration that may be useful in the methods described herein include, but are not limited to, direct delivery to a desired organ, intramuscular, subcutaneous, intradermal, and other parental routes of administration.

The active ingredients of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the composition in the particular host.

These methods described herein are by no means all-inclusive, and further methods to suit the specific application will be apparent to the ordinary skilled artisan. Moreover, the effective amount of the compositions can be further approximated through analogy to compounds known to exert the desired effect.

### Methods of Treatment

In one embodiment, the invention includes methods of inducing an immune response in a subject in need thereof comprising administering a Leptospiral VM protein, a toxoid vaccine, an anti-VM antibody or a nucleic acid molecule encoding an anti-VM antibody of the invention.

In one embodiment, a Leptospiral VM protein or peptide or a vector comprising a nucleotide sequence encoding for a Leptospiral VM protein or peptide, serves a toxoid vaccine. Also provided herein is a method of treating, protecting against, and/or preventing disease in a subject in need thereof by administering the toxoid vaccine to the subject. Administration of the vaccine to the subject can induce or elicit an immune response in the subject. The induced immune response can be used to treat, prevent, and/or protect against disease, for example, an infectious disease, including but not limited to pathologies relating to Leptospira infection.

In one embodiment, a fusion protein comprising a Leptospiral VM protein or peptide fused to an antigenic peptide or a vector comprising a nucleotide sequence encoding for fusion protein comprising a Leptospiral VM protein or peptide fused to an antigenic peptide, serves as a therapeutic agent for the treatment of a disease or disorder associated with the antigenic peptide.

In some embodiments, the antigenic peptide is a tumor associated peptide. Therefore, in some embodiments, the induced immune response can be used to treat, prevent, and/or protect against cancer. The following are non-limiting examples of cancers that can be treated by the disclosed methods and compositions: acute lymphoblastic leukemia, acute myeloid leukemia, adrenocortical carcinoma, appendix cancer, basal cell carcinoma, bile duct cancer, bladder cancer, bone cancer, brain and spinal cord tumors, brain stem glioma, brain tumor, breast cancer, bronchial tumors, burkitt lymphoma, carcinoid tumor, central nervous system atypical teratoid/rhabdoid tumor, central nervous system embryonal tumors, central nervous system lymphoma, cerebellar astrocytoma, cerebral astrocytoma/malignant glioma, cerebral astrocytotna/malignant glioma, cervical cancer, childhood visual pathway tumor, chordoma, chronic lymphocytic leukemia, chronic myelogenous leukemia, chronic myeloproliferative disorders, colon cancer, colorectal cancer, craniopharyngioma, cutaneous cancer, cutaneous t-cell lymphoma, endometrial cancer, ependymoblastoma, ependymoma, esophageal cancer, ewing family of tumors, extracranial cancer, extragonadal germ cell tumor, extrahepatic bile duct cancer, extrahepatic cancer, eye cancer, fungoides, gallbladder cancer, gastric (stomach) cancer, gastrointestinal cancer, gastrointestinal carcinoid tumor, gastrointestinal stromal tumor (gist), germ cell tumor, gestational cancer, gestational trophoblastic tumor, glioblastoma, glioma, hairy cell leukemia, head and neck cancer, hepatocellular (liver) cancer, histiocytosis, hodgkin lymphoma, hypopharyngeal cancer, hypothalamic and visual pathway glioma, hypothalamic tumor, intraocular (eye) cancer, intraocular melanoma, islet cell tumors, kaposi sarcoma, kidney (renal cell) cancer, langerhans cell cancer, langerhans cell histiocytosis, laryngeal cancer, leukemia, lip and oral cavity cancer, liver cancer, lung cancer, lymphoma, macroglobulinemia, malignant fibrous histiocvtoma of bone and osteosarcoma, medulloblastoma, medulloepithelioma, melanoma, merkel cell carcinoma, mesothelioma, metastatic squamous neck cancer with occult primary, mouth cancer, multiple endocrine neoplasia syndrome, multiple myeloma, mycosis, myelodysplastic syndromes, myelodysplastic/myeloproliferative diseases, myelogenous leukemia, myeloid leukemia, myeloma, myeloproliferative disorders, nasal cavity and paranasal sinus cancer, nasopharyngeal cancer, neuroblastoma, non-hodgkin lymphoma, non-small cell lung cancer, oral cancer, oral cavity cancer, oropharyngeal cancer, osteosarcoma and malignant fibrous histiocytoma, osteosarcoma and malignant fibrous histiocytoma of bone, ovarian, ovarian cancer, ovarian epithelial cancer, ovarian germ cell tumor, ovarian low malignant potential tumor, pancreatic cancer, papillomatosis, paraganglioma, parathyroid cancer, penile cancer, pharyngeal cancer, pheochromocytoma, pineal parenchymal tumors of intermediate differentiation, pincoblastoma and supratentorial primitive neuroectodermal tumors, pituitary tumor, plasma cell neoplasm, plasma cell neoplasm/multiple myeloma, pleuropulmonary blastoma, primary central nervous system cancer, primary central nervous system lymphoma, prostate cancer, rectal cancer, renal cell (kidney) cancer, renal pelvis and ureter cancer, respiratory tract carcinoma involving the nut gene on chromosome 15, retinoblastoma, rhabdomyosarcoma, salivary gland cancer, sarcoma, sezary syndrome, skin cancer (melanoma), skin cancer (nonmelanoma), skin carcinoma, small cell lung cancer, small intestine cancer, soft tissue cancer, soft tissue sarcoma, squamous cell carcinoma, squamous neck cancer, stomach (gastric) cancer, supratentorial primitive neuroectodermal tumors, supratentorial primitive neuroectodermal tumors and pineoblastoma, T-cell lymphoma, testicular cancer, throat cancer, thymoma and thymic carcinoma, thyroid cancer, transitional cell cancer, transitional cell cancer of the renal pelvis and ureter, trophoblastic tumor, urethral cancer, uterine cancer, uterine sarcoma, vaginal cancer, visual pathway and hypothalamic glioma, vulvar cancer, waldenstrom macroglobulinemia, and wilms tumor.

In one embodiment, the methods of the invention include administering an antigenic protein, peptide to a subject, wherein the antigenic protein, peptide promotes the generation of an immune response against the antigen. In one embodiment, the methods of the invention include administering a nucleic acid molecule to a subject wherein the nucleic acid molecule comprises an expression construct for expression of at least one antigenic protein or peptide, wherein the antigenic protein or peptide promotes the generation of an immune response against the encoded antigenic protein or peptide. In one embodiment, the methods of the invention include administering an antibody to a subject wherein the antibody targets a disease-associated antigen. In one embodiment, the methods of the invention include administering at least one nucleic acid molecule encoding an antibody, or fragment thereof, to a subject wherein the encoded antibody targets a disease-associated antigen.

In some embodiments, an induced immune response can include an induced humoral immune response and/or an induced cellular immune response. The humoral immune response can be induced by about 1.5-fold to about 16-fold, about 2-fold to about 12-fold, or about 3-fold to about 10-fold. The induced humoral immune response can include IgG antibodies and/or neutralizing antibodies. The induced cellular immune response can include a CD8+ T cell response, which is induced by about 2-fold to about 30-fold, about 3-fold to about 25-fold, or about 4-fold to about 20-fold.

The vaccine dose can be between 1 μg to 10 mg active component/kg body weight/time, and can be 20 μg to 10 mg component/kg body weight/time. The vaccine can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of vaccine doses for effective treatment can be 1, 2, 3, 4, 5, 6, 7, 8, 9, or 10.

In one embodiment, the vaccine or antibody of the invention can be administered alone. In one embodiment, the vaccine or antibody of the invention can be administered in combination with another treatment for a disease or disorder.

In one embodiment the vaccine or antibody of the invention is administered in combination with an additional vaccine composition as a prime or a boost vaccine. In one embodiment, a subject who has been immunized with a vaccine (as a priming vaccine) is then administered a vaccine of the invention as a boosting vaccine to increase the immune response.

In one embodiment a vector of the invention may expresses at least two antigenic polypeptides, wherein at least one antigenic polypeptide is a Leptospiral VM protein of the invention.

### Administration

The compositions of the invention can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular subject, and the route of administration. The subject can be a mammal, such as a human, a horse, a cow, a pig, a sheep, a cat, a dog, a rat, or a mouse.

The composition can be administered prophylactically or therapeutically. In prophylactic administration, the compositions can be administered in an amount sufficient to induce an immune response. In therapeutic applications, the compositions are administered to a subject in need thereof in an amount sufficient to elicit a therapeutic effect. An amount adequate to accomplish this is defined as “therapeutically effective dose.” Amounts effective for this use will depend on, e.g., the particular composition of the treatment regimen administered, the manner of administration, the stage and severity of the disease, the general state of health of the patient, and the judgment of the prescribing physician.

The composition can be administered by methods well known in the art as described in Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997), the contents of all of which are incorporated herein by reference in their entirety. One skilled in the art would know that the choice of a pharmaceutically acceptable carrier, including a physiologically acceptable compound, depends, for example, on the route of administration of the expression vector.

The composition can be delivered via a variety of routes. Typical delivery routes include parenteral administration, e.g., intradermal, intramuscular, intratumoral or subcutaneous delivery. Other routes include oral administration, intranasal, and intravaginal routes. For the DNA of the composition in particular, the composition can be delivered to the interstitial spaces of tissues of an individual (Felgner et al., U.S. Pat. Nos. 5,580,859 and 5,703,055, the contents of all of which are incorporated herein by reference in their entirety). The composition can also be administered to muscle, or can be administered via intradermal or subcutaneous injections, or transdermally, such as by iontophoresis. Epidermal administration of the composition can also be employed. Epidermal administration can involve mechanically or chemically irritating the outermost layer of epidermis to stimulate an immune response to the irritant (Carson et al., U.S. Pat. No. 5,679,647, the contents of which are incorporated herein by reference in its entirety).

In one embodiment, the proteins, nucleic acid molecules or antibodies of the present invention can be administered to cells of a mammal including a human.

In some embodiments, the proteins, nucleic acid molecules or antibodies of the present invention can be administered as an injection (subcutaneous, intradermal, or intramuscular injection) to cells of a mammal including a human. The injection can be prepared by a standard method. For example, a culture supernatant containing the virus vector is concentrated, if necessary, and suspended together with an appropriate carrier or excipient in a buffer solution such as PBS or saline. Then, the suspension can be sterilized by filtration through a filter or the like according to the need and subsequently charged into an aseptic container to prepare the injection. The injection may be supplemented with a stabilizer, a preservative, and the like, according to the need. The expression vector thus obtained can be administered as the injection to a subject.

In some embodiments, the proteins, nucleic acid molecules or antibodies can be formulated for administration by way of intradermal (ID) vaccination (e.g., ID injection by the Mantoux technique, use of a hollow microneedle, using a gene gun, using scarification or by other methods for ID delivery). The formulation for ID vaccination can be prepared by a standard method. For example, a culture supernatant containing the proteins, nucleic acid molecules or antibodies is concentrated, if necessary, and suspended together with an appropriate carrier or excipient in a buffer solution such as PBS, a nucleic acid molecule-stabilizing solution, or saline. Then, the suspension can be sterilized by filtration through a filter or the like according to the need and subsequently charged into an aseptic container to prepare the formulation for ID vaccination. The formulation for ID vaccination may be supplemented with a stabilizer, a preservative, and the like, according to the need. The composition thus obtained can be administered intradermally to a subject.

The invention also provides a method for generating an immune response in an animal comprising administering any of the proteins, peptides, nucleic acid molecules or compositions described above to an animal in an amount effective to stimulate the immune response. In one embodiment, the immune response comprises one or more of the production of memory CD8+ T cells specific for an expressed target antigen, the production of memory CD4+ T cells specific for an expressed target antigen, and the production of antibodies specific for an expressed target antigen. In one embodiment, at least some of the antibodies are neutralizing antibodies.

The invention further provides pharmaceutical compositions (e.g., vaccines) comprising the Leptospiral VM-proteins of the invention. In one embodiment, the composition comprises a pharmaceutically acceptable diluent, carrier, or excipient carrier. The composition may also contain an aqueous medium or a water-containing suspension, to increase the activity and/or the shelf life of the composition. The medium/suspension can include salt, glucose, pH buffers, stabilizers, emulsifiers, and preservatives.

In some embodiments, the composition further comprises an adjuvant, e.g., including, but not limited to: muramyl dipeptide; aluminum hydroxide; saponin; polyanions; anamphipatic substances; Bacillus Calmette-Guerin (BCG); endotoxin lipopolysaccharides; keyhole limpet hemocyanin (GKLH); and cytoxan.

In one aspect, the invention provides a method of administering a therapeutically effective compositions according to the invention. The desired therapeutic effect comprises one or more of: reducing or eliminating bacterial load, increasing numbers of CD4+ and/or CD8+ T cells or antibodies which recognize the encoded antigen; increasing overall levels of CD4+ T cells; increasing levels of neutralizing antibodies which recognize the antigen; decreasing the number of or severity of symptoms of a disease; decreasing the expression of a cancer specific marker; decreasing size or rate of growth of a tumor; preventing metastasis of a tumor; preventing infection by a pathogenic organism; and the like. The therapeutic effect may be monitored by evaluating biological markers and/or abnormal physiological responses. Generally, an effective dose of a composition according to the invention comprises a titer that can modulate an immune response against the encoded antigen such that memory T cells are generated which are specific for the encoded antigen.

Both the dose and the administration means can be determined based on the condition of the patient (e.g., age, weight, general health), risk for developing a disease, or the state of progression of a disease.

In one embodiment, an effective amount of recombinant virus ranges from about 10 μl to about 25 μl of saline solution containing concentrations, of from about 1×10¹⁰ to 1×10¹¹ plaque forming units (pfa) virus/ml.

In one embodiment of the invention, a priming immunization is performed, followed, optionally, by a booster immunization at about 3-4 weeks after the priming immunization. However, subsequent immunizations need not be provided until at least about 4 months, about 6 months, about 8 months, about 12 months, about 10 months, about 16 months, about 18 months, or about 24 months after the priming boost. In one aspect, the composition is a prophylactic vaccine, administered to a patient who has not tested positive for the vaccine antigen, e.g., such as to an individual who is at risk of exposure to Leptospira bacteria. In another aspect, the vaccine is administered therapeutically, to a person who is seropositive for the vaccine antigen (although not necessarily displaying symptoms) (i.e., such as to a Leptospria positive individual).

### Kits

The invention also includes a kit comprising one or more of the compositions described herein. For example, in one embodiment, the kit comprises a Leptospiral VM protein, a variant or fragment thereof, a nucleic acid molecule encoding a Leptospiral VM protein, a variant or fragment thereof or a fusion construction comprising a Leptospiral VM domain. In one embodiment, the kit comprises an anti-Leptospiral VM antibody or nucleic acid molecule encoding the same. In one embodiment, the kit comprises instructional material which describes the use of the composition. For instance, in some embodiments, the instructional material describes administering the composition(s), to a subject as a therapeutic treatment or a non-treatment use as described elsewhere herein. In an embodiment, the kit further comprises one or more additional reagents for use in an assay, for example in an immunoassay of the invention.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1: Leptospiral VM (“Virulence Modifying”) Proteins

Leptospiral VM protein vaccines comprising one or multiple VM proteins were designed and tested for their ability to protect mice from Leptospiral challenge. The pan-vaccines comprising multiple VM proteins shows complete protection from death.

Leptospiral VM proteins were expressed in two ways. L. interrogans serovar Lai proteins, LA3490, tLA3490, LA0620 and LA1402 were produced as mCherry fusions. Part II, L. interrogans serovar Copenhageni proteins LIC12340 (conserved ortholog of LA1400) and LIC12985 (conserved ortholog of LA0591) were not produced as mCherry fusions.

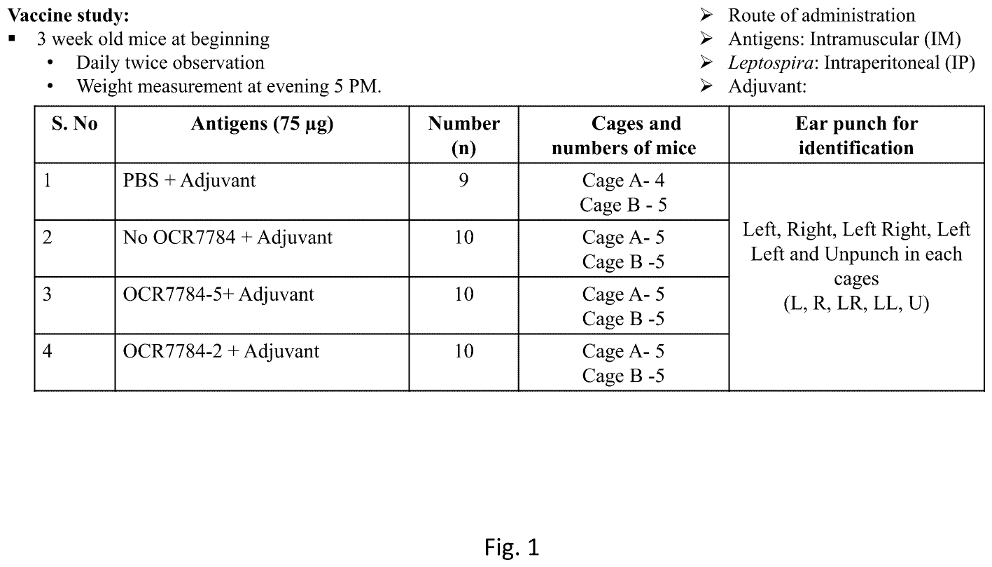

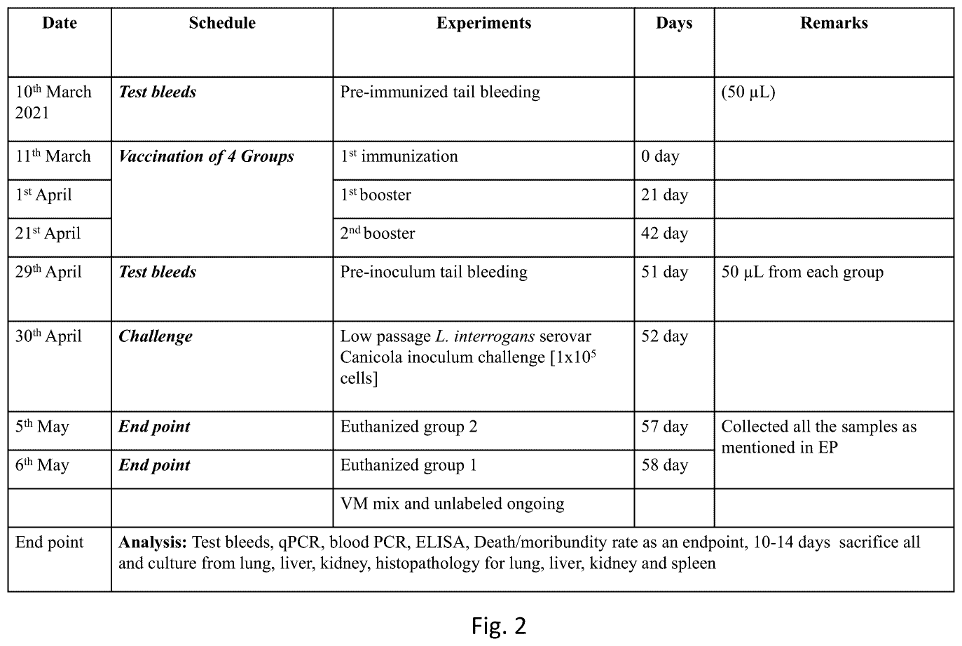

FIGS. 1 and 2 show the pan-vaccine challenge study design and the experimental schedule.

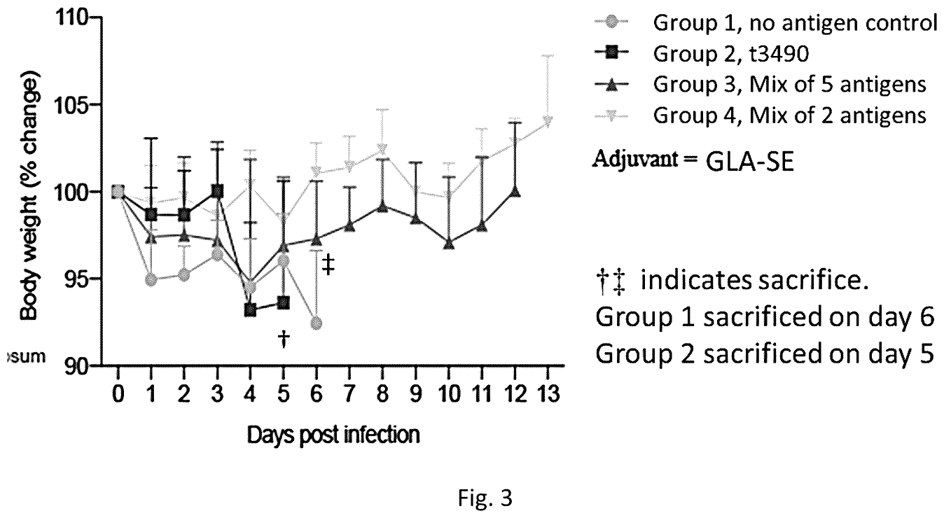

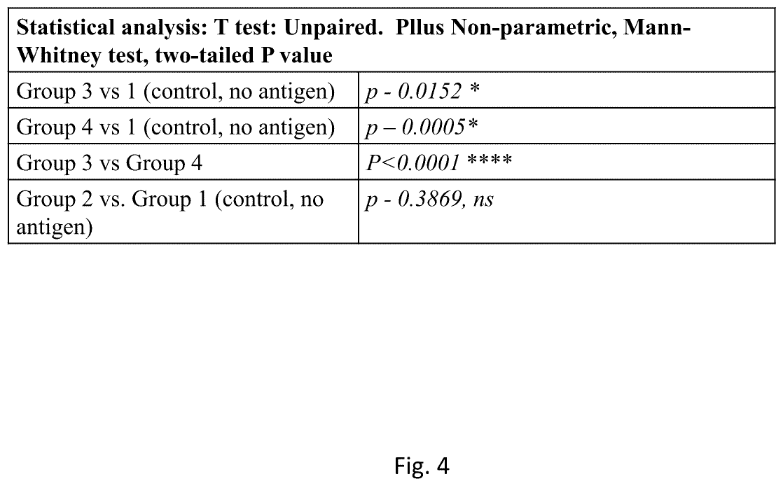

Immunized C3H/HeJ mice are protected from death/weight loss by pan-vaccine after lethal challenge (low passage L. interrogans serovar Canicola) (FIG. 3 and FIG. 4).

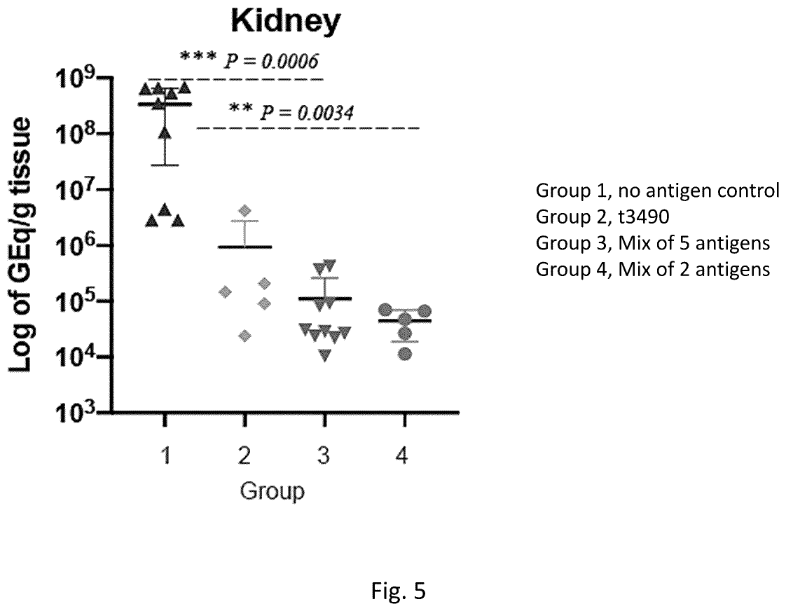

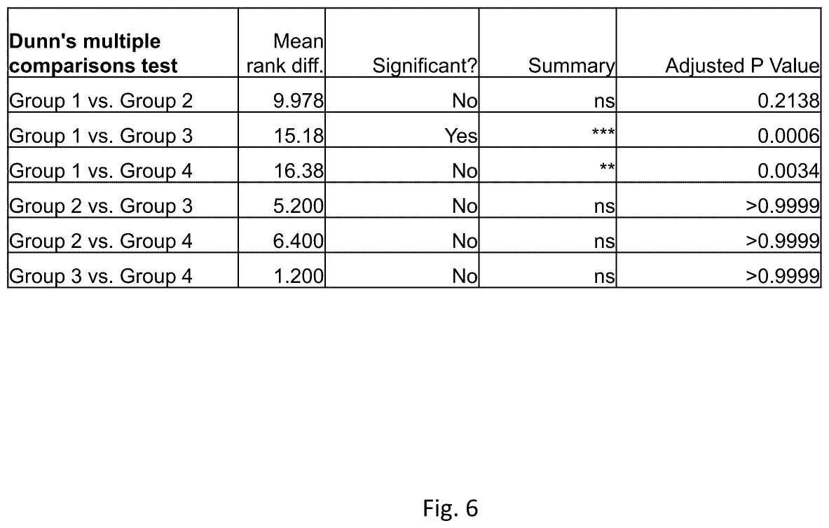

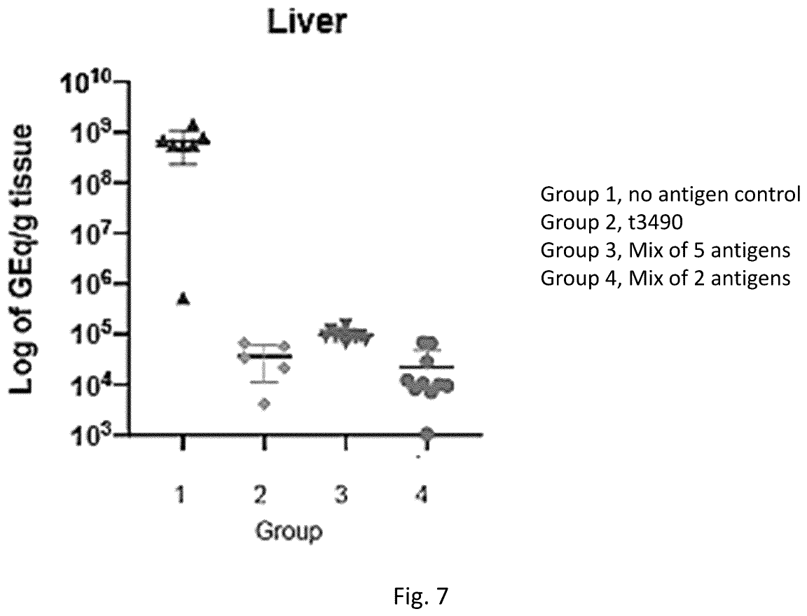

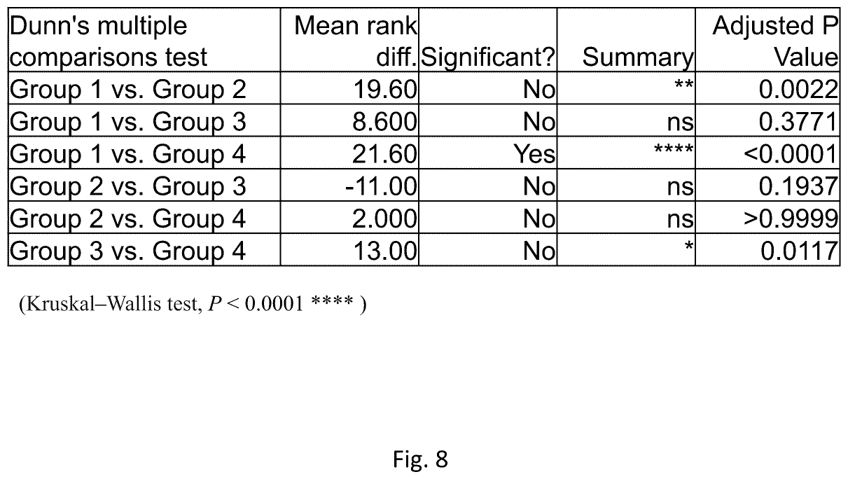

VM protein vaccines reduced bacterial load in kidney (FIG. 5 and FIG. 6) and lung (FIG. 7 and FIG. 8) compared to PBS negative control.

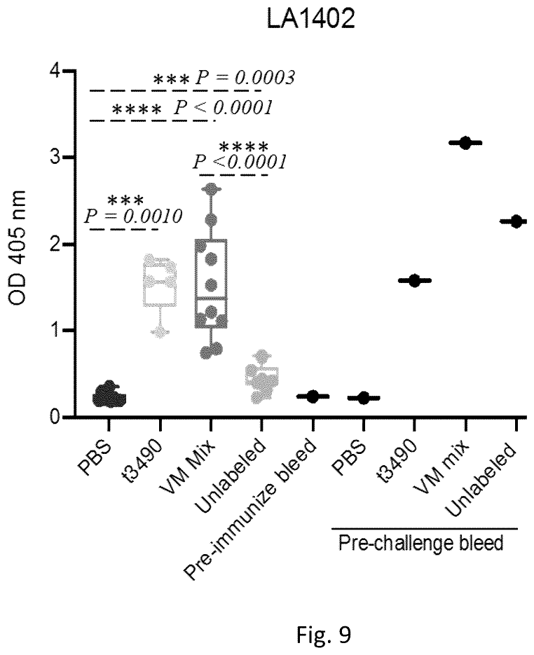

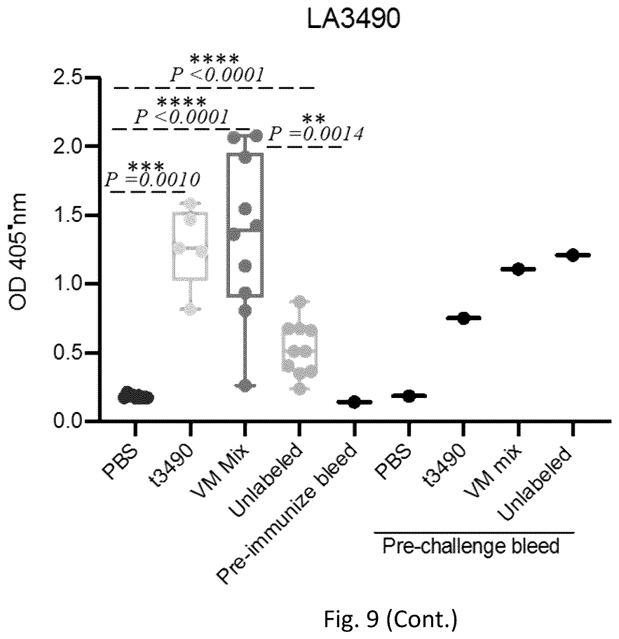

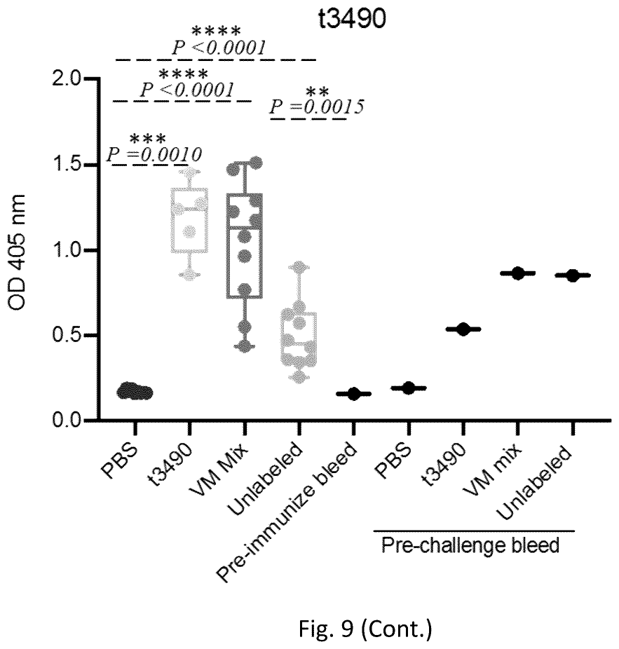

Cross-reactive VM protein antibodies were detected at pre-challenge (FIG. 9).

### VM Protein-mCherry Fusion Protein Vaccine Components

Expression of full length LA3490 (amino acids 40-639), truncated tLA3490 (short, ricin B domain alone, amino acids 40-174), full length LA0620 (amino acids 41-637), and full length LA1402 (amino acids 28-641) into XhoI/NcoI restriction sites of pET32b. These proteins are expressed as a fusion with thioredoxin (TrxA), S-tag, His6 affinity/epitope tag and enterokinase cleavage site at the amino terminus, and with an additional enterokinase cleavage site, and mCherry fusion and His6 affinity/epitope tag at the carboxy terminus. Linker is (Gly₄Ser)₅ as a hinge.

### Challenge Study: Protection and Reduction in Bacterial Load Results

Challenge bacterial inoculum: Leptospira interogans serovar Canicola

Group 1: PBS plus Glucopyranosyl Lipid A (GLA), formulated in a stable oil-in-water nano-emulsion (SE), (GLA-SE) adjuvant. No protection, about 8.5 log 10 bacterial copies per g tissue.

Group 2: t3490 (ricin B domain of LA3490), plus adjuvant. Reduced bacterial load by about 2.5 log 10 magnitude in kidney and reduced bacterial load by about 4 log 10 magnitude in liver, compared to Group 1.

Group 3: (Five proteins) recombinant full length LA3490, LA0620, LA1402 plus LIC12340 (LA1400) and LIC12985 (LA0591 plus GLA-SE adjuvant. Complete protection from death, and reduced bacterial load by about 3.5 log 10 magnitude in kidney and reduced bacterial load by about 3.9 log 10 magnitude in liver, compared to Group 1.

Group 4: (Two proteins) recombinant full length LIC12340 (LA1400) and LIC12985 (LA0591) plus GLA-SE adjuvant. Complete protection from death, and reduced bacterial load by about 4.0 log 10 magnitude in kidney and reduced bacterial load by about 4.1 log 10 magnitude in liver, compared to Group 1.

LA3490-mCherry expressed in pET32b
Full length LA3490 DNA sequence
(E.coli-preferred codons) of
construct cloned into pET32b
(SEQ ID NO: 1)
TTCGAATACGGCGTGAATCATACCCATATTCATGCCCTGAGTAAAATTGA
ATATAGTGTTATTCAGAAGCCGACCGATCCGCCGAAAGATAAACCGATTA
AGGTTATTGTTAGCGATGGTGGCAAATTTTGTTATGGTCCGAATTTTAGC
GGCGGCGAAAGCTATATTATTATTGAACAGTGTTGGCAGATGCATGTGAT
GAATGCACGCTATGATGTGTTTCAGCGTATTAGTTATAACATTAACAACA
CCTGGCTGTGTATTACCGCACCGGAAAAAGTGATTAAGGCAGAAAAGAAT
TGGGATTATGTGCATCTGCGTCCGTGCACCATTAATGATCCGCTGCAGCG
CTGGATTATTAAGAATAATAGTTTTTGGACCGCCAATGGTTTTTATCGTC
TGAAAGATTATAACTGGTACGGCTATATTAGCCGTAATAGTGGTGACCGT
TATAATCATACCCTGGATAGCAGCATGAATGATTGGGTTAATACCATTGC
CACCCCGGGTAATATTAGCATTCAGACCAGTATTGCCTGGAATCTGCAGA
CCACCGAAGGTCAGGAACGTTATTTTATTCGCTGGGGTGGCAGCGATAAA
AATACCACCCCGCTGTATTATAATCCGGAAAATGGCCATCTGGCCCAGTA
TGATCCGATTAGCGGTAGCCTGTATTGCATGTATAGTCAGGTGGATAATT
ATCAGTGGAATTGGGTTAAATGGAAATGGTGTAGCGATCTGCTGGAAAGC
AAAAGTAAAGGCAATCCGACCTTTTGGAATGTGTTTTTCGAAACCGATCA
GGGTGGCATGATTACCGATTATAAAGGTAATGCCCTGCGCGTTACCCGCT
ATGGTAGCAATTGGGGTAGCGCATATACCGCCAAACCGAGTTATCTGGAA
AAAGATACCACCAATAGCCCGACCAGCCTGTTTGTGGTGAATAAGGATCT
GCTGGATTGGACCCGTTATACCGCCAGTAATCTGGGTAAAACCGGTCAGT
ATTGCCCGGCCGGCAAACGCGAAAATATTGTGCATCGCCGCGTTAAACGC
GAACTGCCGCCGGATTTTCAGCTGACCGAAGCCTGGATTCGCCGCCTGTA
TGAAATTGCCACCAGCGTTAGCGCAGAAAGCGAAACCCGTGTGAGCGGTA
TTTGTGGCCCGTGTGCACTGCATAGTTTTCAGATGCTGGCAGAACTGCTG
GAATATCATAGCCGCGAACCGCTGCAGAGTGGTGGCTATTTCTTTGATAC
CGCACCGAATACCGATCCGTTTATTAGTTTTGGCCAGCGTTATCCGCATC
TGGAACGTCTGCTGGAAGATATTCCGAAAAAATATGCACCGTATCCGCAT
TATAGCACCCAGAGCTTTCTGAGCTTTGCCAGTATTGATAGCATGCTGCC
GCAGTATTTTTGGAGTGCAAGTACCGAATTCACTAATCGTGATGAAATTC
TGAGCCATATTAGTAGTCTGATTAATAGTCCGGCAGGCAGCATTTGGCTG
GGTGTGATGGAACAGCAGCATCCGGATGGTACCATTACCGGTCATGCAGC
ACCGATTCTGCGTATTAGTCAGGGTCTGGTTGTGATTCCGACCAATGTTC
ATCTGTGGACCCTGGAAGAATTTCGCCGCTTTCTGATTCCGACCACCGAA
CTGAGCCAGATTGTGGCCAATCTGGAAGGTAGCAATACCCTGATTCGTTT
TACCACCATTCAGAGTCTGGGCATGCTGACCACCAATATGTTTGATAGCA
TGGTTAGCAATCGCAATTGTACCGGTGAAGGTGAAGATCGTCGCGGTAGT
GGCGAATATCCGACCAGTACCAGTGTTAATCAGTGCCCGAGTGGTCGTTG
TGCCCTGCCGTTTGGCGGTGGTGGCAGCGGTGGTGGTGGTAGTGGCGGCG
GTGGCAGCGACGATGATGATAAAATGGTTAGTAAAGGTGAAGAAGATAAC
ATGGCAATTATTAAGGAATTCATGCGCTTTAAAGTGCATATGGAAGGCAG
CGTGAATGGTCATGAATTTGAAATTGAAGGTGAAGGCGAAGGTCGTCCGT
ATGAAGGTACCCAGACCGCAAAACTGAAAGTGACCAAAGGTGGCCCGCTG
CCGTTTGCATGGGATATTCTGAGCCCGCAGTTTATGTATGGCAGTAAAGC
ATATGTTAAGCATCCGGCCGATATTCCGGATTATCTGAAACTGAGTTTTC
CGGAAGGCTTTAAATGGGAACGCGTTATGAATTTTGAAGATGGCGGTGTT
GTGACCGTGACCCAGGATAGCAGTCTGCAGGATGGTGAATTCATCTATAA
AGTGAAACTGCGTGGCACCAATTTTCCGAGCGATGGCCCGGTTATGCAGA
AAAAGACTATGGGTTGGGAAGCCAGTAGTGAACGCATGTATCCGGAAGAT
GGTGCACTGAAAGGTGAAATTAAGCAGCGCCTGAAACTGAAAGATGGTGG
TCATTATGATGCAGAAGTGAAAACCACCTATAAAGCAAAAAAGCCGGTTC
AGCTGCCGGGCGCCTATAATGTGAATATTAAGCTGGATATTACCAGTCAT
AACGAAGATTATACCATTGTTGAACAGTATGAACGCGCCGAAGGCCGCCA
TAGTACCGGCGGTATGGATGAACTGTATAAAtaa
Full length LA3490 amino acid sequence
Name:LA_3490_mCherry, Sequence:Protein,
5′Sequence:, 3′Sequence:, Sequence
length:877
(SEQ ID NO: 2)
FEYGVNHTHIHALSKIEYSVIQKPTDPPKDKPIKVIVSDGGKFCYGPNFS
GGESYIIIEQCWQMHVMNARYDVFQRISYNINNTWLCITAPEKVIKAEKN
WDYVHLRPCTINDPLQRWIIKNNSFWTANGFYRLKDYNWYGYISRNSGDR
YNHTLDSSMNDWVNTIATPGNISIQTSIAWNLQTTEGQERYFIRWGGSDK
NTTPLYYNPENGHLAQYDPISGSLYCMYSQVDNYQWNWVKWKWCSDLLES
KSKGNPTFWNVFFETDQGGMITDYKGNALRVTRYGSNWGSAYTAKPSYLE
KDTTNSPTSLFVVNKDLLDWTRYTASNLGKTGQYCPAGKRENIVHRRVKR
ELPPDFQLTEAWIRRLYEIATSVSAESETRVSGICGPCALHSFQMLAELL
EYHSREPLQSGGYFFDTAPNTDPFISFGQRYPHLERLLEDIPKKYAPYPH
YSTQSFLSFASIDSMLPQYFWSASTEFTNRDEILSHISSLINSPAGSIWL
GVMEQQHPDGTITGHAAPILRISQGLVVIPTNVHLWTLEEFRRFLIPTTE
LSQIVANLEGSNTLIRFTTIQSLGMLTTNMFDSMVSNRNCTGEGEDRRGS
GEYPTSTSVNQCPSGRCALPFGGGGSGGGGSGGGGSDDDDKMVSKGEEDN
MAIIKEFMRFKVHMEGSVNGHEFEIEGEGEGRPYEGTQTAKLKVTKGGPL
PFAWDILSPQFMYGSKAYVKHPADIPDYLKLSFPEGFKWERVMNFEDGGV
VTVTQDSSLODGEFIYKVKLRGTNFPSDGPVMQKKTMGWEASSERMYPED
GALKGEIKQRLKLKDGGHYDAEVKTTYKAKKPVQLPGAYNVNIKLDITSH
NEDYTIVEQYERAEGRHSTGGMDELYK
LA0620-mCherry expressed in pET32b
Full length DNA sequence (E.coli-
preferred codons) of construct
cloned into pET32b
(SEQ ID NO: 3)
AGCAGTAAAATCGAATACAGCGTGATTCAGAAACCGACCGATCCGCCGAA
AGATAAACCGATTAAGGTTATTGTTAGCGATGGCGGTAAATTTTGTTATG
GTCCGAATTTTAGCGGTGGTGAAAGCTATATTATTATTGAACAGTGTTGG
CAGATGCATGTTATGAATGCCCGCTATGATGTTTTTCAGCGTATTAGCTA
TAACATTAACAACACCTGGCTGTGTATTACCGCACCGGAAAAAGTTATTA
AGGCCGAAGAAACCTGGGATTATGTTCATCTGCGTCCGTGCACCATTAAT
GATCCGCTGCAGCGTTGGATTATTAAGAATAATAGTTTCTGGACCGCCAA
TGGCTTTTATCGCCTGAAAGATTATAATTGGTACGGCTATATTAGCCGTA
ATAGTGGTGACCGCTATAATCATACCCTGGACCCTAGTATGAATGATTGG
GTTAATACCATTGCCACCCCGGGCAATATTAGCATTCAGACCAGTATTGC
CTGGAATCTGCAGACCACCGAAGGTCAGGAACGTTATTTTATTCGTTGGG
GCAGTAGTAATAAGAATACCACCCCGCTGTATTATAATCCGGAAAATGGC
CATCTGGCACAGTATGATCCGATTAGTGGTAGTCTGTATTGCATGTATAG
TCAGGTGGATAATTATCAGTGGAATTGGGTGAAATGGAAATGGTGCAGCG
ATAGCCTGGAAAGCAAAAGCAAAGGTAATCCGACCTTTTGGAATGTTTTC
TTTGAAACCGATCAGGGCGGTATGATTACCGATTATAAAGGCAATGCCCT
GCGTGTGACCCGTTATGGTAGTAATTGGGGCGTGGCATATACCGCCAAAC
CGGATTTTGTTAAAACCGATACCAAAAATAGCCCGACCAGCCTGTTTGTG
GTTGATAAAAGTCTGCTGGATTGGACCCGCTATACCAGCAGTAATCTGGG
TAAAACCGAACAGTATTGCCCGGCCGGTAATAAGGAAAGTGTGGTGCATA
AAAAAGCCAAACGCACCCTGCCGCCGGATTTTCAGCTGACCGAAGCCTGG
ATTCGCCGTCTGTATGAAATTGCCCGCACCGATCCGAGCAGTCGCACCAG
TCGCGGTGTTTGCGGTGTTTGTATGCTGCAGGCCCTGCAGATGATTGCAG
AACTGCAGGAATATCATAGTCAGGGCCCGCTGCAGAGCGGTGGTTATTTC
TTTAATACCGCACCGAATACCAATCCGTTTATTAGTTTTGGTCAGCGTTA
TCCGCATCTGGATCGCCTGCTGGTTGATATCTATCGCGTGTTTGATCATT
TCTTTGATACCAGCCATACCCTGGGTTATCTGAGTGCCATGAATCTGCTG
CCGCAGTATGAATGGGGTCGTACCCGCGAATTTTCTACCATGAGTGAAAT
TCGCAGCCATATTCGTAGCCTGATTACCAGCCCGCCGGGTAATATTTGGC
TGGTGCTGATGACCATGATCTATCCGGATGGCACCCGCGGTGGTCATGCA
GTTCCGATTCTGCGTACCCCGCAGGGCCTGGTGGTTATTGAAACCACCAT
GGCAACCGCAACCTTTGAAGAATATCGTGCCGCACTGCGTCCGACCACCG
ATTTTGAACAGATTATTCGTAATCTGCGTGGTCCGAATAATATTCTGATT
GGTCTGAGCACCCTGCAGCTGGGTCGTTTTTATCATAATCCGCTGGATAG
CATGATTAGTAATCGCAATTGTACCGGTGAAGGTAGCGATCGCCGTGGTA
CCGGCGGCTATCCGGCAAGCACCAGCGTGAATCAGTGCAGTAGTAAAAGC
AGCCGTTGCAGCCTGCAGGGCGGTGGTGGTAGTGGTGGCGGCGGTAGCGG
TGGTGGCGGTTCAGATGATGATGATAAAATGGTGAGTAAAGGTGAAGAAG
ATAATATGGCCATTATTAAGGAATTCATGCGCTTTAAAGTGCATATGGAA
GGTAGCGTTAATGGTCATGAATTTGAAATTGAAGGTGAAGGTGAAGGCCG
TCCGTATGAAGGCACCCAGACCGCCAAACTGAAAGTTACCAAAGGTGGCC
CGCTGCCGTTTGCCTGGGATATTCTGAGTCCGCAGTTTATGTATGGTAGT
AAAGCCTATGTTAAGCATCCGGCAGATATTCCGGATTATCTGAAACTGAG
CTTTCCGGAAGGCTTTAAATGGGAACGTGTTATGAATTTTGAGGATGGTG
GCGTTGTTACCGTTACCCAGGATAGCAGCCTGCAGGATGGCGAATTCATC
TATAAAGTGAAACTGCGCGGTACCAATTTTCCGAGTGATGGTCCGGTTAT
GCAGAAAAAGACTATGGGCTGGGAAGCAAGTAGCGAACGTATGTATCCGG
AAGATGGTGCCCTGAAAGGCGAAATTAAGCAGCGTCTGAAACTGAAAGAT
GGCGGCCATTATGATGCCGAAGTTAAAACCACCTATAAAGCAAAAAAGCC
GGTTCAGCTGCCGGGTGCATATAATGTGAATATTAAGCTGGATATCACCA
GCCATAATGAAGATTATACCATTGTTGAACAGTACGAACGCGCCGAAGGC
CGTCATAGCACCGGTGGTATGGATGAACTGTATAAAtaa
Amino acid sequence
Name:Full_LA_0620_mCherry, Sequence:
Protein, 5′Sequence:, 3′Sequence:,
Sequence length:862
(SEQ ID NO: 4)
SSKIEYSVIQKPTDPPKDKPIKVIVSDGGKFCYGPNFSGGESYIIIEQCW
QMHVMNARYDVFQRISYNINNTWLCITAPEKVIKAEETWDYVHLRPCTIN
DPLQRWIIKNNSFWTANGFYRLKDYNWYGYISRNSGDRYNHTLDPSMNDW
VNTIATPGNISIQTSIAWNLQTTEGQERYFIRWGSSNKNTTPLYYNPENG
HLAQYDPISGSLYCMYSQVDNYQWNWVKWKWCSDSLESKSKGNPTFWNVF
FETDQGGMITDYKGNALRVTRYGSNWGVAYTAKPDFVKTDTKNSPTSLFV
VDKSLLDWTRYTSSNLGKTEQYCPAGNKESVVHKKAKRTLPPDFQLTEAW
IRRLYEIARTDPSSRTSRGVCGVCMLQALQMIAELQEYHSQGPLQSGGYF
FNTAPNTNPFISFGQRYPHLDRLLVDIYRVFDHFFDTSHTLGYLSAMNLL
PQYEWGRTREFSTMSEIRSHIRSLITSPPGNIWLVLMTMIYPDGTRGGHA
VPILRTPQGLVVIETTMATATFEEYRAALRPTTDFEQIIRNLRGPNNILI
GLSTLQLGRFYHNPLDSMISNRNCTGEGSDRRGTGGYPASTSVNQCSSKS
SRCSLQGGGGSGGGGSGGGGSDDDDKMVSKGEEDNMAIIKEFMRFKVHME
GSVNGHEFEIEGEGEGRPYEGTQTAKLKVTKGGPLPFAWDILSPQFMYGS
KAYVKHPADIPDYLKLSFPEGFKWERVMNFEDGGVVTVTQDSSLQDGEFI
YKVKLRGTNFPSDGPVMQKKTMGWEASSERMYPEDGALKGEIKQRLKLKD
GGHYDAEVKTTYKAKKPVQLPGAYNVNIKLDITSHNEDYTIVEQYERAEG
RHSTGGMDELYK
t3490 (LA3490, amino acids 40-174,
mCherry fusion, expressed in prSET
DNA sequence (E.coli-preferred codons)
of construct cloned into pRSET
(SEQ ID NO: 5)
CAAAAGCCTACTGACCCACCCAAGGATAAACCAATTAAGGTGATTGTCTC
AGACGGGGGTAAATTTTGCTACGGGCCGAACTTTTCTGGCGGGGAGAGTT
ACATCATTATCGAACAGTGTTGGCAAATGCACGTTATGAACGCTCGTTAT
GATGTATTCCAACGCATCTCTTACAATATCAATAACACATGGCTGTGTAT
CACGGCCCCCGAGAAAGTAATCAAGGCCGAAAAAAACTGGGACTATGTCC
ATCTGCGCCCATGTACGATCAACGACCCACTGCAGCGTTGGATCATCAAA
AATAATAGTTTCTGGACAGCTAATGGATTCTACCGTTTAAAAGATTACAA
TTGGTATGGCTATATCAGCCGCAATTCCGGTGACCGTTATAACCATACTC
TGGATGGGGGTGGAGGCAGCGGTGGGGGCGGTTCAGGGGGAGGAGGCTCA
GATGACGACGACAAAATGGTATCGAAAGGTGAGGAAGATAATATGGCCAT
CATTAAGGAATTTATGCGCTTCAAGGTTCACATGGAAGGATCGGTAAATG
GTCACGAATTTGAGATCGAAGGCGAAGGTGAGGGACGCCCATACGAGGGG
ACCCAAACTGCTAAACTTAAGGTAACAAAGGGTGGTCCGTTGCCGTTCGC
CTGGGACATTCTGTCCCCGCAATTTATGTACGGTTCAAAGGCTTATGTAA
AACACCCAGCGGACATTCCGGACTACCTGAAACTGTCTTTTCCAGAGGGG
TTCAAATGGGAACGTGTGATGAACTTCGAAGATGGCGGTGTTGTAACCGT
TACACAGGATTCTTCATTACAAGACGGAGAGTTCATCTATAAGGTGAAGT
TGCGTGGAACCAACTTTCCTTCAGATGGTCCCGTAATGCAGAAGAAGACA
ATGGGCTGGGAAGCGTCATCTGAACGTATGTATCCAGAAGATGGAGCGCT
GAAGGGAGAAATTAAACAACGTCTGAAGCTTAAGGACGGGGGTCATTATG
ACGCGGAGGTCAAGACGACGTACAAGGCAAAGAAACCCGTCCAGTTACCC
GGTGCGTATAACGTCAACATTAAGCTGGACATTACGAGTCACAATGAAGA
TTACACCATCGTTGAACAGTATGAACGCGCAGAGGGACGCCACTCCACCG
GAGGTATGGACGAGCTGTACAAA
Amino acid sequence
(SEQ ID NO: 6)
QKPTDPPKDKPIKVIVSDGGKFCYGPNFSGGESYIIIEQCWQMHVMNARY
DVFQRISYNINNTWLCITAPEKVIKAEKNWDYVHLRPCTINDPLQRWIIK
NNSFWTANGFYRLKDYNWYGYISRNSGDRYNHTLDGGGGSGGGGSGGGGS
DDDDKMVSKGEEDNMAIIKEFMRFKVHMEGSVNGHEFEIEGEGEGRPYEG
TQTAKLKVTKGGPLPFAWDILSPQFMYGSKAYVKHPADIPDYLKLSFPEG
FKWERVMNFEDGGVVTVTQDSSLQDGEFIYKVKLRGTNFPSDGPVMQKKT
MGWEASSERMYPEDGALKGEIKQRLKLKDGGHYDAEVKTTYKAKKPVQLP
GAYNVNIKLDITSHNEDYTIVEQYERAEGRHSTGGMDELYK
LA1402-mCherry expressed in pET32b
DNA sequence (E.coli-preferred codons)
of construct cloned into pET32b
(SEQ ID NO: 7)
AGCAGCAGTAAAGCCAATTATAGTATTGCACAGAAACCGACCGATCCGCC
GAAAGATAAACCGATTAATATTGTTACCCACGATGGCAAAACCTATTGTT
ATAGTCCGGTGTTTAGTAAAGGTGAAGGCTATGTGTGGATTGAAAAATGT
GGCGATAATACCGCAAAAGCCCGCTATGATGTGTTTCAGCGCATTAGTTA
TAATATTAACAACACCTGGCTGTGTATTACCGCACCGGAACCGGTGGTGA
AAGGTAATGCACGCTGGGGTTATGTGAATCTGCGTCCGTGTACCATTAAT
GATCCGCTGCAGCGTTGGATTGTTAAAGAAAATAGTTTTTGGACCGCCGA
TGGCAAATATCGTCTGAAAGATACCAATTGGTATGGTTATATTAGCAAAA
CCAGCGGTGACAATTATAATCATACCCTGAATAGCAGTATGGATAATTGG
GTTAAAACCGTTGCAACCCCGGGCAATATTAGTATTCGTACCAGCATTAG
CTGGAATAGTGGCTGGGGTGACGGTATTTGGGATATTAATATGGCACCGA
GCGCCTATTTTATTCATAGCAAAGGCAGTAGCAAAGAAGATATTATTCCG
CTGTATTATAACCCGGAAAGTGGTCATATTGCACAGTATGATCCGAGTAG
TGGTCTGCTGAGCTGTATGTATAGTAAAATGACCGATAAATACGACTGGA
ATTGGGTTCAGTGGGGCAAATGCAGCGATGCCCCGATTAAGAAAGAAAAT
CCGGCCTTTTGGAATGTGTATTTTGTGGCAAATGCCGGCGGTATGATTAC
CGATTATAAAGGCAATATTCTGCGTGTTACCAAAGAAGGCCCGAATTGGG
GTGTGGCATATACCGCAAAACCGAGCTATCTGGAAAAAGATACCACCCAT
AGCCCGACCAGCGTGTTTACCGTGGATGTGGATCTGCTGAAATGGATTCG
TTATACCACCAGTAATCTGGGTAAAACCGATCAGTATTGCCCGGCAGGTA
AAAAAGAAAGTCGCATCTATCAGCGTGTGAAACGTAATCTGCCGAGCGAT
TTTCAGCTGAGTGTGGCATGGGTTCAGCGTCTGTATGATATTGCACGCAG
CGCCACCTTTGAAAGTGCCAATCCGGGCGCCATTCCGCAGCGTCATGGTG
CCTGTGGTGTTTGCCTGCTGCATAGTTTTCAGATGATTGCCGAACTGATG
GAATATCATAGCCGCGAACCGCTGACCAGTGGTGGTTATTTCTTTAATAC
CGCAAGTAATCGTGATCCGTTTCTGAGCTTTAGCCAGCGCTATCCGGAAC
TGGATCGTCTGGTTACCAATGTTCCGGTTGATTATGCAAATCGTGGTCGC
GTGCTGGCCTTTGCAAGTGCCATGATTATGCTGCCGCAGTATGAATGGGA
AAGCAGCAGCCCGCTGACCACCCGCAGCGATATTCAGAGTCATATTCGCA
GTCTGATTAATAGCCCGCCGGGTAGCATTTGGCTGGGCCTGCTGCGCCGT
CAGCGTGCAAATGGCAGTATTAGTGGCCATGCAGTGCCGATTCTGCGTAC
CAGCGAAGGCCTGGTGGTTATTCCGACCAATATGCCGACCGCAAGTCTGA
ATACCTATATTCAGAGCCTGGCACCGACCATGGACCCTAATGAAGTGATT
AATCGTCTGGAAAATGGCCGTACCCTGACCACCCTGACCACAATTCGCCC
GGTGGGCACCTATGAAACCCCGTTTAGTCTGACCGTTAGCAGCCGTGATT
GCACCGGTGACGGTGACGATCGTCGTGGTAGCGGTCGCTATCCGATTAGC
AGCCTGATTAATCAGTGCAGTGGTGGTCGCTGTATTCTGCAGGGCGGCGG
CGGTAGCGGCGGTGGTGGTAGCGGTGGTGGCGGTAGTGATGATGATGATA
AAATGGTTAGTAAGGGTGAAGAAGATAATATGGCCATTATTAAGGAATTC
ATGCGCTTTAAAGTGCATATGGAAGGCAGCGTGAATGGTCATGAATTTGA
AATTGAAGGCGAAGGTGAAGGTCGTCCGTATGAAGGCACCCAGACCGCAA
AACTGAAAGTGACCAAAGGCGGCCCGCTGCCGTTTGCATGGGATATTCTG
AGCCCGCAGTTTATGTATGGCAGCAAAGCATATGTGAAACATCCGGCCGA
TATTCCGGATTATCTGAAACTGAGCTTTCCGGAAGGTTTTAAATGGGAAC
GCGTGATGAATTTTGAAGATGGCGGTGTTGTGACCGTTACCCAGGATAGT
AGCCTGCAGGATGGTGAATTCATCTATAAAGTGAAACTGCGCGGTACCAA
TTTTCCGAGCGATGGCCCGGTTATGCAGAAAAAGACTATGGGCTGGGAAG
CAAGCAGCGAACGCATGTATCCGGAAGATGGCGCACTGAAAGGCGAAATT
AAGCAGCGTCTGAAACTGAAAGATGGCGGCCATTATGATGCAGAAGTTAA
AACCACCTATAAAGCAAAAAAGCCGGTTCAGCTGCCGGGTGCATATAATG
TGAATATTAAGCTGGATATCACCAGTCATAATGAAGATTATACCATTGTT
GAGCAGTATGAACGTGCCGAAGGTCGCCATAGTACCGGTGGTATGGATGA
ACTGTATAAAtaa
Amino acid sequence
Name:LA_1402_mCherry, Sequence:
Protein, 5′Sequence:, 3′Sequence:,
Sequence length:870
(SEQ ID NO: 8)
SSSKANYSIAQKPTDPPKDKPINIVTHDGKTYCYSPVFSKGEGYVWIEKC
GDNTAKARYDVFQRISYNINNTWLCITAPEPVVKGNARWGYVNLRPCTIN
DPLQRWIVKENSFWTADGKYRLKDTNWYGYISKTSGDNYNHTLNSSMDNW
VKTVATPGNISIRTSISWNSGWGDGIWDINMAPSAYFIHSKGSSKEDIIP
LYYNPESGHIAQYDPSSGLLSCMYSKMTDKYDWNWVQWGKCSDAPIKKEN
PAFWNVYFVANAGGMITDYKGNILRVTKEGPNWGVAYTAKPSYLEKDTTH
SPTSVFTVDVDLLKWIRYTTSNLGKTDQYCPAGKKESRIYQRVKRNLPSD
FQLSVAWVQRLYDIARSATFESANPGAIPQRHGACGVCLLHSFQMIAELM
EYHSREPLTSGGYFFNTASNRDPFLSFSQRYPELDRLVTNVPVDYANRGR
VLAFASAMIMLPQYEWESSSPLTTRSDIQSHIRSLINSPPGSIWLGLLRR
QRANGSISGHAVPILRTSEGLVVIPTNMPTASLNTYIQSLAPTMDPNEVI
NRLENGRTLTTLTTIRPVGTYETPFSLTVSSRDCTGDGDDRRGSGRYPIS
SLINQCSGGRCILQGGGGSGGGGSGGGGSDDDDKMVSKGEEDNMAIIKEF
MRFKVHMEGSVNGHEFEIEGEGEGRPYEGTQTAKLKVTKGGPLPFAWDIL
SPQFMYGSKAYVKHPADIPDYLKLSFPEGFKWERVMNFEDGGVVTVTQDS
SLQDGEFIYKVKLRGTNFPSDGPVMQKKTMGWEASSERMYPEDGALKGEI
KQRLKLKDGGHYDAEVKTTYKAKKPVQLPGAYNVNIKLDITSHNEDYTIV
EQYERAEGRHSTGGMDELYK

VM Proteins without mCherry Fusion

Genes encoding L. interrogans serovar Copenhageni proteins encoding amino acids 31-627 of LIC12340 (>99% conserved ortholog of LA1400) and amino acids 23-314 of LIC12985 (>99% conserved ortholog of LA0591) were synthesized in E. coli-preferred codons and cloned into XhoI/NcoI restriction sites of pET32b. These proteins were expressed as a fusion with thioredoxin (TrxA), S-tag, His6 affinity/epitope tag and enterokinase cleavage site at the amino terminus, and His6 affinity/epitope tag at the carboxy terminus, all fusion partners encoded by the pET32b vector.

LIC12340 (conserved ortholog of LA1400)
DNA sequence (E.coli-preferred codons)
of construct cloned into pET32b
(SEQ ID NO: 9)
AGCAGTAAAAGCGATTATAGTATCGCACAGAAACCGGCAGATCAGCCGAA
AGATAAAAGTATTCAGGTGGTTATGCATGGTGGCAGCAATTATTGCTATA
GTCCGGTGTTTACCAAAGGTGAAGGTTATATTTGGATTGATTACTGCAGC
GATAATACCGCCAAAGCACGTTATGATGTGTTTCAGCGCATTAGCTATAA
TATTAATAACACCTGGCTGTGCATTACCGCACCGGAAACCGTTGTTAAAG
GCGAAGAAACCTGGAATTATGTGAATCTGCGTCCGTGTACCATTAATGAT
CCGCTGCAGCGCTGGATTGTTAAAGATAATAGTTTTTGGACCGCAAATGG
CCGTTATCGCCTGAAAGATACCAATTGGTATGGCTATATTAGTCGCAATA
GCGGCGATCGTTATGATCATACCCTGAATAGCAGTATGGATGATTGGATT
AAGACCGTGGCAATTCCGGGCAATATTAGTATTCAGACCAGCATTGCCTG
GGATCTGCAGACCACCGAAGGCAATGAACGTTATTTTATTCGTTGGGGCA
GTAGTAATAAGAATACCACCCCGCTGTATTATAATCCGGAAAGTGGTCAT
ATTGCACAGTATGATCCGAGTAGCGGCCTGCTGAATTGCATGTATAGTAA
AATGACCGATAAGTATGACTGGAATTGGGTGAAATGGGGCAAATGCAGCG
ATGCACCGATTAAGAAAGATAATCCGGCATTTTGGAATGTTTTCTTTGAA
ACCGATAAGGAAGGTGCAATTACCGATTATAAAGGTAATGTGCTGCGCGT
TACCCGTTATGGCCTGAATTGGGGCGTTGCATATACCGTGAAACCGAGTT
ATCTGGAAAAAGATACCACCCATAGCCCGACCAGTCTGTTTGTGATTGAT
AAAGATCTGCTGGATTGGACCCGTTATACCTATAGTAATCTGGGCAAAAC
CGATCAGTATTGTCCGGCAGGCAATAAGGAAAGCCTGGTTCGCAAACGCG
TTAAACGTAATCTGAATCTGCCGAGCGATTTTCAGCTGACCCGCGAATGG
ATTCAGCGCCTGTATGAAATTGCCCGCAGCAGTATTAGCCGTGCAATTCC
GTGTCGTGGCGTGTGTGGCGTTTGTATGCTGCATAGTTATCAGATGATTG
CAGAACTGCTGGAATATCATAGTCGTGGTCCGCTGACCGGCGGTGGTTAT
TTCTTTGATACCGCCCCGAATCGTGATCCGTTTATTAGCTTTAATCAGCG
TTATCCGCAGCTGAATGCCCTGCTGACCAATGTGCCGAGTTATGCAAATC
GCCCGGGCTTTGGCAGTACCCTGGTTATGCTGCCGCAGTATGAATGGACC
AGTAGCGATACCATTACCACCCGTAGTGGTCGCCTGCTGCATGCCCGTAG
CCTGATTAATAGCCCGCCGGGCAGTATTTGGCTGGGCCTGCTGCGCGGCC
GCGATGCAGATGGTAGCACCTGGGGTCATGCAGTGCCGATTCTGCGCACC
AGCCAGGGCATTGTGGTTATTCCGACCAATAGTCCGACCATGAGCCTGAA
TACCTATATTCGCAGCCTGGCACCGACCATGGATCCGAATGAAGTGATTA
ATCGTCTGGAAAATGGCAGTACCTTAACCGAACTGACCACCATTCAGCCG
GTTCGCATCTATGATATTCCGTTTAGCCTGACCGTGAGTACCCGTGATTG
CACCGGTGACGGCGATGGTCGTCGTGGTAGTGGCCGTTATCCGACCAGTA
GTCTGATTAATCAGTGTAGTGGTGGTCGCTGTATTCTGCAGtaa
Amino acid sequence
Name:LIC_12340 (LA_1400 ortholog),
Sequence:Protein, 5′Sequence:,
3′Sequence:, Sequence length:597
(SEQ ID NO: 10)
SSKSDYSIAQKPADQPKDKSIQVVMHGGSNYCYSPVFTKGEGYIWIDYCS
DNTAKARYDVFQRISYNINNTWLCITAPETVVKGEETWNYVNLRPCTIND
PLQRWIVKDNSFWTANGRYRLKDTNWYGYISRNSGDRYDHTLNSSMDDWI
KTVAIPGNISIQTSIAWDLQTTEGNERYFIRWGSSNKNTTPLYYNPESGH
IAQYDPSSGLLNCMYSKMTDKYDWNWVKWGKCSDAPIKKDNPAFWNVFFE
TDKEGAITDYKGNVLRVTRYGLNWGVAYTVKPSYLEKDTTHSPTSLFVID
KDLLDWTRYTYSNLGKTDQYCPAGNKESLVRKRVKRNLNLPSDFQLTREW
IQRLYEIARSSISRAIPCRGVCGVCMLHSYQMIAELLEYHSRGPLTGGGY
FFDTAPNRDPFISFNQRYPQLNALLTNVPSYANRPGFGSTLVMLPQYEWT
SSDTITTRSGRLLHARSLINSPPGSIWLGLLRGRDADGSTWGHAVPILRT
SQGIVVIPTNSPTMSLNTYIRSLAPTMDPNEVINRLENGSTLTELTTIQP
VRIYDIPFSLTVSTRDCTGDGDGRRGSGRYPTSSLINQCSGGRCILQ
LIC12985 (highly conserved ortholog
of LA0591) DNA sequence (E.coli-
preferred codons) of construct
cloned into pET32b
(SEQ ID NO: 11)
GTTAACCCGGATCAGTATTGTCCGGCAAGTAAAAAAGAAAATCATAATAT
CCGCATCAAGCGCACCCTGCCGCCGGATTTTCAGCTGACCGAAGAATGGC
TGCGTCGCCTGTATGATATTGCAACCAGTGCCAGTCTGACCGAAGGTCAG
ATTCATGGCATTTGTGGCGTTTGTCTGCTGCAGACCTTTCAGATGCTGGC
AGAACTGCAGGAATATCATAGTCATGGCCCGCTGCAGGGTGGCGGCTATT
TCTTTAATACCGCACCGGATACCGATCCGTTTGATAGTTTTCGTCAGCGC
TATCCGGAACTGGATACCATGCTGACCGATGCAGCCACCGCATATGGTCC
GGCATATAATACCACCCGTCTGCTGACCCTGGTGAGCGCCATGACCATGA
TGCCGCAGTATGAATGGACCCCGAGCCGTGAATTCACTACCCGTAGCGAT
ATGCATAGCCATATTCGCAGTCTGATTGATAGCCCGCCGGGTAGTATTTG
GCTGGGTCTGATGCAGCGTCGCGAAAGTGATGAAACCCTGCGCTGGCATG
CCCTGCCGATTCTGCGCACCAGTCAGGGTCTGATTGTTATTCAGACCCGT
GTTAGCACCATGAGCTTTGAACTGTATCGCCTGTATCTGACCCCGAGCAC
CAGTATTGTGCAGATTATTAATGATTACCTGGAAGAAGCAGATCGCACCC
TGACCGTTCTGGTTACCATTCAGCTGGAACAGGCATATCAGAATCTGTTT
GATTTTATGGTTAGCAATATGAACTGCACCGGCGAAGGTGAAAATCGCCG
TGGCAGTGGCGGCTATCCGACCAGCGCAACCGTGAATCAGTGTAGCGGTG
GTCGCTGCGCACTGCCGAATTGGtaa
Amino acid sequence
Name:LIC_12985 (LA_0591 ortholog),
Sequence:Protein, 5′Sequence:,
3′Sequence:, Sequence length:291
(SEQ ID NO: 12)
VNPDQYCPASKKENHNIRIKRTLPPDFQLTEEWLRRLYDIATSASLTEGQ
IHGICGVCLLQTFQMLAELQEYHSHGPLQGGGYFFNTAPDTDPFDSFRQR
YPELDTMLTDAATAYGPAYNTTRLLTLVSAMTMMPQYEWTPSREFTTRSD
MHSHIRSLIDSPPGSIWLGLMQRRESDETLRWHALPILRTSQGLIVIQTR
VSTMSFELYRLYLTPSTSIVQIINDYLEEADRTLTVLVTIQLEQAYQNLF
DFMVSNMNCTGEGENRRGSGGYPTSATVNQCSGGRCALPNW
Q8F6A9_LEPIN Ricin B-type lectin
domain-containing protein OS = Leptospira
interrogans serogroup Icterohaemorrhagiae
serovar Lai (strain 56601) OX = 189518
GN = LA_1400 PE = 4 SV = 2
(SEQ ID NO: 13)
MHGGSNYCYSPVFTKGEGYIWIDYCRDNTAKARYDVFQRISYNINNTWLC
ITAPETVVKGEETWNYVNLRPCTINDPLQRWIVKDNSFWTANGRYRLKDT
NWYGYISRNSGDRYDHTLDSSMDDWIETVAIPGNISIQTSIAWDLQTTEG
NERYFIRWGSSNKNTTPLYYNPESGHIAQYDPSSGLLNCMYSKMTDKYDW
NWVKWGKCSDAPIKKDNPAFWNVFFETDKEGAITDYKGNVLRVTRYGLNW
GVAYTVKPSYLEKDTTHSPTSLFVIDKDLLDWTRYTYSNLGKTDQYCPAG
NKESLGRKRVKRNLNLPSDFQLTREWIQRFYEIARSNISGTIPRRGVCGV
CMLHSYQMIAELLEYHSRGPLTSGGYFFDTAPNRDPFISFNQRYPQLNTL
LTDVPNYANRLGFGSTLVMLPQYEWTSSDTITTRSGRLLHARTLINSPPG
SIWLGLLRGRNANGSTWGHAVPILRTSQGIVVIPTNVLTMSLNTYIRSLA
PTMDPNEVINRLENGNTLTELTTIQPVRIYDIPFSLTVSTRDCTGDGDGR
RGSGRYPTSSLINQCSGGRCILQ
Q72PX7_LEPIC Uncharacterized protein
OS = Leptospira interrogans serogroup
Icterohaemorrhagiae serovar copenhageni
(strain Fiocruz L1-130) GN = LIC_12340
PE = 4 SV = 1 Signal sequence (bold) is
not included in the recombinant protein
construct.
(SEQ ID NO: 14)
MGRWIVLRVSLLVLIGIGFEYGINHTSINASSKSDYSIAQKPADQPKDKS
IQVVMHGGSNYCYSPVFTKGEGYIWIDYCSDNTAKARYDVFQRISYNINN
TWLCITAPETVVKGEETWNYVNLRPCTINDPLQRWIVKDNSFWTANGRYR
LKDTNWYGYISRNSGDRYDHTLNSSMDDWIKTVAIPGNISIQTSIAWDLQ
TTEGNERYFIRWGSSNKNTTPLYYNPESGHIAQYDPSSGLLNCMYSKMTD
KYDWNWVKWGKCSDAPIKKDNPAFWNVFFETDKEGAITDYKGNVLRVTRY
GLNWGVAYTVKPSYLEKDTTHSPTSLFVIDKDLLDWTRYTYSNLGKTDQY
CPAGNKESLVRKRVKRNLNLPSDFQLTREWIQRLYEIARSSISRAIPCRG
VCGVCMLHSYQMIAELLEYHSRGPLTGGGYFFDTAPNRDPFISFNQRYPQ
LNALLTNVPSYANRPGFGSTLVMLPQYEWTSSDTITTRSGRLLHARSLIN
SPPGSIWLGLLRGRDADGSTWGHAVPILRTSQGIVVIPTNSPTMSLNTYI
RSLAPTMDPNEVINRLENGSTLTELTTIQPVRIYDIPFSLTVSTRDCTGD
GDGRRGSGRYPTSSLINQCSGGRCILQ

### Example 2: Computational Intelligence Based Reassessment of the Leptospiral Novel PF07598 VM Gene Family of AB Toxin to Infer Pathogenesis Mechanisms

Pathogenetic mechanism and biology of Leptospira remains challenging despite identifying several leptospiral virulence factors, informative in vitro and small animal models. Vascular instability, liver and renal dysfunction and pulmonary hemorrhage are majorly seen in severe leptospirosis postulated to be caused by circulating toxins secreted by pathogenic Leptospira. Virulence Modifying (VM) proteins (PF07598), are a distinctive feature of group I pathogenic Leptospira and experimentally validated bona fide R-type lectin domain-containing exotoxins. VM proteins comprises tandem N-terminal ricin B chain-like β-trefoil domains and C-terminal DNase activity which led rapidly to chromosomal fragmentation and cell death.

Here, using computational tools and artificial intelligence-derived high-resolution three-dimensional structure of VM proteins using the DeepMind AlphaFold algorithm, the mechanisms of action of novel leptospiral toxins are explored at the sequence, atomic and structural level. The current finding shows that the PF07598 protein family share remarkable degree of characteristic conserved sequence motifs and structure similarities with plant derived ricin B-chain [N-terminal unique (QxW)₃ motifs], bacterial CARDs toxin [D3 domain, aromatic patches] and mammalian DNase [C-terminal catalytic residues] all exclusively encoded by one gene and one protein. Evolutionary divergent origins of the unique VM proteins provides major insights in pathogenicity and host tropism. Structure-function validation of VM proteins by mutagenesis approach offer unique opportunities to understand the pathogenesis mechanism and to develop the novel therapeutic and preventive measurement of leptospirosis.

### Leptospiral Novel R-Type Lectins: Characteristic Carbohydrate Binding Aromatic Patches and Presence of Sequence Motif (QXW); Strengthen VM Proteins are Bona Fide R-Type Lectins

The R-type lectins are members of a superfamily of proteins and all of them contain a carbohydrate-recognition domain (CRD) and binding functions for complex carbohydrates of glycoconjugates (glycoproteins, proteoglycans/glycosaminoglycans, and glycolipids) such as ricin B chain (Cummings et al., 2017, Cold Spring Harbor Laboratory Press). Ricin, a toxic protein from the castor bean (Ricinus communis) is the very first lectin discovered in plants (Olsnes et al., 1974, Nature, 249(458):627-31). R-type lectins domain is associated with binding domain (B-chain) of AB toxin (Varki et al., 2015, Cold Spring Harbor). The greater heterogeneity in the composition and structure of the B-chain most likely evolved to recognize a broad range of target cells (DiRienzo et al., 2014, New Journal of Science, 26.)

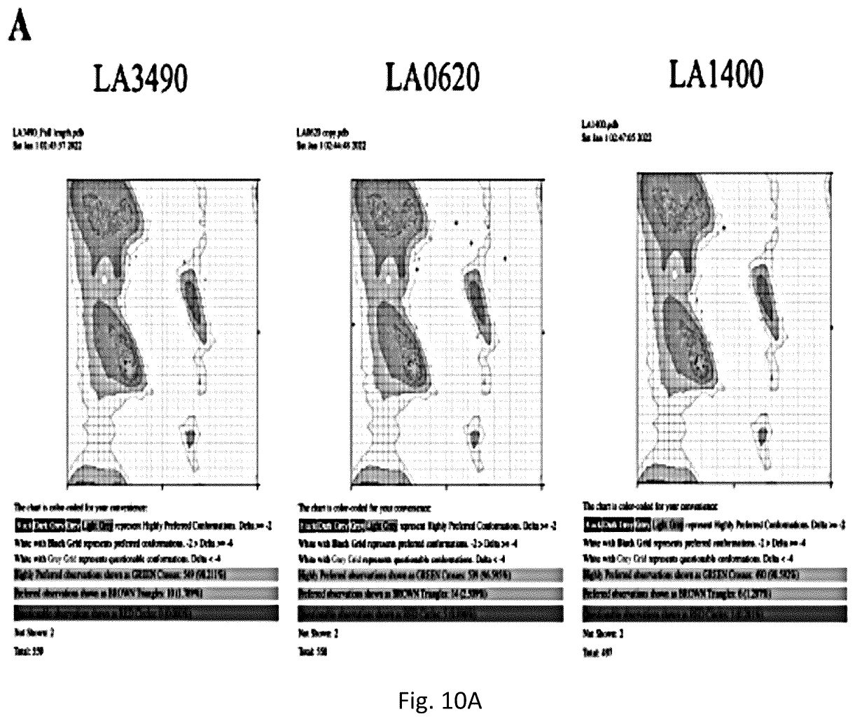

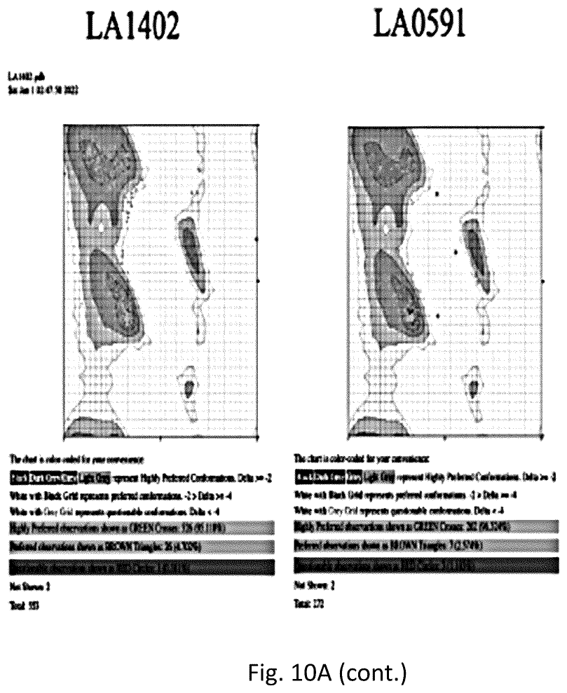

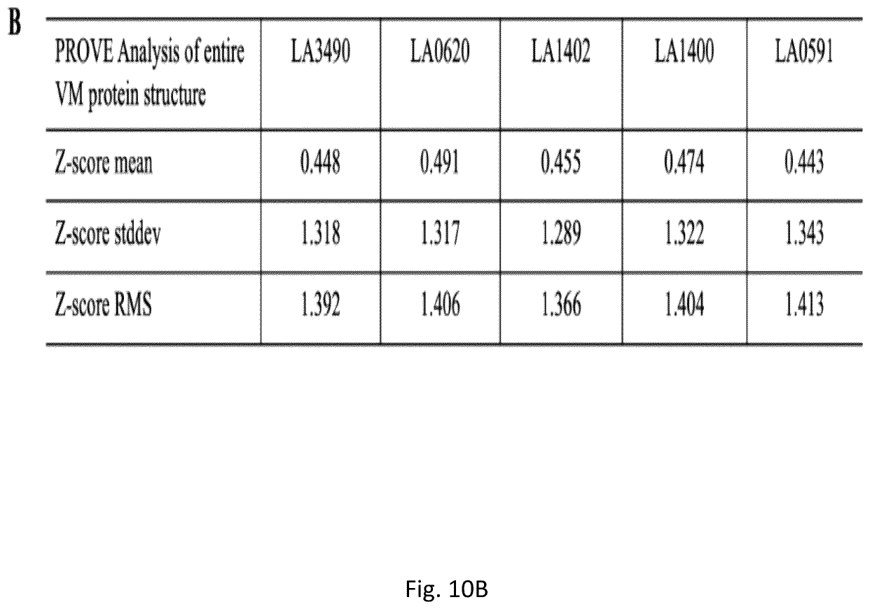

AlphaFold algorithm derived high resolution three-dimensional (3D) structural framework of VM proteins allowed us to understand the importance of structure-function relationship of the multi-globular proteins (in press) (Callaway et al., 2020, Nature, 588(7837):203-4; Jumper et al., 2020, predictioncenterorg/casp14/doc/CASP14_Abstracts; Senior et al., 2020, Nature, 577(7792):706-10). Pathogenic Leptospira encode ˜640 aa molecular weight, 12+ paralogs VM protein and they encode single polypeptide transcribed from a single genetic locus, distinct from most other bacterial AB toxins. The 3D structure of VM protein were validated by Ramachandran plot followed by Verify3D (doc-mbi.ucla.edu/verify3d/) [LA3490: 89.20%, LA0620: 92.15%, LA1402: 90.02%, LA1400: 98.60%, and LA0591: 84.98% of the residues have averaged 3D-1D score >=0.2 and passed the score] (Pontius et al., 1996, J Mol Biol, 264(1): 121-36). The structure was further validated by program PROVE (PROtein Volume Evaluation) (saves.mbi.ucla.edu/Jobs/1016444/prove/PROVE_PLOT.ps) and computed Z-score mean, Z-score stddev and Z-score RMS (FIG. 10).

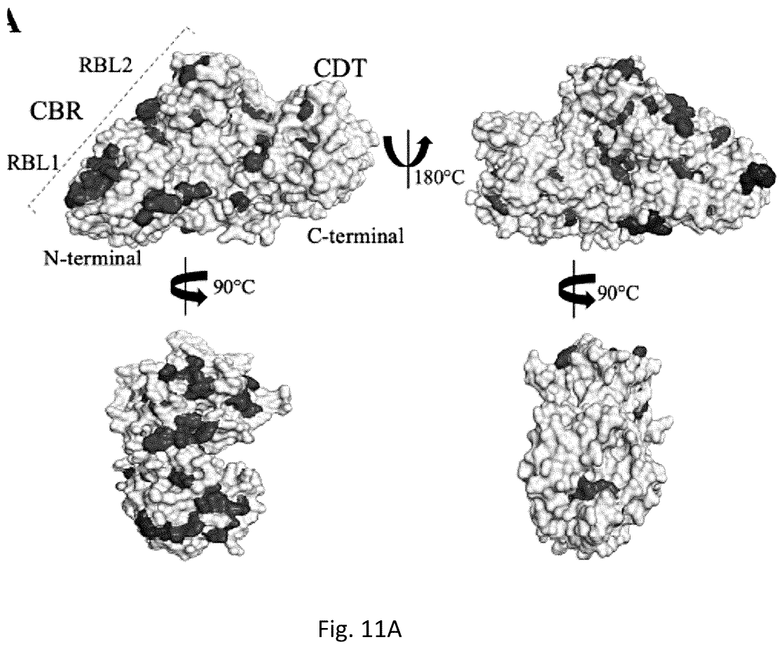

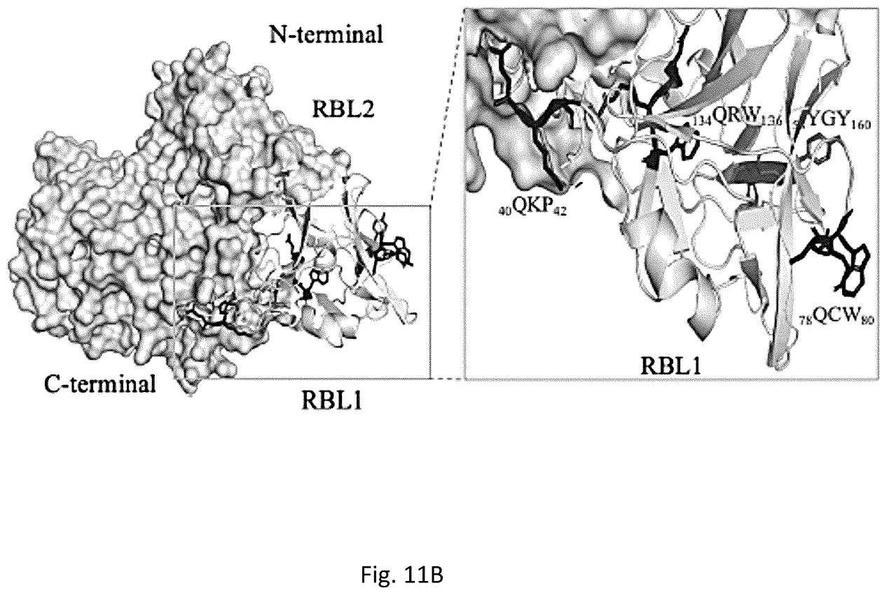

Paralogs of VM proteins comprising highly conserved tandem N-terminal trefoil-like lectin (RBL1 and RBL2) domain and variable C-terminal domain (Chaurasia et al., 2022, Front Microbiol, 13:859680). Notably, L. interrogans comprises natural CBR deletion variants (˜313 aa) containing a predicted signal sequence. Computational and in vitro experimental validation confirmed that only RBL1 domain (N-terminal region of LA3490, 40 aa-150 aa) structurally superimposed with ricin B-chain (PBD; 2AAI-B: 7 aa to 129 aa) of having RMSD-1.796 and solely responsible for binding to N-terminal galactosyl glycoprotein moiety present on host cell receptor (Chaurasia et al., 2022, Front Microbiol, 13:859680). RBL1 and RBL2 are rich in aromatic patches due to presence of surface exposed aromatic (tyrosine), and heterocyclic (phenylalanine and tryptophan) amino acids (FIG. 11A). These aromatic patches appear to play an important role in host receptor/carbohydrate recognition. In addition to aromatic patches, RBL1 domain comprises three sequences conserved QxW motif (₄₀QKP₄₂, ₇₈QCW₈₀, and ₁₃₄QRW₁₃₆) in β-trefoil domain similar to ricin B chain (FIG. 11B). Interestingly, multiple-sequence alignment of RBL1 with ricin B-chain shows that only 134QRW136 motif is conserved in both RBL1 and ricin B-chain and notably, tryptophan is replaced by proline in first QxW motif (40QKP42) in RBL1 domain. The QxW sequence conserved motif play an important role in receptor recognition and contributes to structural stabilization by hydrogen bonding within carbohydrate-binding motif (Hatakeyama et al., 2007, J Biol Chem, 282(52):37826-35; Hazes et al., 1995, Nat Struct Biol, 2(5):358-9; Hazes et al., 1996, Protein Sci, 5(8): 1490-501). In RBL1 domain, the sequence motif 158 YGY 160 is highly conserved in ricin B chain and assume to possess functional carbohydrate-binding ability similar to ricin B-chain. LA1402 and LA1400 are an ancestral VM protein in Group I pathogenic Leptospira and they belong to same cluster A. Computational analysis prediction suggest that these two proteins (LA1402 and LA1400) lack ₇₈QCW₈₀ motif which might explain the evolution of VM protein by successive gene duplications acquired the essentiality of ₇₈QCW₈₀ motif for binding to host cell surface/host tropism (Chaurasia et al., 2022, Front Microbiol, 13:859680; Fouts et al., 2016, PLOS Negl Trop Dis, 10(2):e0004403). The essentiality of binding of VM protein to the host receptor is the key step in understanding the mechanism of host-pathogen interaction. The well-studied ricin B chain (RTB) is a galactose-specific lectin comprises two identical sugar binding sites preferably oligosaccharides (Frankel et al., 1996, Biochemistry, 35(47): 14749-56). One terminal galactose is bound by binding site 1 (W37) of RTB while the other terminal galactose can bind to the binding site 2 (Y248) of another molecule of RTB without any steric hindrance and make a strong hydrophobic interaction which stabilizes the protein-sugar complexes (Sphyris et al., 1995, J Biol Chem, 270(35):20292-7). Therefore, with reference to ricin B chain, without being bound by theory, it was hypothesize that deletion of aromatic patches or replacement of tyrosine and tryptophan in (QxW)₃ or ₁₅₈YGY₁₆₀ motif of VM protein would likely destabilizes the basic structure of ß-trefoil fold. This led to develop a hypothesis to inhibit the binding of VM proteins to host cell surface and most likely to inhibit toxin assembly and block the prominent virulence factor. The fundamental study of these motifs or carbohydrate binding domain could be achieve by mutagenesis and glycan microarrays which is an excellent tool to examine glycan-protein (host-pathogen) interactions or in identification of host receptor/innate immune receptor (Geissner et al., 2019, Proc Natl Acad Sci USA, 116(6):1958-67).

Sequence and Structure Similarity of RBL2 Domain of VM Protein with CARDS Toxin which Rationalize the Functional Similarity in Binding and Internalization

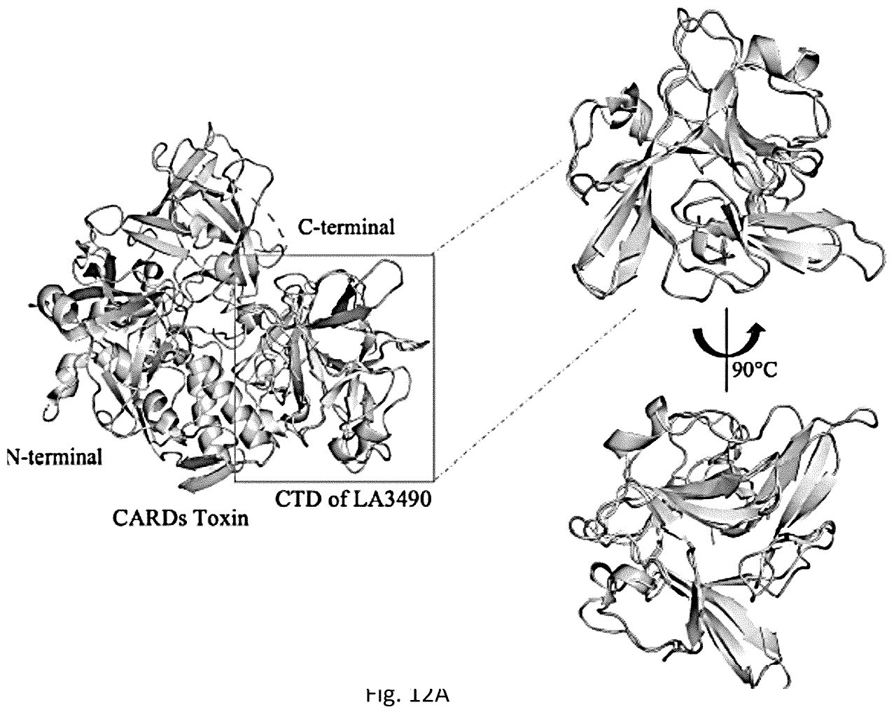

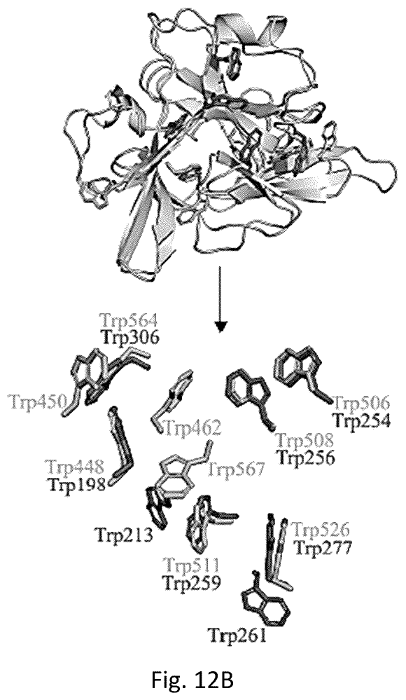

Unrevealing the structure and function of RBL2 of VM protein, the amino acid sequences of VM proteins were subjected to high-throughput Predictprotein online server (predictprotein.org) (Bernhofer et al., 2021, Nucleic Acids Res, 49(W1):W535-W40). The software uses machine learning algorithm with evolutionary information and predict structure and function of the proteins. Predictprotein aligned 32 proteins in which 31 matches were belongs to PF07598 protein family and other hit was CARDs toxin (PDB: 4TLV_A Chain), which showed considerable matches with identity 0.55, expected value: 2e-94 and match length (310 aa). Full length of VM proteins and CARDs toxin (PDB: 4TLV) was structurally superimposed and visualized by PyMOL 2.4.0 (pymol.org/2/). Only the RBL2 (196 aa-335 aa) of VM protein was superimposed at C-terminal of CARDs toxin (PDB: 4TLV, D3 domain: 447 aa-591 aa) with RMSD—1.218 Å (FIG. 12A, B). Amino acid sequence of RBL2 (196 aa-335 aa) of PF07598 protein family and CARDs toxin (PDB: 4TLV, 447 aa-591 aa) were aligned using MAFFT (Multiple Alignment using Fast Fourier Transform) with L-INS-i (Accuracy-oriented) algorithm and visualized in Jalview v2.11.5 (jalview.org). D3 domain of CARDs toxin comprises 8 tryptophan and RBL2 comprises 9 tryptophan, intriguingly 6 tryptophan were conserved at sequence and structure level in both RBL2 and CARDS toxin. CARDs toxins (D2+D3 trefoil) do not have galactose-binding sites suggest RBL1 of VM proteins are plausibly the sole carbohydrate binding partner. Mutagenesis of residues 571 aa-591 aa of CARDs toxins, which is integral to the proper folding of D3 and formation of its aromatic patch, lacked internalization by HeLa cells therefore suggest CARDs toxin entry into host cells mediated by D3 domain (Becker et al., 2015, Proc Natl Acad Sci USA, 112(16):5165-70; Ramasamy et al., 2018, mBio, 9(1)). Superimposition of RBL2 of VM proteins and CARDs (D3 domain) with RMSD=1.218 Å and six conserved tryptophan in their aromatic patch strengthening the RBL2 function as translocation domain in VM proteins which let the VM protein internalize into the host cell. With this information, without being bound by theory, it was hypothesized that the mutation in these conserved tryptophan/aromatic patches abolished the internalization of the protein into host cells. Collectively, mutagenesis, glycan microarray and surface plasma resonance (SPR) would open a window to characterize the function of RBLs domains and identifying their carbohydrate binding partner and translocation of VM proteins inside the host cells.

### Architecture of Intramolecular Disulfide Bond Similar to Ricin Toxin Confirms VM Proteins are Bona Fide AB-Toxin

Literature studies showed several exotoxins such as diphtheria Toxin [Corynebacterium diphtheriae: AB (Murphy et al., 2011, Toxins (Basel), 3(3):294-308)], Pertussis toxin [Bordetella pertussis: A(S1)-B (S2-S5) (Stein et al., 1994, Structure, 2(1):45-57)], shiga toxins [Shigella dysenteriac: AB5 (Johannes, 2017, Toxins (Basel), 9(11))], exotoxin A [Pseudomonas aeruginosa AB (Ogata et al., 1990, J Biol Chem, 265(33):20678-85)] and plant-derived ricin-toxin [Ricinus communis: AB (Lord et al., 2011, Toxins (Basel), 3(7):787-801)] belongs to the class of AB-type toxins (Cherubin et al., 2018, Sci Rep, 8(1):2494). These are known as AB toxins because they contain at least one subunit or polypeptide (B-chain) that recognizes a specific receptor on the cell surface and one subunit or polypeptide (A-chain) that enters the cell to gain access to the target site. These AB toxins specifically modify the host targets by ADP-ribosylation, glycosylation, deamidation, deadenylation, proteolysis, and acetylation etc. These modifications often result in an inactivation of the target, which altered cell physiology or may lead to necrotic or apoptotic cell death (Odumosu et al., 2010, Toxins (Basel), 2(7): 1612-45; Biernbaum et al., 2022, Toxins (Basel), 14(1)). The formation of disulfide bonds in AB toxins regulates the folding and stability needs for the activation of their functional roles therefore they influence the structural and functional properties of AB toxin (Hogg, 2003, Trends Biochem Sci, 28(4):210-4). Reduction in disulfide bond in A-chain and B-chain of ricin decreases its toxicity in mice and also decreases ability to inhibit protein synthesis of Hela cells (Lappi et al., 1978, Proc Natl Acad Sci USA, 75(3): 1096-100).

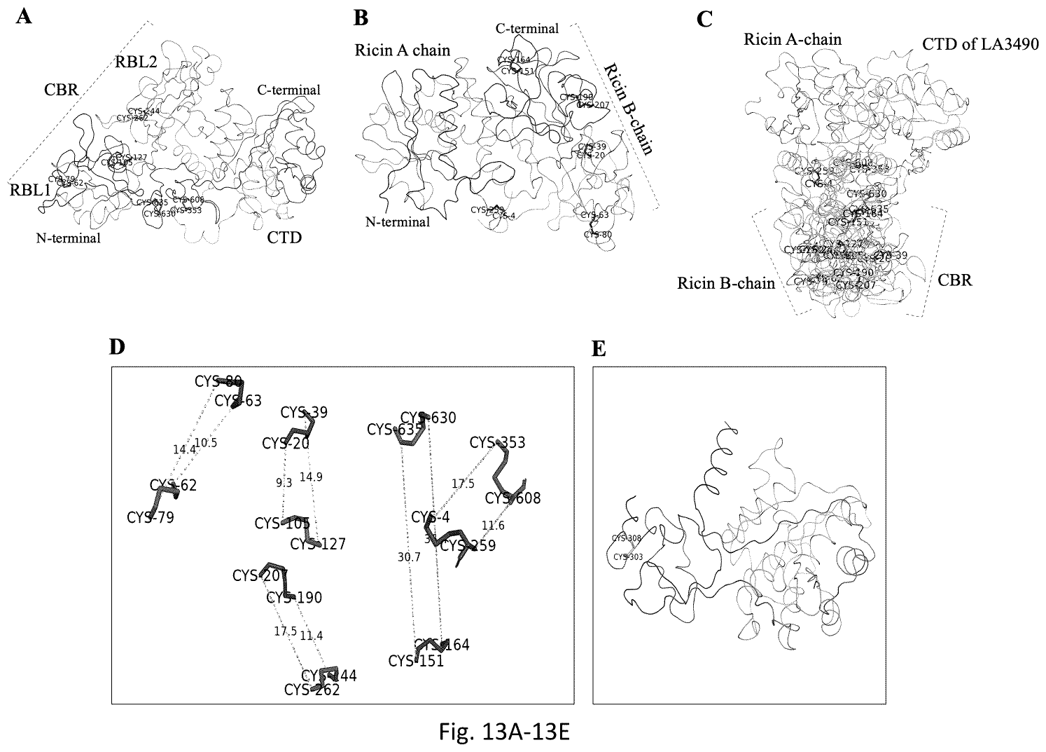

AlphaFold algorithm derived structural architecture of VM proteins shows they comprises twelve cysteine residues, ten cysteine involved in the formation five disulfide bridges similar to ricin toxin (5-disulfide bond) (Chaurasia et al., 2022, Front Microbiol, 13:859680; Lappi et al., 1978, Proc Natl Acad Sci USA, 75(3):1096-100) (FIG. 13A, B, C). Interestingly, RBL1 of VM protein comprised two disulfide bonds (62 aa-79 aa, 105 aa-127 aa), RBL2 domain comprised one disulfide bonds (244 aa-262 aa) and C-terminal globular domain encode for two disulfide bonds (353 aa-608 aa and 630 aa-635 aa) (FIG. 13A-D). VM genes encodes single polypeptide chain unlike ricin toxin therefore proteolytic cleavage of disulfide bond plays a key role in processing of RBL1, RBL2 and C-terminal into their functional domains. Ricin A-chain (RTA) contains two cysteine residues (Cys171 and Cys259), Cys 259 forms the interchain disulfide bond of ricin holotoxin with Cys4 of ricin B chain (RTB). Interestingly, disruption of this disulfide bond by site-directed mutagenesis (cysteine at 259 aa in ricin replaced with alanine) in A-chain reduces the cytotoxicity (Mohanraj et al., 1995, Biochim Biophys Acta, 1243(3):399-406) or introduction of new disulfide bond into ricin A-chain decreases the cytotoxicity of ricin (Argent et al., 1994, J Biol Chem, 269(43):26705-10). Based on structural superimposition of ricin with VM proteins, the disulfide bond at position 353 aa-608 aa (4 aa-259 aa in ricin) believe to be crucial for C-terminal proteolysis or hydrolysis in endosome where biologically active C-terminal domain subsequently release into cytosol and translocated to nucleus. Interestingly, LA0591 (313 aa) naturally lacks RBLs (RBL1 and RBL2), suggest that this natural mutant variant does not require binding and internalization to the host cell therefore speculate their intercellular role in the pathogenesis. The two cysteine in LA0591 form disulfide bond at C-terminal (303 aa-308 aa), the mutagenesis study would be informative about the functional role of disulfide bond in VM proteins and their orthologs (FIG. 13E).

CARDs toxins encode for 6 cysteines residues at amino acid positions 230, 247, 324, 406, 425 and 548 and the disulfide bond formation occurs between residues C230 and C247 which is essential for its cytotoxicity. Mutagenesis study revealed that the disulfide bond protects ADPRT (D1) domain of CARDS toxin from proteases and disrupted disulfide bond does not affect cell binding, internalization, and intracellular trafficking (Balasubramanian et al., 2019, Cell Microbiol, 21(8):e13032). In A-chain of Shiga toxin, the disulfide bond stabilizing the toxin subunit after protease cleavage in the endosome or trans Golgi network (Garred et al., 1995, J Biol Chem, 270(18): 10817-21; Tam et al., 2007, Microbiology (Reading), 153(Pt 8):2700-10), however disulfide linkage-lacking mutant of shiga toxin was more susceptible to degradation by protease and less cytotoxic to cells (Garred et al., 1997, J Biol Chem, 272(17): 11414-9). Likewise, in pertussis toxin, reduction of the disulfide bond alters its conformation which is required for the toxin to exhibit NAD glycohydrolase and ADPRT activities (Moss et al., 1983, J Biol Chem, 258(19): 11879-82; Burns et al., 1989, J Biol Chem, 264(1):564-8). In diphtheria toxin and cholera toxin, reduction of the disulfide bond results in the release of active fragment from the endosomes into the cytosol (Falnes et al., 1994, J Biol Chem, 269(11):8402-7; Collier, 2001, Toxicon, 39(11): 1793-803; Tsai et al., 2001, Cell, 104(6):937-48; Sandvig et al., 2002 FEBS Lett, 529(1):49-53). Significance of disulfide bonds in bacterial toxin and their role in pathogenesis strengthen the computational information of RBLs and disulfide bond architecture of PF07598 protein family (FIG. 13). The approach towards site-directed mutagenesis of cysteine residues or engineering new disulfide bond in VM proteins could be used to reduce the cytotoxicity hence could be used as a vaccine candidate. Taken together, the data leads to the hypothesis that the critical role of the disulfide bond is in the activation of VM toxin and subsequent cytopathological events.

### Comparative Computational Analysis Deciphering the Hot Spot Residues and Active Sites at C-Terminal of PF07598 Protein Family

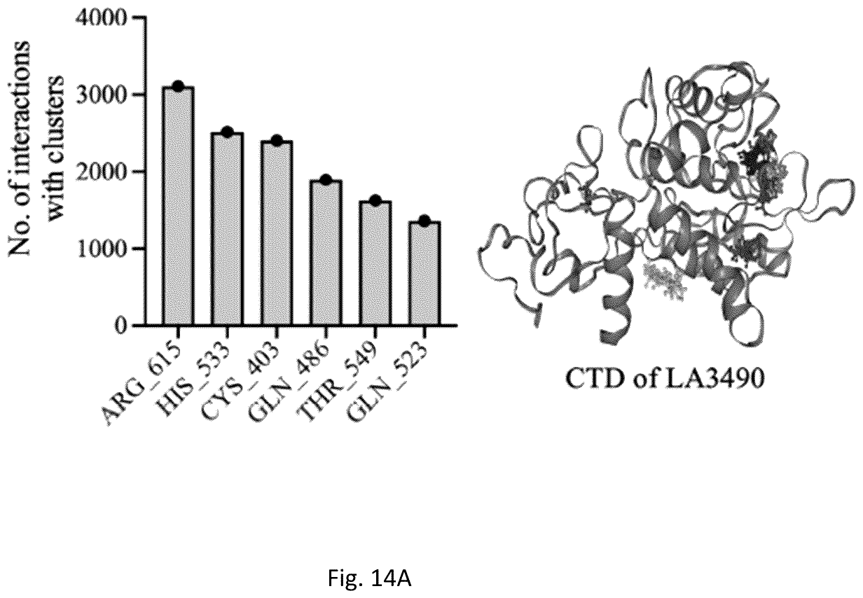

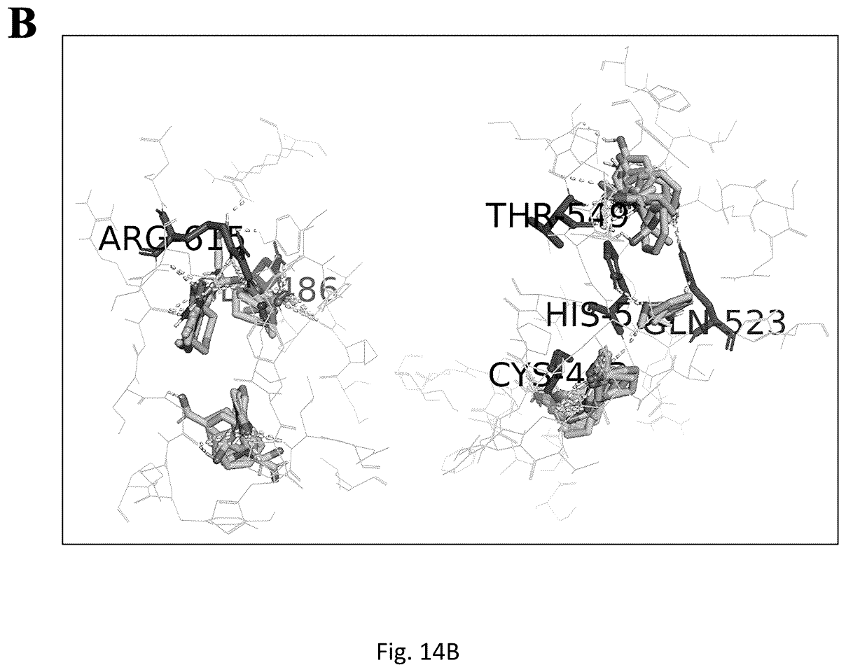

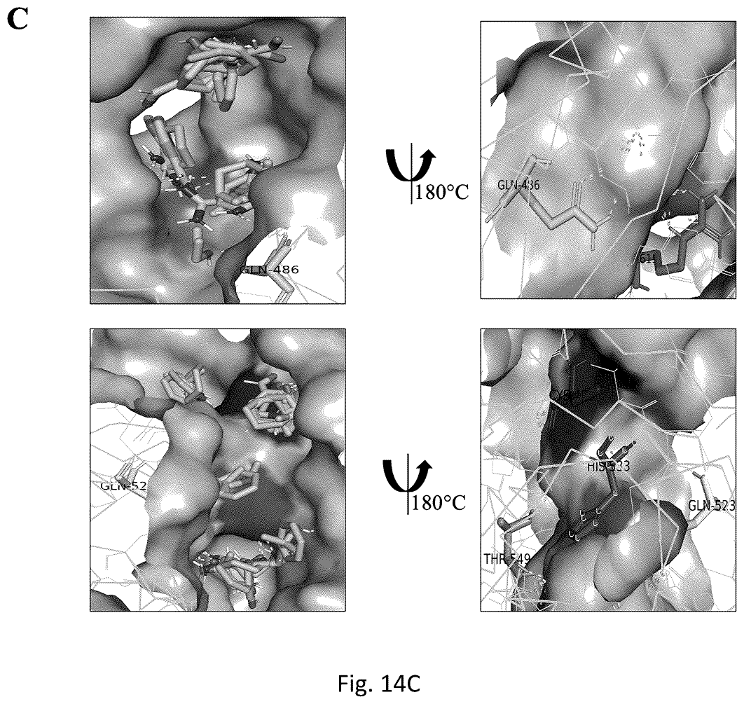

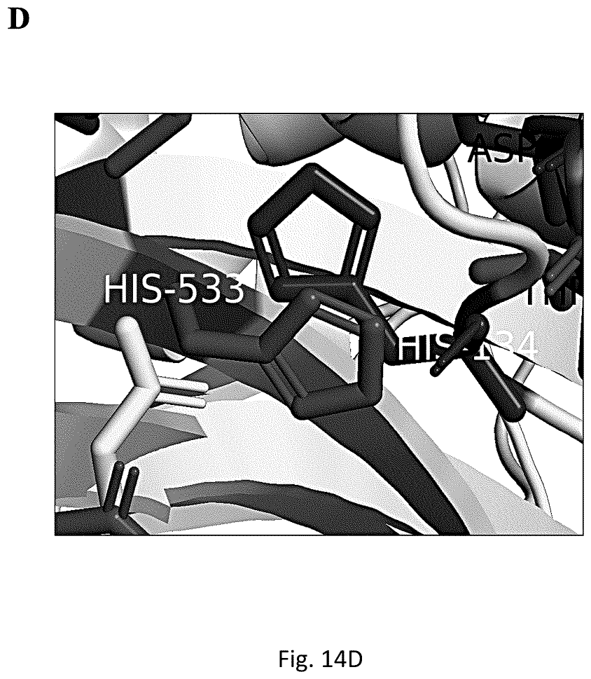

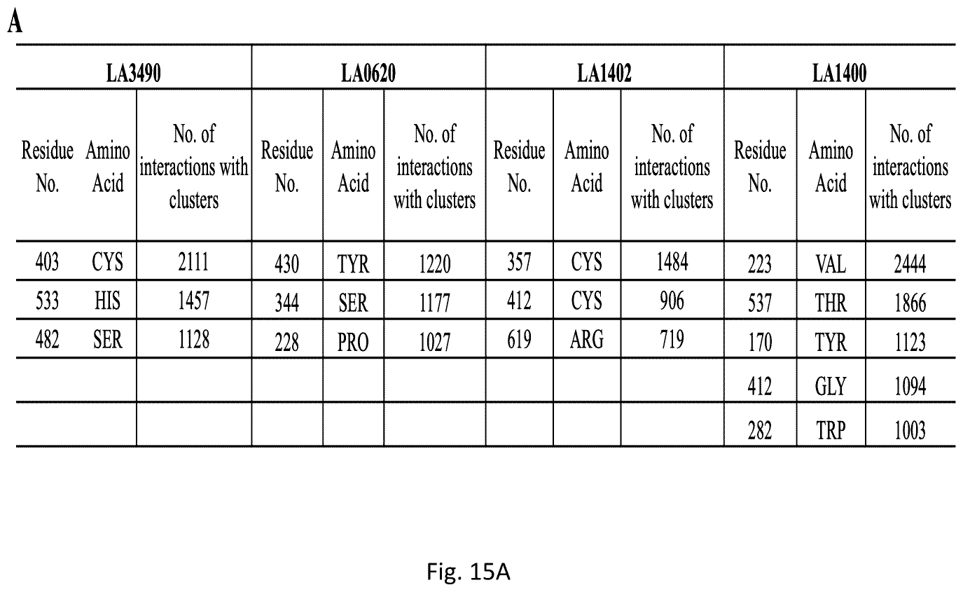

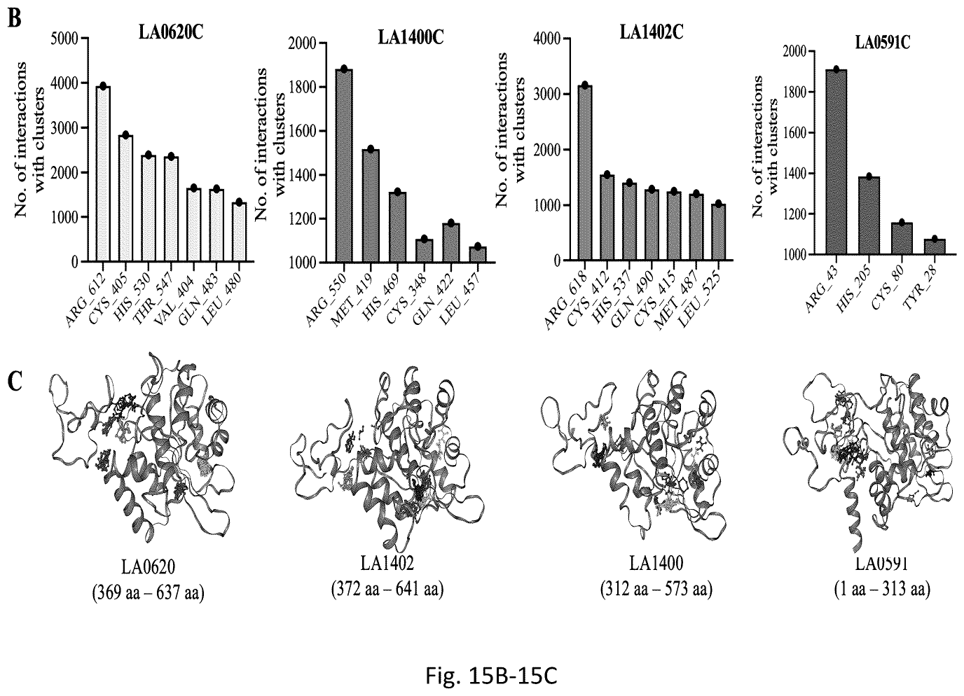

The study was carried out to identify the functionally important regions of amino acids of VM proteins which actively participates in the substrate or ligand binding. AlphaFold algorithm generated 3D structure of VM proteins (full lengths and C-terminal domain) were subjected to online machine learning based servers such as FTMap server (ftmap.bu.cdu) (Kozakov et al., 2015, Nat Protoc, 10(5):733-55), Prank Web (prankweb.cz) (Jendele et al., 2019, Nucleic Acids Res, 47(W1):W345-W9), and Deepsite (playmolecule.com/deepsite/) (Jimenez et al., 2017, Bioinformatics, 33(19):3036-42) for structure-based ligand binding site prediction and to determine the comparative conserved hot spot residues which are actively participated in ligand binding. The analysis of ligand binding sites is often used for function identification and 3D structure-based drug discovery. FTMap server uses 16 small molecules as probes (ethanol, isopropanol, isobutanol, acetone, acetaldehyde, dimethyl ether, cyclohexane, ethane, acetonitrile, urea, methylamine, phenol, benzaldehyde, benzene, acetamide, and N, N-dimethylformamide) and identify hot spot regions which are major contributors to the binding free energy therefore they are the key regions to the binding of any ligands (Kozakov et al., 2015, Nat Protoc, 10(5):733-55; Ngan et al., 2012, Nucleic Acids Res, 40:W271-5). FTMap analysis revealed that amino acid Cys403, His533 and Ser482 are the hot spot residues in the full length LA3490 protein and shown 2111, 1457 and 1128 number of interactions with clusters, however C-terminal domain of LA3490 (368 aa-369 aa) showed higher number of hot spot residues and interactions (Arg615-3109, His533-2510, Cys403-2400, Gln486-1890, Thr549-1622 and Gln523-1357) with clusters (FIG. 14 and FIG. 15). Interestingly, with respect to LA3490, His533 has shown high binding energy as well as consistent in other VM proteins (LA0620: His530, LA1400: His469, LA1402: His537, LA0591:His205) as a best hot spot residue (FIG. 15B). The current study suggest that His533 (LA3490) is crucial amino acid, and its functional role in catalysis could be revealed by mutagenesis approach.

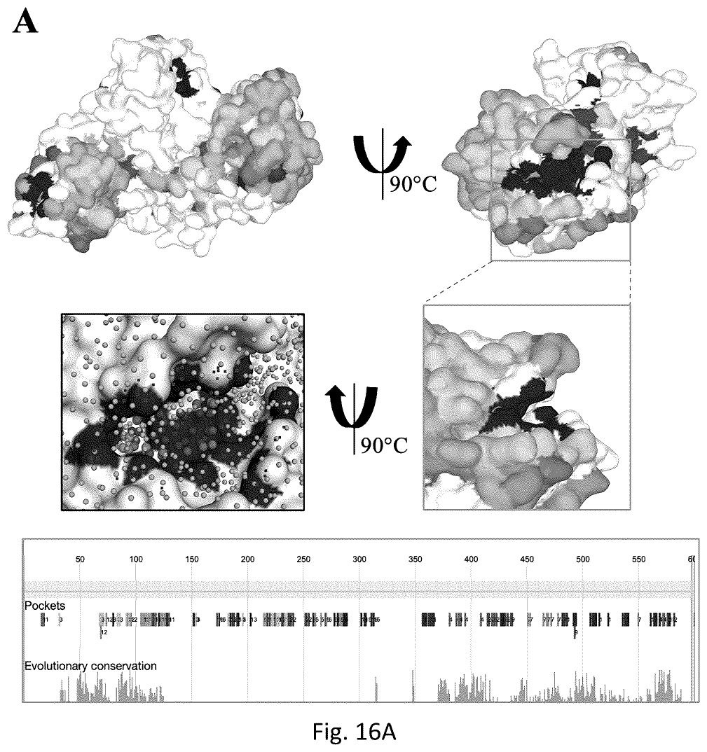

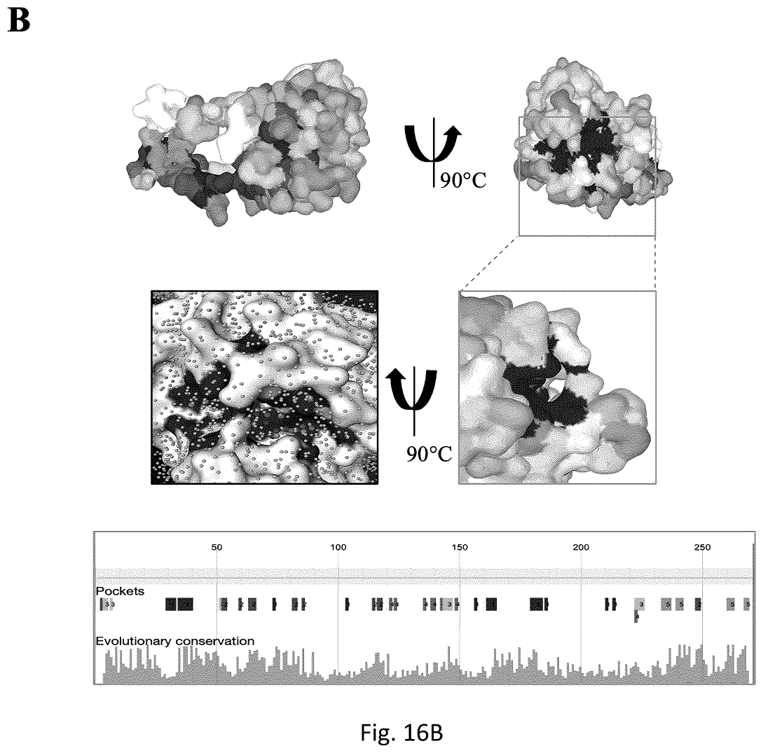

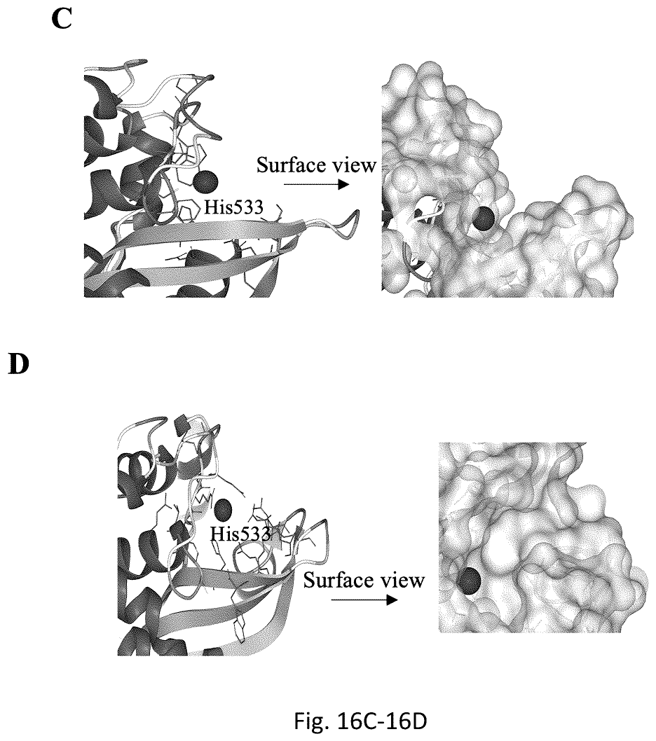

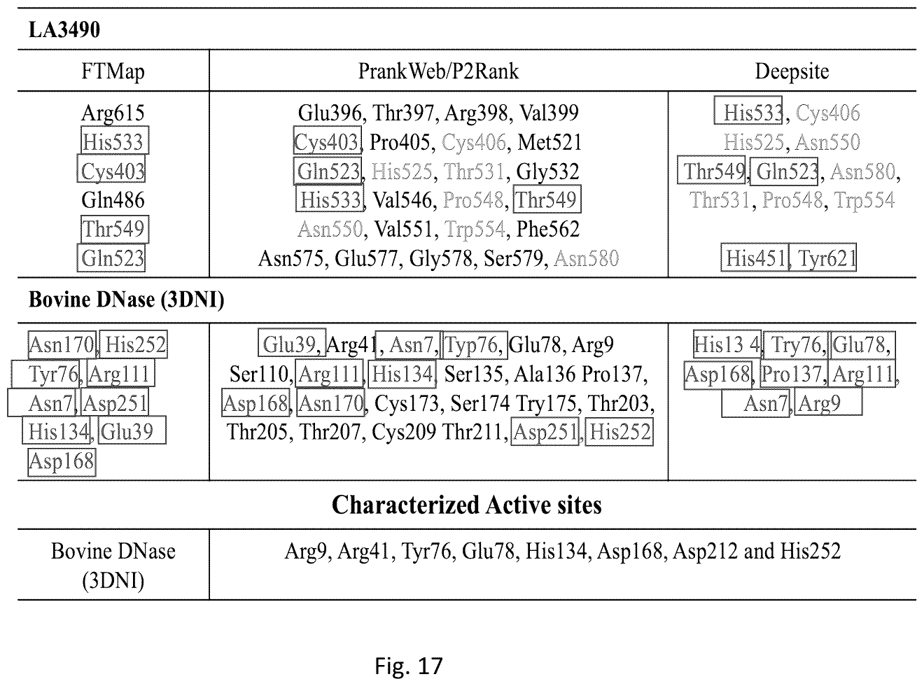

Prank Web and Deepsite are another template free online machine learning based algorithm for structure-based ligand binding site prediction (Jendele et al., 2019, Nucleic Acids Res, 47(W1):W345-W9; Jimenez et al., 2017, Bioinformatics, 33(19):3036-42; Krivak et al., 2018, J Cheminform, 10(1):39). Prank Web identified 14 pockets in full-length LA3490, and they were ranked 1 to 14 based on the probability and solvent accessible surface (SAS points). Pocket 1 scored 18.30 with 0.817 highest probability and 106 Solvent Accessible Surface (SAS points) among the rest pockets (FIG. 16A, 16C). Notably, highest score pocket 1 is located at C-terminal groove and importantly comprising amino acid Cys403, Gln523, His533, and Thr549 which was also screened by FTMap server (FIG. 17). Deepsite machine learning based algorithm identified two deep-pockets, His533, Thr549 and Gln523 located at C-terminal in pocket 1 and His451, Tyr621 located at pocket 2. The amino acids from packet I were also identified by both FTMap and Prank Web. Notably, amino acid Cys406, His525, Thr531, Pro548, Asn550, Trp554 and Asn580 amino acids identified and shared by both Prank Web and Deepsite but not by FTMap server (FIG. 17). This comparative study suggests that His533, Thr549 and Gln523 has high conservation, high confidence and actively involved in ligand binding therefore these amino acids could be useful for their functional study by site directed mutagenesis. The natural mutant variant LA0591 which lacks RBLs, shows His205 identified by all the three online servers (FTMap, Prank Web and Deepsite) involve in ligand binding (FIG. 16B, 16D and FIG. 17).

Because of DNase activity, the comparative study of identifying ligand binding sites in VM protein were validated with 3D structure of bovine DNase (PDB: 3DNI) and its active site was superimposed with VM protein (FIG. 17). Notably, in bovine DNase, Asn7, Glu39, Tyr76, Arg111, Asp251, His134, Asp168, Asn170 and His252 were identified as hot spot residues by FPMap and these amino acids were shared by Prank Web and Deepsite (FIG. 17). The crystalized structure of bovine DNase (PDB: 3DNI) and human DNase (4AWN) suggest that Arg9, Arg41, Tyr76, Glu78, His134, Asp168, Asp212 and His252 are the amino acids present at active site (Suck et al., 1984, EMBO J, 3(10):2423-30; Parsiegla et al., 2012, Biochemistry, 51(51): 10250-8). The list of shared amino acids in VM proteins by three independent server FTMap Prank Web and Deepsite and identification of ligand binding sites of bovine and human DNase which overlap with an active site suggest that these machines learning based algorithm are reliable to screen the hot spot residues/ligand binding sites/active sites in proteins. In bovine and human, His134 and His252 and their hydrogen-bond pairs Glu 78 and Asp 212 are crucial for the functional DNase I activity (Pan et al., 1998, Protein Sci, 7(3):628-36) and mutations at any of the four catalytic amino acids (His 134, His 252, Glu 78, and Asp 212) drastically reduced the hydrolytic activity of DNase I (Pan et al., 1998, Protein Sci, 7(3):628-36). Superimposition of heterocyclic His134 of bovine DNase with His533 (LA3490) of PF07598 gene family strengthen the catalytic site in VM protein is mediated by His533 which is highly conserved at C-terminal of VM proteins (LA0620: His530, LA1400: His469, LA1402: His537, LA0591:His205) (FIG. 14D, FIG. 15). Approach towards site-directed mutagenesis will help to reveal the functional catalytic residues at C-terminal of VM proteins. Also, the Predictprotein, (predictprotein.org) online server further provided informative knowledge about structure-function and strengthen the computational analysis by showing the DNA binding domain at C-terminal of VM proteins (FIG. 15).

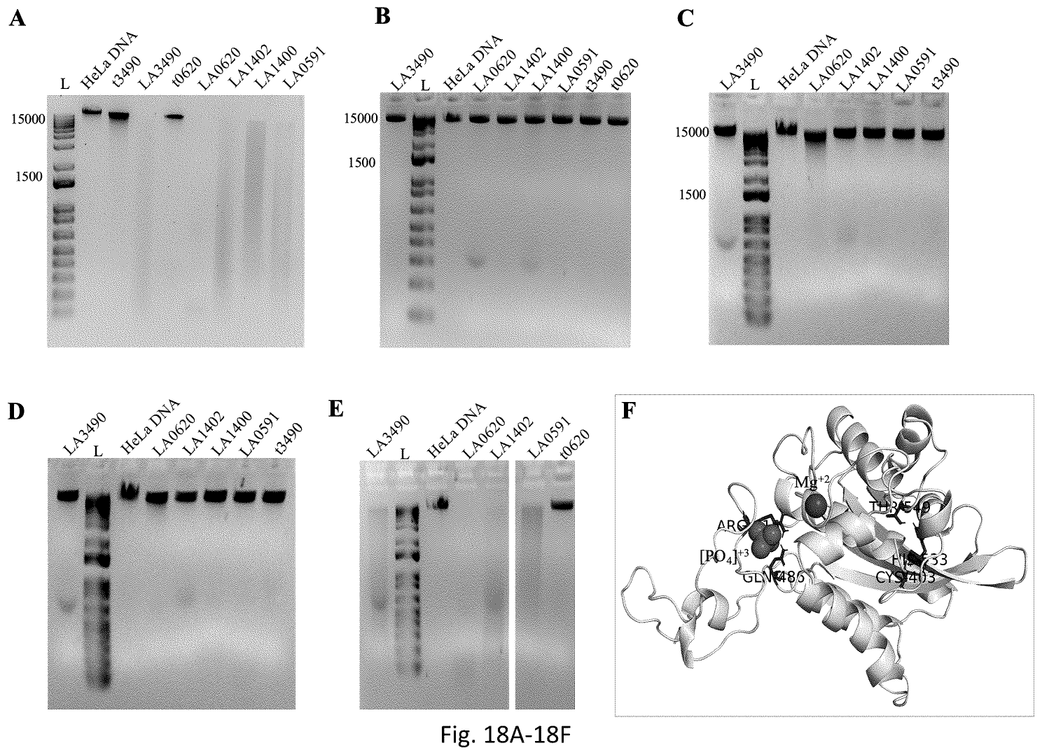

The DNase activity of VM proteins is dependent on Mg⁺² ion, however presence of Zn⁺², Ca⁺² or absence of Mg⁺² ion abolishes the catalytic activity of VM proteins (FIG. 18). The docking study was performed to the C-terminal of LA3490 with phosphate and magnesium ion using MGLTools 1.5.7. Magnesium ions interact with hotspot residue Gln412 and shown binding energy −0.95 kCal/mol, however phosphate ions interact with hotspot residue Arg615 and shown binding energy −2.58 kcal/mol (FIG. 18). The hot spot residues or ligand binding residues are the best targets for site-directed mutagenesis approach leading to functional characterization of active sites in PF07598 protein family.

### Example 3: Vaccination with Leptospira interrogans PF07598 Gene Family-Encoded Virulence Modifying Proteins Protects Mice from Severe Leptospirosis and Reduces Bacterial Load in Liver and Kidney

Mechanisms by which pathogenic Leptospira cause severe disease has remained elusive ever since the initial descriptions of the etiology of leptospirosis (Noguchi et al., 1917, J Exp Med, 25(5):755-63; Inada et al., 1915, The Journal of Experimental Medicine, XXIII:377-402). Although historically the nomenclature of Leptospira has been confusing, recent genomic and molecular approaches have clarified the relationships among species and serovars. Of high importance is the discovery that the PF07598 gene family is present only in pathogenic, Group 1, Leptospira, and expanded in the most pathogenic species, L. interrogans, as well as in L. kirschneri and L. noguchii. Severe human disease is primarily attributed to infection by serovars belonging to L. interrogans; such data are limited because of insufficient isolates obtained from cases of severe leptospirosis that enable definitive identification of infecting Leptospira in such cases. Because current gene knockout approaches to Leptospira remain limited, especially as applied to multi-gene families, an immunological approach was used to demonstrate whether leptospiral PF07598 gene family-encoded VM proteins might be virulence factors contributing to severe leptospirosis disease manifestations in a mouse model. The data presented here support the hypothesis that VM proteins have central importance as virulence factors in the pathogenesis of severe leptospirosis.

Vaccination of C3H/HeJ mice with as few as two L. interrogans serovar Lai VM proteins (G-IV, LA1400 and LA0591) but as many as five (G-III, LA1400 and LA0591, plus LA3490, LA0620 and LA1402) protected mice from any clinical manifestations of disease and led to ˜3-4 log 10 reduction in bacterial load in liver and kidney, two key organs in pathogenesis of leptospirosis and transmission of Leptospira, respectively. Previous data indicate that all PF07598 gene family members are variably upregulated in the hamster model of acute, severe leptospirosis (Lehmann et al., 2013, PLOS Negl Trop Dis, 7(10): e2468). Specific VM protein antigens for groups G-III and G-IV were chosen based on previously data describing those with the highest and lowest expression in vivo (Lehmann et al., 2013, PLOS Negl Trop Dis, 7(10): e2468). The present findings suggest that VM protein vaccination with a minimum complement of cross-reactive VM proteins might confer protective immunity but, as of yet, whether LA1400 and LA0591 are both needed as immunogens remains unknown. Curiously, post-immunization/pre-challenge sera from G-IV heterologously cross-reacted with highest titers against with LA1402 and LA3490 despite low titers against their homologous proteins. LA1400, an ancestral VM protein in Group 1 pathogenic Leptospira belonging to cluster A (Fouts et al., 2016, PLOS Negl Trop Dis. 10(2):e0004403), has 2 N-terminal, tandemly repeated ricin B-like lectin domains (RBLs), and a C-terminal toxin domain (CTD). LA0591 has a CTD but lacks RBLs. These domains might a priori be expected to cross react most strongly with homologous VM proteins, but the experimental data indicate that heterologous cross-reactivity was instead found to be strongest. Further experiments are in progress to determine further whether immunization with either of these proteins alone, concatenated, or isolated subdomains of various VM proteins, or even one more full length VM proteins such as LA1402 and LA3490, might confer pan-leptospiral immunity. A likely scenario is that the general cross-reactivity to VM proteins induced by vaccination with LA1400 and LA0591 mediates protection against lethal challenge infection and tissue colonization. This possibility is suggested by bioinformatic analysis indicating that VM proteins are highly conserved at the amino acid level within L. interrogans and is supported experimentally (Chaurasia et al., 2022, Frontiers in Microbiology, 13:859680).

While immunization of leptospirosis disease-susceptible C3H/HeJ mice (Viriyakosol et al., 2006, Infect Immun. 74(2):887-95) with full-length leptospiral VM proteins protected against severe disease, vaccination with an isolated RBL, t3490, a recombinant protein containing only the N-terminal ricin B domain (G-II), led to disease enhancement while simultaneously reducing the bacterial load in liver and kidney. Multiplex cytokine analysis on serum showed that Group II mice had unique elevations in pro-inflammatory cytokine markers (IL-ß, IL-6, IL-10, IFN-γ, TNF-α and KC/GRO (Wolpe et al., 1989, Proc Natl Acad Sci USA. 86(2):612-6) (neutrophil chemoattractant related to IL-8 in rodents) which suggest that these cytokine storm might be responsible to lead mice death. The mechanism by which RBD-domain-induced immune enhancement leads to severe disease is unclear. Without being bound by theory, it was speculated that one potential mechanism by which t3490 immunization led to disease enhancement could be the induction of antibodies to the N-terminus RBLs of Leptospira-secreted VM proteins that, in vivo, carry the full-length protein to the pro-inflammatory pathway in Fc receptor-containing cells, but this hypothesis requires experimental testing. Nonetheless, cross-reactive antibody generated against RBD in G-II immunized mice did not protect against severe disease. These observations suggest that the N-terminal RBD alone should not be used for a VM protein-based leptospirosis vaccine studies. Further work to study RBD-mediated immune enhancement is needed.

In the present study, ELISA and Western blot analysis of post-vaccination sera on using recombinant VM proteins and osmolarity-induced in vitro cultivated L. interrogans serovar Lai indicates that vaccination resulted in both homologous and heterologous VM protein recognition associated with protective immunity. These experimental results confirm bioinformatic predictions of cross-reactivity of polyclonal antisera for VM proteins within the genus L. interrogans. Further work to confirm protection against challenge infection in rodent models by other L. interrogans serovars is essential. Cross-species protection experiments after VM protein vaccination against challenge with virulent isolates of closely related L. kirschneri and L. noguchii, the other group 1 highly pathogenic Leptospira species (Lehmann et al, 2013, PLOS Negl Trop Dis, 7(10): e2468; Fouts et al., 2012, J Hepatol, 56(6): 1283-92) are planned. Cross-species protection experiments after homologous, or heterologous VM protein vaccination followed by challenge infection with virulent isolates of other group 1 pathogens such as L. borgpetersenii, which have few PF07598 paralogs in their genomes, will contribute to determining which VM proteins might be appropriate for further development into a pan-leptospirosis vaccine.

A serovar-independent pan-leptospirosis vaccine that confers protection against leptospirosis is a major priority in the leptospirosis field (Wunder et al., 2021, Elife., 10; Beutler et al., 2000, Eur Cytokine Netw, 11(2): 143-52). Various inactivated whole bacterial cell-based vaccines (bacterins) are serovar-specific and limited to animal use, where this legacy technology remains incompletely effective. Subunit vaccines, and more recently a spontaneously-arising attenuation mutant of L. interrogans serovar Copenhageni (Wunder et al., 2021, Elife., 10) have been proposed amidst search for pan-leptospirosis vaccine candidates (Haake et al., 1999, Infect Immun, 67(12):6572-8223; Conrad et al., 2017, PLOS Negl Trop Dis, 11(3):e0005441; Techawiwattanaboon et al., 2019, Vaccines (Basel), 7(3); Govindan et al., 2021, Appl Nanosci, 1-15; Phoka et al., 2021, Vet Microbiol, 262:109220; de Oliveira et al., 2021, Vaccine, 39(39):5626-34; Haake et al., Front Immunol, 11:579907; Teixeira et al., 2020, Front Immunol, 11:568694; Coutinho et al., 2011, PLOS Negl Trop Dis, 5(12):e1422). Bacterins are limited from wider use because of adverse effects and suboptimal efficacy including the lack of durable protective and sterilizing immunity (Felix et al., 2020, Expert Opin Drug Discov. 15(2): 179-88; Techawiwattanaboon et al., 2019, Vaccines (Basel), 7(3); Levett, 2001, Clin Microbiol Rev, 14(2):296-326; Zaugg et al., 2021, Schweiz Arch Tierheilkd, 163(9):545-52).

The present report demonstrates the protective immunity induced by vaccination with a subset of L. interrogans VM proteins against lethal challenge infection. The immunization strategy to induce anti-VM protein antibodies validates the VM proteins' role in mediating leptospirosis pathogenesis.

The Materials and Methods used for the experiments are now described

### Bacterial Cultures

Leptospira interrogans serovar Canicola strain LOCaS46 were grown at 30° C. in liquid Ellinghausen-Mccullough-Johnson-Harris (EMJH, BD Biosciences, USA) (Ellinghausen et al., 1965, Am J Vet Res, 26:39-44). Leptospira were grown under conditions mimicking the in vivo host environment known to induce virulence gene expression in vitro (Matsunaga et al., 2005, Infect Immun, 73(1):70-8). Briefly, mid-logarithmic cultures in unmodified EMJH medium were harvested by centrifugation at 18,514 g. Pelleted cells were washed twice with 1× phosphate buffered saline, resuspended in liquid EMJH medium supplemented with 120 mM NaCl, and then incubated at 37° C. for 4 h (Sigma Aldrich, USA). The LD50 of LOCaS46 strain has a median lethal dose LD50<100 (Salinas et al., 2020, Vaccines (Basel), 8(4)).

Chemically competent E. coli strain DH5α (New England Biolabs, Ipswich, MA) was used for gene cloning, and strain SHuffle®T7 competent E. coli cells (New England Biolabs, USA) was used for protein expression and purification. E. coli were grown in Luria-Bertani (LB) medium (BD Biosciences, Sparks, MD) supplemented with 100 μg/mL ampicillin (Sigma-Aldrich, St. Louis, MO).

The L. interrogans serovars Lai, Canicola, Copenhageni, and the non-pathogenic serovar L. biflexa serovar Patoc were grown in liquid EMJH medium and harvested by centrifugation at 18,514 g for 10 mins. Cells were washed twice with 1×PBS pH 7.4, and pellet were resuspended in 5 mL/gram of BugBuster® Protein Extraction Reagent (Sigma-Aldrich, St. Louis, MO) containing “Protease Inhibitor Cocktail with EDTA” (Roche, USA). Cell lysates were incubated on a rotating mixer for 15 minutes at room temperature. Insoluble cell debris was removed by centrifugation at 18,514 g for 20 minutes at 4° C. Supernatant were stored at −20° C. until analysis.

### Computational Biology

N- and C-terminal amino acid sequences (LA3490, LA0620, LA1402, LA1400 and LA0591) of PF07598 family were aligned using MAFFT (Multiple Alignment using Fast Fourier Transform) with using L-INS-i (Accuracy-oriented) and visualized in Jalview v2.11.5 (jalview.org). The originally deposited LA1400 sequence was found to be incomplete in that it lacks sequence encoding the first 54 amino acids of the complete encoded protein. This conclusion was based on the use of clustal analysis to compare the amino acid sequences of L. interrogans serovar Lai LA1400 to LIC12340, the LA1400 ortholog in L. interrogans serovar Copenhageni strain FioCruz L1-130 (Supplementary Information). The recombinant protein referred to in the present work as LA1400 is comprised of amino acids 31 to 54 derived from LIC12340, then LA1400-derived amino acids from position 55 to the end.

### Animals

Three-week-old, specific pathogen-free, female C3H/HeJ mice (The Jackson Laboratory, ME, USA) were purchased from the Jackson Laboratories (ME, USA) and maintained in a specific-pathogen-free environment at Yale Animal Resources Center. The mice were housed in individually ventilated microisolator cages with sterile, absorbent beddings changed twice weekly. The animals were fed and watered throughout the course of the experiment. Following L. interrogans serovar Canicola challenge, mice were weighed and monitored daily twice until the final endpoint. They were observed for loss of appetite, severe lassitude, difficulty in breathing, prostration, ruffled fur, and weight loss of 10%. Mice with these manifestations were euthanized by CO2 according to AAALAC/AVMA-approved procedures and considered to have met the endpoint of severe/lethal leptospirosis.

### Plasmid Constructs and Cloning

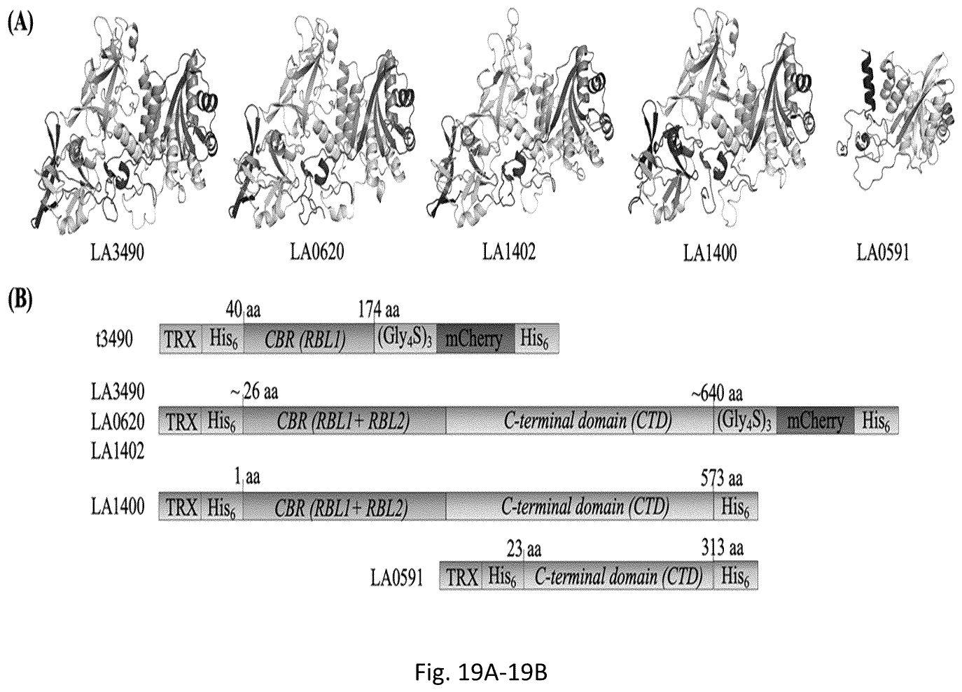

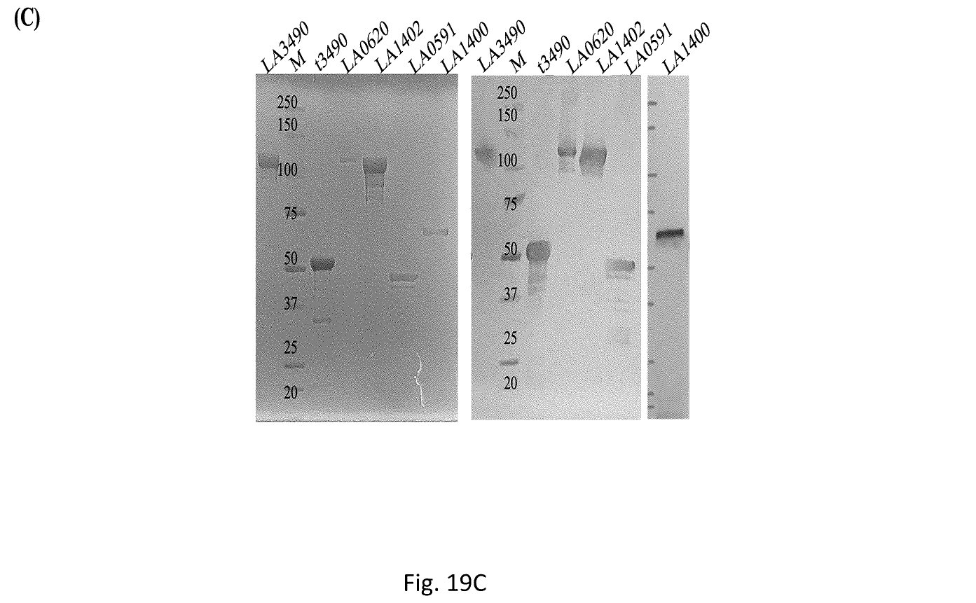

Synthetic E. coli codon-optimized genes were constructed by Gene Universal (geneuniversal.com) consisting of either the complete PF07598 genes encoding NCBI locus tag LA3490 (Uniprot: Q8F0K3), LA0620 (Q8F8D7) and LA1402 (Q8F6A7) from serovar Lai, and locus tag LIC12340 (Q72PX7) (Lai ortholog: LA1400), and LIC12985 (Q72N53) (Lai ortholog: LA0591) from serovar Copenhageni, coding sequence minus the predicted signal peptide or truncated 3490, an N-terminal domain, were synthesized and cloned into pET32b (+) (Gene Universal Inc., USA). LA3490, LA0620, LA1402 and t3490 were linked to mCherry (AST15061.1) via a glycine-serine hinge (Gly4Ser)3 and cloned into pET32b (+) (Gene Universal Inc., USA) between enterokinase cleavage sites for convenient removal of the mCherry fluorescent tag. Full-length LA1400 and LA0591 constructs were made without the mCherry fusion (FIG. 19A). Prior to use, the sequence, and the orientation of the genes in the constructs were verified by restriction digestion and sequencing.

### Expression and Purification of Recombinant Soluble PF07598 Antigens

Recombinant PF07598 protein constructs were expressed in SHuffle®T7 competent E. coli cells (New England Biolabs, USA). Transformants were sub-cultured into Luria-Bertani (LB) medium containing 100 μg/mL ampicillin. Expression of PF07598 proteins were induced at OD of 0.6 via addition of 1 mM isopropyl-ß-D-thiogalactoside (IPTG; Sigma-Aldrich, USA) and allowed to incubate at 16° C. and 250 rpm for 24 hours. Upon induction, cells were harvested, and pellet were lysed in CelLytic™ B (Cell Lysis Reagent; Sigma-Aldrich, USA) containing 50 units benzonase nuclease (Sigma-Aldrich, USA), 0.2 μg/mL lysozyme, non-EDTA protease inhibitor cocktail (Roche, USA) plus 100 mM PMSF (Sigma-Aldrich, USA) for 30 minutes at 37° C. Supernatants and pellets were separated, and then analyzed by 4-12% bis-tris sodium dodecyl sulfate-polyacrylamide gel electrophoresis (SDS-PAGE). Protein concentrations were determined by BCA assay (Bio-Rad, Hercules, CA).

Recombinant PF07598 fusion and without fusion proteins were purified using a 5 mL pre-packed Ni-Sepharose AKTA Hi-TRAP column (GE Healthcare, USA) pre-equilibrated with a buffer containing 100 mM NaH₂PO₄, 10 mM Tris-HCl, 25 mM imidazole, pH 8.0. PF07598 proteins bound to Hi-TRAP column were then eluted in the presence of 500 mM imidazole, pH 8.0. Eluates were pooled, concentrated via a 10 kDa Amicon® Ultra centrifugal filter and further dialyzed overnight against 1×PBS (pH 7.4) with gentle stirring (350 rpm) at 4° C. (10 kDa cutoff, Slide-A-Lyzer, Thermo Scientific™, USA). Purified recombinant PF07598 proteins was resolved in SDS-PAGE, verified by immunoblotting with mouse anti-His monoclonal-ALP conjugate (1:2,000 dilution; Santa Cruz Biotechnology, USA). Aliquot for boosters and SDS-PAGE were prepared from the single preparation and stored at −80° C. to prevent repeated freeze-thawing.

### Animal Immunization, Leptospira Challenge and Sample Collection

C3H/HeJ mice were immunized via intramuscular (IM) route with recombinant PF07598 proteins (Viriyakosol et al., 2006, Infect Immun, 74(2):887-95). GLA-squalene-oil-in-water emulsion adjuvants (0.25 mg/mL) were procured from Infectious Disease Research Institute (IDRI), Seattle, WA, USA (idri.org). Immediately before injections, adjuvant was added to the recombinant protein or PBS to a final volume of 100 μL and mixed by brief vortexing (Patra et al., 2015, Infect Immun, 83(5):1799-1808).

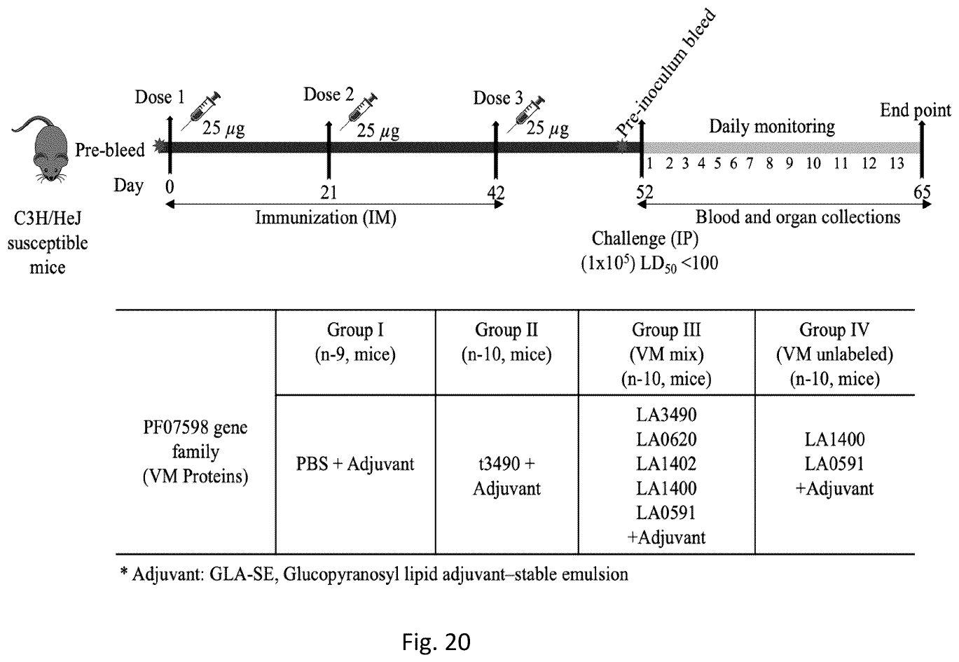

Mice were divided into four groups; G-I served as negative control and was injected with 1× phosphate buffer saline (PBS) mixed with adjuvant (EM082; 5 μg GLA-squalene-oil-in-water emulsion). Similarly, G-II (t3490), G-III [VM mix, (LA3490, LA0620, LA1400, LA1402 and LA0591] and G-IV [VM unlabeled, (LA1400 and LA0591] were immunized with 25 μg total antigen in equimolar ratio along with adjuvant (5 μg GLA-squalene-oil-in-water emulsion) followed by two injections of 25 μg of total antigen at 3-week intervals (FIG. 20). Immunized mice were bled two weeks after the final immunization, and, to smooth out individual differences with groups, serum samples were pooled and measured for anti-VM antibodies in a serum known as pre-challenged bleed. All the group were experimentally infected by intraperitoneal (IP) injection with 1×10⁵ organisms of a virulent, low passage isolate of L. interrogans serovar Canicola, strain LOCaS46. Mice that survived infection were euthanized 13 days after infectious challenge. Blood was collected by terminal cardiac puncture and serum was isolated from whole blood. Serum was allowed to clot at room temperature and stored overnight at 4° C. Samples were then centrifuged at 11, 292 g for 15 minutes at 4° C. Serum was collected and stored at −80° C. Organs were collected and stored in RNALater at 4° C. Kidney and liver tissues were used for quantification of L. interrogans by quantitative PCR (qPCR).

### Evaluation of PF07598 Proteins-Induced Immunity by ELISA

Serum antibody responses to recombinant PF07598 proteins in immunized groups were quantified by ELISA (61). Briefly, PF07598 antigens (LA3490, LA0620, LA1402, LA1400, and LA0598, respectively) in 100 μL of bicarbonate/carbonate coating buffer were coated (250 ng) in 96-well microtiter ELISA plate (Corning, USA) and incubated at 4° C. for overnight. Each set of antigens were incubated with pre- and post-immunized serum group (Group I-IV, 1:1000) for 1 hour followed by goat anti-mouse IgG (Fc specific)-alkaline phosphatase conjugate (1:5000; KPL, USA) for 1 h, washed thrice with TBST, and developed with p-Nitrophenyl phosphate (1-Step™ PNPP Substrate Solution; KPL, USA). The reaction was stopped with 2 M NaOH, and absorbance was read at 405 nm using a SpectraMax® M2e Microplate Reader (Molecular Devices, USA). For whole cell ELISA, plate was coated with 500 ng/well cell free lysates. The controls included pre-bleed, pre-immunized serum samples and antigen and antibody blanks.

### Sodium Dodecyl Sulfate-Polyacrylamide Gel Electrophoresis (SDS-PAGE) and Western Immunoblot Analysis

SDS-PAGE was done according to the method of Laemmli (Laemmli, 1970, Nature, 227(5259):680-5). Immunoblot analysis was performed to determine whether sera from immunized animals recognize recombinant or native leptospiral PF07598 proteins. Purified recombinant PF07598 proteins or leptospiral whole cell lysate (120 mM NaCl induced and without induced) were transferred to nitrocellulose membranes and blocked for 2 h with 5% nonfat dry milk dissolved in 1×TBST buffer (AmericanBio, USA). The membrane was incubated with pooled sera from immunized groups (Group I-IV, 1:100) and controlled as pre-bleed and pre-immunize bleed for overnight at 4° C. on rocker. They were probed with goat anti-mouse IgG (Fc specific)-alkaline phosphatase conjugate (1:5000; KPL, USA) for 2½ hours and washed thrice with TBST and developed with p-Nitrophenyl phosphate (1-Step™ PNPP Substrate Solution; KPL, USA). Monoclonal LipL32 antibody served as loading control (1,10,000 dilution).

### Quantitative PCR

DNA was extracted by dicing 40±50 mg of kidney and liver tissues and suspending in 500 μL of 1×PBS; all work was done under positive pressure in a location separate from other handling of Leptospira and PCR products to reduce risk of cross-contamination. Following tissue homogenization, total genomic DNA was extracted from equivalents of 25 mg tissue using the DNeasy Blood and Tissue Kit (Qiagen, USA) per manufacturer's instructions and eluted in 50 μL of elution buffer. L. interrogans serovar Canicola at a density of 2×10⁷ leptospires/mL grown in 5 mL of EMJH culture medium. Cells were harvested and DNA was extracted for standard curves using the same DNeasy Blood and Tissue Kit (Qiagen, USA).

The concentration of eluted DNA was determined using a NanoDrop Spectrophotometer ND-1000 (NanoDrop Technologies, DE, USA). All DNA samples were kept at −80° C. until use. Serial dilution (1×10⁰ to 1×10⁷ genomic equivalents (GEq)/5 μL) of DNA was prepared and L. interrogans Serovar Canicola genome was quantified by qPCR using 2× iQ5 SYBR Green supermix (Bio-Rad, CA, USA) with 5 pmol forward (5′-TCTGTGATCAACTATTACGGATAC-3′; SEQ ID NO: 19) and reverse (5′-ATCCAAGTATCAAACCAATGTGG-3′; SEQ ID NO:20) LipL32 primer. Four microliters of standard or sample DNA was added to 10 μL PCR mix and the reaction was subjected to amplification in the CFX96 Real-time PCR Detection System (Bio-Rad, USA) using the following program: 3 min at 95° C., 0.10 min at 95° C., 0.30 min at 62° C., followed by 44 cycles at 1.00 min at 72° C. then final extension 7 min at 72° C. A standard curve was generated using Bio-Rad iQCycler5 software, and the number of GEq was extrapolated from the threshold cycle (CT) values. A negative result was assigned where no amplification occurred or if the CT value was greater than 3 SD+Ct. Data are presented as the number of L. interrogans GEq per gram of tissue.

### Statistical Analysis

All experiments were performed in triplicate and repeated twice. The Kruskal-Wallis test was used to determine significant differences in the number of bacteria in kidney or liver among the survivors from different immunization groups. The results were analyzed by the non-parametric Mann-Whitney test to determine significant differences between individual groups and were considered as statistically significant when, p<0.05, p<0.001. All analyses and graphs were generated using Graph Prism version 8 (GraphPad Software, Inc., La Jolla, CA).

The Experimental Results are now provided

Conservation of PF07598 Protein Family and their Orthologs in Pathogenic Leptospira

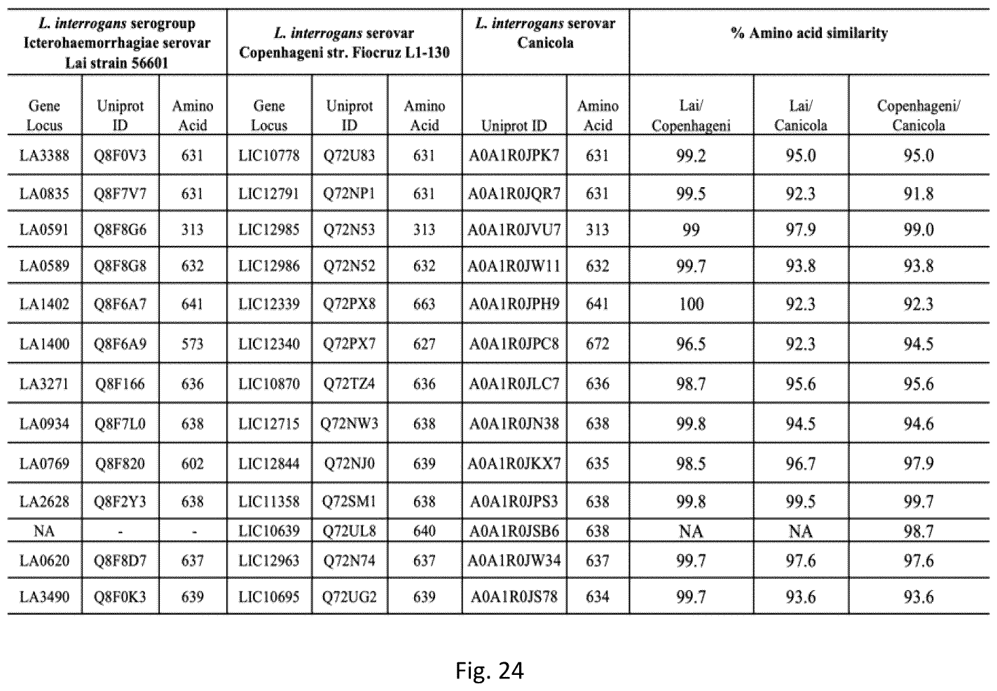

The PF07598-encoded VM paralogous protein family has an expanded repertoire within L. interrogans, with at least 12 distinct paralogs in serovars Lai, Copenhageni and Canicola. Orthologs have >90% amino acid amino acid identity (FIG. 24). Most VM proteins are comprised of ˜640 amino acids with an AB domain architecture comprised of two tandemly arrayed β-trefoil, N-terminal ricin B-like lectin domains, and a C-terminal toxin domain that has DNase activity. L. interrogans serovars also encode a single unique ortholog that lacks a N-terminal ricin B-like domain (typified by LA0591, of ˜313 aa) but which contains a signal sequence.

Immunization with Full Length VM Proteins Prevented Severe Leptospirosis in Mice

Full length recombinant VM proteins LA3490, LA0620, LA1402, LA1400, LA0591 (following L. interrogans serovar Lai nomenclature) were expressed in E. coli as N-terminal fusions with thioredoxin (TRX)-His6 affinity tags to facilitate solubility and affinity purification, and C-terminal fusions with mCherry-His6 to facilitate affinity purification and fluorescence microscopy visualization of the protein, respectively (FIG. 19A). The homogeneity of recombinant VM proteins was verified by SDS-PAGE and Western immunoblot (FIG. 19B).

Mice were injected intramuscularly with recombinant proteins or PBS control mixed with glucopyranosyl lipid A/squalene oil-in-water (GLA-SE) adjuvant (schematically depicted in FIG. 20). This adjuvant was chosen for the present experiments because it is compatible for human use, hence useful to test in animal models towards eventual vaccine development for humans. The GLA component (a synthetic, non-toxic moiety with six acyl chains on a disaccharide backbone and a single phosphate group (Pantel et al., 2012, Eur J Immunol, 42(1): 101-9)) would not be expected to have a TLR4 agonist immunostimulatory effect in C3H/HeJ mice which are genetically hyporesponsive to lipid A due to a mutation in the gene encoding a functional Toll-like receptor (TLR4) (Beutler et al., 2000, Eur Cytokine Netw, 11(2):143-52).

The primary outcome of this immunization study was whether mice developed severe manifestations of leptospirosis after lethal challenge infection (10⁵ organisms of a low passage (P3) with L. interrogans serovar Canicola strain LOCaS46 strain, which has a median lethal dose LD50<100 (Salinas et al., 2020, Vaccines (Basel), 8(4)). Mice were euthanized and considered having arrived at a severe disease endpoint if they developed severe manifestations after challenge infection as defined by weight loss of >15% from the beginning of the experiment, or if they were unable to groom, eat, drink, or developed severe lassitude/hunching. The secondary outcomes were 1) quantitative bacterial load in liver and kidney as measured by quantitative real time PCR, and 2) antibody responses measured by ELISA and Western immunoblots.

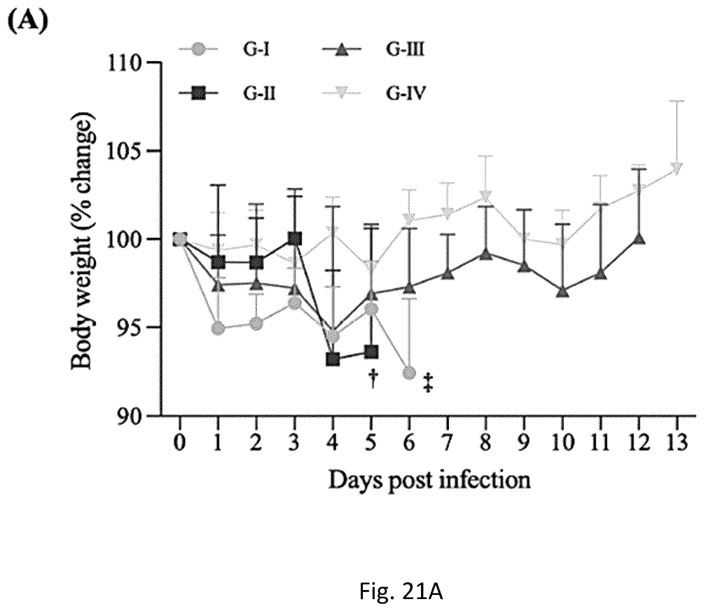

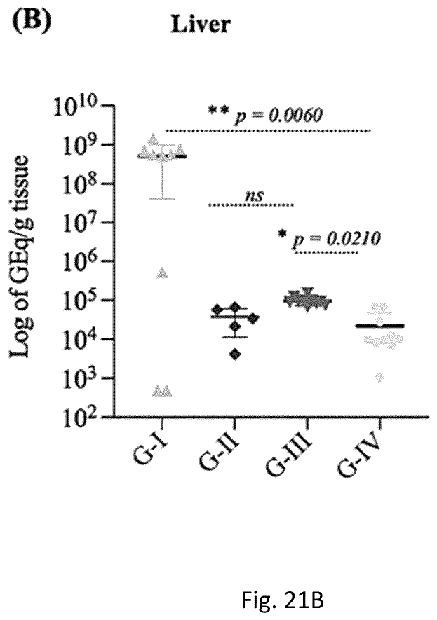

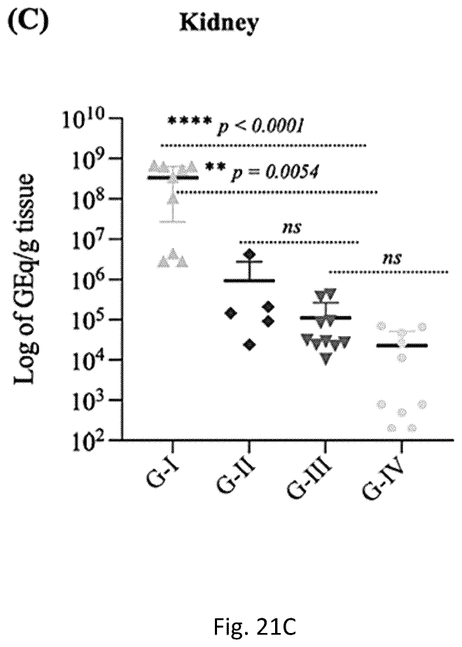

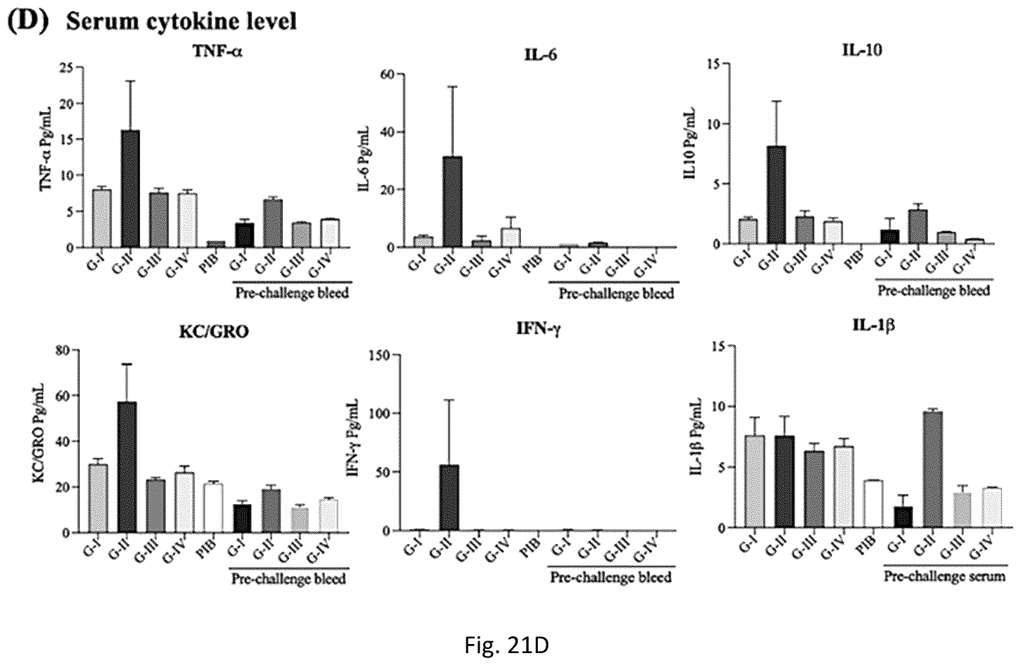

No mouse developed severe disease after the immunization protocol. Mouse groups receiving PBS (G-I) plus adjuvant or the ricin B-domain RBL1 [t3490, (G-II)] plus adjuvant showed a modest decrease body weight after challenge infection but had to be euthanized on days 6 and 5, respectively, because of severe illness manifested by lethargy and inability to feed/drink. Vaccination with full-length VM proteins, either a mix of 5 (G-III) or a mix of 2 (G-IV), prevented all observable clinical illness (FIG. 21A). This observation indicates that protection from severe leptospirosis required full length VM proteins.

Immunization with rVM Proteins Significantly Reduced Bacterial Load in Liver and Kidney

The leptospiral load of Leptospira in liver and kidney in the four experimental groups was quantified by qPCR. After challenge infection, the three groups immunized with recombinant proteins plus adjuvant (G-II, G-III and G-III) had ˜10³-10⁴-fold fewer genome equivalents (Geq) per gram of tissue, in liver and kidney (Kruskal-Wallis test, ANOVA result: liver p<0.0001, kidney p=0.0003) compared to the PBS control group (G-I) (FIG. 21B and FIG. 21C). Dunn's multiple comparisons statistical test with control group PBS (G-I), VM mix (G-III) p=0.0054, and VM unlabeled protein (G-IV) p<0.0001 confirmed this statistically significant difference.

Immunization with t3490 LED to Severe Disease Caused by Pro-Inflammatory Cytokines Despite Significantly Reducing Bacterial Load in Liver and Kidney

To determine whether immunization with the first highly conserved ricin B-like domain (RBL1) would confer protection from lethal challenge, and as a control for the full-length VM protein, LA3490, E. coli-produced recombinant RBL1 domain (truncated 3490, t3490) was produced and purified using identical procedures as for full length LA3490, and used for the immunization study. Surprisingly, mice (G-2) immunized with t3490 developed accelerated clinical disease after challenge infection yet had decreased bacterial load in liver and kidney (FIG. 21B, FIG. 21C). Disease enhancement in G-2 was associated with high levels of TNF-alpha, IFN-gamma, IL-6 and IL-10, and the chemokine KC/GRO compared to the PBS and full-length protein recipient groups (FIG. 21D).

### Antibody Profile and Cross-Reactivity of Mice Response to PF07598 (VM) Proteins Pre- and Post-Challenge

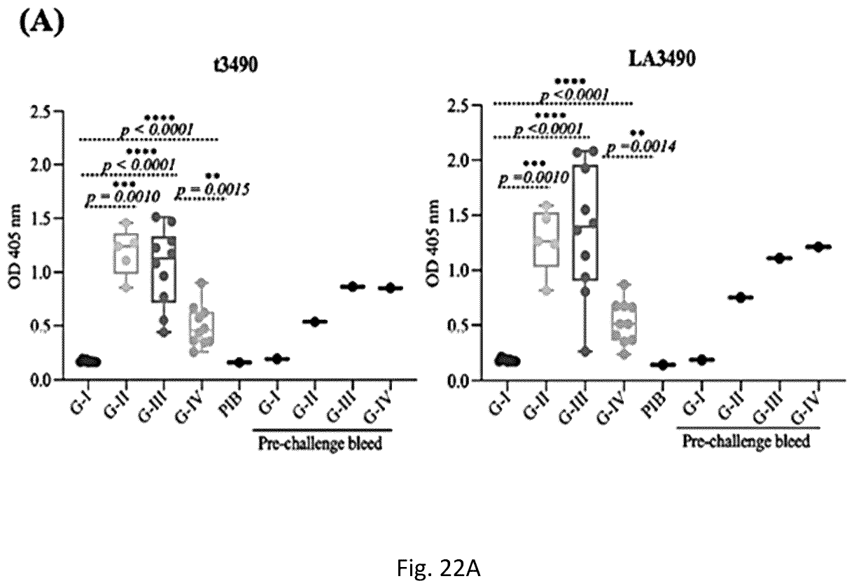

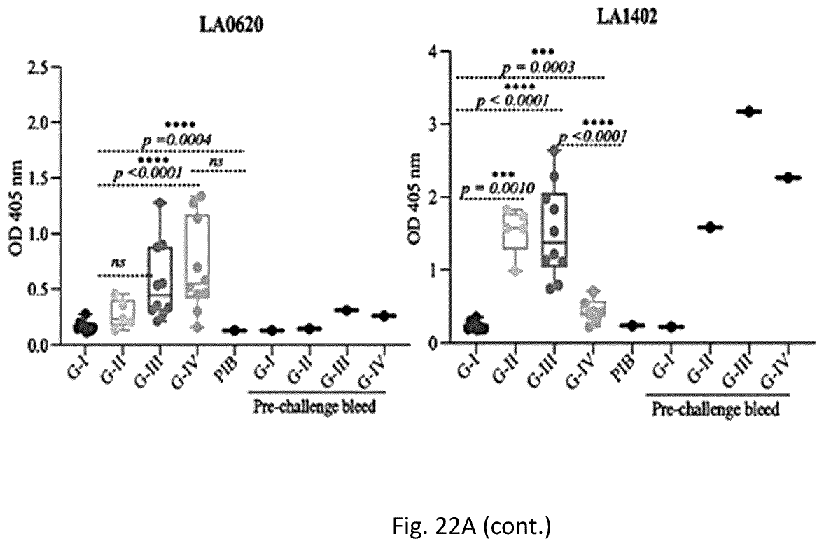

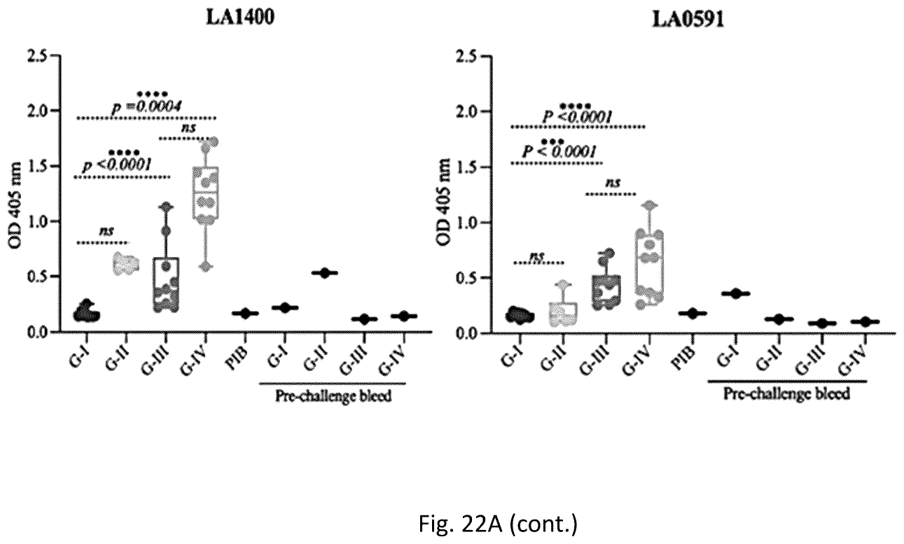

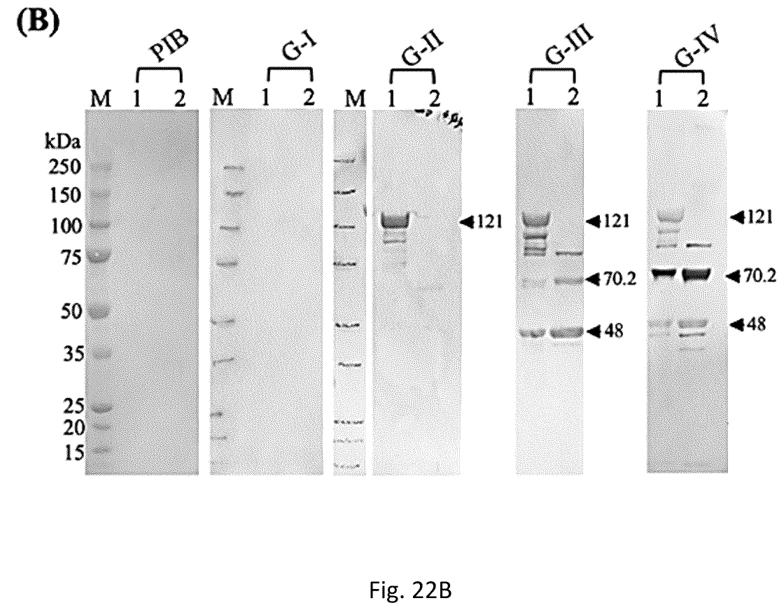

To determine whether mice immunized with VM proteins developed an IgG antibody response, sera from pre- and post-immunized mice were collected and antibody profile were examined by ELISA using all 6 antigens used in the study (FIG. 22A). Control group (G-I) and pre-immunized sera did not show detectable IgG antibody against any of the VM antigens. The antibody response against t3490 antigens was observed in sera from t3490 immunized mice (G-II) and cross-reactivity was seen with LA3490 (p=0.0010) and LA1402 (p=0.0010) antigens.

Sera from the VM mix-immunized mice (G-III) reacted with all VM antigens tested [t3490, LA3490, LA0620, LA1402, LA1400 and LA0591, (p<0.0001)]; highest titers were seen against LA3490 and LA1402 antigens. The antibody responses against each antigen in the VM mix antigen group [t3490 (p=0.0015), LA3490 (p<0.0001), LA0620 (p=0.0004), LA1402 (p<0.0001), LA1400 (p=0.0003), LA0591 (p<0.0001)] were observed with sera from VM unlabeled group (G-IV) and highest titer was detected with LA1400, LA0591 and LA0620 antigens. Antibody responses against t3490, LA3490 and LA1402 antigens were also observed in post-immunized, pre-challenge mice. Pre-challenge antibody titers to LA1400, LA0591 and LA0620 were lower than post-infection titers after challenge with live L. interrogans serovar Canicola. Further experimental investigation of the direct effect of VM proteins and their immune profiling in vivo is warranted. Despite having >90% amino acid similarity, each VM proteins shows unique reactivity with pre- and post-challenge sera and may well have different in vivo function. The differences in the reactivity of VM proteins indicates differences in immunogenicity, and because of high level of amino acid similarity they cross-react with pre- and post-challenge sera. Generation of VM protein-specific monoclonal antibodies and identification of protective epitopes would help to distinguish the roles of and mechanisms by which different VM proteins contribute to leptospirosis pathogenesis.

Cross-reactivity was confirmed by Western immunoblot analysis, probing recombinant VM proteins immobilized on nitrocellulose membrane, with pooled sera from immunized animals (FIG. 22B). Pre-bleed sera and PBS control (G-1) group did not show reactivity with a cocktail of VM mix and VM unlabeled recombinant antigens (5 and 2 proteins, respectively). Sera from t3490 immunized mice showed significant antibody titers against t3490 antigens (not shown) and cross-reacted with full-length VM proteins but faint with LA1400 and no reactivity with LA0591, which lack N-terminal, ricin B-domain, suggest that t3490 only cross react with the epitope shared at N-terminal region of VM proteins. Sera from VM mix group (G-III) cross-reacted with all five antigens and the reactivity pattern was consistent with each. The reactivity of LA1400 with sera from VM unlabeled group (G-IV) was highest among all the VM proteins and in the same cocktail lot of VM antigens immunized to G-III and G-IV mice. The finding that high titer antibodies against the LA1400 antigen (as determined by both ELISA and Western blot) were induced in the VM unlabeled group sera (G-IV) suggests that LA1400 elicits the strongest humoral immune response in mice compared to other VM proteins and may be responsible mediating protective immunity. These data do provide strong confidence, however, that one or more of these VM proteins mediate the pathogenesis in this animal model. Future optimization of which VM proteins should be used for vaccination based on these observations is supported by these data.

### VM Protein Expression in In Vitro and In Vivo and Cross-React Among Pathogenic Serovars

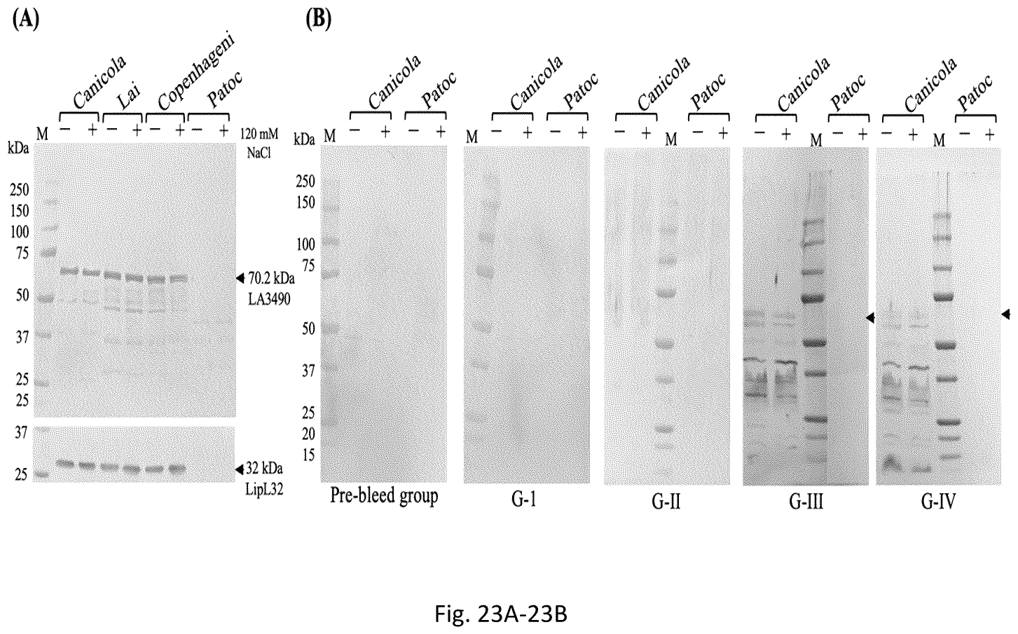

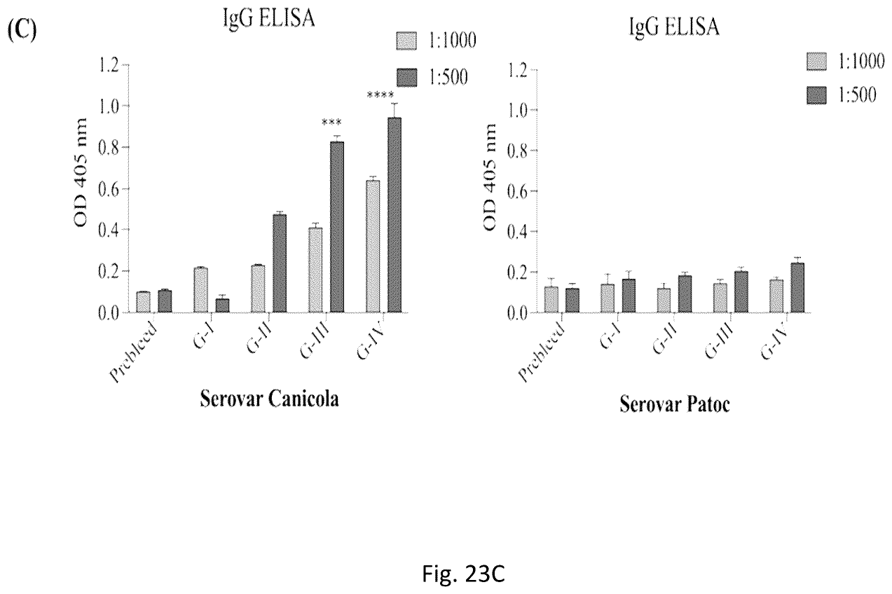

Protein extracts from L. interrogans serovar Lai, Canicola and Copenhageni and non-pathogenic strain L. biflexa serovar Patoc induced with and without 120 mM NaCl were probed on Western blots using polyclonal anti-LA3490 antibodies. Native VM protein expression was seen at the expected size of ˜70 kDa molecular weight by pathogenic serovars Lai, Canicola and Copenhageni but not with serovar Patoc (a negative control given the absence of PF07598 gene family members in this saprophytic species) (FIG. 23A).

To determine whether immunization with a limited set of VM proteins leads to broadly cross-reactive anti-VM protein antibodies, in vivo VM protein expression and cross-reactive serovar immune profiles was inferred by Western immunoblot analysis using cell-free protein extracts from Leptospira interrogans serovar Canicola and the non-infectious saprophyte L. biflexa serovar Patoc. Antibodies from the immunized groups showed antibodies against serovar Canicola recognized the predicted size of VM proteins (˜70 kDa), suggesting in vivo VM protein expression during challenge infection. This antibody reactivity also reacted with post-challenge G-III and G-IV sera. However, reactivity was not seen with the negative control, serovar Patoc cell-free lysate (FIG. 23B). Lower molecular weight reactive proteins were detected with sera from G-III and G-IV, suggesting the possibility that VM proteins undergo proteolytic processing (FIG. 23B). Further study is warranted to determine whether these low molecular weight proteins play a role in leptospiral pathogenesis.

IgG antibody profiles were quantified against homologous and heterologous VM proteins. Cell-free protein extracts from L. interrogans serovar Canicola and the non-pathogenic strain L. biflexa serovar Patoc were used as solid phase antigen adsorbed to ELISA plates. ELISA confirmed reactivity of serovar Canicola with sera from the VM mix (G-III) and VM unlabeled (G-IV) post-challenge mouse groups. L. interrogans serovar Lai-encoded VM proteins that were used to immunize the G-III and G-IV groups cross-reacted with lysates of serovar Canicola. Notably, orthologs of VM protein are highly conserved in L. interrogans serovars, consistent with this observed cross reactivity. Non-pathogenic serovar Patoc did not cross-react with sera from control and immunized mouse groups mice either pre- or post-challenge (FIG. 23C).

### Example 4: Mouse Monoclonal Work

Scouting was performed with five clones from YUMSIB against the target antigen LA0591 at 500 nM concentration. All five clones were determined to be positive with affinities in the range of pM to two-digit nM. Clones are ranked from highest to lowest affinity in the above table. Please note that KD from scouting at a single analyte concentration is a rough estimate only, it may differ by up to 10-fold higher or lower when compared to KD determined by full kinetics at 5-6 analyte concentration range.

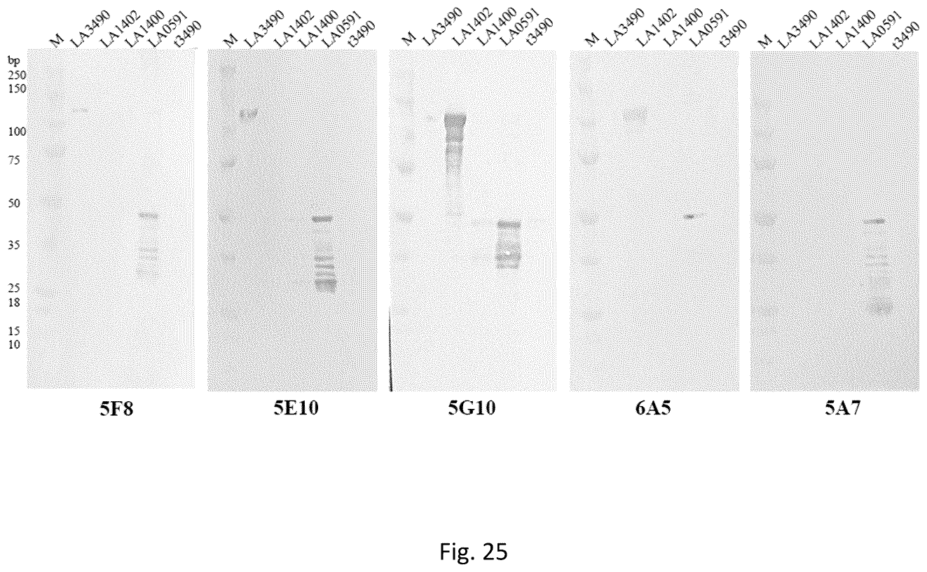

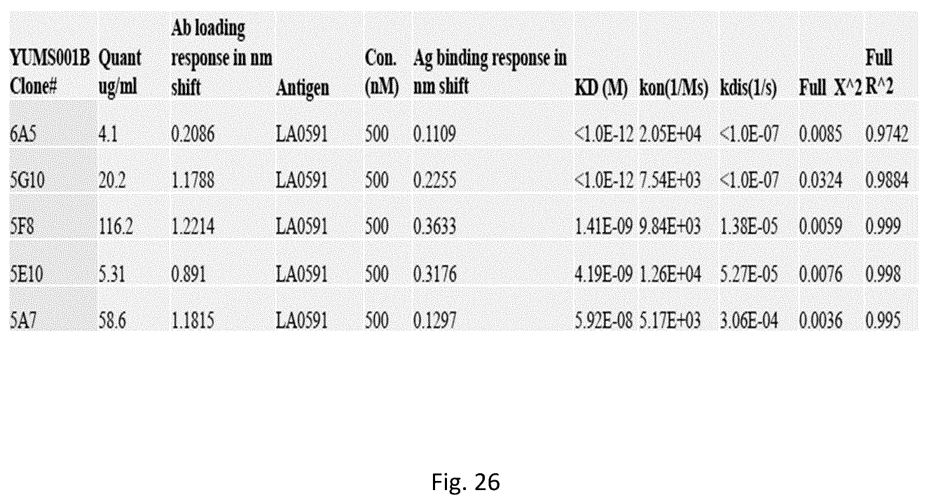

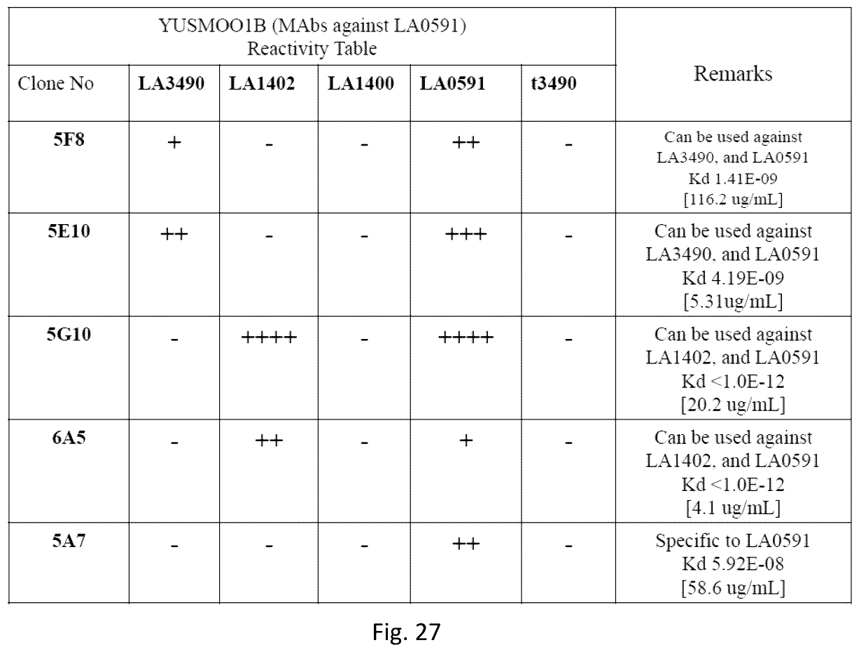

FIGS. 25-27 show the Monoclonal supernatant (YUSM001B) reactivity with recombinant VM proteins.

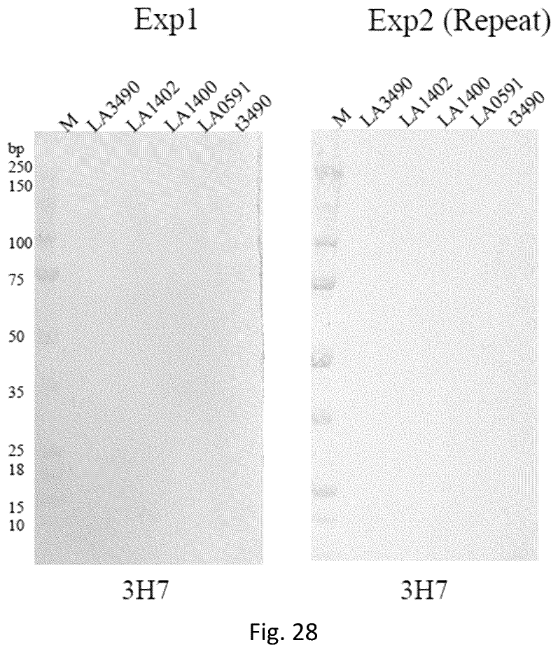

FIG. 28 shows the Monoclonal supernatant (YUSM001A, LA1400) reactivity with recombinant VM proteins.

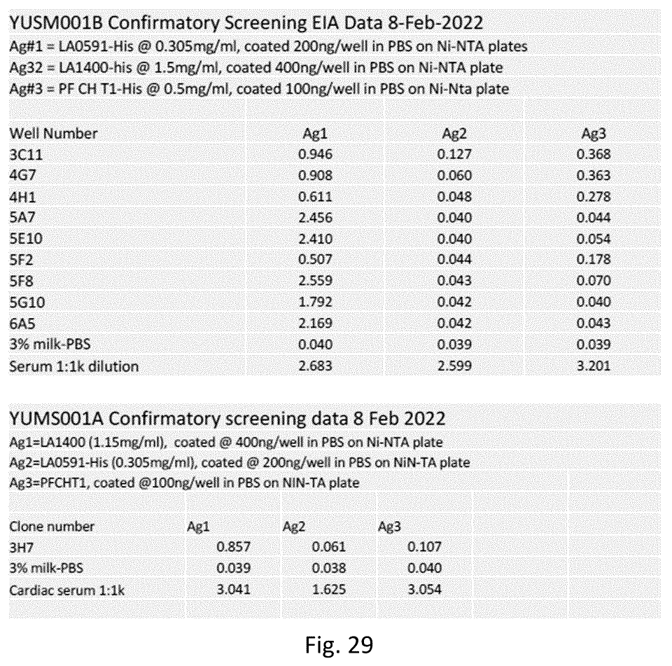

FIG. 29 provides confirmatory screening data.

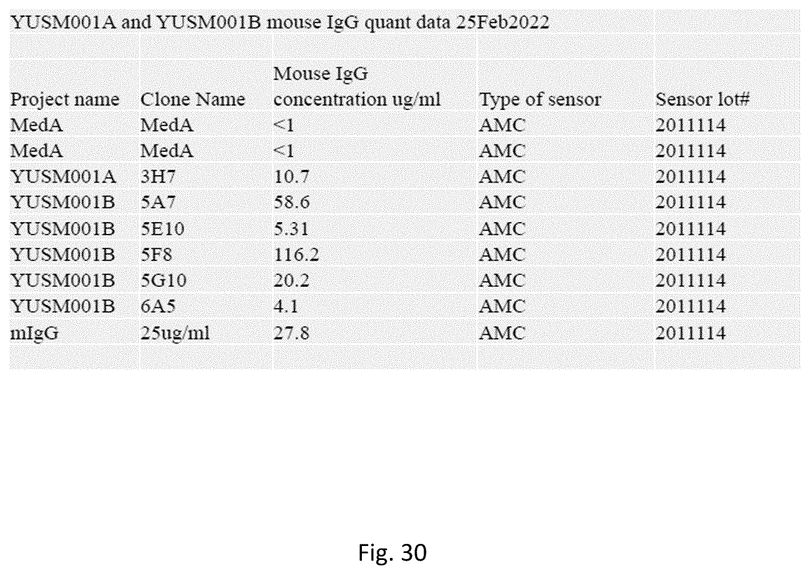

FIG. 30 provides a table of the mouse IgG quant data.

### Example 5: Pathogenic Leptospira Evolved a Unique Gene Family Comprised of Ricin B-Like Lectin Domain-Containing Cytotoxins

Here it is demonstrated that Leptospira virulence-modifying (VM) proteins, epitomized by LA3490 (Q8F0K3), are bona fide R-type lectin domain-containing cytotoxins—the first experimentally validated Leptospira exotoxins. rLA3490 binds to, and is quickly internalized by, Hela cells via an N-terminal R-type lectin domain with specificity for terminal galactosyl residues. After binding/internalization, it is translocated to the HeLa cell nucleus via a nuclear targeting signal and twin LxxLL motifs for nuclear receptor binding. Cell surface binding and internalization were shown to be rapid, occurring within 30 min after exposure. rLA3490 produced pleiotropic effects on HeLa cells, including actin depolymerization, caspase-3 activation, nuclear fragmentation, and ultimately blebbing and cell death. One mechanism of cell death appears to be originated with genomic DNA degradation, which occurs after nuclear localization of the VM protein. Corroborating in vitro experiments using purified HeLa cell genomic DNA, and supercoiled and linearized bacterial plasmid DNA indicates that rLA3490, and at least four other VM proteins tested so far, possess endo- and exo-DNase activities.

Most VM proteins, with the exception of the CBR deletion variants, fit the classical AB toxin paradigm (Odumosu et al., 2010, Toxins (Basel), 2:1612-1645). The entire leptospiral VM protein gene contains domains that are commonly encoded by two or three separate genes in other bacteria. VM proteins have at least two functionally distinct regions, with the N-terminal partly responsible for host cell targeting (binding and internalization) and the C-terminal partly mediating cytotoxicity (intracellular trafficking/enzymatic activity). The N-terminal segment is reasonably well conserved among Leptospira serovars (˜78% average pairwise amino acid identity) and contains a confirmed R-type lectin domain (amino acid positions 40-174) that shares binding specificity with ricin B chain for terminal galactosyl residues of glycoproteins. In contrast, the C-terminal segment is less conserved (˜63% average pairwise amino acid identity) and appears to mediate cytotoxicity. This sequence diversity is thought to influence VM protein cell targeting specificity (i.e., successful binding/internalization and intracellular trafficking) rather than catalytic activity, as other VM proteins tested so far exhibit DNase activity in vitro. Despite the evidence of expanded family clusters of paralogs, the reasons for the diversification of the PF07598 gene family remains uncertain, although one leading hypothesis is that paralog expansion has enabled adaption of different Leptospira to different hosts. Further experimentation and in silico analysis to compare VM protein structure and function is needed to determine any possible sequence motifs that might indicate virulence differences among PF07598 family members.

Comparison of intragenomic distances has revealed that the expanded VM proteins repertoire in virulent group I pathogenic Leptospira arose from a series of gene duplication events followed by autonomous evolution of N- and C-terminal segments, occurring more rapidly in the latter. Based upon available data, it appears that an initial duplication event produced LA1402/LA1400, which constitute the only L. interrogans VM proteins identified so far with close orthologs in less virulent group I pathogenic species (Fouts et al., 2016, PLOS Negl Trop Dis. 10(2):e0004403). This initial event was followed by successive duplications likely originating from LA1400 that formed three discrete gene clusters [A, B, and C (Fouts et al., 2016, PLOS Negl Trop Dis. 10(2):e0004403)], the largest comprising seven VM protein encoding genes, including LA3490 and LA0620. Some serovars have seemingly lost specific VM protein genes, e.g., LICRS03300 from serovar Lai and LA3271 from Hardjo, whereas others contain various CBR deletion variants. This uneven distribution of VM proteins among Leptospira serovars and their implied differences in host cell-targeting specificity is the first definitive evidence that some are intrinsically more virulent than others and, therefore, of heightened clinical and public health significance. Most VM protein-encoding genes are found only in L. interrogans and its sister species. As some VM proteins, e.g., LA3490 (Q8F0K3) have proven to be particularly toxic to human cells, their presence in serum could foreshadow severe disease complications, providing prognostic information—a cornerstone for effective clinical risk assessment.

Like ricin, for which toxicity and pathology are clearly linked and route dependent (inhalation and severe respiratory compromise being most lethal), site-specific expression of certain VM proteins coupled with their presumed differences in host cell specificity (i.e., host cell exposure and susceptibility) might explain the variable clinical presentation of severe leptospirosis. Indeed, the effort to understand the molecular and cellular pathogenesis of leptospirosis remains in its infancy, and approaches to prevent leptospirosis or ameliorate its pathogenesis are predicated on mechanistic understandings of the biology of Leptospira-host interactions. For example, pulmonary hemorrhage and refractory shock are particularly important clinical manifestation of leptospirosis (Sehgal et al., 1995, Indian J. Med. Res, 102:9-12; Marotto et al., 1999, Clin. Infect. Dis, 29:1561-1563; Segura et al., 2005, Clin. Infect. Dis, 40:343-351; Gouveia et al., 2008, Emerg. Infect. Dis, 14:505-508; Truong and Coburn, 2011, Front. Cell Infect. Microbiol, 1:24; Helmerhorst et al., 2012, Neth. J. Med. 70:215-221; Ruwanpura et al., 2012, Med. J. Malaysia, 67:595-600). Indirect evidence—that these serious manifestations are ameliorated by hemodialysis/hemofiltration (Andrade et al., 2007; Cleto et al., 2016)—suggests that there may be a circulating soluble toxin or toxins in leptospirosis. Histopathological analysis of lung tissues in severe pulmonary leptospirosis syndrome do not find intact Leptospira (Nicodemo et al., 1997, Am. J. Trop. Med. Hyg, 56:181-187), but rather damage to alveolar epithelial and activation of endothelial cells, with deposition of immunoglobulin and complement as secondary events (Nally et al., 2004; Croda et al., 2010, Clin. Microbiol. Infect, 16:593-599; De Brito et al., 2013, PLOS One, 8:e71743). Nonetheless, apart from various sphingomyelinases/hemolysins (Narayanavari et al., 2015, PLOS Negl. Trop. Dis. 9:e0003952; Chaurasia and Sritharan, 2020, Microbiology (Reading) 166, 1065-1073) and a collagenase (Kassegne et al., 2014, Leptospira species. J. Infect. Dis, 209:1105-1115), a few potential leptospiral toxins have been identified, and none adequately explain the pathogenetic features of the diverse clinical spectrum of leptospirosis. Nonetheless, hemodialysis and hemofiltration remain life-saving interventions but exceed the clinical resources and/or capabilities in the vast majority of leptospirosis-endemic regions. By contrast, as with other AB toxins (Odumosu et al., 2010, Toxins (Basel), 2(7): 1612-45), e.g., CARDS (Somarajan et al., 2014, mBio, 5:001497-c01514; Becker et al., 2015, Proc Natl Acad Sci USA, 112(16):5165-70) and ricin (Yermakova et al., 2014, mBio, 5:e00995; Gal et al., 2017, Toxins (Basel), 9:311), mitigating VM protein toxicity using monoclonal antibody (mAb)-based biologics or small molecule inhibitors (Benz and Barth, 2017, Curr. Top. Microbiol. Immunol, 406:229-256) that perturb VM protein cell-surface binding/cell entry and/or intracellular trafficking/toxicity would constitute more generally accessible alternatives.

The data presented here build upon previously published observations (Matsunaga et al, 2007; Infect Immun, 75(6): 2864-2874; Lehmann et al, 2013, PLOS Negl Trop Dis, 7(10): e2468; Fouts et al., 2016, PLOS Negl Trop Dis. 10(2):e0004403) indicating that leptospiral VM proteins are major virulence factors presumably involved in the molecular and cellular pathogenesis of leptospirosis. Transposon mutagenesis screens have since shown that multiple VM proteins, particularly Q8F6G8 (gene ID, LA0589), contribute to lethal disease in hamsters (Murray et al., 2009, Infect. Immun, 77:810-816; Truong and Coburn, 2011, Front. Cell Infect. Microbiol, 1:24). Nonetheless, until now, VM proteins were uninformatively classified as PF07598, a protein family of unknown function. Here, Phyre2-based predictions are confirmed that these proteins belong to a superfamily of proteins, the R-type lectins, which possess carbohydrate-binding activity named for and structurally similar to ricin B chain, and are found in plants, animals, and bacteria (Cummings et al., 2017, Cold Spring Harbor Laboratory Press). Ricin and its B chain (and other R-type lectins) bind to terminal galactoses or other related glycans of a diverse range of host cell surface glycoconjugates, which facilitates translocation and internalization of the ricin A chain into target cells, resulting in cell death via inhibition of protein synthesis (Montanaro et al., 1973, Biochem. J, 136:677-683; Sperti et al., 1973, Biochem. J, 136:813-815; Lord et al., 2003, Toxicol. Rev, 22:53-64; Sowa-Rogozinska et al., 2019, Toxins (Basel), 11:350). Likewise, bacterial AB toxins, such as Shiga, pertussis toxins, and diphtheria toxins, mediate cell death either by ADPribosylation of 28S rRNA, or ai subunits of the heterotrimeric G protein or by inactivation of elongation factor 2 (Brown et al., 1980 FEBS Lett. 117, 84-88; Cemal, 1999, Design And Construction Of Membrane-Acting Immunotoxins For Intracellular And Secreted Protein Expression In Pichia Pastoris. Ph.D. Thesis, University of Cambridge, Cambridge; Coutte and Locht, 2015, Future Microbiol, 10:241-254; Cherubin et al., 2018, Sci Rep, 8(1):2494). Genotoxins like Leptospira VM proteins, e.g., cytolethal distending toxin (CDT), are less well studied. With the characterization of Leptospira VM proteins, some general features of bacterial genotoxins have come into focus. For example, they exhibit DNase activity, are translocated to the nucleus via a nuclear localization signal (McSweeney and Dreyfus, 2004, Cell Microbiol, 6:447-458), and have pleiotropic effects on target cells, including causing cell death via caspase 3-dependent and -independent mechanisms (Ohara et al., 2008, Infect. Immun, 76:4783-4791).

By recontextualizing the data presented here within the classical AB toxin paradigm, it is proposed that following cell surface binding—possibly via mannose receptor, as has been reported for ricin B (Simmons et al., 1986, J. Biol. Chem, 261:7912-7920; Lord et al., 1992, Biochem. Soc. Trans, 20:734-738; Newton et al., 1992, J. Biol. Chem, 267:11917-11922) given overlapping VM protein-ricin B carbohydrate-binding specificities, VM proteins are endocytosed—as are ricin and most AB toxins, e.g., Shiga toxin (Arfilli et al., 2010, Biochem. J, 432:173-180); released to the cytoplasm then ferried to the nucleus via an internal nuclear localization signal gaining entry after binding at the nuclear pore complex via one or more LxxLL motif-containing amphipathic a-helices; and then actively translocated through the pore into the nucleoplasm, leading to nuclear fragmentation via intrinsic exonuclease activity; possibly inducing caspase-3 activation and cytoskeleton disassembly via yet unknown mechanisms during transit, when free in the cytosol.

While it is demonstrated that ricin B-like lectin domains of distinct VM proteins (rLA3490, t3490, and t0620) bound to immobilized asialofetuin (Wales et al., 1994, Glycoconj J, 11:274-281; Frankel et al., 1996, Biochemistry, 35:14749-14756; Dawson et al., 1999, J. Appl. Toxicol, 19:307-312), native target ligands and cellular targets are yet to be defined. Second, differences in cytopathic potential among VM proteins, their host cell targeting specificities, and molecular pathways by which they exert their pleiotropic effects remain to be fully explored, although these initial experiments indicate that they are likely AB-type cytotoxic genotoxins. Third, while it is becoming increasingly evident that leptospiral VM proteins arose from successive gene duplication events, the reasons for the expanded repertoire in L. interrogans, L. kirschneri, and L. noguchii (Lehmann et al, 2013, PLOS Negl Trop Dis, 7(10): e2468) compared with other pathogenic Leptospira species (Lehmann et al, 2013, PLOS Negl Trop Dis, 7(10): e2468; Fouts et al., 2016, PLOS Negl Trop Dis. 10(2):e0004403) is not understood, nor is the reason for the uneven distribution of VM paralogs among Leptospira serovars types understood, although yet unknown ecological niche specialization is quite plausible, presumably as a defense against eukaryotic predation in soil/surface water, akin to Shiga toxin production in E. coli (Lainhart et al., 2009, J. Bacteriol, 191:5116-5122).

The materials and methods used for the experiments are now described

### Computational Analysis

To identify functional subdomains, the amino acid sequences of Q8F0K3 and its closest paralog Q8F8D7-encoded by LA3490 and LA0620 in L. interrogans serovar Lai, respectively-were submitted separately to the Phyre2 remote homology search portal1 (Kelley et al . . . 2015). Short functional regions and motifs, including amphipathic potentially membrane-binding a-helices, putative eukaryotic protein-sorting signals, proteolytic cleavage, and phosphorylation sites, and binding/docking motifs, were identified via HeliQuest2 (Gautier et al., 2008, Bioinformatics, 24:2101-2102) and the Eukaryotic Linear Motif (ELM) resource3 (Kumar et al., 2020, Nucleic Acids Res. 48. D296-D306). About 3,000 PF07598|VM proteins representing all clinically relevant Leptospira species—as well as L. alexanderi and L. alstonii, were aligned against a custom-built HMM model, which was based upon the full PF07598 reference alignment. Following visual inspection of the aligned amino acid residues, any (VM protein) sequence that contained ambiguous amino acids was annotated as partial-if derived from draft genomes; those that did not span at least one presumed functional subdomain [i.e., any of RBL1/RBL2/CTD (carboxy terminal domain)] were excluded. For clustering analysis, subalignments encompassing the CBR (carbohydrate-binding region) and CTD (i.e., aa positions 23-343 and 344-639, respectively, with respect to Q8F0K3) were removed from the curated global alignment and used as input for computation of discrete all vs. all pairwise distance matrices using the R package, bio2mds (Pele et al., 2012, BMC Bioinformatics, 13:133), excluding “gappy” columns (i.e., containing >50% gaps). Pairwise distance matrices demonstrating close amino acid relatedness of the full-length alignment and CBR- and CTD-subalignments are provided for six leptospiral serovars of public health importance (L. interrogans serovars Copenhageni, Canicola, Hardjo, Lai, Manilae, and Pomona and L. kirschneri Pomona). Poorly aligned columns were improved manually via visual inspection in Jalview v2.11.4. Fragmented VM proteins (i.e., that did not span at least one functional subdomain) were removed. The curated multiple sequence alignment was used as input for HMM profile-based remote homology searches against PDB, SCOPe70, SMARTv6, and UniProt-SissProt-viral70 databases via HHpred4. A consensus secondary structure was predicted using Ali2D5 and the 3D (protein) structure predicted using AlphaFold (Callaway et al., 2020, Nature, 588(7837):203-4; Jumper et al., 2020, predictioncenterorg/casp14/doc/CASP14_Abstracts; Senior et al., 2020, Nature, 577(7792):706-10). Separate distance matrices for the amino terminal half, encompassing the predicted lectin domain, and the C-terminal half containing the putative toxin subdomain were generated to infer their evolutionary relatedness and clustering relationships.

### Leptospira Culture and Virulence Gene Expression In Vitro

Low-passage, virulent L. interrogans serovar Lai strain 56,601 that had been passaged through hamsters to recover high virulence (LD50<100) (Lehmann et al, 2013, PLOS Negl Trop Dis, 7(10): e2468) were maintained at 30° C. in semisolid Ellinghausen, Mccullough, Johnson, and Harris medium (EMJH, BD Biosciences, United States) (Ellinghausen et al., 1965, Am J Vet Res, 26:39-44). Because the published data showed that VM proteins are transcriptionally upregulated in vivo in a hamster model of acute leptospirosis (Lehmann et al, 2013, PLOS Negl Trop Dis, 7(10): e2468) and to maximize their expression in vitro, Leptospira were grown under conditions mimicking the in vivo host environment known to induce virulence gene expression in vitro (Matsunaga et al., 2005, Infect Immun, 73(1):70-8). Mid-logarithmic cultures (2×10⁸ leptospires/ml) in EMJH medium were harvested by centrifugation at 18,514 g. Pelleted cells were washed twice with 1×PBS, resuspended in liquid EMJH medium supplemented with 120 mM NaCl, and then incubated at 37° C. for 4 h (Sigma Aldrich, United States).

Following virulence gene induction, culture supernatants were collected by centrifugation at 18,514×g for 20 min, clarified by filtration through a 0.22-mm membrane filter (Merck Millipore, Germany), and then concentrated via a 30-kDa Amicon® Ultra centrifugal filter (Merck Millipore, Germany). Induced and uninduced (corresponding to baseline in vitro expression) culture supernatants were analyzed by Western blot probed with rabbit anti-LA3490 polyclonal antiserum. Total protein was estimated by BCA assay (Pierce™ BCA Protein Assay Kit, Thermo Fisher Scientific, United States).

### Mammalian Cell Culture

HeLa cells obtained from the American Type Culture Collection (ATCC, United States) were grown as monolayers in tissue culture plates in Dulbecco's modified Eagle medium (DMEM; Sigma-Aldrich, United States) supplemented with 10% fetal bovine serum and 1% antibiotic-antimycotic solution (penicillin, 100 units/ml; streptomycin, 100 mg/ml; and amphotericin, 25 mg/ml; Invitrogen, United States) at 37° C. in a humidified incubator containing 5% CO₂. Antibiotic-containing medium was replaced with fresh, antibiotic-free medium prior to each experiment.

### Plasmid Constructs and Cloning

Escherichia coli codon-optimized gene fusions consisting of either the complete LA3490 coding sequence (NP_713670.1) minus the predicted signal peptide (i.e., corresponding to nucleotide positions 57-1,917 bp) or an N-terminal truncation inclusive of positions 40-174 bp and encompassing the Phyre2-predicted ricin B-like lectin subdomain linked to mCherry (AST15061.1) via a glycine-serine hinge (GGGGSGGGGSGGGGS; SEQ ID NO:93) were synthesized and cloned into pET32b (+) (Gene Universal Inc., United States). Prior to use, constructs were verified by sequencing.

### Recombinant Protein Expression and Purification

Because PF07598 proteins are cysteine-rich [LA3490 encode 12 cysteine], recombinant proteins were expressed in SHuffleR T7-competent E. coli cells (New England Biolabs, United States) owing to their capacity to promote disulfide bonds in the cytoplasm ensuring proper protein folding. Transformants were subcultured into Luria-Bertani (LB) medium containing 100 mg/ml of ampicillin. When cultures had reached an OD of 0.6, expression was induced at 16° C. and 250 rpm for 24 h via addition of 1 mM isopropyl-b-D-thiogalactoside (IPTG; Sigma-Aldrich, United States).

Following induction, cells were pelleted by centrifugation and then lysed in CelLytic™ B (Cell Lysis Reagent; Sigma-Aldrich, United States) containing 50 U of benzonase nuclease (Sigma-Aldrich, United States), 0.2 mg/ml of lysozyme, non-EDTA protease inhibitor cocktail (Roche, United States) plus 1 mM PMSF (Sigma-Aldrich, United States) for 30 min at 37° C. Lysates were centrifuged at 4° C. and 18,514×g for 10 min. Supernatants and pellets were separated, and then analyzed by 4-12% bis-tris sodium dodecyl sulfate-polyacrylamide gel electrophoresis (SDS-PAGE).

As above, protein concentrations were determined by BCA assay. Recombinant thioredoxin (TRX)-His6-VM protein-(GGGGSGGGGSGGGGS; SEQ ID NO:93)-mCherry-His6 fusion proteins were isolated using a 5-ml pre-packed Ni-Sepharose AKTA Hi-TRAP column (GE Healthcare, United States) pre-equilibrated with a buffer containing 100 mM NaH2PO4, 10 mM Tris-HCl, and 25 mM imidazole, pH 8.0. Bound fusion protein was then eluted from the column in the presence of 500 mM imidazole, pH 8.0. Eluates were pooled, concentrated via a 30 kDa Amicon® Ultra centrifugal filter, and then centrifuged using a high-capacity endotoxin-removal spin column (Thermo Fisher Scientific, United States) to eliminate lipopolysaccharide contamination. Recombinant protein preparations were dialyzed overnight against 1×PBS (pH 7.4) with gentle stirring (350 rpm) at 4° C. (30-kDa cutoff, Slide-A-Lyzer, Thermo Scientific™, United States), followed by size exclusion via a 40-kDa Zeba™ desalting spin column (Thermo Fisher Scientific, United States) to remove imidazole, and then stored at −80° C. until use.

### Asialofetuin Binding and Ricin B-Chain Competitive Binding Assays

To confirm binding specificity of the Phyre2 predicted ricin B-like lectin domain, it was tested whether E. coli-produced, recombinant full-length, and truncated Q8F0K3 (i.e., rLA3490 and t3490, respectively) and Leptospira secreted VM proteins bound to immobilized asialofetuin as does ricin B. Binding assays using rLA3490/t3490 were done using ImmulonR 2HB flat-bottom microtiter plates (Thermo Fisher Scientific, United States). Plates were precoated with asialofetuin (5 ng/ml in carbonate-bicarbonate buffer, pH 9.4), incubated at 4° C. overnight. Prior to use, plates were blocked for 2 h at 37° C. with 5% non-fat dry milk in 1×TBST. After blocking, rLA3490, t3490, and recombinant ricin B chain (Vector Laboratories, Inc., United States) were added separately and in triplicate at molar concentrations of 0.9, 4.50, and 9.05 nM in 1×TBST. Plates were incubated for 2 h, washed thrice with 1×TBST, and then incubated for 1 h with anti-LA3490 polyclonal antibodies or anti-ricin B-chain monoclonal antibody, 1:1,000 in TBST (Invitrogen, United States). To quantify bound rLA3490/t3490, plates were incubated with goat anti-mouse IgG (1:5,000; KPL, United States) for 1 h, washed thrice with TBST, and developed with p-nitrophenyl phosphate (1-Step™ PNPP Substrate Solution; KPL, United States). The reaction was stopped with 2 M NaOH, and absorbance was read at 405 nm using a SpectraMaxR M2e Microplate Reader (Molecular Devices, United States). For competitive binding assays, precoated with asialofetuin (2.5 ng/ml) plates were preincubated with either 25 or 50 nM recombinant ricin B chain (Vector Laboratories, United States) for 2 h prior to the addition of 50 nM of rLA3490/t3490 and a final 2-h incubation. Bound recombinant protein was quantified using anti-LA3490 polyclonal antibodies. The capacity of Leptospira-secreted VM proteins to bind asialofetuin was evaluated using coated SepharoseR beads. Commercially available asialofetuin (1 mg/ml), (Sigma-Aldrich, United States) dissolved in 0.1 M NaHCO₃, was coupled with PBS-washed, NHS-activated Sepharose beads (GE Healthcare, United States). The suspension was agitated slowly at room temperature for 1 h, and unoccupied NHS groups blocked with 1 M ethanolamine, pH 9 for 1 h. Washed beads were incubated with 250 mg of clarified Leptospira culture supernatant containing secreted proteins for 1 h, and then washed twice with MEPBS (4 mM b-mercaptoethanol, 2 mM EDTA, and 20 mM Naphosphate, pH 7.2) buffer at 200×g for 1 min. Bound proteins were eluted with 0.5 M lactose and analyzed by 4-10% bis-tris SDS-PAGE followed by Western blot with mouse anti-LA3490 polyclonal antibodies (1:2,000 dilution) as above.

rLA3490-Mediated HeLa Cell Cytotoxicity

HeLa cells (35,000 cells/200 ml) were seeded in eight-well chamber slides (LabTek, United States) and incubated at 37° C. in a humidified atmosphere containing 5% CO₂ for 24 h. Cells were treated with a pre-optimized concentration of 45 nM rLA3490; t3490- and BSA-treated and untreated Hela cells served as controls. Slides were incubated for up to 4 h, and timelapse images were taken at ×40 objective lens using a Leica DMi8 inverted microscope (Leica Microsystems, Germany). Adherent cells, before and after exposure to either rLA3490, t3490, or BSA or untreated HeLa cells were captured via a _10 objective, and the cells were counted using LAS AF 2D quantitative image analysis software (Leica Application Suite X, LAS X; Leica Microsystems, Germany). A grayscale prefilter was applied to improve image clarity. Detachment was quantified as 100×(#cells at 4 h/#cells at 0 h) and is reported as the average of two or more replicate experiments.

### Live/Dead and LDH Assay, and F-Actin Staining

HeLa cells were exposed to 45 nM of rLA3490 or t3490 for 4 h. Monolayers were then washed twice with 1×PBS, pH 7.4; 200 ml of 2 mM calcein AM/4 mM ethidium homodimer-1 dissolved in PBS (Live/Dead® Viability Kit, Invitrogen, United States) was added to each well, and plates were incubated for 30 min in the dark. Monolayers were washed with PBS pH 7.4 to mitigate non-specific, background fluorescence. BSA and untreated HeLa cells were used as controls. Images were taken using a Leica DMi8 microscope via a 10× objective with appropriate excitation and emission filters for green (live cell) and for red (dead) fluorescence. Cell lysis was quantified by assaying the concentration of lactate dehydrogenase in culture supernatants (CyQUANT™ LDH Cytotoxicity Assay, Invitrogen, United States). For F-actin staining, cell monolayers were exposed for up to 1 h, washed twice with PBS, pH 7.4, and then fixed with 4% paraformaldehyde (Sigma-Aldrich, United States) for 30 min at room temperature. Following aspiration of the fixative, monolayers were washed twice with PBS, then 0.1% Triton X-100 in PBS was added to each well for 5 min prior to repeat washes with PBS. Monolayers were incubated with phalloidin Alexa_488 nm conjugate (Invitrogen, United States) at room temperature for 30 min in the dark per manufacturer's directions. Nuclei were stained with 0.1 mg/ml of ProLong™ Gold Antifade Mount with DAPI for 10 min. All images were taken using a Leica DMi8 microscope with appropriate filters [Alexa_488 nm (green), DAPI (blue)] at ×40 objective lens.

Internalization of rLA3490 by HeLa Cells

HeLa cells were seeded in eight-well chamber slides (LabTek, United States) and incubated as described above. Monolayers were treated with 45 nM of rLA3490 or t3490 for up to 60 min, washed twice with PBS, and then stained with CellMask™ Green Plasma Membrane Stain (Invitrogen, United States) per manufacturer's directions; nuclei were stained with 0.1 mg/ml of ProLong™ Gold Antifade Mount with DAPI for 10 min. Images were taken using a Leica SP8 Gated STED 3×superresolution confocal microscope (Leica Microsystems, Germany) via a ×100 objective lens with oil immersion. Three dimensional z projections were obtained from 24-42 crosssectional images (depth, 6.87 mm; separation, 298.5 nm) via the LAS X software.

### DNA Fragmentation/Laddering Assays

DNase assays were done in a final volume of 15 ml of 1× Trismagnesium Chloride™ sample buffer containing 10 mM Tris and 3 mM MgCl₂ (PH 7.5) (Nakamura and Wisnieski, 1990. J. Biol. Chem, 265:5237-5241) using isolated HeLa cell genomic DNA (QiAmp DNesay Blood and Tissue kit; Qiagen, Invitrogen, United States); monolayers were trypsinized when 90% confluent, and genomic DNA isolated per manufacturer-recommended protocol for nucleated mammalian cells. Recombinant proteins rLA3490 or t3490 (as negative control) were diluted in TM and allowed to equilibrate to 22° C. prior to use. Triplicate reactions containing either 3, 10, 30, or 100 nM of recombinant protein were initiated with 150 ng of genomic DNA (pre-equilibrated to 22° C.), terminated upon addition of premixed loading buffer, gel loading dye, purple (6×) (New England Biolabs, United States), at different time points and then analyzed by 1% agarose gel electrophoresis; gels were stained with ethidium bromide (0.5 ml/ml). Gel images were taken using Gel Doc UV illumination (Gel Logic 212 Pro, Carestream Molecular imaging, United States). Endonuclease activity was assessed using 400 ng of undigested (i.e., supercoiled) pET plasmid vector or HindIII-digested pET plasmid vector as input. These assays were repeated using a complementary, fluorescence-based approach utilizing a dual-labeled oligonucleotide probe, labeled with fluorescein at its 50 end and black hole quencher, BHQ-1R, at its 30 end, that fluoresces intensely (excitation/emission 495/520 nm) in the presence of DNase. As before, recombinant proteins were diluted in TM (3, 10, or 30 nM), and allowed to equilibrate to 22° C. for 10 min prior to use. Twenty-microliter reactions containing master mix, oligonucleotide probe, detection buffer, ROX reference dye, and 10 ml of pre-equilibrated recombinant protein (in TM buffer) were subjected to a two-step thermocycling protocol consisting of 36° C., 10 s and 37° C., 50 s for 30 cycles using a CFX96 Touch Real-Time PCR Detection System (Bio-Rad, United States). Fluorescence was recorded in 5-min increments. DNase I (0.02 units/ml) was used as positive control, and PCR grade water as negative control.

### Statistical Analysis and Image Editing

All experiments were performed in triplicate and repeated at least twice to assess reproducibility. Results are expressed as mean and standard deviation. An unpaired, two-tailed student's t-test was used to assess statistical significance. Data were visualized via Graph Prism 8. All figures were produced using Adobe Illustrator.

The Experimental Results are now described

### Unique Architecture of Leptospiral Virulence-Modifying Proteins

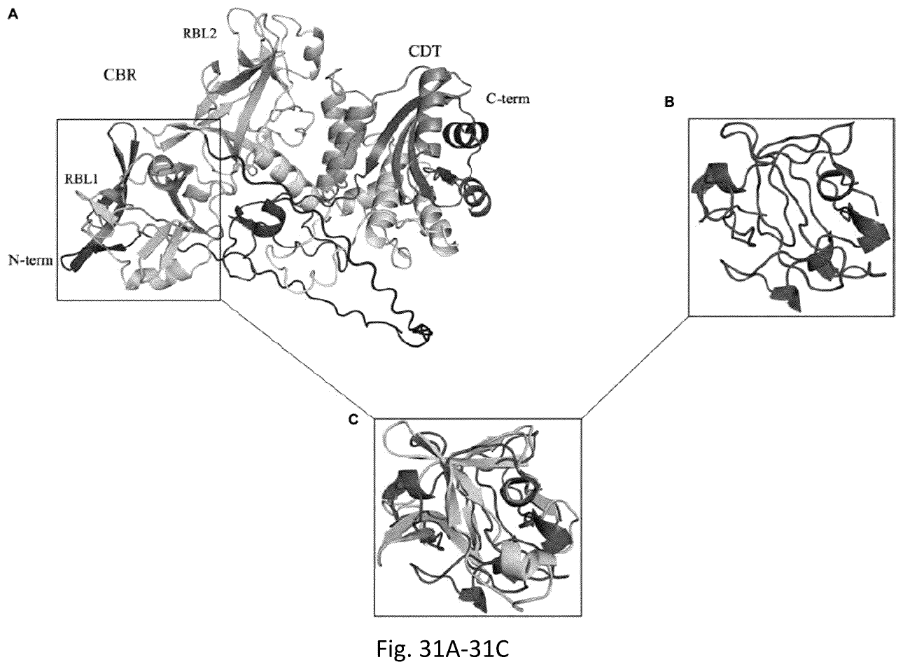

Virulent Leptospira spp. encode 10 to 12 multidomain VM proteins of ˜640 amino acids (aa), each containing tandem N-terminal ricin B-like lectin (RBL) subdomains: RBL1 and RBL2-collectively referred to as the carbohydrate-binding region (CBR) or lectin domain (FIG. 31). The presence of these tandemly repeated RBLs is similar to the Mycoplasma pneumoniae community-acquired respiratory distress syndrome (i.e., CARDS) toxin (Becker et al., 2015, Proc Natl Acad Sci USA, 112(16):5165-70)—although in reverse orientation (FIG. 31), but the C-terminal regions of the Leptospira VM proteins and the CARDS toxin are not related. Leptospira VM proteins are contained in a single polypeptide transcribed from a single genetic locus, distinct from most other bacterial AB toxins, which are typically encoded by two or more genes—hence the A-B designation—and assembled into multimeric protein complexes (Brown et al., 1980 FEBS Lett. 117, 84-88; Nakamura and Wisnieski, 1990. J. Biol. Chem. 265:5237-5241; McSweeney and Dreyfus, 2004, Cell Microbiol, 6:447-458; Coutte and Locht, 2015, Future Microbiol, 10:241-254; Cherubin et al., 2018, Sci Rep, 8(1):2494). Unique to L. interrogans, natural CBR deletion variants (˜313 aa) containing a predicted signal sequence are present (see below).

### Distribution and Evolution of Leptospira Virulence-Modifying Protein Variants

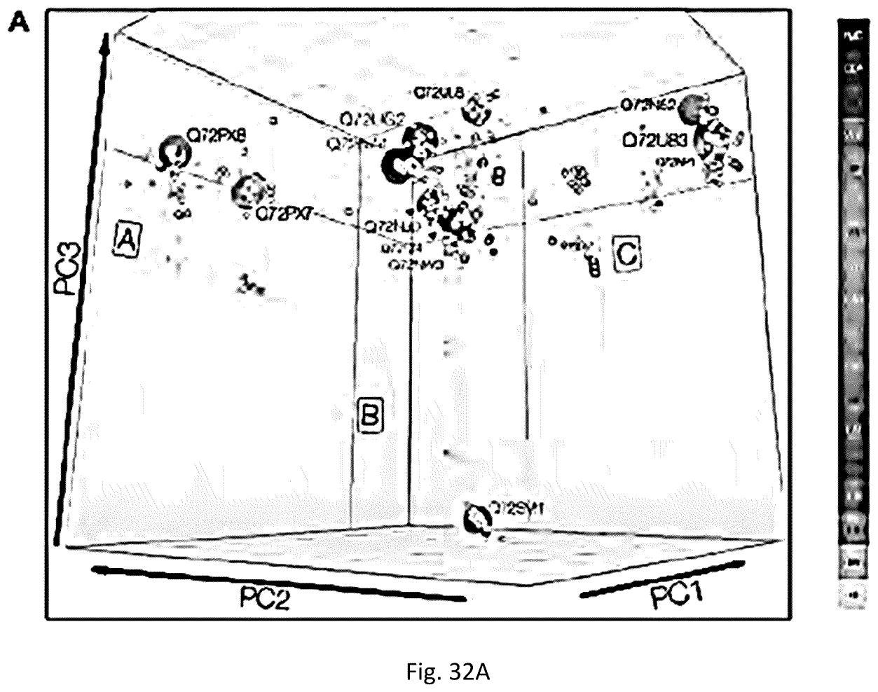

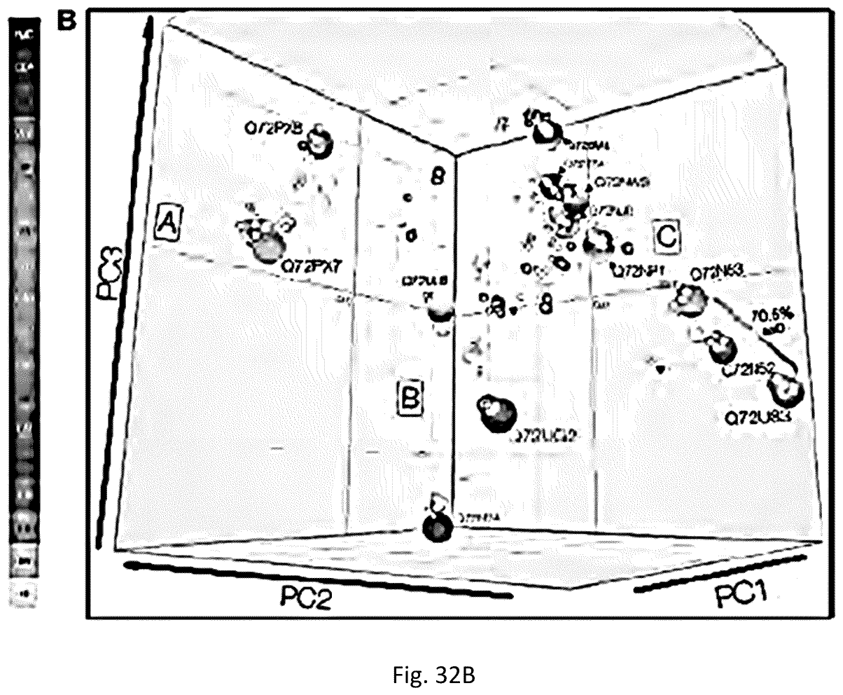

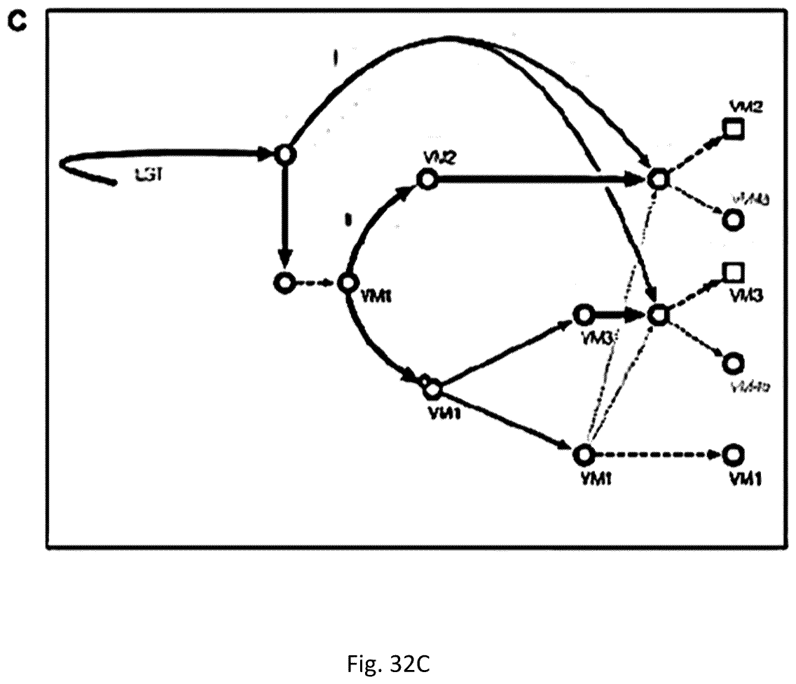

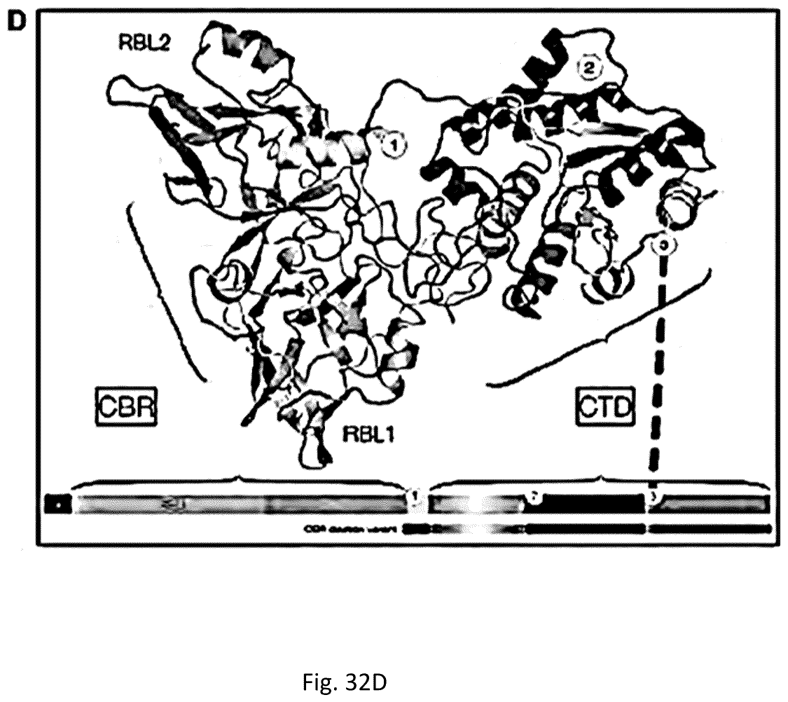

Comparative whole-genome analyses of all (at the time) recognized pathogenic Leptospira spp. suggested that gene duplications and decay produced an uneven distribution of VM protein variants among the virulent group I Leptospira, which radiated further only in L. interrogans, L. kirschneri, and L. noguchii (Fouts et al, 2016, PLOS Negl Trop Dis. 10(2): e0004403). Following distance matrix computation, three-dimensional (3D) metric multidimensional scaling (MMDS) was used to define and visualize orthologous clusters using customized R scripts and bio2mds (FIGS. 32A,B). For consistency, clusters are named using the relevant Copenhageni UniProtKB IDs. VM protein paralogs (and their derivative CBRs and CTDs) are referenced using assigned (orthologous) cluster ID, e.g., Q8F0K3 (cluster ID, Q72UG2) (FIGS. 32A,B).

To further explore the evolutionary implications of these observations, phylogenomic analysis of ˜3,000 leptospiral VM proteins derived from pathogenic Leptospira species was carried out. Amino acid sequences were aligned via hmmalign against a custom-built HMMER v3 (Potter et al., 2018, Nucleic Acids Res, 46:W200-W204) profile based upon a comprehensive full PF07598 reference alignment. Within the pathogenic clade of the genus Leptospira, VM proteins are extraordinarily diverse, comprising at least 36 discrete orthologous clusters but with important amino acid similarities (FIGS. 32A,B). Clusters limited to a few leptospiral species are moderately sized containing between 5 and 50 individual VM proteins. Clusters containing L. interrogans-, L. kirschneri-, and L. noguchii-specific proteins are larger, ranging in size from 80 to 375 member proteins, probably reflecting a bias toward these species in accessible genomic databases.

Inspection of the pairwise distance matrices and MMDS cluster membership allows us to draw important conclusions. First, among paralogs, the N-terminal segment encompassing the CBR is more conserved ˜78% pairwise amino acid identity, Lai intragenomic range 66-99% in comparison with the CTD at ˜63 pairwise amino acid identity, Lai range, 62-72%. By contrast, the CTD is more conserved among orthologs with a minimum amino acid identity >75%, even between distantly related species allowing easy discernment of paralogs and orthologs. Second, of the many orthologous clusters defined (FIG. 32B), only one-Q72PX8-contains proteins (N=375) originating from all medically important species (as well as L. alexanderi and L. alstonii). The clustering pattern around Q72PX8 likely indicates that this protein is an ancestral VM protein in Group I pathogenic Leptospira. Conversely, most species contain at least one defining cluster, e.g., the CBR deletion variants of L. interrogans (n=139). Third, the scope of carbohydrate binding and toxin functionalities may vary with serovar, even among those belonging to the same species. For instance, both CBRUG2 and CTDUG2 corresponding to the CBR and CTD of Q8F0K3[UG2], respectively, are present—at one instance per genome—in L. interrogans serovars Copenhageni (represented by 8 strains and 98 proteins), Canicola (8 and 95), Hardjo (2 and 21), Lai (3 and 28), Manilae (2 and 26), and Pomona (5 and 62), each with >99% amino acid identity. Whereas Lai and Copenhageni orthologs feature the expected CBRUG2/CTDUG2 combination, some strains of Canicola contain the conserved chimeric variants CBRUG2/CTDTZ4 and CBRUL8/CTDUG2. While the cognate Q8F0K3[UG2] CBR and CTD occur in alternate tandem pairings, to be considered a genuine chimera, a variant must occur in multiple genomes, and CBRs and CTDs must each share >99% amino acid identity within a designated cluster center. Such variants are more common in serovars possessing paralogs with identical or nearly identical CBRs (e.g., Q72U83 and Q72NP1 in Copenhageni) (FIGS. 32A,B). Taken together, these observations suggest that evolution of both CBR and CTD domains and their reassortment in certain lineages—likely via recombination—have contributed to the diversity of leptospiral VM proteins and, specifically to understanding group I pathogen adaptation to the mammalian niche (FIG. 32C).

### Locating the Fusion Junction of Chimeric Virulence-Modifying Proteins: Functional Consequences

Having demonstrated that leptospiral VM proteins radiated and diversified within virulent species via gene duplication and lineage-dependent reassortment of CBR and CTD (FIG. 32), evidence was sought to indicate whether fusion junctions of the VM protein domains were functionally conserved. To test whether all or parts of the CTD are replaced, chimeric VM proteins were first identified using complementary sequence-based approaches. Chimeric variants occur in medically important Leptospira, but occur only among recently diverged paralog pairs belonging to the same supercluster (FIG. 32D). Only two types of chimeric variants were detected regardless of the paralogs involved, suggesting that chimeric variant development depends on some specific structural features critical to in vivo function. Inspection of the chimeric junctions revealed that fusion junctions are remarkably consistent (corroborated via comparison of multiple strains), suggesting that these replacements are functionally constrained. Accordingly, chimeric VM proteins are novel subdomain haplotypes wherein trafficking motifs present in the amino terminal CTD segment of one paralog become paired with DNase activity located in the second CTD segment of a related paralog (FIG. 32D). These observations suggest that these chimeric proteins likely have altered cell-targeting competencies (relative to their cognate donors and recipients), i.e., may reflect leptospiral adaption to different hosts.

The First Ricin B-Like Lectin Subdomain of Leptospiral Virulence-Modifying Proteins (RBL1) has Carbohydrate-Binding Specificity Similar to that of Ricin B Chain

Because remote homology searches identified tandem ricin B-like lectin subdomains in the amino-terminal regions of leptospiral VM proteins (FIG. 31), the hypothesis was tested that, like ricin B chain, leptospiral VM proteins bind to terminal galactose and N-acetyl-galactosamine residues, such as asialofetuin, a model protein (Dawson et al., 1999, J. Appl. Toxicol, 19:307-312; Blome et al., 2010, Anal. Biochem, 396:212-216; Falach et al., 2020. Sci. Rep. 10:9007). Initial experiments focused on recombinant Q8F0K3—referred to here by its locus tag rLA3490—because previous studies showed that LA3490 is both highly transcriptionally upregulated in vivo and implicated in virulence (Lehmann et al, 2013, PLOS Negl Trop Dis, 7(10): e2468). For consistency, all full-length recombinant VM proteins [i.e., complete CDS minus SS (Signal Sequence)] will be referred to similarly. Recombinant N-terminal truncations, e.g., of Q8F0K3, containing RBL1 alone, will be referred to as follows: t3490 for truncated LA3490. For experiments using native VM proteins, Leptospira cells were grown under conditions mimicking the internal host environment to promote virulence gene expression in vitro (Matsunaga et al., 2005, Infect Immun, 73(1):70-8; Lo et al., 2006, Infect. Immun. 74:5848-5859; Matsunaga et al, 2007; Infect Immun, 75(6): 2864-2874).

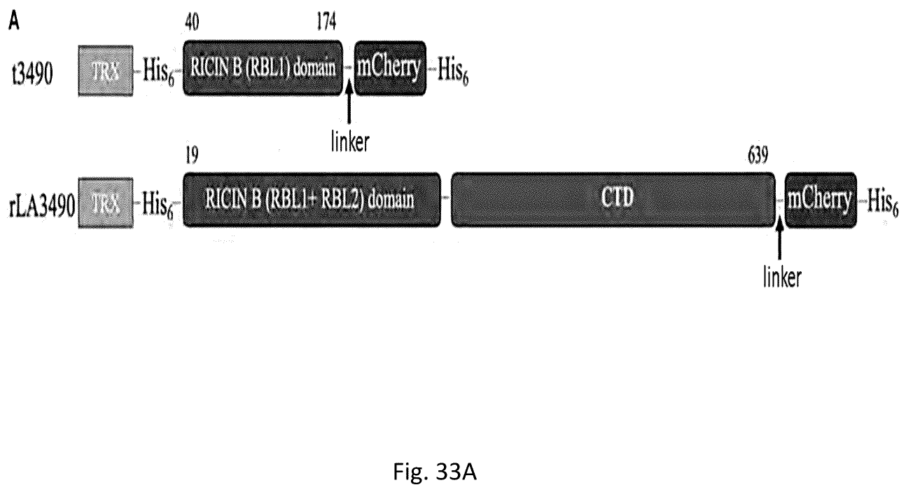

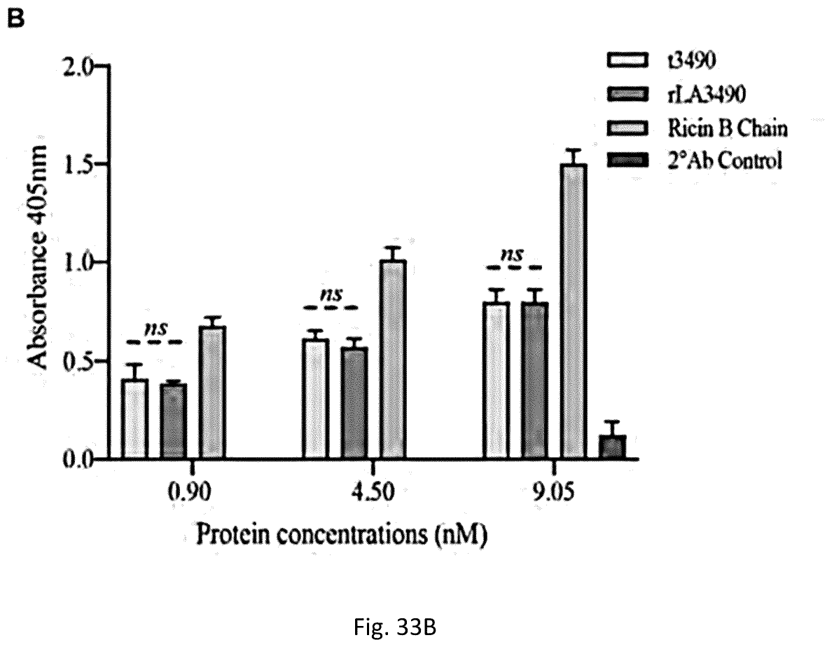

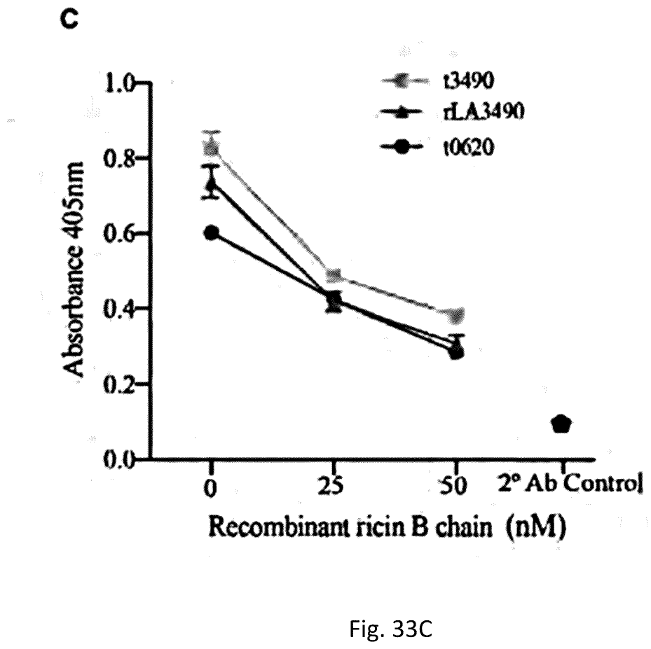

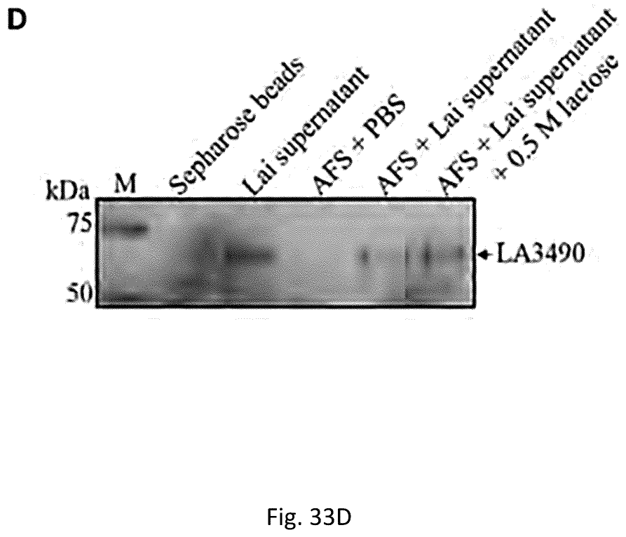

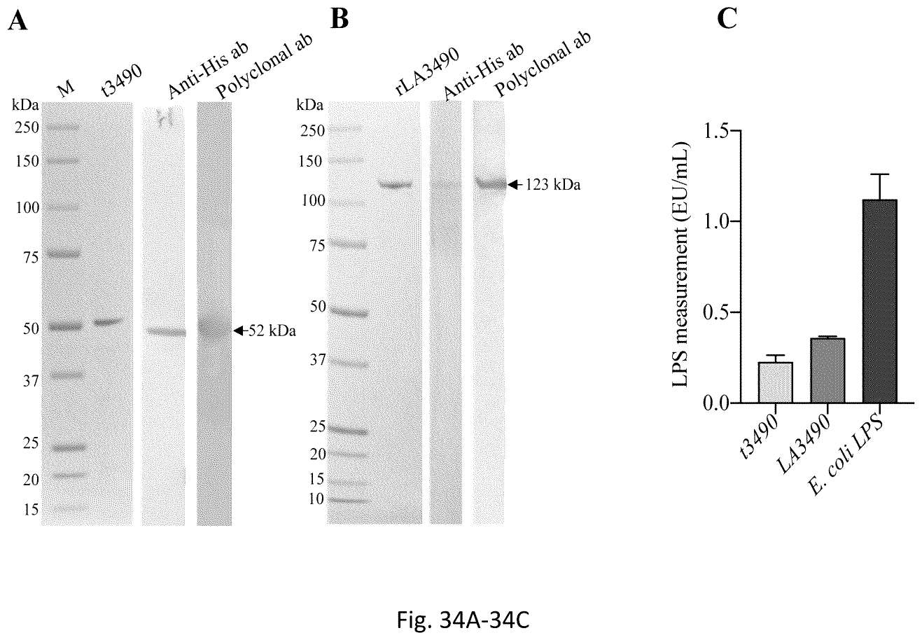

Recombinant VM proteins were expressed in E. coli as N-terminal fusions with thioredoxin-His6 (TRX) to improve solubility. The C-terminal were fused with mCherry-His6 to facilitate affinity purification and visualization of the protein using fluorescence microscopy (FIG. 33A). Western immunoblot, which was shown to be endotoxinfree by a limulus amebocyte lysate assay, ruling out the potential cytotoxicity of contaminating lipopolysaccharide (FIG. 34). LA3490 and t3490 both bound to asialofetuin (FIG. 33B). VM proteins have similar carbohydrate-binding specificity to ricin B chain as determined by competitive asialofetuin-binding assays (Sehnke et al., 1994, J. Biol. Chem. 269, 22473-22476; Dawson et al., 1999, J. Appl. Toxicol, 19:307-312; Blome et al., 2010, Anal. Biochem, 396:212-216; FIG. 33C). As predicted based upon the presence of a predicted secretory signal, Q8F0K3[rLA3490]—and likely other VM proteins—was found as a soluble protein in Leptospira-conditioned medium, with such secretion inducible by physiologic osmolarity and temperature. Solid phase-binding assays using asialofetuin-conjugated Sepharose beads confirmed that Q8F0K3 bound to asialofetuin (FIG. 33D). Like recombinant ricin B, asialofetuin-bound Q8F0K3 could be eluted using 0.5 M lactose, further supporting the conclusion that Leptospira VM proteins have bona fide ricin B-like lectin carbohydrate-binding activity.

These observations indicate that the CBD (RBL1) of LA3490 and, by analogy, CBDs of other VM proteins, are bona fide R-type lectins.

Dose-Dependent Cytotoxicity of rLA3490, on HeLa Cell Monolayers

Having confirmed that the RBL1 is an R-type lectin, without being bound by theory it was hypothesized that VM proteins are cytotoxins with effects mediated by the as-yet uncharacterized C-terminal region.

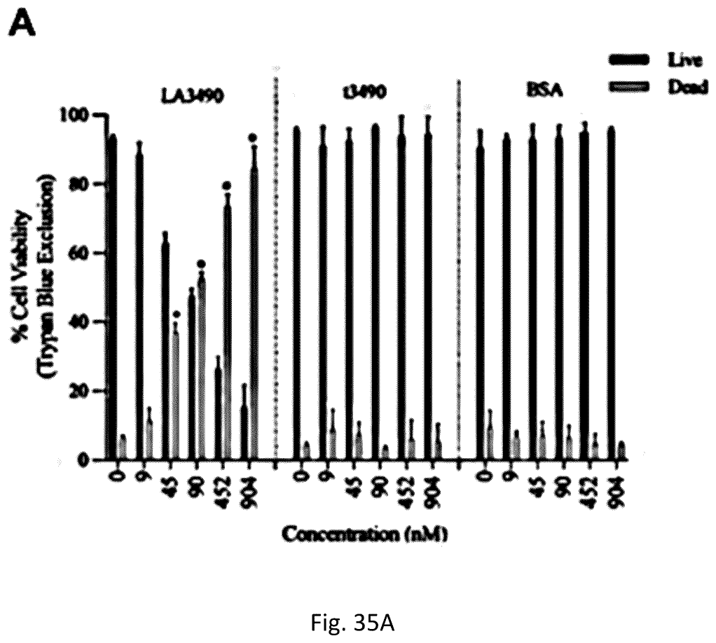

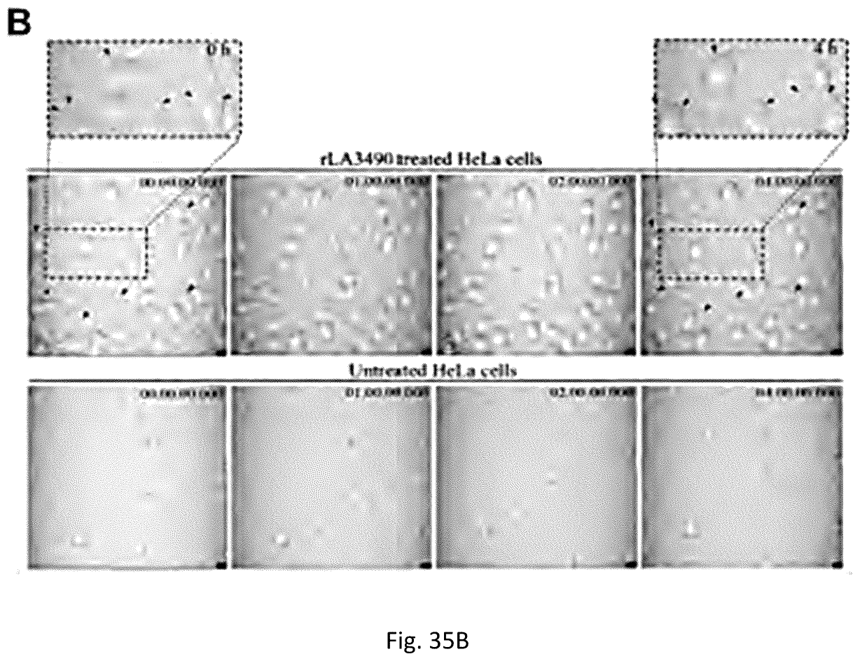

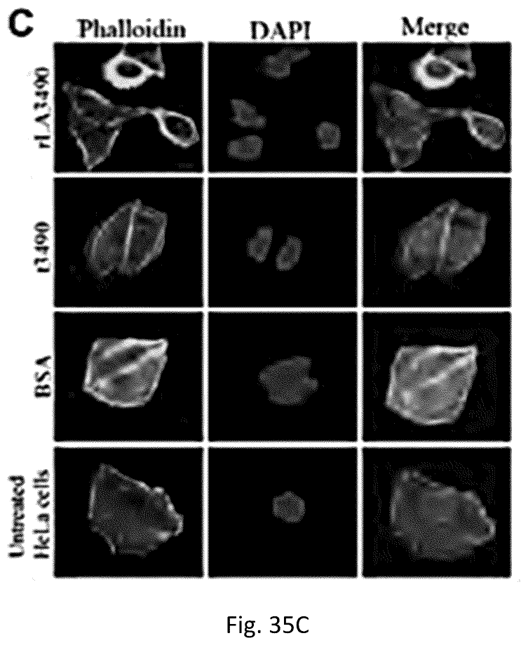

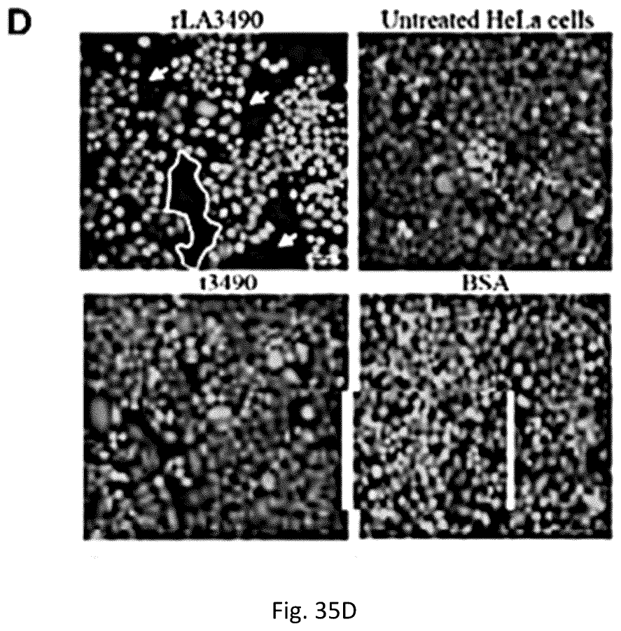

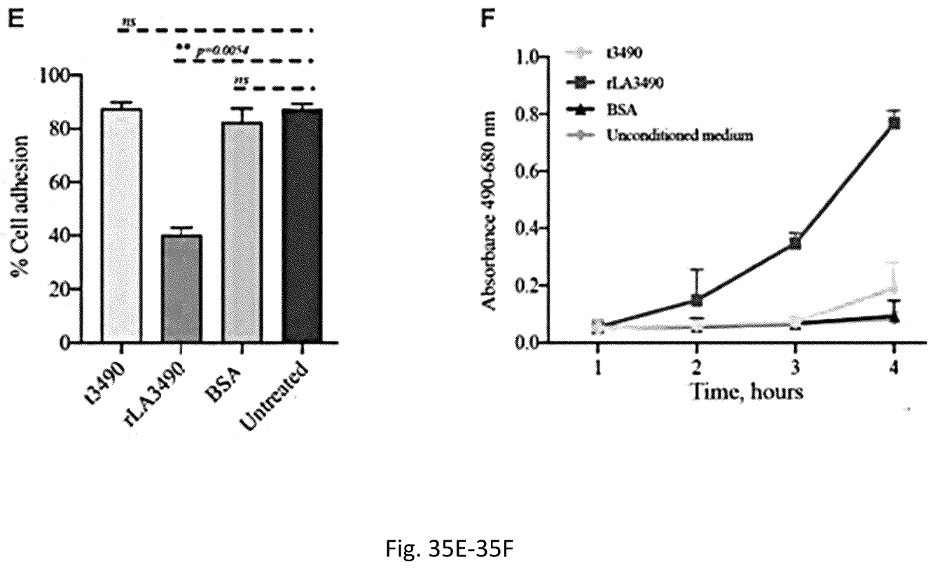

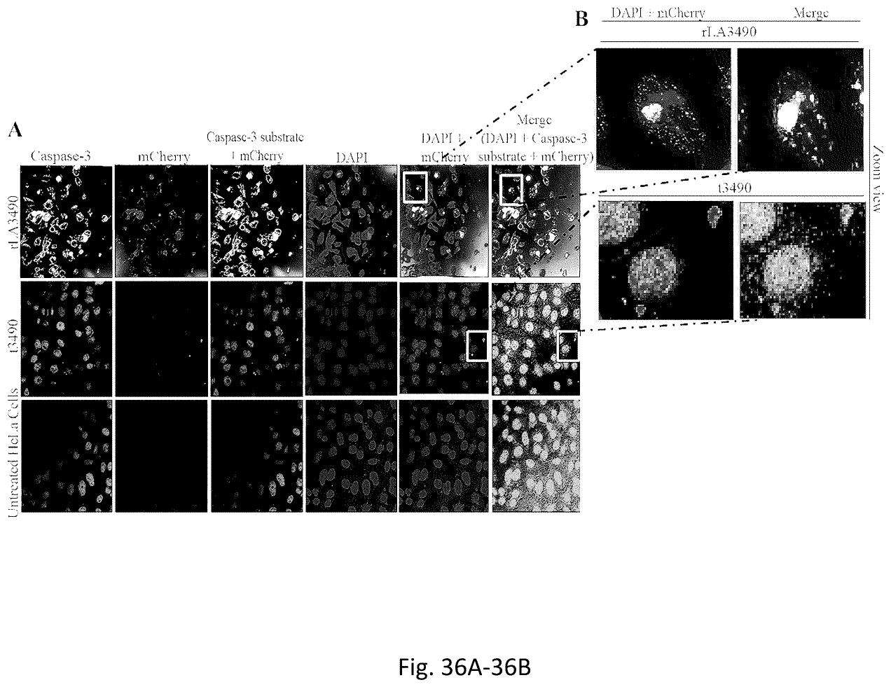

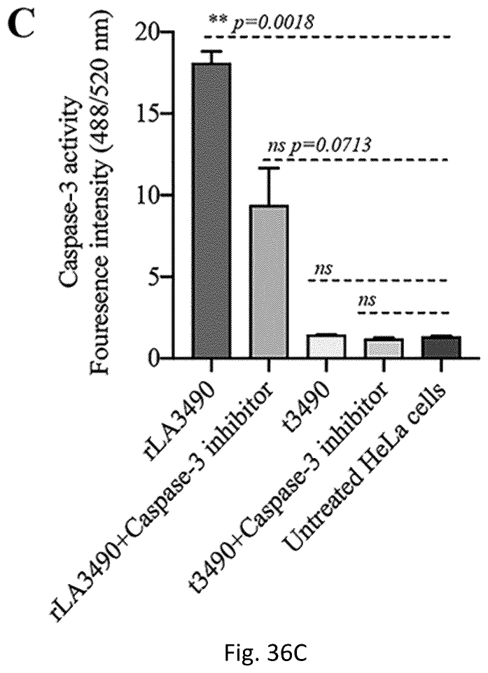

Exposure of HeLa cells to rLA3490 induced dose-dependent cytopathic effect and HeLa cell monolayer destruction (FIG. 35), as demonstrated by trypan blue exclusion, phase-contrast microscopy, fluorescent live/dead staining, and release of lactate dehydrogenase. Actin depolymerization (FIG. 35C) and caspase activation (FIG. 36) were observed. No such changes were observed with negative controls, including t3490, bovine serum albumin (BSA), and no treatment, confirming that cell death was a direct result of rLA3490 treatment and not to fusion protein affinity/epitope tags or an artifact of the culture conditions.

### Ricin B-Like Lectin 1 Alone is Sufficient for Attachment to the HeLa Cell Surface but not for Internalization

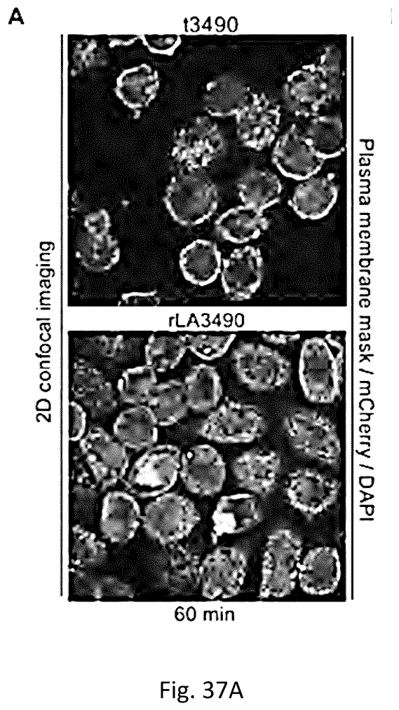

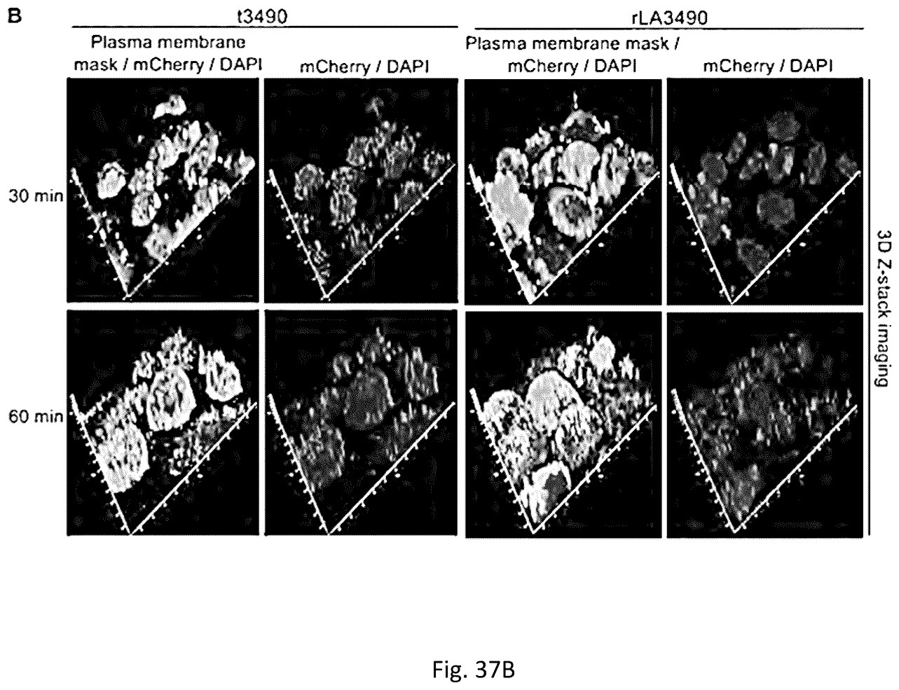

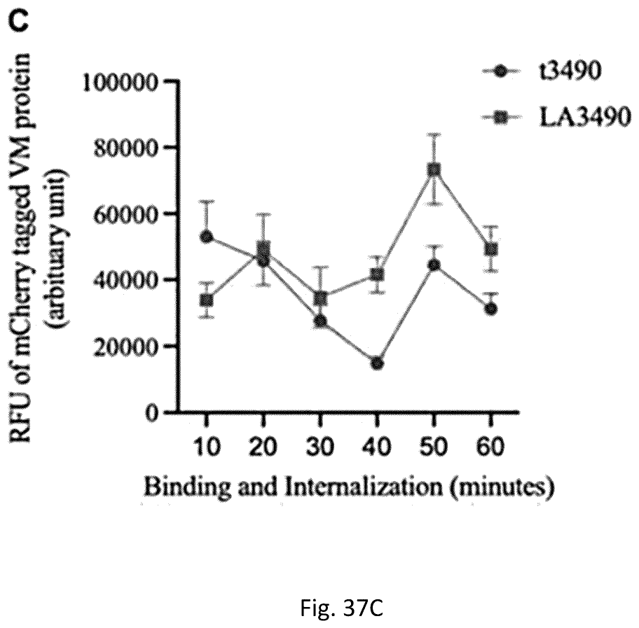

Internalization and/or intracellular trafficking of rLA3490- and t3490-mCherry fusion proteins were monitored using super-resolution fluorescent confocal microscopy (FIG. 37). Both fusion proteins bound to the cell surface, but only rLA3490 internalized and localized to the cell nucleus (FIGS. 37A,B). While RBL1 alone was sufficient for binding—both to immobilized asialofetuin (above) and at the HeLa cell surface—internalization depended on protein folds beyond the RBL domains. Binding of both rLA3490 and t3490 at the cell surface occurred 30-60 min after treatment, with internalization, translocation, and nuclear fragmentation evident from 30 min onward (FIG. 37B, right panels). Maximal accumulation of rLA3490 was dependent on the full-length protein-dependent internalization; maximum binding and accumulation of t3490 was at 10 min, while rLA3490 continued to accumulate in HeLa cells (FIG. 37C). t3490 bound to the surface of HeLa cells but was not internalized (FIG. 37B, left panels), and was not cytotoxic. Animated orthogonal and z-stacks show binding and internalization of rLA3490 and/or t3490 by HeLa cells at 30 and 60 min.

Leptospiral Virulence-Modifying Proteins have In Vitro Endo- and Exo-DNase Activity

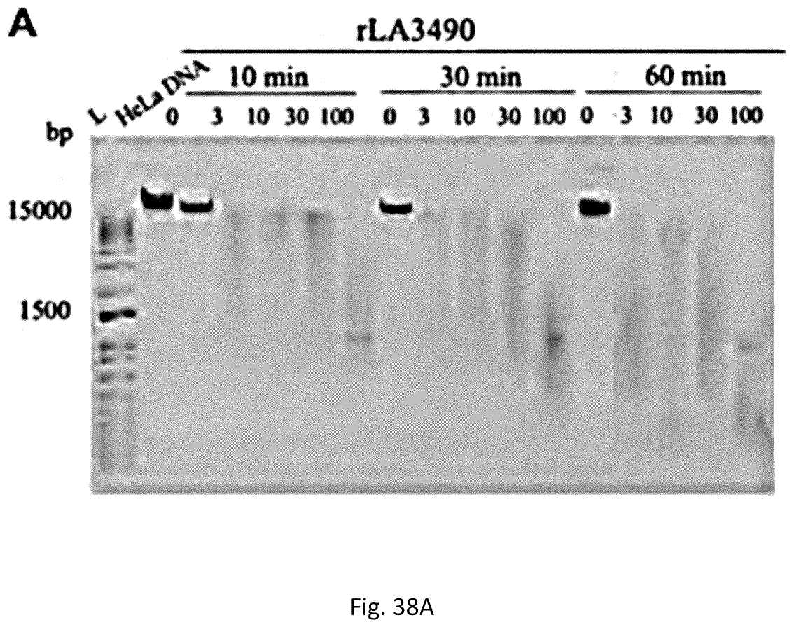

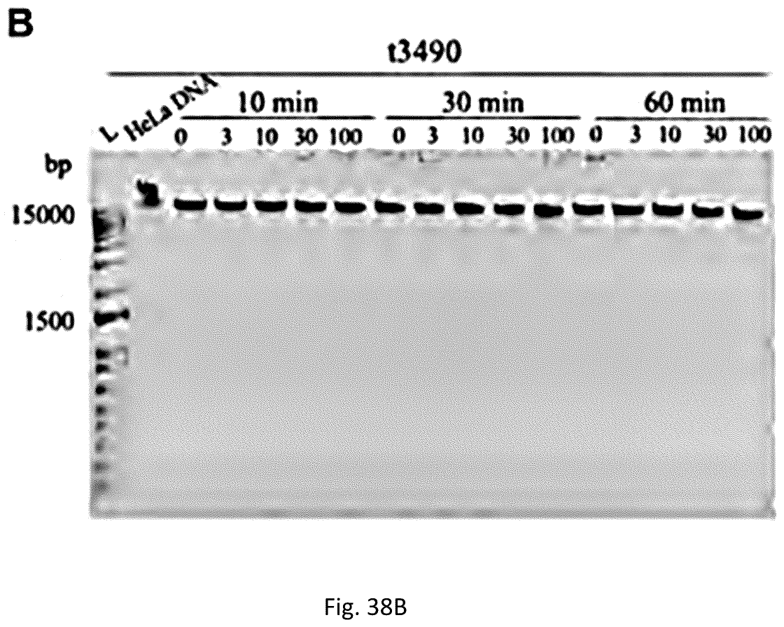

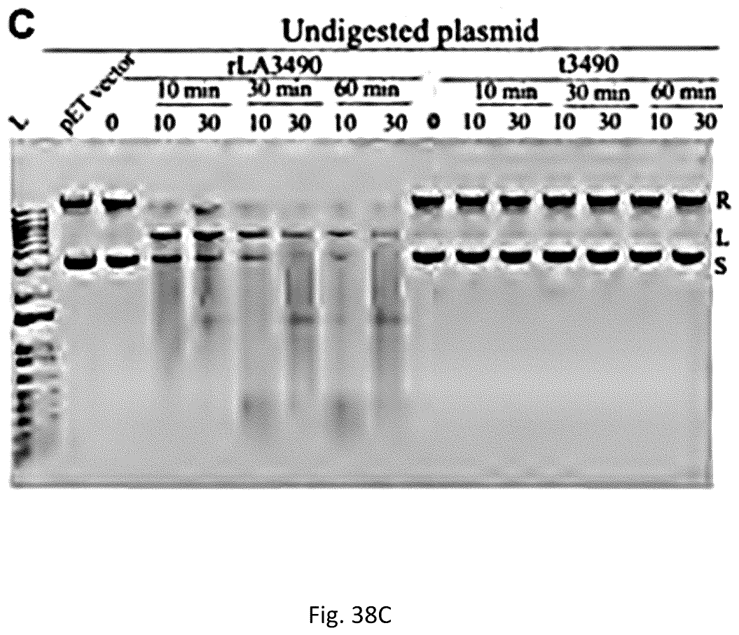

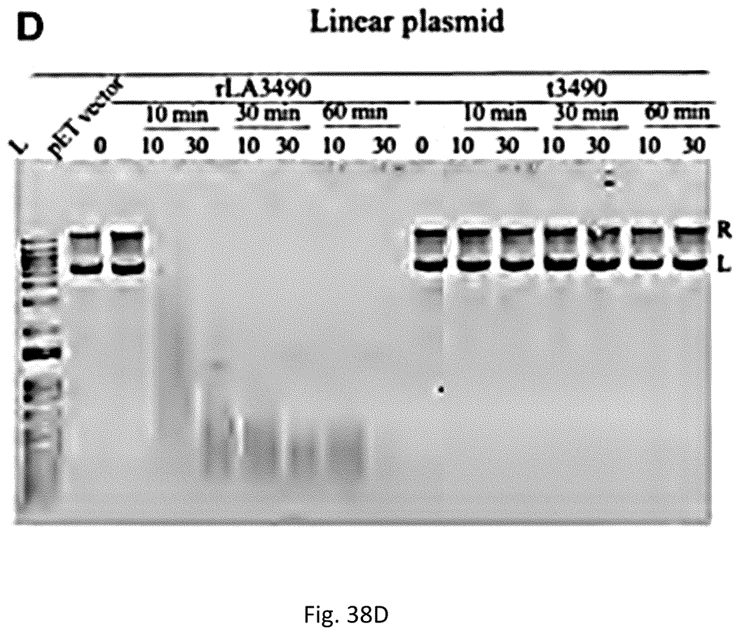

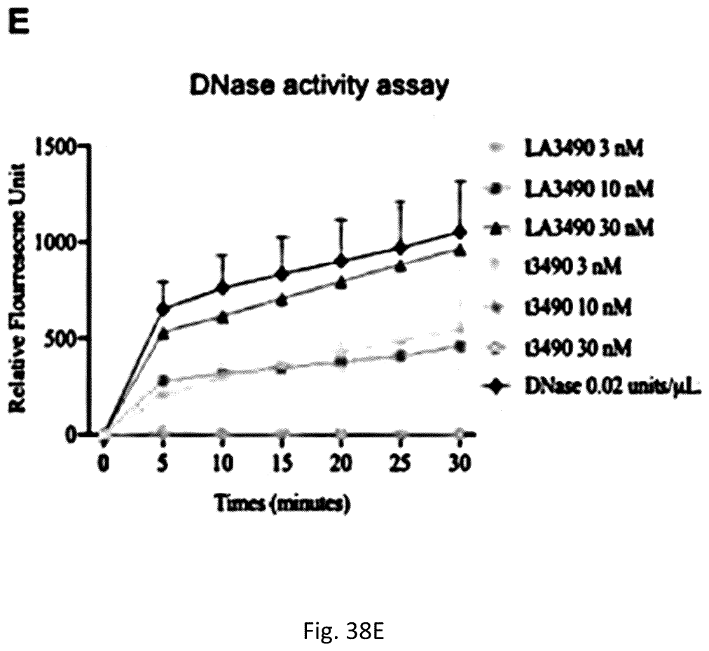

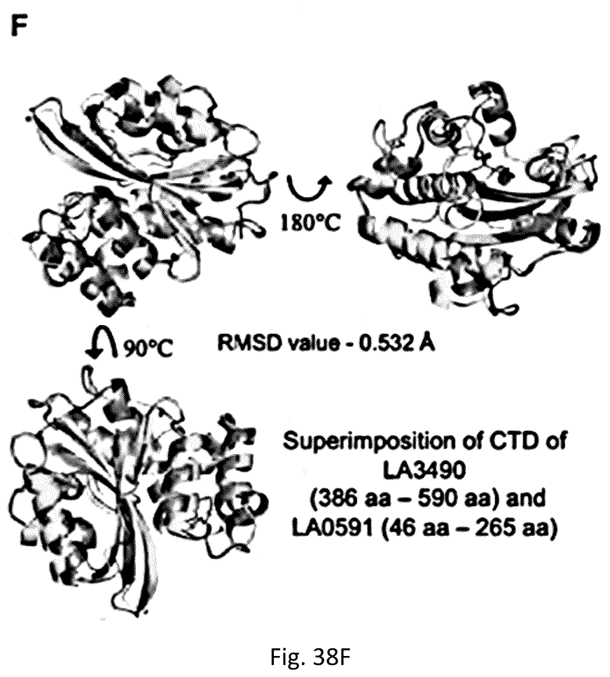

Having demonstrated that rLA3490 localizes to HeLa cell nuclei leading to chromosomal fragmentation, without being bound by theory, it was hypothesized that Q8F0K3 (and, by extension, all VM proteins) possesses DNase activity that might contribute to the mechanism of cell death. Cell-free assays demonstrated that rLA3490 has potent, dose-dependent DNase activity on purified HeLa cell genomic DNA (FIG. 38A), and dose-dependent nicking, endo-, and exonuclease activity on supercoiled plasmid DNA producing relaxed and linearized plasmid (FIGS. 38C,D). Linearized plasmids were completely digested (FIG. 38D) confirming exo-DNase activity comparable with that of recombinant bovine DNase I (FIG. 38E). Other recombinant VM proteins, LA0591 [serovar Lai CBR deletion variant (FIG. 38F)], rLA0620, rLA1400, and rLA1402 also exhibited exo-DNase activity (data not shown). Negative controls, t3490 (and t0620, not shown), had no detectable DNase activity (FIG. 38).

Sequence, Structural Similarity of Leptospiral Carboxy Terminal Domains of LA3490 with Bovine DNase I

After experimental demonstration of DNase activity in recombinant full length VM proteins, a new sequence alignment was performed and phylogenetic relation analysis focused on comparing the CTD of VM protein amino acid sequences with various mammalian DNases and the E. coli cytolethal distending toxin (CdtB) sequences. These analyses were consistent with the Leptospira VM proteins having identifiable C-terminal DNase domains. In attempts to superimpose the CTD of LA3490 with bovine DNase I, there was structural resemblance specifically in the active site, but only an RMSD value of 9.012 Å was obtained because the overall structural similarity was low outside of the predicted active site (FIG. 39).

Leptospiral Virulence-Modifying Protein Structure-Function Relationships Based on the demonstration of VM protein cytotoxic and DNase activity, a better understanding of the diversity of these proteins and their radiation among virulent Leptospira spp was sought. HHpred homology searches indicated that RBL1 and RBL2 share high sequence and structural homology with D2 and D3 of CARDS (Becker et al., 2015, Proc Natl Acad Sci USA, 112(16):5165-70) (E=2.7e⁻⁹⁶) and appear to be functionally analogous. AlphaFold structural modeling suggests that VM protein CTDs appear to contain functionally distinct subdomains that explain experimentally demonstrated intracellular trafficking, cytotoxicity, and DNase activity (FIG. 35).

All VM CTDs, including CBR deletion variants, contain several statistically well supported (i.e., p<1e-4) human Short Linear Motif (SLIM) mimics (Samano-Sanchez and Gibson, 2020, Trends Biochem. Sci. 45:526-544), identified using the Eukaryotic Linear Motif (ELM) resource, that likely facilitate VM protein trafficking and nuclear translocation including a nuclear localization signal, NLS. Of 13 L. interrogans paralogs, only CBR deletion variants share a common NLS with another paralog, likely reflecting their evolutionary origins. Most members of a given ortholog cluster belonging to the same species contain an invariant NLS. Compared with its flanks, the NLS containing subdomain (14 aa) is especially variable suggesting strong selection. Following this subdomain are consecutive amphipathic a-helices, containing a putative LxxLL nuclear receptor motif [LIG_NRBOX(ELME000045): 451HLERLLE457; SEQ ID NO:94, and a SH2-binding motif a LIG_SH2_SRC(ELME000474): Yxxx8 (385YEIAT389); SEQ ID NO:95]. Also, present is an LxxLFD motif characteristic of EF2 transcription factors (absent from Q8F0K3) and a single LIG_AP2alpha_2 mimic (440DPF442) for binding accessory endocytic proteins (ELME000190). Q72UG2 cluster members, including Q8F0K3, contain multiple distinct SLIM mimics, including a second LxxLL motif (413MLAELLE419; SEQ ID NO:96).

A pLxIS motif (536PILRIS541; SEQ ID NO:97) may mediate immune escape via interference with IRF-3-dependent signaling and an adaptin-binding endosome-lysosome-basolateral sorting signal (p<8.4e-5): TRG_DiLeu_BaEn_3[ELME000525] and 557EEFRRFL563 (SEQ ID NO:98).

VM protein C-termini appear to be disordered and are usually terminated by a CAAX motif (i.e., MOD_CAAXbox[ELME000059], p<2.4e6) reminiscent of the F-box proteins produced by the intracellular bacterium Legionella pneumophila (Perpich et al., 2017). These motifs act as prenylation substrates and usually mediate membrane attachment. Q8F0K3[UG2] and Q8F6G6[CBR deletion variant] lack a canonical CAAX box but are terminated by a LIG_PDZ_Class_2[ELME000091] motif: 634RCALPF639 (SEQ ID NO:99) (p<7.9e-5), for binding of PDZ domains, which are globular protein modules found in eukaryotic regulatory proteins.

### Example 6: Lectin-DNAse VM Protein Antigen Sequences

Wildtype-Q04T47_12844_0769
(SEQ ID NO: 15)
CCATGGGCGTGAATAGCGCACTGATTGATATTGTTAGCCGCAGTATTCCGGGCAGTATTATTCAGAA
ACCGACCGATACCCCGCAGGATCGTGCAATTAAGATTGTTATTCATGATCGCGGCAAATTTTGTTAT
GCACCGGTTTTTAGTGGCGGTGAAAGCTATATTCGCATTCTGCAGTGCTGGGAACAGGATGTGAGCG
ATGCACGCTATGATGTGTTTCAGCGTATTAGTTATTACATTAAGAACACCTGGCTGTGTATTACCGC
CCCGGAAAGCGTGGTTCAGGGTCGTGTTAATTGGGATTATGTTAATCTGCGCCCGTGCACCATTAAT
GATCCGCTGCAGCGTTGGATTGTGAAAGAAAATGCATTTTGGACCGCAGATGAACGTTATCGCCTGA
AAGATGTGGGTTGGTATGCCTATATTAGTAAAACCAGTAGCGATTATTACAACCATACCCTGGATAG
CAGCATGAATAATTGGGTGCGTACCGTTAGCACCCCGGGCAATATTAGTATTAAGACCAGTATTGCC
TGGCATCTGGGTAGTGCACGTTATTTTATTAATAGTCGCGGCAGCAAAAAGAATACCACCCCGATCT
ATTATAATCCGGAAAGCGGTCATCTGGCACAGTATAATCCGATGAGTGGTCGTCTGAGCTGCATGTA
TAGTAAAGTGGGTAGTTATCAGTGGAATTGGATTCAGTGGGCACTGTGTAGCGATGCACCGATTAGT
AAAGATAATCCGGCATATTGGAATGTTTATCTGGAAACCGAAGAAGGCGGCATGCTGACCGATTATA
AAGGTAATATTCTGCGCGTGACCCGCTATGGTCCGAATTGGGGCGTGGCCTATGCCGTTAAACCGAG
TTTTCTGGTGAAAGATGCCACCCATAGCCCGACCAGTCTGTTTGTTGTGGATAAAGATCTGCTGGAT
TGGATTCGCTATACCACCGGTAATCTGGGCAAAACCGAACAGTATTGCCCGGCCGGCTATCGCAGCG
TTTATAAACGTGTGAAACGCACCCTGCCGCCGGATTTTCAGCTGAGCGATGATTGGATTCGTCGCCT
GTATGAAATTGCAGTTAGTACCATTGATACCGCACAGGGCAGCGGCATTTGTGGCGTGTGCATGCTG
CAGAGTTTTCAGATGCTGGCCGAACTGCAGGAATATCATTTTCAGGGTCCGCTGCAGAGTGGTGGTT
ATTTCTTTGATACCGCACGCGATCGTGATCCGTTTATTAGTTTTCGTCAGCGTTATAGCTATCTGGA
TCGTCTGCTGGCAGATGTTCCGTATGTGTATACCAGTGCAGGTGACACCAATCGTCAGATGGTTCTG
GCAACCGCACGTACCATGCTGCCGCAGTATAATTGGACCCTGAGTAGTGCATTCACTACCCGCAGCG
AAATTATGAGCCATATTAATAGTCTGATCCATTTTCCGCCGGGCAGCATTTGGCTGGGTGTTCTGGG
TCGCCGCCGTCCGGATGGCACCTTAAGCTGGCATGCAGTGCCGATTCTGCGTACCAGTCAGGGCCTG
GTTGTGATTCGCACCCGTGTGCCGAGTGCCCCGTTTGATCTGTATCGCCAGGCACTGACCCCGACCA
CCGATCTGCTGCAGGTTATTAATAATCTGGAAACCCCGAATCGCATTCTGACCACCTTTATGACCAT
TCAGCTGGGTGACGTTTATCGTAATACCTTTGATTTTGCCATTAGTAATCGTAATTGCACCGGCGAA
GGTGACGATCGCCGCGGCACCGGTGGTTATCCGGCCGGTACCAGTGTTAATCAGTGCAGTTGGCGTG
GTGGTCGTTGTGCCCTGCAGGGTGGCGGCGGTAGCGGTGGTGGTGGTAGCGGTGGCGGTGGTAGTGA
TGATGATGATAAAATGGTGAGCAAAGGCGAAGAAGATAATATGGCAATTATTAAGGAGTTCATGCGC
TTTAAAGTTCATATGGAAGGTAGTGTGAATGGTCATGAATTTGAAATTGAAGGTGAAGGTGAAGGCC
GCCCGTATGAAGGTACCCAGACCGCCAAACTGAAAGTGACCAAAGGTGGTCCGCTGCCGTTTGCATG
GGATATTCTGAGCCCGCAGTTTATGTATGGTAGCAAAGCCTATGTTAAACATCCGGCAGATATTCCG
GATTATCTGAAACTGAGTTTTCCGGAAGGCTTTAAATGGGAACGCGTTATGAATTTTGAAGATGGCG
GTGTTGTTACCGTGACCCAGGATAGCAGCCTGCAGGATGGTGAATTCATCTATAAAGTGAAACTGCG
CGGCACCAATTTTCCGAGCGATGGCCCGGTTATGCAGAAAAAGACTATGGGCTGGGAAGCAAGCAGT
GAACGCATGTATCCGGAAGATGGTGCACTGAAAGGTGAAATTAAGCAGCGTCTGAAACTGAAAGATG
GTGGTCATTATGATGCCGAAGTGAAAACCACCTATAAAGCAAAAAAGCCGGTTCAGCTGCCGGGTGC
ATATAATGTTAATATTAAGCTGGATATCACCAGTCATAATGAAGATTATACCATTGTTGAGCAGTAT
GAACGCGCCGAAGGTCGCCATAGCACCGGCGGTATGGATGAACTGTATAAACTCGAG
Mutant-Q04T47_12844_0769
(SEQ ID NO: 16)
CCATGGGCGTGAATAGTGCCCTGATTGATATTGTTAGTCGTAGCATTCCGGGTAGCATTATTCAGAA
ACCGACCGATACCCCGCAGGATCGTGCAATTAAGATTGTTATTCATGATCGTGGCAAATTTTGCTAT
GCCCCGGTTTTTAGTGGCGGTGAAAGCTATATTCGTATTCTGCAGTGCTGGGAACAGGATGTTAGCG
ATGCACGCTATGATGTTTTTCAGCGTATTAGTTATTACATCAAGAACACCTGGCTGTGCATTACCGC
ACCGGAAAGTGTTGTGCAGGGCCGTGTGAATTGGGATTATGTTAATCTGCGCCCGTGCACCATTAAT
GATCCGCTGCAGCGTGCCATTGTGAAAGAAAATGCCTTTTGGACCGCAGATGAACGCTATCGCCTGA
AAGATGTGGGCTGGTATGCCGCCATTAGTAAAACCAGTAGCGATTATTATAACCATACCCTGGATAG
CAGCATGAATAATTGGGTGCGCACCGTGAGCACCCCGGGCAATATTAGCATTAAGACCAGTATTGCC
TGGCATCTGGGTAGCGCCCGTTATTTTATTAATAGCCGCGGCAGCAAAAAGAATACCACCCCGATCT
ATTATAATCCGGAAAGTGGTCATCTGGCACAGTATAATCCGATGAGTGGCCGCCTGAGTTGTATGTA
TAGTAAAGTGGGCAGTTATCAGTGGAATTGGATTCAGTGGGCCCTGTGCAGCGATGCACCGATTAGT
AAAGATAATCCGGCATATTGGAATGTTTATCTGGAAACCGAAGAAGGCGGCATGCTGACCGATTATA
AAGGCAATATTCTGCGCGTTACCCGTTATGGCCCGAATTGGGGTGTTGCATATGCAGTGAAACCGAG
CTTTCTGGTGAAAGATGCAACCCATAGCCCGACCAGCCTGTTTGTTGTTGATAAAGATCTGCTGGAT
TGGATTCGCTATACCACCGGCAATCTGGGTAAAACCGAACAGTATTGCCCGGCCGGCTATCGTAGTG
TTTATAAACGCGTTAAACGTACCCTGCCGCCGGATTTTCAGCTGAGTGATGATTGGATTCGTCGCCT
GTATGAAATTGCAGTTAGTACCATTGATACCGCCCAGGGCAGCGGCATTTGCGGTGTTTGTATGCTG
CAGAGCTTTCAGATGCTGGCAGAACTGCAGGAATATGCATTTCAGGGTCCGCTGCAGAGTGGCGGTT
ATTTCTTTGATACCGCACGCGATCGTGATCCGTTTATTAGCTTTCGTCAGCGCTATAGTTATCTGGA
TCGTCTGCTGGCAGATGTGCCGTATGTGTATACCAGCGCCGGCGATACCAATCGCCAGATGGTGCTG
GCCACCGCACGCACCATGCTGCCGCAGTATAATTGGACCCTGAGCAGTGCATTCACTACCCGCAGCG
AAATTATGAGTGCCATTAATAGCCTGATTCATTTTCCGCCGGGTAGCATCTGGCTGGGTGTTCTGGG
TCGTCGTCGTCCGGATGGCACCCTGAGTTGGGCAGCCGTGCCGATTCTGCGCACCAGTCAGGGTCTG
GTTGTTATTCGCACCCGCGTGCCGAGCGCCCCGTTTGATCTGTATCGCCAGGCACTGACCCCGACCA
CCGATCTGCTGCAGGTTATTAATAATCTGGAAACCCCGAATCGTATTCTGACCACCTTTATGACCAT
TCAGCTGGGCGATGTGTATCGCAATACCTTTGATTTTGCAATTAGTAATCGCAATTGCACCGGTGAA
GGTGACGATGCCCGTGGTACCGGTGGTTATCCGGCCGGCACCAGCGTGAATCAGTGCAGTTGGCGTG
GCGGTCGCTGCGCCCTGCAGGGTGGTGGTGGTAGTGGTGGCGGTGGCAGTGGCGGTGGTGGCAGTGA
TGATGATGATAAAATGGTTAGTAAGGGCGAAGAAGATAATATGGCAATTATTAAGGAGTTCATGCGT
TTTAAAGTGCATATGGAAGGCAGCGTTAATGGTCATGAATTTGAAATTGAAGGTGAAGGCGAAGGTC
GTCCGTATGAAGGCACCCAGACCGCCAAACTGAAAGTTACCAAAGGTGGTCCGCTGCCGTTTGCCTG
GGATATTCTGAGCCCGCAGTTTATGTATGGCAGCAAAGCATATGTTAAACATCCGGCAGATATTCCG
GATTATCTGAAACTGAGCTTTCCGGAAGGTTTTAAATGGGAACGTGTTATGAATTTTGAGGATGGCG
GCGTTGTTACCGTGACCCAGGATAGTAGTCTGCAGGATGGTGAATTCATCTATAAAGTTAAACTGCG
TGGCACCAATTTTCCGAGCGATGGCCCGGTTATGCAGAAAAAGACTATGGGCTGGGAAGCCAGCAGC
GAACGCATGTATCCGGAAGATGGTGCACTGAAAGGTGAAATTAAGCAGCGTCTGAAACTGAAAGATG
GTGGCCATTATGATGCCGAAGTTAAAACCACCTATAAAGCAAAAAAGCCGGTTCAGCTGCCGGGCGC
ATATAATGTGAATATTAAGCTGGATATCACCAGTCATAATGAAGATTATACCATTGTTGAGCAGTAT
GAACGCGCCGAAGGTCGCCATAGCACCGGCGGTATGGATGAACTGTATAAACTCGAG
Wildtype-Q04V07_12339_1402
(SEQ ID NO: 17)
CCATGGGCGGTAGCAGCATTAGCAGTAGTGGCATTCCGGGCAGCGTGGTGCAGAAACCGACCGATAA
ACCGAATGATAAACCGATTAAGATTGTTGCCCATAATGGTAAAAACTATTGCTATAGCCCGATGTTT
GGCCGTAATGAAAGTTATATTCAGCTGGAACAGTGCGGTGACAAAACCCTGAAAGCCCGTTATGATG
TTTTTCAGCGCATTAGCTATTATATTAACAACACCTGGCTGTGTATTACCACCCCGGAAAAAGTTAT
TAAGGGCGAAACCAATTGGGATTATGTGAATCTGCGCCCGTGCACCATTAATAATCCGGGCCAGCGT
TGGATTGTTAAAAATAATGCATTTTGGACCGCCAATGGTCGTTATCAGCTGAAAGATGTGCATTGGC
TGGCCTATATTAGCAAAACCAGCAGTGATTATTACAATCATACCCTGGATACCACCATGAATGATTG
GATTAAGACCGTGGCAACCCCGGGTAATGTTAGCGTTCAGACCAGCATTGCATGGGATAGTAATTGG
GGCGATGGCTTTTGGAATGGTACCCCGGGTCGTTATTTTATTCGTAGCGGCGGCAGTAATCGTAATA
CCACCCCGATCTATTATAATCCGGAAAATGGCCATCTGGCCCAGTATAATCCGGCCAATGGCCTGCT
GAGCTGCATGTATAGCAAAATTGGTAAATATAACTGGAACTGGGTGCAGTGGGCCAGTTGCAGCGAT
GCACCGGTGAGCAGTAAAGAAAATAGCGCCTATTGGAATGTGTATTTTGAAACCGCAGAAGGTGGTA
TGATTACCGATTATCAGGGCAATATTCTGCGTGTGACCAAAACCGGTCCGAATTGGGGCGTTGCCTA
TACCGTTCGTCCGAGTTATCTGCAGGCAGATACCACCCATAGCCCGACCAGCCTGTTTATTGTGGAT
AAAGATCTGCTGAATTGGGTTCGTTATACCACCGGTAATCTGGGCAATACCGAACAGTATTGCCCGG
CAGGCAATAAGAAAAATCATACCTATAAACGCGTGAAACGTAATCTGCCGAGCGATTTTGAACTGAC
CGATGATTGGATTCGTCGCCTGTATGATATTGCCCGCAGCGCAACCTATGCCAGTGCCCGTCCGGGC
TATCGTCCGCAGCAGGCAGGCGTGTGTGGTACCTGTCTGCTGCATAGCTTTCAGATGCTGGCAGAAC
TGCATGAATATCATAGCCAGCGCCCGCTGCAGAGCGGTGGCTATTTCTTTGATACCGCCATTGATCG
CGATCCGTTTATTAGCTTTAGTGTTCGTTATCGCGAACTGTATCGCCTGCTGACCAATGTGCCGGCC
ACCTATGCCAATCGCGGTCGTGTTCTGGCCTATGCCAGTACCGCACTGATGATGCCGCAGTATGAAT
GGGAAAGCAGTCCGCCGATTACCATTCGCAGCGAAATGCTGAGTCATATTCGCAGTATGATTAATAG
CCCGACCGGCAGCATTTGGCTGGCAATTATGCGTCGCCAGCGTACCGATGGCACCATTAGTGGTCAT
GCAGTGCCGATTCTGCGCACCAGCCAGGGTCTGGTTGTGATTCGCACCAATACCCCGACCACCAGCT
TTAATGATTATCGTAGCAGTCTGATTCCGCGCATGAATCCGGATGAAATTCTGGCCAATCTGGAAGG
CCCGGGCCGCACCATGCTGACCTTTACCACCATTCAGCCGGTTGGCGCCTATGAAAATGCCTTTGAT
CTGATGGTGAGTACCAAAGATTGTACCGGCGAAGGTGACGATCGCCGCGGCAGTGGCCGTTTTCCGA
TTAGCAGTCTGGTGAATCAGTGTAGTGGCGGTCGTTGCATTCTGCAGGGCGGTGGTGGTAGTGGCGG
CGGTGGTAGTGGTGGTGGTGGCAGCGATGATGATGATAAAATGGTGAGCAAAGGCGAAGAAGATAAT
ATGGCAATTATTAAGGAGTTCATGCGCTTTAAAGTTCATATGGAAGGCAGCGTTAATGGTCATGAAT
TTGAAATTGAAGGCGAAGGCGAAGGTCGTCCGTATGAAGGTACCCAGACCGCCAAACTGAAAGTTAC
CAAAGGTGGCCCGCTGCCGTTTGCATGGGATATTCTGAGCCCGCAGTTTATGTATGGCAGCAAAGCA
TATGTGAAACATCCGGCAGATATTCCGGATTATCTGAAACTGAGCTTTCCGGAAGGCTTTAAATGGG
AACGCGTTATGAATTTTGAAGATGGCGGTGTGGTGACCGTGACCCAGGATAGCAGTCTGCAGGATGG
TGAATTCATCTATAAAGTGAAACTGCGTGGTACCAATTTTCCGAGCGATGGTCCGGTTATGCAGAAA
AAGACTATGGGTTGGGAAGCCAGCAGCGAACGCATGTATCCGGAAGATGGCGCACTGAAAGGCGAAA
TTAAGCAGCGCCTGAAACTGAAAGATGGCGGTCATTATGATGCAGAAGTGAAAACCACCTATAAAGC
AAAAAAGCCGGTGCAGCTGCCGGGTGCATATAATGTTAATATTAAGCTGGATATCACCAGTCATAAT
GAAGATTATACCATTGTTGAGCAGTATGAACGCGCAGAAGGCCGCCATAGTACCGGCGGTATGGATG
AACTGTATAAACTCGAG
Mutant-Q04V07_12339_1402
(SEQ ID NO: 18)
CCATGGGCGGTAGCAGCATTAGCAGTAGCGGTATTCCGGGTAGTGTTGTGCAGAAACCGACCGATAA
ACCGAATGATAAACCGATTAAGATTGTTGCCCATAATGGTAAAAACTATTGCTATAGTCCGATGTTT
GGTCGCAATGAAAGCTATATTCAGCTGGAACAGTGTGGCGATAAAACCCTGAAAGCACGTTATGATG
TGTTTCAGCGTATTAGTTATTACATTAACAACACCTGGCTGTGTATTACCACCCCGGAAAAAGTGAT
TAAGGGTGAAACCAATTGGGATTATGTGAATCTGCGTCCGTGCACCATTAATAATCCGGGCCAGCGC
GCCATTGTGAAAAATAATGCATTTTGGACCGCCAATGGCCGTTATCAGCTGAAAGATGTTCATTGGC
TGGCAGCCATTAGCAAAACCAGCAGCGATTATTATAATCATACCCTGGATACCACCATGAATGATTG
GATTAAGACCGTTGCAACCCCGGGCAATGTTAGTGTTCAGACCAGTATTGCATGGGATAGTAATTGG
GGTGACGGCTTTTGGAATGGTACCCCGGGTCGCTATTTTATTCGTAGTGGTGGCAGTAATCGCAATA
CCACCCCGATCTATTATAATCCGGAAAATGGCCATCTGGCACAGTATAATCCGGCAAATGGTCTGCT
GAGTTGCATGTATAGCAAAATTGGTAAATACAACTGGAACTGGGTGCAGTGGGCCAGTTGCAGTGAT
GCACCGGTTAGCAGTAAAGAAAATAGTGCCTATTGGAATGTTTACTTTGAAACCGCAGAAGGCGGTA
TGATTACCGATTATCAGGGTAATATTCTGCGTGTTACCAAAACCGGCCCGAATTGGGGCGTTGCATA
TACCGTGCGTCCGAGCTATCTGCAGGCCGATACCACCCATAGCCCGACCAGTCTGTTTATTGTGGAT
AAAGATCTGCTGAATTGGGTTCGTTATACCACCGGCAATCTGGGCAATACCGAACAGTATTGCCCGG
CAGGTAATAAGAAAAATCATACCTATAAGCGTGTGAAACGTAATCTGCCGAGCGATTTTGAACTGAC
CGATGATTGGATTCGTCGCCTGTATGATATTGCACGCAGCGCAACCTATGCCAGCGCCCGCCCTGGT
TATCGTCCGCAGCAGGCCGGTGTTTGCGGCACCTGCCTGCTGCATAGCTTTCAGATGCTGGCCGAAC
TGCATGAATATGCCAGCCAGCGTCCGCTGCAGAGTGGCGGTTATTTCTTTGATACCGCAATTGATCG
TGATCCGTTTATTAGTTTTAGTGTGCGTTATCGCGAACTGTATCGTCTGCTGACCAATGTGCCGGCA
ACCTATGCAAATCGCGGTCGCGTTCTGGCCTATGCAAGTACCGCCCTGATGATGCCGCAGTATGAAT
GGGAAAGCAGTCCGCCGATTACCATTCGCAGTGAAATGCTGAGTGCAATTCGCAGCATGATTAATAG
TCCGACCGGCAGTATTTGGCTGGCAATTATGCGCCGTCAGCGTACCGATGGTACCATTAGCGGCGCC
GCCGTTCCGATTCTGCGTACCAGTCAGGGTCTGGTTGTTATTCGCACCAATACCCCGACCACCAGTT
TTAATGATTATCGTAGTAGTCTGATCCCGCGCATGAATCCGGATGAAATTCTGGCAAATCTGGAAGG
CCCGGGTCGTACCATGCTGACCTTTACCACCATTCAGCCGGTGGGTGCCTATGAAAATGCCTTTGAT
CTGATGGTGAGTACCAAAGATTGCACCGGTGAAGGCGATGATGCCCGCGGTAGCGGTCGTTTTCCGA
TTAGCAGTCTGGTGAATCAGTGTAGCGGTGGCCGCTGCATTCTGCAGGGTGGTGGCGGCAGCGGTGG
CGGTGGTTCAGGTGGTGGCGGTAGCGATGATGATGATAAAATGGTTAGTAAAGGCGAAGAAGATAAT
ATGGCAATTATTAAGGAGTTCATGCGCTTTAAAGTTCATATGGAAGGTAGCGTTAATGGCCATGAAT
TTGAAATTGAAGGTGAAGGCGAAGGCCGTCCGTATGAAGGCACCCAGACCGCCAAACTGAAAGTGAC
CAAAGGTGGTCCGCTGCCGTTTGCCTGGGATATTCTGAGCCCGCAGTTTATGTATGGTAGTAAAGCC
TATGTTAAGCATCCGGCAGATATTCCGGATTATCTGAAACTGAGTTTTCCGGAAGGTTTTAAATGGG
AACGTGTGATGAATTTTGAAGATGGTGGTGTGGTGACCGTGACCCAGGATAGTAGTCTGCAGGATGG
CGAATTCATCTATAAAGTGAAACTGCGTGGTACCAATTTTCCGAGTGATGGTCCGGTTATGCAGAAA
AAGACTATGGGCTGGGAAGCAAGCAGCGAACGTATGTATCCGGAAGATGGCGCACTGAAAGGTGAAA
TTAAGCAGCGTCTGAAACTGAAAGATGGTGGTCATTATGATGCAGAAGTGAAAACCACCTATAAAGC
AAAAAAGCCGGTTCAGCTGCCGGGCGCCTATAATGTTAATATTAAGCTGGATATCACCAGTCATAAT
GAAGATTATACCATTGTTGAGCAGTATGAACGTGCAGAAGGTCGTCATAGTACCGGCGGTATGGATG
AACTGTATAAACTCGAG

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition comprising at least one Leptospiral virulence modifying (VM) protein or fragment thereof selected from the group consisting of:
a) a VM protein selected from the group consisting of LA_3388, LA_0835, LA_0591, LA_0589 (v), LA_1402, LA_1400, LA_3271, LA_0934, LA_0769, LA_2628, LA_0620, LA_3490, LIC_10778, LIC_12791, LIC_12985, LIC_12986, LIC_12339, LIC_12340, LIC_10870, LIC_12715, LIC_12844, LIC_11358, LIC_10639, LIC_12963, LIC_10695, LMANV2_260038, LMANV2_240079, LMANV2_70075, LMANV2_70078, LMANV2_210058, LMANV2_210056, LMANV2_80114, LMANV2_320010, LMANV2_240142, LMANV2_150103, LMANV2_170032, LMANV2_70050, and LMANV2_170091; and
b) a fragment of a VM protein comprising the DNase domain of a VM protein selected from the group consisting of LA_3388, LA_0835, LA_0591, LA_0589 (v), LA_1402, LA_1400, LA_3271, LA_0934, LA_0769, LA_2628, LA_0620, LA_3490, LIC_10778, LIC_12791, LIC_12985, LIC_12986, LIC_12339, LIC_12340, LIC_10870, LIC_12715, LIC_12844, LIC_11358, LIC_10639, LIC_12963, LIC_10695, LMANV2_260038, LMANV2_240079, LMANV2_70075, LMANV2_70078, LMANV2_210058, LMANV2_210056, LMANV2_80114, LMANV2_320010, LMANV2_240142, LMANV2_150103, LMANV2_170032, LMANV2_70050, and LMANV2_170091.

2. The composition of claim 1, wherein the composition is a fusion protein comprising a Leptospiral VM protein or a fragment of a VM protein comprising the DNase domain fused to a targeting domain specific for binding to a target molecule.

3. The composition of claim 2, wherein the target molecule is selected from the group consisting of a bacterial antigen, viral antigen, parasitic antigen, cancer antigen, tumor-associated antigen, and tumor-specific antigen.

4. The composition of claim 1, wherein the composition comprises a combination of two or more Leptospiral VM proteins or fragments of a VM protein comprising the DNase domain.

5. The composition of claim 4 comprising a combination of LIC_12340 and LIC_12985.

6. The composition of claim 4 comprising a combination of LIC_12340, LIC_12985, LA_3490, LA_0620, and LA_1402.

7. The composition of claim 1, wherein the composition comprises at least one Leptospiral VM protein comprising an amino acid sequence selected from the group consisting of SEQ ID NO:2, SEQ ID NO:4, SEQ ID NO:6, SEQ ID NO:8, SEQ ID NO:10 and SEQ ID NO:12.

8. The composition of claim 5, wherein the composition comprises a combination of VM proteins comprising sequences as set forth in SEQ ID NO: 10 and SEQ ID NO:12.

9. The composition of claim 6, wherein the composition comprises a combination of VM proteins comprising sequences as set forth in SEQ ID NO:2, SEQ ID NO:4, SEQ ID NO:8, SEQ ID NO: 10 and SEQ ID NO:12.

10. The composition of claim 1, wherein the composition comprises at least one lipid nanoparticle (LNP) comprising at least one VM protein or fragment of a VM protein comprising the DNase domain.

11. The composition of claim 10, wherein the composition comprises a combination of at least two LNP comprising at least two VM proteins or fragments of a VM protein comprising the DNase domain.

12. A composition comprising at least one nucleic acid molecule encoding at least one Leptospiral virulence modifying (VM) protein or fragment thereof selected from the group consisting of:
a) a VM protein selected from the group consisting of LA_3388, LA_0835, LA_0591, LA_0589 (v), LA_1402, LA_1400, LA_3271, LA_0934, LA_0769, LA_2628, LA_0620, LA_3490, LIC_10778, LIC_12791, LIC_12985, LIC_12986, LIC_12339, LIC_12340, LIC_10870, LIC_12715, LIC_12844, LIC_11358, LIC_10639, LIC_12963, LIC_10695, LMANV2_260038, LMANV2_240079, LMANV2_70075, LMANV2_70078, LMANV2_210058, LMANV2_210056, LMANV2_80114, LMANV2_320010, LMANV2_240142, LMANV2_150103, LMANV2_170032, LMANV2_70050, and LMANV2_170091; and
b) a fragment comprising a DNase domain of a VM protein selected from the group consisting of LA_3388, LA_0835, LA_0591, LA_0589 (v), LA_1402, LA_1400, LA_3271, LA_0934, LA_0769, LA_2628, LA_0620, LA_3490, LIC_10778, LIC_12791, LIC_12985, LIC_12986, LIC_12339, LIC_12340, LIC_10870, LIC_12715, LIC_12844, LIC_11358, LIC_10639, LIC_12963, LIC_10695, LMANV2_260038, LMANV2_240079, LMANV2_70075, LMANV2_70078, LMANV2_210058, LMANV2_210056, LMANV2_80114, LMANV2_320010, LMANV2_240142, LMANV2_150103, LMANV2_170032, LMANV2_70050, and LMANV2_170091.

13. The composition of claim 12, wherein the nucleic acid molecule encodes a fusion protein comprising a Leptospiral VM protein or a fragment comprising a DNase domain of a VM protein fused to a targeting domain specific for binding to a target molecule.

14. The composition of claim 13, wherein the target molecule is selected from the group consisting of a bacterial antigen, viral antigen, parasitic antigen, cancer antigen, tumor-associated antigen, and tumor-specific antigen.

15. The composition of claim 12, wherein composition comprises one or more nucleic acid molecule encoding a combination of LIC_12340 and LIC_12985.

16. The composition of claim 12, wherein composition comprises one or more nucleic acid molecule encoding a combination of LIC_12340, LIC_12985, LA_3490, LA_0620, and LA_1402.

17. The composition of claim 12, wherein the nucleic acid molecule encodes at least one amino acid sequence selected from the group consisting of SEQ ID NO:2, SEQ ID NO:4, SEQ ID NO:6, SEQ ID NO:8, SEQ ID NO: 10 and SEQ ID NO:12.

18. The composition of claim 12, wherein the nucleic acid molecule comprises at least one nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:15, SEQ ID NO:16, SEQ ID NO:17 and SEQ ID NO:18.

19. The composition of claim 15, wherein the composition comprises one or more nucleic acid molecule encoding a combination of SEQ ID NO: 10 and SEQ ID NO:12.

20. The composition of claim 19, wherein the composition comprises one or more nucleic acid molecule comprising a combination of SEQ ID NO:9 and SEQ ID NO:11.

21. The composition of claim 16, wherein the composition comprises one or more nucleic acid molecule encoding a combination of SEQ ID NO:2, SEQ ID NO:4, SEQ ID NO:8, SEQ ID NO:10 and SEQ ID NO:12.

22. The composition of claim 21, wherein the composition comprises one or more nucleic acid molecule comprising a combination of SEQ ID NO:1, SEQ ID NO:3, SEQ ID NO:7, SEQ ID NO:9 and SEQ ID NO:11.

23. The composition of claim 12, wherein the composition comprises at least one lipid nanoparticle (LNP) comprising at least one nucleic acid molecule encoding at least one VM protein or fragment thereof comprising the DNase domain.

24. The composition of claim 23, wherein the nucleic acid molecule comprises an mRNA molecule encoding the at least one VM protein or fragment thereof comprising the DNase domain.

25. The composition of any one of claims 1-24, wherein the composition comprises a vaccine.

26. The composition of any one of claims 1-25, wherein the composition comprises an adjuvant.

27. The composition of claim 26, wherein the adjuvant is Glucopyranosyl Lipid A (GLA), formulated in a stable oil-in-water nano-emulsion (SE).

28. The composition of any one of claims 1-27, wherein the composition comprises a toxoid vaccine.

29. An anti-Leptospiral VM protein antibody comprising CDR sequences selected from the group consisting of:
a) the HCDR sequences as set forth in SEQ ID NO:21, SEQ ID NO:22 and SEQ ID NO:23 and the LCDR sequences as set forth in SEQ ID NO:24, SEQ ID NO:25 and SEQ ID NO:26;
b) the HCDR sequences as set forth in SEQ ID NO:21, SEQ ID NO:22 and SEQ ID NO:23 and the LCDR sequences as set forth in SEQ ID NO:37, SEQ ID NO:38 and SEQ ID NO:39;
c) the HCDR sequences as set forth in SEQ ID NO:45, SEQ ID NO:46 and SEQ ID NO:47 and the LCDR sequences as set forth in SEQ ID NO:48, SEQ ID NO:49 and SEQ ID NO:50;
d) the HCDR sequences as set forth in SEQ ID NO:61, SEQ ID NO:62 and SEQ ID NO:63 and the LCDR sequences as set forth in SEQ ID NO:64, SEQ ID NO:65 and SEQ ID NO:66; and
e) the HCDR sequences as set forth in SEQ ID NO:77, SEQ ID NO:78 and SEQ ID NO:79 and the LCDR sequences as set forth in SEQ ID NO:80, SEQ ID NO:81 and SEQ ID NO:82.

30. The anti-Leptospiral VM protein antibody of claim 29, wherein the antibody comprises a heavy chain and light chain sequence selected from the group consisting of:
a) the HC sequence as set forth in SEQ ID NO:27 and the LC sequence as set forth in SEQ ID NO:28;
b) the HC sequence as set forth in SEQ ID NO:27 and the LC sequence as set forth in SEQ ID NO:43;
c) the HC sequence as set forth in SEQ ID NO:51 and the LC sequence as set forth in SEQ ID NO:52;
d) the HC sequence as set forth in SEQ ID NO:67 and the LC sequence as set forth in SEQ ID NO:68; and
e) the HC sequence as set forth in SEQ ID NO:83 and the LC sequence as set forth in SEQ ID NO:84.

31. A nucleic acid molecule encoding an antibody of any one of claims 29-30, or a fragment thereof.

32. The nucleic acid molecule of claim 31, wherein the nucleic acid molecule comprises a nucleotide sequence selected from the group consisting of:
a) a nucleotide sequence comprising SEQ ID NO:29, SEQ ID NO:30 and SEQ ID NO:31 encoding HCDRs;
b) a nucleotide sequence comprising SEQ ID NO:32, SEQ ID NO:33 and SEQ ID NO:34 encoding LCDRs;
c) a nucleotide sequence comprising SEQ ID NO:40, SEQ ID NO:41 and SEQ ID NO:42 encoding LCDRs;
d) a nucleotide sequence comprising SEQ ID NO:53, SEQ ID NO:54 and SEQ ID NO:55 encoding HCDRs;
e) a nucleotide sequence comprising SEQ ID NO:56, SEQ ID NO:57 and SEQ ID NO:58 encoding LCDRs;
f) a nucleotide sequence comprising SEQ ID NO:69, SEQ ID NO:70 and SEQ ID NO:71 encoding HCDRs;
g) a nucleotide sequence comprising SEQ ID NO:72, SEQ ID NO:73 and SEQ ID NO:74 encoding LCDRs;
h) a nucleotide sequence comprising SEQ ID NO:85, SEQ ID NO:86 and SEQ ID NO:87 encoding HCDRs; and
i) a nucleotide sequence comprising SEQ ID NO:88, SEQ ID NO:89 and SEQ ID NO:90 encoding LCDRs.

33. The nucleic acid molecule of claim 32, wherein the nucleic acid molecule comprises a nucleotide sequence selected from the group consisting of: SEQ ID NO:35, SEQ ID NO:36, SEQ ID NO:44, SEQ ID NO:59, SEQ ID NO:60, SEQ ID NO:75, SEQ ID NO:76, SEQ ID NO:91 and SEQ ID NO:92.

34. A composition comprising a combination of at least two nucleic acid molecules of claim 32.

35. The composition of claim 34, comprising a combination of nucleic acid molecules selected from the group consisting of:
a) a first nucleic acid molecule comprising SEQ ID NO:29, SEQ ID NO:30 and SEQ ID NO:31 encoding HCDRs; and a second nucleotide sequence comprising SEQ ID NO:32, SEQ ID NO:33 and SEQ ID NO:34 encoding LCDRs;
b) a first nucleic acid molecule comprising SEQ ID NO:29, SEQ ID NO:30 and SEQ ID NO:31 encoding HCDRs; and a second nucleotide sequence comprising SEQ ID NO:40, SEQ ID NO:41 and SEQ ID NO:42 encoding LCDRs;
c) a first nucleic acid molecule comprising SEQ ID NO:53, SEQ ID NO:54 and SEQ ID NO:55 encoding HCDRs; and a second nucleotide sequence comprising SEQ ID NO:56, SEQ ID NO:57 and SEQ ID NO:58 encoding LCDRs;
d) a first nucleic acid molecule comprising SEQ ID NO:69, SEQ ID NO:70 and SEQ ID NO:71 encoding HCDRs; and a second nucleotide sequence comprising SEQ ID NO:72, SEQ ID NO:73 and SEQ ID NO:74 encoding LCDRs;
e) a first nucleic acid molecule comprising SEQ ID NO:85, SEQ ID NO:86 and SEQ ID NO:87 encoding HCDRs; and a second nucleotide sequence comprising SEQ ID NO:88, SEQ ID NO:89 and SEQ ID NO:90 encoding LCDRs.

36. The composition of claim 34, comprising a combination of nucleic acid molecules selected from the group consisting of:
a) a first nucleic acid molecule comprising SEQ ID NO:35; and a second nucleotide sequence comprising SEQ ID NO:36;
b) a first nucleic acid molecule comprising SEQ ID NO:35; and a second nucleotide sequence comprising SEQ ID NO:44;
c) a first nucleic acid molecule comprising SEQ ID NO:59; and a second nucleotide sequence comprising SEQ ID NO:60;
d) a first nucleic acid molecule comprising SEQ ID NO:75; and a second nucleotide sequence comprising SEQ ID NO:76; and
e) a first nucleic acid molecule comprising SEQ ID NO:91; and a second nucleotide sequence comprising SEQ ID NO:92.

37. A method of inducing an immune response in a subject, the method comprising administering the composition of any one of claims 1-28, the antibody of any one of claims 29-30, the nucleic acid molecule of any one of claims 31-33, or the composition of any one of claims 34-36 to the subject.

38. The method of claim 37, wherein the subject is currently infected with Leptospira sp and the composition induces an immune response against Leptospira sp.

39. A method of treating or preventing a disease or disorder in a subject, comprising administering the composition of any one of claims 1-28, the antibody of any one of claims 29-30, the nucleic acid molecule of any one of claims 31-33, or the composition of any one of claims 34-36 to the subject.

40. The method of claim 39, wherein the disease or disorder is at least one selected from the group consisting of cancer, a bacterial infection, a viral infection, and a parasitic infection.


SEQUENCE LISTING (99 sequences)

SEQ ID NO: 1 - DNA, 2634 bp, synthetic construct
  source 1..2634: mol_type = other DNA
ttcgaatacggcgtgaatcatacccatattcatgccctgagtaaaattgaatatagtgttattcagaagccgaccgatccgccgaaagataaaccgattaaggttattgttagcgatggtggcaaattttgttatggtccgaattttagcggcggcgaaagctatattattattgaacagtgttggcagatgcatgtgatgaatgcacgctatgatgtgtttcagcgtattagttataacattaacaacacctggctgtgtattaccgcaccggaaaaagtgattaaggcagaaaagaattgggattatgtgcatctgcgtccgtgcaccattaatgatccgctgcagcgctggattattaagaataatagtttttggaccgccaatggtttttatcgtctgaaagattataactggtacggctatattagccgtaatagtggtgaccgttataatcataccctggatagcagcatgaatgattgggttaataccattgccaccccgggtaatattagcattcagaccagtattgcctggaatctgcagaccaccgaaggtcaggaacgttattttattcgctggggtggcagcgataaaaataccaccccgctgtattataatccggaaaatggccatctggcccagtatgatccgattagcggtagcctgtattgcatgtatagtcaggtggataattatcagtggaattgggttaaatggaaatggtgtagcgatctgctggaaagcaaaagtaaaggcaatccgaccttttggaatgtgtttttcgaaaccgatcagggtggcatgattaccgattataaaggtaatgccctgcgcgttacccgctatggtagcaattggggtagcgcatataccgccaaaccgagttatctggaaaaagataccaccaatagcccgaccagcctgtttgtggtgaataaggatctgctggattggacccgttataccgccagtaatctgggtaaaaccggtcagtattgcccggccggcaaacgcgaaaatattgtgcatcgccgcgttaaacgcgaactgccgccggattttcagctgaccgaagcctggattcgccgcctgtatgaaattgccaccagcgttagcgcagaaagcgaaacccgtgtgagcggtatttgtggcccgtgtgcactgcatagttttcagatgctggcagaactgctggaatatcatagccgcgaaccgctgcagagtggtggctatttctttgataccgcaccgaataccgatccgtttattagttttggccagcgttatccgcatctggaacgtctgctggaagatattccgaaaaaatatgcaccgtatccgcattatagcacccagagctttctgagctttgccagtattgatagcatgctgccgcagtatttttggagtgcaagtaccgaattcactaatcgtgatgaaattctgagccatattagtagtctgattaatagtccggcaggcagcatttggctgggtgtgatggaacagcagcatccggatggtaccattaccggtcatgcagcaccgattctgcgtattagtcagggtctggttgtgattccgaccaatgttcatctgtggaccctggaagaatttcgccgctttctgattccgaccaccgaactgagccagattgtggccaatctggaaggtagcaataccctgattcgttttaccaccattcagagtctgggcatgctgaccaccaatatgtttgatagcatggttagcaatcgcaattgtaccggtgaaggtgaagatcgtcgcggtagtggcgaatatccgaccagtaccagtgttaatcagtgcccgagtggtcgttgtgccctgccgtttggcggtggtggcagcggtggtggtggtagtggcggcggtggcagcgacgatgatgataaaatggttagtaaaggtgaagaagataacatggcaattattaaggaattcatgcgctttaaagtgcatatggaaggcagcgtgaatggtcatgaatttgaaattgaaggtgaaggcgaaggtcgtccgtatgaaggtacccagaccgcaaaactgaaagtgaccaaaggtggcccgctgccgtttgcatgggatattctgagcccgcagtttatgtatggcagtaaagcatatgttaagcatccggccgatattccggattatctgaaactgagttttccggaaggctttaaatgggaacgcgttatgaattttgaagatggcggtgttgtgaccgtgacccaggatagcagtctgcaggatggtgaattcatctataaagtgaaactgcgtggcaccaattttccgagcgatggcccggttatgcagaaaaagactatgggttgggaagccagtagtgaacgcatgtatccggaagatggtgcactgaaaggtgaaattaagcagcgcctgaaactgaaagatggtggtcattatgatgcagaagtgaaaaccacctataaagcaaaaaagccggttcagctgccgggcgcctataatgtgaatattaagctggatattaccagtcataacgaagattataccattgttgaacagtatgaacgcgccgaaggccgccatagtaccggcggtatggatgaactgtataaataa

SEQ ID NO: 2 - protein, 877 aa, synthetic construct
FEYGVNHTHIHALSKIEYSVIQKPTDPPKDKPIKVIVSDGGKFCYGPNFSGGESYIIIEQCWQMHVMNARYDVFQRISYNINNTWLCITAPEKVIKAEKNWDYVHLRPCTINDPLQRWIIKNNSFWTANGFYRLKDYNWYGYISRNSGDRYNHTLDSSMNDWVNTIATPGNISIQTSIAWNLQTTEGQERYFIRWGGSDKNTTPLYYNPENGHLAQYDPISGSLYCMYSQVDNYQWNWVKWKWCSDLLESKSKGNPTFWNVFFETDQGGMITDYKGNALRVTRYGSNWGSAYTAKPSYLEKDTTNSPTSLFVVNKDLLDWTRYTASNLGKTGQYCPAGKRENIVHRRVKRELPPDFQLTEAWIRRLYEIATSVSAESETRVSGICGPCALHSFQMLAELLEYHSREPLQSGGYFFDTAPNTDPFISFGQRYPHLERLLEDIPKKYAPYPHYSTQSFLSFASIDSMLPQYFWSASTEFTNRDEILSHISSLINSPAGSIWLGVMEQQHPDGTITGHAAPILRISQGLVVIPTNVHLWTLEEFRRFLIPTTELSQIVANLEGSNTLIRFTTIQSLGMLTTNMFDSMVSNRNCTGEGEDRRGSGEYPTSTSVNQCPSGRCALPFGGGGSGGGGSGGGGSDDDDKMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 3 - DNA, 2589 bp, synthetic construct
  source 1..2589: mol_type = other DNA
agcagtaaaatcgaatacagcgtgattcagaaaccgaccgatccgccgaaagataaaccgattaaggttattgttagcgatggcggtaaattttgttatggtccgaattttagcggtggtgaaagctatattattattgaacagtgttggcagatgcatgttatgaatgcccgctatgatgtttttcagcgtattagctataacattaacaacacctggctgtgtattaccgcaccggaaaaagttattaaggccgaagaaacctgggattatgttcatctgcgtccgtgcaccattaatgatccgctgcagcgttggattattaagaataatagtttctggaccgccaatggcttttatcgcctgaaagattataattggtacggctatattagccgtaatagtggtgaccgctataatcataccctggaccctagtatgaatgattgggttaataccattgccaccccgggcaatattagcattcagaccagtattgcctggaatctgcagaccaccgaaggtcaggaacgttattttattcgttggggcagtagtaataagaataccaccccgctgtattataatccggaaaatggccatctggcacagtatgatccgattagtggtagtctgtattgcatgtatagtcaggtggataattatcagtggaattgggtgaaatggaaatggtgcagcgatagcctggaaagcaaaagcaaaggtaatccgaccttttggaatgttttctttgaaaccgatcagggcggtatgattaccgattataaaggcaatgccctgcgtgtgacccgttatggtagtaattggggcgtggcatataccgccaaaccggattttgttaaaaccgataccaaaaatagcccgaccagcctgtttgtggttgataaaagtctgctggattggacccgctataccagcagtaatctgggtaaaaccgaacagtattgcccggccggtaataaggaaagtgtggtgcataaaaaagccaaacgcaccctgccgccggattttcagctgaccgaagcctggattcgccgtctgtatgaaattgcccgcaccgatccgagcagtcgcaccagtcgcggtgtttgcggtgtttgtatgctgcaggccctgcagatgattgcagaactgcaggaatatcatagtcagggcccgctgcagagcggtggttatttctttaataccgcaccgaataccaatccgtttattagttttggtcagcgttatccgcatctggatcgcctgctggttgatatctatcgcgtgtttgatcatttctttgataccagccataccctgggttatctgagtgccatgaatctgctgccgcagtatgaatggggtcgtacccgcgaattttctaccatgagtgaaattcgcagccatattcgtagcctgattaccagcccgccgggtaatatttggctggtgctgatgaccatgatctatccggatggcacccgcggtggtcatgcagttccgattctgcgtaccccgcagggcctggtggttattgaaaccaccatggcaaccgcaacctttgaagaatatcgtgccgcactgcgtccgaccaccgattttgaacagattattcgtaatctgcgtggtccgaataatattctgattggtctgagcaccctgcagctgggtcgtttttatcataatccgctggatagcatgattagtaatcgcaattgtaccggtgaaggtagcgatcgccgtggtaccggcggctatccggcaagcaccagcgtgaatcagtgcagtagtaaaagcagccgttgcagcctgcagggcggtggtggtagtggtggcggcggtagcggtggtggcggttcagatgatgatgataaaatggtgagtaaaggtgaagaagataatatggccattattaaggaattcatgcgctttaaagtgcatatggaaggtagcgttaatggtcatgaatttgaaattgaaggtgaaggtgaaggccgtccgtatgaaggcacccagaccgccaaactgaaagttaccaaaggtggcccgctgccgtttgcctgggatattctgagtccgcagtttatgtatggtagtaaagcctatgttaagcatccggcagatattccggattatctgaaactgagctttccggaaggctttaaatgggaacgtgttatgaattttgaggatggtggcgttgttaccgttacccaggatagcagcctgcaggatggcgaattcatctataaagtgaaactgcgcggtaccaattttccgagtgatggtccggttatgcagaaaaagactatgggctgggaagcaagtagcgaacgtatgtatccggaagatggtgccctgaaaggcgaaattaagcagcgtctgaaactgaaagatggcggccattatgatgccgaagttaaaaccacctataaagcaaaaaagccggttcagctgccgggtgcatataatgtgaatattaagctggatatcaccagccataatgaagattataccattgttgaacagtacgaacgcgccgaaggccgtcatagcaccggtggtatggatgaactgtataaataa

SEQ ID NO: 4 - protein, 862 aa, synthetic construct
SSKIEYSVIQKPTDPPKDKPIKVIVSDGGKFCYGPNFSGGESYIIIEQCWQMHVMNARYDVFQRISYNINNTWLCITAPEKVIKAEETWDYVHLRPCTINDPLQRWIIKNNSFWTANGFYRLKDYNWYGYISRNSGDRYNHTLDPSMNDWVNTIATPGNISIQTSIAWNLQTTEGQERYFIRWGSSNKNTTPLYYNPENGHLAQYDPISGSLYCMYSQVDNYQWNWVKWKWCSDSLESKSKGNPTFWNVFFETDQGGMITDYKGNALRVTRYGSNWGVAYTAKPDFVKTDTKNSPTSLFVVDKSLLDWTRYTSSNLGKTEQYCPAGNKESVVHKKAKRTLPPDFQLTEAWIRRLYEIARTDPSSRTSRGVCGVCMLQALQMIAELQEYHSQGPLQSGGYFFNTAPNTNPFISFGQRYPHLDRLLVDIYRVFDHFFDTSHTLGYLSAMNLLPQYEWGRTREFSTMSEIRSHIRSLITSPPGNIWLVLMTMIYPDGTRGGHAVPILRTPQGLVVIETTMATATFEEYRAALRPTTDFEQIIRNLRGPNNILIGLSTLQLGRFYHNPLDSMISNRNCTGEGSDRRGTGGYPASTSVNQCSSKSSRCSLQGGGGSGGGGSGGGGSDDDDKMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 5 - DNA, 1173 bp, synthetic construct
  source 1..1173: mol_type = other DNA
caaaagcctactgacccacccaaggataaaccaattaaggtgattgtctcagacgggggtaaattttgctacgggccgaacttttctggcggggagagttacatcattatcgaacagtgttggcaaatgcacgttatgaacgctcgttatgatgtattccaacgcatctcttacaatatcaataacacatggctgtgtatcacggcccccgagaaagtaatcaaggccgaaaaaaactgggactatgtccatctgcgcccatgtacgatcaacgacccactgcagcgttggatcatcaaaaataatagtttctggacagctaatggattctaccgtttaaaagattacaattggtatggctatatcagccgcaattccggtgaccgttataaccatactctggatgggggtggaggcagcggtgggggcggttcagggggaggaggctcagatgacgacgacaaaatggtatcgaaaggtgaggaagataatatggccatcattaaggaatttatgcgcttcaaggttcacatggaaggatcggtaaatggtcacgaatttgagatcgaaggcgaaggtgagggacgcccatacgaggggacccaaactgctaaacttaaggtaacaaagggtggtccgttgccgttcgcctgggacattctgtccccgcaatttatgtacggttcaaaggcttatgtaaaacacccagcggacattccggactacctgaaactgtcttttccagaggggttcaaatgggaacgtgtgatgaacttcgaagatggcggtgttgtaaccgttacacaggattcttcattacaagacggagagttcatctataaggtgaagttgcgtggaaccaactttccttcagatggtcccgtaatgcagaagaagacaatgggctgggaagcgtcatctgaacgtatgtatccagaagatggagcgctgaagggagaaattaaacaacgtctgaagcttaaggacgggggtcattatgacgcggaggtcaagacgacgtacaaggcaaagaaacccgtccagttacccggtgcgtataacgtcaacattaagctggacattacgagtcacaatgaagattacaccatcgttgaacagtatgaacgcgcagagggacgccactccaccggaggtatggacgagctgtacaaa

SEQ ID NO: 6 - protein, 391 aa, synthetic construct
QKPTDPPKDKPIKVIVSDGGKFCYGPNFSGGESYIIIEQCWQMHVMNARYDVFQRISYNINNTWLCITAPEKVIKAEKNWDYVHLRPCTINDPLQRWIIKNNSFWTANGFYRLKDYNWYGYISRNSGDRYNHTLDGGGGSGGGGSGGGGSDDDDKMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 7 - DNA, 2613 bp, synthetic construct
  source 1..2613: mol_type = other DNA
agcagcagtaaagccaattatagtattgcacagaaaccgaccgatccgccgaaagataaaccgattaatattgttacccacgatggcaaaacctattgttatagtccggtgtttagtaaaggtgaaggctatgtgtggattgaaaaatgtggcgataataccgcaaaagcccgctatgatgtgtttcagcgcattagttataatattaacaacacctggctgtgtattaccgcaccggaaccggtggtgaaaggtaatgcacgctggggttatgtgaatctgcgtccgtgtaccattaatgatccgctgcagcgttggattgttaaagaaaatagtttttggaccgccgatggcaaatatcgtctgaaagataccaattggtatggttatattagcaaaaccagcggtgacaattataatcataccctgaatagcagtatggataattgggttaaaaccgttgcaaccccgggcaatattagtattcgtaccagcattagctggaatagtggctggggtgacggtatttgggatattaatatggcaccgagcgcctattttattcatagcaaaggcagtagcaaagaagatattattccgctgtattataacccggaaagtggtcatattgcacagtatgatccgagtagtggtctgctgagctgtatgtatagtaaaatgaccgataaatacgactggaattgggttcagtggggcaaatgcagcgatgccccgattaagaaagaaaatccggccttttggaatgtgtattttgtggcaaatgccggcggtatgattaccgattataaaggcaatattctgcgtgttaccaaagaaggcccgaattggggtgtggcatataccgcaaaaccgagctatctggaaaaagataccacccatagcccgaccagcgtgtttaccgtggatgtggatctgctgaaatggattcgttataccaccagtaatctgggtaaaaccgatcagtattgcccggcaggtaaaaaagaaagtcgcatctatcagcgtgtgaaacgtaatctgccgagcgattttcagctgagtgtggcatgggttcagcgtctgtatgatattgcacgcagcgccacctttgaaagtgccaatccgggcgccattccgcagcgtcatggtgcctgtggtgtttgcctgctgcatagttttcagatgattgccgaactgatggaatatcatagccgcgaaccgctgaccagtggtggttatttctttaataccgcaagtaatcgtgatccgtttctgagctttagccagcgctatccggaactggatcgtctggttaccaatgttccggttgattatgcaaatcgtggtcgcgtgctggcctttgcaagtgccatgattatgctgccgcagtatgaatgggaaagcagcagcccgctgaccacccgcagcgatattcagagtcatattcgcagtctgattaatagcccgccgggtagcatttggctgggcctgctgcgccgtcagcgtgcaaatggcagtattagtggccatgcagtgccgattctgcgtaccagcgaaggcctggtggttattccgaccaatatgccgaccgcaagtctgaatacctatattcagagcctggcaccgaccatggaccctaatgaagtgattaatcgtctggaaaatggccgtaccctgaccaccctgaccacaattcgcccggtgggcacctatgaaaccccgtttagtctgaccgttagcagccgtgattgcaccggtgacggtgacgatcgtcgtggtagcggtcgctatccgattagcagcctgattaatcagtgcagtggtggtcgctgtattctgcagggcggcggcggtagcggcggtggtggtagcggtggtggcggtagtgatgatgatgataaaatggttagtaagggtgaagaagataatatggccattattaaggaattcatgcgctttaaagtgcatatggaaggcagcgtgaatggtcatgaatttgaaattgaaggcgaaggtgaaggtcgtccgtatgaaggcacccagaccgcaaaactgaaagtgaccaaaggcggcccgctgccgtttgcatgggatattctgagcccgcagtttatgtatggcagcaaagcatatgtgaaacatccggccgatattccggattatctgaaactgagctttccggaaggttttaaatgggaacgcgtgatgaattttgaagatggcggtgttgtgaccgttacccaggatagtagcctgcaggatggtgaattcatctataaagtgaaactgcgcggtaccaattttccgagcgatggcccggttatgcagaaaaagactatgggctgggaagcaagcagcgaacgcatgtatccggaagatggcgcactgaaaggcgaaattaagcagcgtctgaaactgaaagatggcggccattatgatgcagaagttaaaaccacctataaagcaaaaaagccggttcagctgccgggtgcatataatgtgaatattaagctggatatcaccagtcataatgaagattataccattgttgagcagtatgaacgtgccgaaggtcgccatagtaccggtggtatggatgaactgtataaataa

SEQ ID NO: 8 - protein, 870 aa, synthetic construct
SSSKANYSIAQKPTDPPKDKPINIVTHDGKTYCYSPVFSKGEGYVWIEKCGDNTAKARYDVFQRISYNINNTWLCITAPEPVVKGNARWGYVNLRPCTINDPLQRWIVKENSFWTADGKYRLKDTNWYGYISKTSGDNYNHTLNSSMDNWVKTVATPGNISIRTSISWNSGWGDGIWDINMAPSAYFIHSKGSSKEDIIPLYYNPESGHIAQYDPSSGLLSCMYSKMTDKYDWNWVQWGKCSDAPIKKENPAFWNVYFVANAGGMITDYKGNILRVTKEGPNWGVAYTAKPSYLEKDTTHSPTSVFTVDVDLLKWIRYTTSNLGKTDQYCPAGKKESRIYQRVKRNLPSDFQLSVAWVQRLYDIARSATFESANPGAIPQRHGACGVCLLHSFQMIAELMEYHSREPLTSGGYFFNTASNRDPFLSFSQRYPELDRLVTNVPVDYANRGRVLAFASAMIMLPQYEWESSSPLTTRSDIQSHIRSLINSPPGSIWLGLLRRQRANGSISGHAVPILRTSEGLVVIPTNMPTASLNTYIQSLAPTMDPNEVINRLENGRTLTTLTTIRPVGTYETPFSLTVSSRDCTGDGDDRRGSGRYPISSLINQCSGGRCILQGGGGSGGGGSGGGGSDDDDKMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 9 - DNA, 1794 bp, synthetic construct
  source 1..1794: mol_type = other DNA
agcagtaaaagcgattatagtatcgcacagaaaccggcagatcagccgaaagataaaagtattcaggtggttatgcatggtggcagcaattattgctatagtccggtgtttaccaaaggtgaaggttatatttggattgattactgcagcgataataccgccaaagcacgttatgatgtgtttcagcgcattagctataatattaataacacctggctgtgcattaccgcaccggaaaccgttgttaaaggcgaagaaacctggaattatgtgaatctgcgtccgtgtaccattaatgatccgctgcagcgctggattgttaaagataatagtttttggaccgcaaatggccgttatcgcctgaaagataccaattggtatggctatattagtcgcaatagcggcgatcgttatgatcataccctgaatagcagtatggatgattggattaagaccgtggcaattccgggcaatattagtattcagaccagcattgcctgggatctgcagaccaccgaaggcaatgaacgttattttattcgttggggcagtagtaataagaataccaccccgctgtattataatccggaaagtggtcatattgcacagtatgatccgagtagcggcctgctgaattgcatgtatagtaaaatgaccgataagtatgactggaattgggtgaaatggggcaaatgcagcgatgcaccgattaagaaagataatccggcattttggaatgttttctttgaaaccgataaggaaggtgcaattaccgattataaaggtaatgtgctgcgcgttacccgttatggcctgaattggggcgttgcatataccgtgaaaccgagttatctggaaaaagataccacccatagcccgaccagtctgtttgtgattgataaagatctgctggattggacccgttatacctatagtaatctgggcaaaaccgatcagtattgtccggcaggcaataaggaaagcctggttcgcaaacgcgttaaacgtaatctgaatctgccgagcgattttcagctgacccgcgaatggattcagcgcctgtatgaaattgcccgcagcagtattagccgtgcaattccgtgtcgtggcgtgtgtggcgtttgtatgctgcatagttatcagatgattgcagaactgctggaatatcatagtcgtggtccgctgaccggcggtggttatttctttgataccgccccgaatcgtgatccgtttattagctttaatcagcgttatccgcagctgaatgccctgctgaccaatgtgccgagttatgcaaatcgcccgggctttggcagtaccctggttatgctgccgcagtatgaatggaccagtagcgataccattaccacccgtagtggtcgcctgctgcatgcccgtagcctgattaatagcccgccgggcagtatttggctgggcctgctgcgcggccgcgatgcagatggtagcacctggggtcatgcagtgccgattctgcgcaccagccagggcattgtggttattccgaccaatagtccgaccatgagcctgaatacctatattcgcagcctggcaccgaccatggatccgaatgaagtgattaatcgtctggaaaatggcagtaccttaaccgaactgaccaccattcagccggttcgcatctatgatattccgtttagcctgaccgtgagtacccgtgattgcaccggtgacggcgatggtcgtcgtggtagtggccgttatccgaccagtagtctgattaatcagtgtagtggtggtcgctgtattctgcagtaa

SEQ ID NO: 10 - protein, 597 aa, synthetic construct
SSKSDYSIAQKPADQPKDKSIQVVMHGGSNYCYSPVFTKGEGYIWIDYCSDNTAKARYDVFQRISYNINNTWLCITAPETVVKGEETWNYVNLRPCTINDPLQRWIVKDNSFWTANGRYRLKDTNWYGYISRNSGDRYDHTLNSSMDDWIKTVAIPGNISIQTSIAWDLQTTEGNERYFIRWGSSNKNTTPLYYNPESGHIAQYDPSSGLLNCMYSKMTDKYDWNWVKWGKCSDAPIKKDNPAFWNVFFETDKEGAITDYKGNVLRVTRYGLNWGVAYTVKPSYLEKDTTHSPTSLFVIDKDLLDWTRYTYSNLGKTDQYCPAGNKESLVRKRVKRNLNLPSDFQLTREWIQRLYEIARSSISRAIPCRGVCGVCMLHSYQMIAELLEYHSRGPLTGGGYFFDTAPNRDPFISFNQRYPQLNALLTNVPSYANRPGFGSTLVMLPQYEWTSSDTITTRSGRLLHARSLINSPPGSIWLGLLRGRDADGSTWGHAVPILRTSQGIVVIPTNSPTMSLNTYIRSLAPTMDPNEVINRLENGSTLTELTTIQPVRIYDIPFSLTVSTRDCTGDGDGRRGSGRYPTSSLINQCSGGRCILQ

SEQ ID NO: 11 - DNA, 876 bp, synthetic construct
  source 1..876: mol_type = other DNA
gttaacccggatcagtattgtccggcaagtaaaaaagaaaatcataatatccgcatcaagcgcaccctgccgccggattttcagctgaccgaagaatggctgcgtcgcctgtatgatattgcaaccagtgccagtctgaccgaaggtcagattcatggcatttgtggcgtttgtctgctgcagacctttcagatgctggcagaactgcaggaatatcatagtcatggcccgctgcagggtggcggctatttctttaataccgcaccggataccgatccgtttgatagttttcgtcagcgctatccggaactggataccatgctgaccgatgcagccaccgcatatggtccggcatataataccacccgtctgctgaccctggtgagcgccatgaccatgatgccgcagtatgaatggaccccgagccgtgaattcactacccgtagcgatatgcatagccatattcgcagtctgattgatagcccgccgggtagtatttggctgggtctgatgcagcgtcgcgaaagtgatgaaaccctgcgctggcatgccctgccgattctgcgcaccagtcagggtctgattgttattcagacccgtgttagcaccatgagctttgaactgtatcgcctgtatctgaccccgagcaccagtattgtgcagattattaatgattacctggaagaagcagatcgcaccctgaccgttctggttaccattcagctggaacaggcatatcagaatctgtttgattttatggttagcaatatgaactgcaccggcgaaggtgaaaatcgccgtggcagtggcggctatccgaccagcgcaaccgtgaatcagtgtagcggtggtcgctgcgcactgccgaattggtaa

SEQ ID NO: 12 - protein, 291 aa, synthetic construct
VNPDQYCPASKKENHNIRIKRTLPPDFQLTEEWLRRLYDIATSASLTEGQIHGICGVCLLQTFQMLAELQEYHSHGPLQGGGYFFNTAPDTDPFDSFRQRYPELDTMLTDAATAYGPAYNTTRLLTLVSAMTMMPQYEWTPSREFTTRSDMHSHIRSLIDSPPGSIWLGLMQRRESDETLRWHALPILRTSQGLIVIQTRVSTMSFELYRLYLTPSTSIVQIINDYLEEADRTLTVLVTIQLEQAYQNLFDFMVSNMNCTGEGENRRGSGGYPTSATVNQCSGGRCALPNW

SEQ ID NO: 13 - protein, 573 aa, Leptospira interrogans
MHGGSNYCYSPVFTKGEGYIWIDYCRDNTAKARYDVFQRISYNINNTWLCITAPETVVKGEETWNYVNLRPCTINDPLQRWIVKDNSFWTANGRYRLKDTNWYGYISRNSGDRYDHTLDSSMDDWIETVAIPGNISIQTSIAWDLQTTEGNERYFIRWGSSNKNTTPLYYNPESGHIAQYDPSSGLLNCMYSKMTDKYDWNWVKWGKCSDAPIKKDNPAFWNVFFETDKEGAITDYKGNVLRVTRYGLNWGVAYTVKPSYLEKDTTHSPTSLFVIDKDLLDWTRYTYSNLGKTDQYCPAGNKESLGRKRVKRNLNLPSDFQLTREWIQRFYEIARSNISGTIPRRGVCGVCMLHSYQMIAELLEYHSRGPLTSGGYFFDTAPNRDPFISFNQRYPQLNTLLTDVPNYANRLGFGSTLVMLPQYEWTSSDTITTRSGRLLHARTLINSPPGSIWLGLLRGRNANGSTWGHAVPILRTSQGIVVIPTNVLTMSLNTYIRSLAPTMDPNEVINRLENGNTLTELTTIQPVRIYDIPFSLTVSTRDCTGDGDGRRGSGRYPTSSLINQCSGGRCILQ

SEQ ID NO: 14 - protein, 627 aa, Leptospira interrogans
MGRWIVLRVSLLVLIGIGFEYGINHTSINASSKSDYSIAQKPADQPKDKSIQVVMHGGSNYCYSPVFTKGEGYIWIDYCSDNTAKARYDVFQRISYNINNTWLCITAPETVVKGEETWNYVNLRPCTINDPLQRWIVKDNSFWTANGRYRLKDTNWYGYISRNSGDRYDHTLNSSMDDWIKTVAIPGNISIQTSIAWDLQTTEGNERYFIRWGSSNKNTTPLYYNPESGHIAQYDPSSGLLNCMYSKMTDKYDWNWVKWGKCSDAPIKKDNPAFWNVFFETDKEGAITDYKGNVLRVTRYGLNWGVAYTVKPSYLEKDTTHSPTSLFVIDKDLLDWTRYTYSNLGKTDQYCPAGNKESLVRKRVKRNLNLPSDFQLTREWIQRLYEIARSSISRAIPCRGVCGVCMLHSYQMIAELLEYHSRGPLTGGGYFFDTAPNRDPFISFNQRYPQLNALLTNVPSYANRPGFGSTLVMLPQYEWTSSDTITTRSGRLLHARSLINSPPGSIWLGLLRGRDADGSTWGHAVPILRTSQGIVVIPTNSPTMSLNTYIRSLAPTMDPNEVINRLENGSTLTELTTIQPVRIYDIPFSLTVSTRDCTGDGDGRRGSGRYPTSSLINQCSGGRCILQ

SEQ ID NO: 15 - DNA, 2603 bp, synthetic construct
  source 1..2603: mol_type = other DNA
ccatgggcgtgaatagcgcactgattgatattgttagccgcagtattccgggcagtattattcagaaaccgaccgataccccgcaggatcgtgcaattaagattgttattcatgatcgcggcaaattttgttatgcaccggtttttagtggcggtgaaagctatattcgcattctgcagtgctgggaacaggatgtgagcgatgcacgctatgatgtgtttcagcgtattagttattacattaagaacacctggctgtgtattaccgccccggaaagcgtggttcagggtcgtgttaattgggattatgttaatctgcgcccgtgcaccattaatgatccgctgcagcgttggattgtgaaagaaaatgcattttggaccgcagatgaacgttatcgcctgaaagatgtgggttggtatgcctatattagtaaaaccagtagcgattattacaaccataccctggatagcagcatgaataattgggtgcgtaccgttagcaccccgggcaatattagtattaagaccagtattgcctggcatctgggtagtgcacgttattttattaatagtcgcggcagcaaaaagaataccaccccgatctattataatccggaaagcggtcatctggcacagtataatccgatgagtggtcgtctgagctgcatgtatagtaaagtgggtagttatcagtggaattggattcagtgggcactgtgtagcgatgcaccgattagtaaagataatccggcatattggaatgtttatctggaaaccgaagaaggcggcatgctgaccgattataaaggtaatattctgcgcgtgacccgctatggtccgaattggggcgtggcctatgccgttaaaccgagttttctggtgaaagatgccacccatagcccgaccagtctgtttgttgtggataaagatctgctggattggattcgctataccaccggtaatctgggcaaaaccgaacagtattgcccggccggctatcgcagcgtttataaacgtgtgaaacgcaccctgccgccggattttcagctgagcgatgattggattcgtcgcctgtatgaaattgcagttagtaccattgataccgcacagggcagcggcatttgtggcgtgtgcatgctgcagagttttcagatgctggccgaactgcaggaatatcattttcagggtccgctgcagagtggtggttatttctttgataccgcacgcgatcgtgatccgtttattagttttcgtcagcgttatagctatctggatcgtctgctggcagatgttccgtatgtgtataccagtgcaggtgacaccaatcgtcagatggttctggcaaccgcacgtaccatgctgccgcagtataattggaccctgagtagtgcattcactacccgcagcgaaattatgagccatattaatagtctgatccattttccgccgggcagcatttggctgggtgttctgggtcgccgccgtccggatggcaccttaagctggcatgcagtgccgattctgcgtaccagtcagggcctggttgtgattcgcacccgtgtgccgagtgccccgtttgatctgtatcgccaggcactgaccccgaccaccgatctgctgcaggttattaataatctggaaaccccgaatcgcattctgaccacctttatgaccattcagctgggtgacgtttatcgtaatacctttgattttgccattagtaatcgtaattgcaccggcgaaggtgacgatcgccgcggcaccggtggttatccggccggtaccagtgttaatcagtgcagttggcgtggtggtcgttgtgccctgcagggtggcggcggtagcggtggtggtggtagcggtggcggtggtagtgatgatgatgataaaatggtgagcaaaggcgaagaagataatatggcaattattaaggagttcatgcgctttaaagttcatatggaaggtagtgtgaatggtcatgaatttgaaattgaaggtgaaggtgaaggccgcccgtatgaaggtacccagaccgccaaactgaaagtgaccaaaggtggtccgctgccgtttgcatgggatattctgagcccgcagtttatgtatggtagcaaagcctatgttaaacatccggcagatattccggattatctgaaactgagttttccggaaggctttaaatgggaacgcgttatgaattttgaagatggcggtgttgttaccgtgacccaggatagcagcctgcaggatggtgaattcatctataaagtgaaactgcgcggcaccaattttccgagcgatggcccggttatgcagaaaaagactatgggctgggaagcaagcagtgaacgcatgtatccggaagatggtgcactgaaaggtgaaattaagcagcgtctgaaactgaaagatggtggtcattatgatgccgaagtgaaaaccacctataaagcaaaaaagccggttcagctgccgggtgcatataatgttaatattaagctggatatcaccagtcataatgaagattataccattgttgagcagtatgaacgcgccgaaggtcgccatagcaccggcggtatggatgaactgtataaactcgag

SEQ ID NO: 16 - DNA, 2603 bp, synthetic construct
  source 1..2603: mol_type = other DNA
ccatgggcgtgaatagtgccctgattgatattgttagtcgtagcattccgggtagcattattcagaaaccgaccgataccccgcaggatcgtgcaattaagattgttattcatgatcgtggcaaattttgctatgccccggtttttagtggcggtgaaagctatattcgtattctgcagtgctgggaacaggatgttagcgatgcacgctatgatgtttttcagcgtattagttattacatcaagaacacctggctgtgcattaccgcaccggaaagtgttgtgcagggccgtgtgaattgggattatgttaatctgcgcccgtgcaccattaatgatccgctgcagcgtgccattgtgaaagaaaatgccttttggaccgcagatgaacgctatcgcctgaaagatgtgggctggtatgccgccattagtaaaaccagtagcgattattataaccataccctggatagcagcatgaataattgggtgcgcaccgtgagcaccccgggcaatattagcattaagaccagtattgcctggcatctgggtagcgcccgttattttattaatagccgcggcagcaaaaagaataccaccccgatctattataatccggaaagtggtcatctggcacagtataatccgatgagtggccgcctgagttgtatgtatagtaaagtgggcagttatcagtggaattggattcagtgggccctgtgcagcgatgcaccgattagtaaagataatccggcatattggaatgtttatctggaaaccgaagaaggcggcatgctgaccgattataaaggcaatattctgcgcgttacccgttatggcccgaattggggtgttgcatatgcagtgaaaccgagctttctggtgaaagatgcaacccatagcccgaccagcctgtttgttgttgataaagatctgctggattggattcgctataccaccggcaatctgggtaaaaccgaacagtattgcccggccggctatcgtagtgtttataaacgcgttaaacgtaccctgccgccggattttcagctgagtgatgattggattcgtcgcctgtatgaaattgcagttagtaccattgataccgcccagggcagcggcatttgcggtgtttgtatgctgcagagctttcagatgctggcagaactgcaggaatatgcatttcagggtccgctgcagagtggcggttatttctttgataccgcacgcgatcgtgatccgtttattagctttcgtcagcgctatagttatctggatcgtctgctggcagatgtgccgtatgtgtataccagcgccggcgataccaatcgccagatggtgctggccaccgcacgcaccatgctgccgcagtataattggaccctgagcagtgcattcactacccgcagcgaaattatgagtgccattaatagcctgattcattttccgccgggtagcatctggctgggtgttctgggtcgtcgtcgtccggatggcaccctgagttgggcagccgtgccgattctgcgcaccagtcagggtctggttgttattcgcacccgcgtgccgagcgccccgtttgatctgtatcgccaggcactgaccccgaccaccgatctgctgcaggttattaataatctggaaaccccgaatcgtattctgaccacctttatgaccattcagctgggcgatgtgtatcgcaatacctttgattttgcaattagtaatcgcaattgcaccggtgaaggtgacgatgcccgtggtaccggtggttatccggccggcaccagcgtgaatcagtgcagttggcgtggcggtcgctgcgccctgcagggtggtggtggtagtggtggcggtggcagtggcggtggtggcagtgatgatgatgataaaatggttagtaagggcgaagaagataatatggcaattattaaggagttcatgcgttttaaagtgcatatggaaggcagcgttaatggtcatgaatttgaaattgaaggtgaaggcgaaggtcgtccgtatgaaggcacccagaccgccaaactgaaagttaccaaaggtggtccgctgccgtttgcctgggatattctgagcccgcagtttatgtatggcagcaaagcatatgttaaacatccggcagatattccggattatctgaaactgagctttccggaaggttttaaatgggaacgtgttatgaattttgaggatggcggcgttgttaccgtgacccaggatagtagtctgcaggatggtgaattcatctataaagttaaactgcgtggcaccaattttccgagcgatggcccggttatgcagaaaaagactatgggctgggaagccagcagcgaacgcatgtatccggaagatggtgcactgaaaggtgaaattaagcagcgtctgaaactgaaagatggtggccattatgatgccgaagttaaaaccacctataaagcaaaaaagccggttcagctgccgggcgcatataatgtgaatattaagctggatatcaccagtcataatgaagattataccattgttgagcagtatgaacgcgccgaaggtcgccatagcaccggcggtatggatgaactgtataaactcgag

SEQ ID NO: 17 - DNA, 2630 bp, synthetic construct
  source 1..2630: mol_type = other DNA
ccatgggcggtagcagcattagcagtagtggcattccgggcagcgtggtgcagaaaccgaccgataaaccgaatgataaaccgattaagattgttgcccataatggtaaaaactattgctatagcccgatgtttggccgtaatgaaagttatattcagctggaacagtgcggtgacaaaaccctgaaagcccgttatgatgtttttcagcgcattagctattatattaacaacacctggctgtgtattaccaccccggaaaaagttattaagggcgaaaccaattgggattatgtgaatctgcgcccgtgcaccattaataatccgggccagcgttggattgttaaaaataatgcattttggaccgccaatggtcgttatcagctgaaagatgtgcattggctggcctatattagcaaaaccagcagtgattattacaatcataccctggataccaccatgaatgattggattaagaccgtggcaaccccgggtaatgttagcgttcagaccagcattgcatgggatagtaattggggcgatggcttttggaatggtaccccgggtcgttattttattcgtagcggcggcagtaatcgtaataccaccccgatctattataatccggaaaatggccatctggcccagtataatccggccaatggcctgctgagctgcatgtatagcaaaattggtaaatataactggaactgggtgcagtgggccagttgcagcgatgcaccggtgagcagtaaagaaaatagcgcctattggaatgtgtattttgaaaccgcagaaggtggtatgattaccgattatcagggcaatattctgcgtgtgaccaaaaccggtccgaattggggcgttgcctataccgttcgtccgagttatctgcaggcagataccacccatagcccgaccagcctgtttattgtggataaagatctgctgaattgggttcgttataccaccggtaatctgggcaataccgaacagtattgcccggcaggcaataagaaaaatcatacctataaacgcgtgaaacgtaatctgccgagcgattttgaactgaccgatgattggattcgtcgcctgtatgatattgcccgcagcgcaacctatgccagtgcccgtccgggctatcgtccgcagcaggcaggcgtgtgtggtacctgtctgctgcatagctttcagatgctggcagaactgcatgaatatcatagccagcgcccgctgcagagcggtggctatttctttgataccgccattgatcgcgatccgtttattagctttagtgttcgttatcgcgaactgtatcgcctgctgaccaatgtgccggccacctatgccaatcgcggtcgtgttctggcctatgccagtaccgcactgatgatgccgcagtatgaatgggaaagcagtccgccgattaccattcgcagcgaaatgctgagtcatattcgcagtatgattaatagcccgaccggcagcatttggctggcaattatgcgtcgccagcgtaccgatggcaccattagtggtcatgcagtgccgattctgcgcaccagccagggtctggttgtgattcgcaccaataccccgaccaccagctttaatgattatcgtagcagtctgattccgcgcatgaatccggatgaaattctggccaatctggaaggcccgggccgcaccatgctgacctttaccaccattcagccggttggcgcctatgaaaatgcctttgatctgatggtgagtaccaaagattgtaccggcgaaggtgacgatcgccgcggcagtggccgttttccgattagcagtctggtgaatcagtgtagtggcggtcgttgcattctgcagggcggtggtggtagtggcggcggtggtagtggtggtggtggcagcgatgatgatgataaaatggtgagcaaaggcgaagaagataatatggcaattattaaggagttcatgcgctttaaagttcatatggaaggcagcgttaatggtcatgaatttgaaattgaaggcgaaggcgaaggtcgtccgtatgaaggtacccagaccgccaaactgaaagttaccaaaggtggcccgctgccgtttgcatgggatattctgagcccgcagtttatgtatggcagcaaagcatatgtgaaacatccggcagatattccggattatctgaaactgagctttccggaaggctttaaatgggaacgcgttatgaattttgaagatggcggtgtggtgaccgtgacccaggatagcagtctgcaggatggtgaattcatctataaagtgaaactgcgtggtaccaattttccgagcgatggtccggttatgcagaaaaagactatgggttgggaagccagcagcgaacgcatgtatccggaagatggcgcactgaaaggcgaaattaagcagcgcctgaaactgaaagatggcggtcattatgatgcagaagtgaaaaccacctataaagcaaaaaagccggtgcagctgccgggtgcatataatgttaatattaagctggatatcaccagtcataatgaagattataccattgttgagcagtatgaacgcgcagaaggccgccatagtaccggcggtatggatgaactgtataaactcgag

SEQ ID NO: 18 - DNA, 2630 bp, synthetic construct
  source 1..2630: mol_type = other DNA
ccatgggcggtagcagcattagcagtagcggtattccgggtagtgttgtgcagaaaccgaccgataaaccgaatgataaaccgattaagattgttgcccataatggtaaaaactattgctatagtccgatgtttggtcgcaatgaaagctatattcagctggaacagtgtggcgataaaaccctgaaagcacgttatgatgtgtttcagcgtattagttattacattaacaacacctggctgtgtattaccaccccggaaaaagtgattaagggtgaaaccaattgggattatgtgaatctgcgtccgtgcaccattaataatccgggccagcgcgccattgtgaaaaataatgcattttggaccgccaatggccgttatcagctgaaagatgttcattggctggcagccattagcaaaaccagcagcgattattataatcataccctggataccaccatgaatgattggattaagaccgttgcaaccccgggcaatgttagtgttcagaccagtattgcatgggatagtaattggggtgacggcttttggaatggtaccccgggtcgctattttattcgtagtggtggcagtaatcgcaataccaccccgatctattataatccggaaaatggccatctggcacagtataatccggcaaatggtctgctgagttgcatgtatagcaaaattggtaaatacaactggaactgggtgcagtgggccagttgcagtgatgcaccggttagcagtaaagaaaatagtgcctattggaatgtttactttgaaaccgcagaaggcggtatgattaccgattatcagggtaatattctgcgtgttaccaaaaccggcccgaattggggcgttgcatataccgtgcgtccgagctatctgcaggccgataccacccatagcccgaccagtctgtttattgtggataaagatctgctgaattgggttcgttataccaccggcaatctgggcaataccgaacagtattgcccggcaggtaataagaaaaatcatacctataagcgtgtgaaacgtaatctgccgagcgattttgaactgaccgatgattggattcgtcgcctgtatgatattgcacgcagcgcaacctatgccagcgcccgccctggttatcgtccgcagcaggccggtgtttgcggcacctgcctgctgcatagctttcagatgctggccgaactgcatgaatatgccagccagcgtccgctgcagagtggcggttatttctttgataccgcaattgatcgtgatccgtttattagttttagtgtgcgttatcgcgaactgtatcgtctgctgaccaatgtgccggcaacctatgcaaatcgcggtcgcgttctggcctatgcaagtaccgccctgatgatgccgcagtatgaatgggaaagcagtccgccgattaccattcgcagtgaaatgctgagtgcaattcgcagcatgattaatagtccgaccggcagtatttggctggcaattatgcgccgtcagcgtaccgatggtaccattagcggcgccgccgttccgattctgcgtaccagtcagggtctggttgttattcgcaccaataccccgaccaccagttttaatgattatcgtagtagtctgatcccgcgcatgaatccggatgaaattctggcaaatctggaaggcccgggtcgtaccatgctgacctttaccaccattcagccggtgggtgcctatgaaaatgcctttgatctgatggtgagtaccaaagattgcaccggtgaaggcgatgatgcccgcggtagcggtcgttttccgattagcagtctggtgaatcagtgtagcggtggccgctgcattctgcagggtggtggcggcagcggtggcggtggttcaggtggtggcggtagcgatgatgatgataaaatggttagtaaaggcgaagaagataatatggcaattattaaggagttcatgcgctttaaagttcatatggaaggtagcgttaatggccatgaatttgaaattgaaggtgaaggcgaaggccgtccgtatgaaggcacccagaccgccaaactgaaagtgaccaaaggtggtccgctgccgtttgcctgggatattctgagcccgcagtttatgtatggtagtaaagcctatgttaagcatccggcagatattccggattatctgaaactgagttttccggaaggttttaaatgggaacgtgtgatgaattttgaagatggtggtgtggtgaccgtgacccaggatagtagtctgcaggatggcgaattcatctataaagtgaaactgcgtggtaccaattttccgagtgatggtccggttatgcagaaaaagactatgggctgggaagcaagcagcgaacgtatgtatccggaagatggcgcactgaaaggtgaaattaagcagcgtctgaaactgaaagatggtggtcattatgatgcagaagtgaaaaccacctataaagcaaaaaagccggttcagctgccgggcgcctataatgttaatattaagctggatatcaccagtcataatgaagattataccattgttgagcagtatgaacgtgcagaaggtcgtcatagtaccggcggtatggatgaactgtataaactcgag

SEQ ID NO: 19 - DNA, 24 bp, synthetic construct
  source 1..24: mol_type = other DNA
tctgtgatcaactattacggatac

SEQ ID NO: 20 - DNA, 23 bp, synthetic construct
  source 1..23: mol_type = other DNA
atccaagtatcaaaccaatgtgg

SEQ ID NO: 21 - protein, 8 aa, synthetic construct
GYTFTTYG

SEQ ID NO: 22 - protein, 8 aa, synthetic construct
INTYSGIP

SEQ ID NO: 23 - protein, 10 aa, synthetic construct
SRSTVYALDQ

SEQ ID NO: 24 - protein, 6 aa, synthetic construct
QDISNY

SEQ ID NO: 25 - skipped

SEQ ID NO: 26 - protein, 9 aa, synthetic construct
QQGYTLPPT

SEQ ID NO: 27 - protein, 117 aa, synthetic construct
QIQLVQSGPELKKPGETVKISCKASGYTFTTYGMSWVKQAPGKGLKWMGWINTYSGIPTFADDFKGRLVFSLETSASTAYLQINNLKNEDTATYFCSRSTVYALDQWGQGTSVTVSS

SEQ ID NO: 28 - protein, 107 aa, synthetic construct
DIQMTQTTSSLSASLGDRVTITCRASQDISNYLNWYQEKPDGTIKLLIYSTSRLHSGVPSRFSGSGSGTDYSLTISNLGQEDIATYFCQQGYTLPPTFGGGTKLEIK

SEQ ID NO: 29 - DNA, 24 bp, synthetic construct
  source 1..24: mol_type = other DNA
gggtataccttcacgacctatgga

SEQ ID NO: 30 - DNA, 24 bp, synthetic construct
  source 1..24: mol_type = other DNA
ataaacacctactctggaattcca

SEQ ID NO: 31 - DNA, 30 bp, synthetic construct
  source 1..30: mol_type = other DNA
tcaagatcgacggtttatgctttggaccaa

SEQ ID NO: 32 - DNA, 18 bp, synthetic construct
  source 1..18: mol_type = other DNA
caggacattagtaattat

SEQ ID NO: 33 - skipped

SEQ ID NO: 34 - DNA, 27 bp, synthetic construct
  source 1..27: mol_type = other DNA
caacagggttatacgcttcctcccacg

SEQ ID NO: 35 - DNA, 351 bp, synthetic construct
  source 1..351: mol_type = other DNA
cagatccagttggtacagtctggacctgagctgaagaaacctggagagacagtcaagatctcctgcaaggcttctgggtataccttcacgacctatggaatgagctgggtgaaacaggctccaggaaagggtttaaagtggatgggctggataaacacctactctggaattccaacatttgctgatgacttcaagggacggcttgtcttctctttggaaacctctgccagcactgcctatttgcagatcaacaacctcaaaaatgaggacacggctacatatttctgttcaagatcgacggtttatgctttggaccaatggggtcaaggaacctcagtcaccgtctcctca

SEQ ID NO: 36 - DNA, 321 bp, synthetic construct
  source 1..321: mol_type = other DNA
gatatccagatgacacagactacatcctccctgtctgcctctctgggagacagagtcaccatcacttgcagggcaagtcaggacattagtaattatttaaactggtatcaggagaaaccagatggaactattaaactcctgatctactccacatcaagattacactcaggagtcccatcaaggttcagtggcagtgggtctggaacagattattctctcaccattagcaacctggggcaagaagatattgccacttacttttgccaacagggttatacgcttcctcccacgttcggaggggggaccaagttggaaataaaa

SEQ ID NO: 37 - protein, 11 aa, synthetic construct
QSIVHSNGNTY

SEQ ID NO: 38 - skipped

SEQ ID NO: 39 - protein, 9 aa, synthetic construct
FQGSHVPYT

SEQ ID NO: 40 - DNA, 33 bp, synthetic construct
  source 1..33: mol_type = other DNA
cagagcattgtacatagtaatggaaacacctat

SEQ ID NO: 41 - skipped

SEQ ID NO: 42 - DNA, 27 bp, synthetic construct
  source 1..27: mol_type = other DNA
tttcaaggttcacatgttccgtacacg

SEQ ID NO: 43 - protein, 112 aa, synthetic construct
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIK

SEQ ID NO: 44 - DNA, 336 bp, synthetic construct
  source 1..336: mol_type = other DNA
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtacacgttcggaggggggaccaagctggaaataaaa

SEQ ID NO: 45 - protein, 8 aa, synthetic construct
GYTFTAYG

SEQ ID NO: 46 - protein, 8 aa, synthetic construct
INTYSGVP

SEQ ID NO: 47 - protein, 9 aa, synthetic construct
ARGGELFDY

SEQ ID NO: 48 - protein, 5 aa, synthetic construct
SSINY

SEQ ID NO: 49 - skipped

SEQ ID NO: 50 - protein, 8 aa, synthetic construct
QRSSYPYT

SEQ ID NO: 51 - protein, 116 aa, synthetic construct
QIQLVQSGPELKKPGETVKISCKASGYTFTAYGMSWVMQAPGKGLKWKGWINTYSGVPTYADDFKGRFAFSLETSASTAYLQINLLKNEDTATYFCARGGELFDYWGQGTTLTVSS

SEQ ID NO: 52 - protein, 106 aa, synthetic construct
QIVLTQSPAIMSASPGEKVTMTCSASSSINYMHWYQQKPGTSPKRWIYDTSKLASGVPARFSGSGSGTSYSLTISSMEAEDAATYYCHQRSSYPYTFGGGTKLEIK

SEQ ID NO: 53 - DNA, 24 bp, synthetic construct
  source 1..24: mol_type = other DNA
gggtataccttcacagcctatgga

SEQ ID NO: 54 - DNA, 24 bp, synthetic construct
  source 1..24: mol_type = other DNA
ataaacacctactctggagtgcca

SEQ ID NO: 55 - DNA, 27 bp, synthetic construct
  source 1..27: mol_type = other DNA
gcaagagggggggaactctttgactac

SEQ ID NO: 56 - DNA, 15 bp, synthetic construct
  source 1..15: mol_type = other DNA
tcaagtataaattac

SEQ ID NO: 57 - skipped

SEQ ID NO: 58 - DNA, 27 bp, synthetic construct
  source 1..27: mol_type = other DNA
catcagcggagtagttacccgtacacg

SEQ ID NO: 59 - DNA, 348 bp, synthetic construct
  source 1..348: mol_type = other DNA
cagatccagctggtacagtctggacctgagctgaagaagcctggagagacagtcaagatctcctgcaaggcttctgggtataccttcacagcctatggaatgagctgggtgatgcaggctccaggaaagggtttaaagtggaagggctggataaacacctactctggagtgccaacatatgctgatgacttcaagggacggtttgccttctccttggaaacctctgccagcactgcctatttgcagatcaatctcctcaaaaatgaggacacggctacatatttctgtgcaagagggggggaactctttgactactggggccaaggcaccactctcacagtctcctca

SEQ ID NO: 60 - DNA, 318 bp, synthetic construct
  source 1..318: mol_type = other DNA
caaattgttctcacccagtctccagcaatcatgtctgcatctccaggggagaaggtcaccatgacctgcagtgccagctcaagtataaattacatgcactggtaccagcagaagccaggcacctcccccaaaagatggatttatgacacatccaaactggcttctggagtccctgctcgcttcagtggcagtgggtctgggacctcttattctctcacaatcagcagcatggaggctgaagatgctgccacttattactgccatcagcggagtagttacccgtacacgttcggtggggggaccaagctggaaataaaa

SEQ ID NO: 61 - protein, 8 aa, synthetic construct
GYTFTRYW

SEQ ID NO: 62 - protein, 8 aa, synthetic construct
IDPSGRYG

SEQ ID NO: 63 - protein, 12 aa, synthetic construct
VREQGVLYTTDY

SEQ ID NO: 64 - protein, 13 aa, synthetic construct
QSLLNSGNQKNYL

SEQ ID NO: 65 - skipped

SEQ ID NO: 66 - protein, 8 aa, synthetic construct
QNDYSDPT

SEQ ID NO: 67 - protein, 119 aa, synthetic construct
QVQLQQPGAEFVKTGASVKLSCKASGYTFTRYWMQWVKQRPGQGLEWIGEIDPSGRYGNYNQKFKGKATLTVDPSSRTAYMQLSSLTSEDSAVYYCVREQGVLYTTDYWGQGTSVTVSS

SEQ ID NO: 68 - protein, 112 aa, synthetic construct
DIVMTQSPSSLTVTAGEKVTMSCKSSQSLLNSGNQKNYLTWFQQKPGQPPKLLIYWASARQSGVPDRFTGSGSGTDFTLTISSVQAEDLAVYYCQNDYSDPTFGAGTKLELK

SEQ ID NO: 69 - DNA, 24 bp, synthetic construct
  source 1..24: mol_type = other DNA
ggctacaccttcaccaggtactgg

SEQ ID NO: 70 - DNA, 24 bp, synthetic construct
  source 1..24: mol_type = other DNA
attgatccttctggtagatatggt

SEQ ID NO: 71 - DNA, 36 bp, synthetic construct
  source 1..36: mol_type = other DNA
gtaagagagcagggggtcctctatactacggactac

SEQ ID NO: 72 - DNA, 36 bp, synthetic construct
  source 1..36: mol_type = other DNA
cagagtctgttaaatagtggaaatcaaaagaactac

SEQ ID NO: 73 - skipped

SEQ ID NO: 74 - DNA, 24 bp, synthetic construct
  source 1..24: mol_type = other DNA
cagaatgattatagtgatcccacg

SEQ ID NO: 75 - DNA, 357 bp, synthetic construct
  source 1..357: mol_type = other DNA
caggtccaactgcagcagcctggggctgagtttgtgaagactggggcttcagtgaagctgtcctgcaaggcttctggctacaccttcaccaggtactggatgcagtgggtaaaacagaggcctggacagggccttgagtggatcggagagattgatccttctggtagatatggtaactacaatcaaaagttcaagggcaaggccacattgactgtagacccatcctccagaacagcctacatgcagctcagcagcctgacatctgaggactctgcggtctattactgtgtaagagagcagggggtcctctatactacggactactggggtcaaggaacctcagtcaccgtctcctca

SEQ ID NO: 76 - DNA, 336 bp, synthetic construct
  source 1..336: mol_type = other DNA
gacattgtgatgacacagtctccatcctccctgactgtgacagcaggagagaaggtcactatgagctgcaagtccagtcagagtctgttaaatagtggaaatcaaaagaactacttgacctggttccagcagaaaccaggccagcctcctaaactgttgatctactgggcatccgctaggcaatctggggtccctgatcgcttcacaggcagtggatctggaacagattttactctcaccatcagcagtgtgcaggctgaagacctggcagtttattactgtcagaatgattatagtgatcccacgttcggtgctgggaccaagctggagctgaaa

SEQ ID NO: 77 - protein, 8 aa, synthetic construct
GFIFSSYG

SEQ ID NO: 78 - protein, 8 aa, synthetic construct
ISTGGSYT

SEQ ID NO: 79 - protein, 10 aa, synthetic construct
ARYGPYGMDY

SEQ ID NO: 80 - protein, 11 aa, synthetic construct
QSIVHSNGNTY

SEQ ID NO: 81 - skipped

SEQ ID NO: 82 - protein, 9 aa, synthetic construct
LQGSHVPWT

SEQ ID NO: 83 - protein, 117 aa, synthetic construct
EVQLVESGGDVVKPGGSLKLSCAASGFIFSSYGMSWVRQTPDKRLEWVATISTGGSYTYYPDSVKGRFTISRDNAKDTLYLQMSSLKSEDTAMYYCARYGPYGMDYWGQGTSVTVSS

SEQ ID NO: 84 - protein, 112 aa, synthetic construct
DVLMTQTPVSLPVSLGDQASISCRSSQSIVHSNGNTYLEWYLQKPGQSPKLLIHRVSNRFSGVPDRFSGSGSGTDFTLKISRVEAEDLGVYYCLQGSHVPWTFGGGTKVEIK

SEQ ID NO: 85 - DNA, 24 bp, synthetic construct
  source 1..24: mol_type = other DNA
ggattcattttcagtagctatggc

SEQ ID NO: 86 - DNA, 24 bp, synthetic construct
  source 1..24: mol_type = other DNA
attagtactggtggtagttacacc

SEQ ID NO: 87 - DNA, 30 bp, synthetic construct
  source 1..30: mol_type = other DNA
gcaaggtatgggccctatggtatggactac

SEQ ID NO: 88 - DNA, 33 bp, synthetic construct; identical to SEQ ID NO: 40
  source 1..33: mol_type = other DNA

SEQ ID NO: 89 - skipped

SEQ ID NO: 90 - DNA, 27 bp, synthetic construct
  source 1..27: mol_type = other DNA
cttcaaggttcacacgttccgtggacg

SEQ ID NO: 91 - DNA, 351 bp, synthetic construct
  source 1..351: mol_type = other DNA
gaggtgcagctggtggagtctgggggagacgtagtgaagcctggagggtccctgaaactctcctgtgcagcctctggattcattttcagtagctatggcatgtcttgggttcggcagactccagacaagaggctggagtgggtcgcaaccattagtactggtggtagttacacctactatccagacagtgtgaaggggcgattcaccatttccagagacaatgccaaggataccctgtacctgcaaatgagcagtctgaagtctgaggacacagccatgtattactgtgcaaggtatgggccctatggtatggactactggggtcaaggaacctcagtcaccgtctcctca

SEQ ID NO: 92 - DNA, 336 bp, synthetic construct
  source 1..336: mol_type = other DNA
gatgttttgatgacccaaactccagtctccctgcctgtcagtcttggagatcaagcctccatctcttgcagatctagtcagagcattgtacatagtaatggaaacacctatttagaatggtacctgcagaaaccaggccagtctccaaagctcctgatccacagagtttccaatcgattttctggggtcccagacaggttcagtggcagtggatcagggacagatttcacactcaagatcagcagagtggaggctgaggatctgggagtttattactgccttcaaggttcacacgttccgtggacgttcggtggaggcaccaaggtggaaatcaaa

SEQ ID NO: 93 - protein, 15 aa, synthetic construct
GGGGSGGGGSGGGGS

SEQ ID NO: 94 - protein, 7 aa, synthetic construct
HLERLLE

SEQ ID NO: 95 - protein, 5 aa, synthetic construct
YEIAT

SEQ ID NO: 96 - protein, 7 aa, synthetic construct
MLAELLE

SEQ ID NO: 97 - protein, 6 aa, synthetic construct
PILRIS

SEQ ID NO: 98 - protein, 7 aa, synthetic construct
EEFRRFL

SEQ ID NO: 99 - protein, 6 aa, synthetic construct
RCALPF